SEQUENCE LISTING (125 sequences)

SEQ ID NO: 1 - DNA, 2154 bp, Arabidopsis thaliana
  source 1..2154: mol_type = genomic DNA
atggaccaaattgaagcaatgctatgcggtggtggtgaaaagaccaaggtggccgtaacgacaaaaactcttgcagatcctttgaattggggtctggcagctgaccagatgaaaggtagccatctggatgaagttaagaagatggttgaggaatacagaagaccagtcgtaaatctaggcggcgagacattgacgataggacaggtagctgctatttcgaccgttggcggttcagtgaaggtagaacttgcagaaacaagtagagccggagttaaggcttcatcagattgggtcatggaaagtatgaacaagggcacagattcctatggcgttaccacaggctttggtgctacctctcatagaagaactaaaaatggcactgctttgcaaacagaactgatcagattccttaacgccggtattttcggtaatacaaaggaaacttgccatacattaccccaatcggcaacaagagctgctatgcttgttagggtgaacactttgttgcaaggttactctggaataaggtttgaaattcttgaggccatcacttcactattgaaccacaacatttctccttcgttgcccttaagaggaacaataactgccagcggtgatttggttcccctttcatatatcgcaggcttattaacgggaagacctaattcaaaggccactggtccagacggagaatccttaaccgctaaggaagcatttgagaaagctggtatttcaactggtttctttgatttgcaacccaaggaaggtttagccctggtgaatggcaccgctgtcggcagcggtatggcatccatggtgttgtttgaagctaacgtacaagcagttttggccgaagttttgtccgcaatttttgccgaagtcatgagtggaaaacctgagtttactgatcacttgacccacaggttaaaacatcacccaggacaaattgaagcagcagctatcatggagcacattttggacggctctagctacatgaagttagcccagaaggttcatgaaatggaccctttgcaaaaacccaaacaagatagatatgctttaaggacatccccacaatggcttggccctcaaattgaagtaattagacaagctacaaagtctatagaaagagagatcaactctgttaacgataatccacttattgatgtgtcgaggaataaggcaatacatggaggcaatttccagggtacacccataggagtcagtatggataataccaggcttgccatagccgcaattggcaaattaatgtttgcccaattttctgaattggtcaatgacttctacaataacggtttgccttcgaatctgaccgcatcttctaaccctagtcttgattatggtttcaaaggtgctgagatagcaatggcaagctattgttcagagctgcaatatctagccaacccagtaacctctcatgtacaatcagccgaacaacacaatcaggatgttaattctttgggcctgatttcatcaagaaaaacaagcgaggccgttgatatccttaaattaatgtccacaacatttttagtgggtatatgccaggccgtagatttgagacacttggaagagaatttgagacagacagtgaaaaataccgtatcacaggttgcaaaaaaggttctaactacaggtatcaatggtgaattgcacccatcaagattctgtgaaaaagatttattaaaagttgtagatagagaacaagtatttacttacgttgacgatccatgtagcgctacttatccattgatgcagagattgagacaagttattgtagatcacgctttatccaatggtgaaactgagaaaaatgccgttacttcaatattccaaaagataggtgcctttgaagaagaactgaaggcagttttaccaaaggaagtcgaagctgctagagccgcatacggaaatggtactgcccctataccaaatagaatcaaagagtgtaggtcgtaccctttgtacagattcgttagagaagagttgggaaccaaattactaactggtgaaaaagtcgttagcccaggtgaagaatttgacaaggtattcacagctatgtgcgagggaaagttgatagatccacttatggattgcttgaaagagtggaatggtgcacctattccaatctgctaa

SEQ ID NO: 2 - protein, 717 aa, Arabidopsis thaliana
MDQIEAMLCGGGEKTKVAVTTKTLADPLNWGLAADQMKGSHLDEVKKMVEEYRRPVVNLGGETLTIGQVAAISTVGGSVKVELAETSRAGVKASSDWVMESMNKGTDSYGVTTGFGATSHRRTKNGTALQTELIRFLNAGIFGNTKETCHTLPQSATRAAMLVRVNTLLQGYSGIRFEILEAITSLLNHNISPSLPLRGTITASGDLVPLSYIAGLLTGRPNSKATGPDGESLTAKEAFEKAGISTGFFDLQPKEGLALVNGTAVGSGMASMVLFEANVQAVLAEVLSAIFAEVMSGKPEFTDHLTHRLKHHPGQIEAAAIMEHILDGSSYMKLAQKVHEMDPLQKPKQDRYALRTSPQWLGPQIEVIRQATKSIEREINSVNDNPLIDVSRNKAIHGGNFQGTPIGVSMDNTRLAIAAIGKLMFAQFSELVNDFYNNGLPSNLTASSNPSLDYGFKGAEIAMASYCSELQYLANPVTSHVQSAEQHNQDVNSLGLISSRKTSEAVDILKLMSTTFLVGICQAVDLRHLEENLRQTVKNTVSQVAKKVLTTGINGELHPSRFCEKDLLKVVDREQVFTYVDDPCSATYPLMQRLRQVIVDHALSNGETEKNAVTSIFQKIGAFEEELKAVLPKEVEAARAAYGNGTAPIPNRIKECRSYPLYRFVREELGTKLLTGEKVVSPGEEFDKVFTAMCEGKLIDPLMDCLKEWNGAPIPIC

SEQ ID NO: 3 - DNA, 2112 bp, Zea mays
  source 1..2112: mol_type = genomic DNA
atggcgggcaacggcgccatcgtggagagcgacccgctgaactggggcgcggcggcggcggagctggccgggagccacctggacgaggtgaagcgcatggtggcgcaggcccggcagcccgtggtcaagatcgagggctccaccctccgcgtcggccaggtggccgccgtcgcctccgccaaggacgcgtccggcgtcgccgtcgagctcgacgaggaggcccgcccccgcgtcaaggccagcagcgagtggatcctcgactgcatcgcccacggcggcgacatctacggcgtcaccaccggcttcggcggcacctcccaccgccgcaccaaggacgggcccgcgctccaggtcgagctgctcaggcatctcaacgccggaatcttcggcaccggcagcgacgggcacacgctgccgtcggaggtcacccgcgcggcgatgctggtgcgcatcaacaccctcctccagggctactccggcatccgcttcgagatcctcgaggccatcacgaagctgctcaacaccggtgtcagcccctgcctgccgctccggggcaccatcaccgcgtcgggcgacctggtcccgctctcctacatcgccggcctcatcacgggccgccccaacgcgcaggccgtcaccgtcgacggaaggaaggtggacgccgccgaggcgttcaagatcgccggcatcgagggcggcttcttcaagctcaaccccaaggagggcctcgccatcgtcaacggcacgtccgtgggctccgcgctcgcggccaccgtgatgtacgacgccaacgtcctggccgtcctgtcggaggtcctgtccgccgtcttctgcgaggtcatgaacggcaagcccgagtacacggaccacctgacccacaagctgaagcaccacccggggtccatcgaggccgcggccatcatggagcacatcctggatggcagctccttcatgaagcaggccaagaaggtgaacgagctggacccgctgctgaagcccaagcaggacaggtacgcgctccgcacgtcgccgcagtggctgggcccccagatcgaggtcatccgcgccgccaccaagtccatcgagcgcgaggtcaactccgtgaacgacaacccggtcatcgacgtccaccgcggcaaggcgctgcacggcggcaacttccagggcacccccatcggcgtgtccatggacaacgcccgcctcgccatcgccaacatcggcaagctcatgttcgcgcagttctccgagctcgtcaacgagttctacaacaacgggctcacctccaacctggccggcagccgcaaccccagcctggactacggcttcaagggcaccgagatcgccatggcctcctactgctccgagctccagtacctgggcaaccccatcaccaaccacgtgcagagcgcggacgagcacaaccaggacgtgaactccctgggcctcgtctcggccaggaagaccgccgaggcgatcgacatcctgaagctcatgtcgtccacctacatcgtggcgctgtgccaggccgtggacctgcgccacctcgaggagaacatcaaggcgtcggtgaagaacaccgtgacccaggtggccaagaaggtgctgaccatgaacccctcgggcgagctctccagcgcccgcttcagcgagaaggagctgatcagcgccatcgaccgcgaggccgtgttcacgtacgcggaggacgcggccagcgccagcctgccgctgatgcagaagctgcgcgccgtgctggtggaccacgccctcagcagcggcgagcgcggagcgggagccctccgtgttctccaagatcaccaggttcgaggaggagctccgcgcggtgctgccccaggaggtggaggccgcccgcgtggcgtcgccgagggcaccgcccccgtggcgaaccggatcgcggacagccggtcgttcccgctgtaccgcttcgtgcgcgaggagctcggctgcgtgttcctgaccggcgagaggctcaagtcccccggcgaggagtgcaacaaggtgttcgtcggcatcagccagggcaagctcgtggaccccatgctcgagtgcctcaaggagtgggacggcaagccgctgcccatcaacatcaagtaa

SEQ ID NO: 4 - protein, 703 aa, Zea mays
MAGNGAIVESDPLNWGAAAAELAGSHLDEVKRMVAQARQPVVKIEGSTLRVGQVAAVASAKDASGVAVELDEEARPRVKASSEWILDCIAHGGDIYGVTTGFGGTSHRRTKDGPALQVELLRHLNAGIFGTGSDGHTLPSEVTRAAMLVRINTLLQGYSGIRFEILEAITKLLNTGVSPCLPLRGTITASGDLVPLSYIAGLITGRPNAQAVTVDGRKVDAAEAFKIAGIEGGFFKLNPKEGLAIVNGTSVGSALAATVMYDANVLAVLSEVLSAVFCEVMNGKPEYTDHLTHKLKHHPGSIEAAAIMEHILDGSSFMKQAKKVNELDPLLKPKQDRYALRTSPQWLGPQIEVIRAATKSIEREVNSVNDNPVIDVHRGKALHGGNFQGTPIGVSMDNARLAIANIGKLMFAQFSELVNEFYNNGLTSNLAGSRNPSLDYGFKGTEIAMASYCSELQYLGNPITNHVQSADEHNQDVNSLGLVSARKTAEAIDILKLMSSTYIVALCQAVDLRHLEENIKASVKNTVTQVAKKVLTMNPSGELSSARFSEKELISAIDREAVFTYAEDAASASLPLMQKLRAVLVDHALSSGERGAGALRVLQDHQVRGGAPRGAAPGGGGRPRGVAEGTAPVANRIADSRSFPLYRFVREELGCVFLTGERLKSPGEECNKVFVGISQGKLVDPMLECLKEWDGKPLPINIK

SEQ ID NO: 5 - DNA, 1521 bp, Ammi majus
  source 1..1521: mol_type = genomic DNA
atgatggattttgttttgttagaaaaagctcttcttggtttgttcattgcaactatagtagccatcacaatctctaagctaaggggaaagaaacttaagttgcctccaggcccaatccctgtcccagtgtttggtaattggttacaagttggcgacgacttaaaccagaggaatttggtagagtatgctaaaaagttcggcgacttatttctacttaggatgggtcaaagaaacttggtcgtggtttcatcccctgacttagcaaaagacgtactacatacccagggtgtcgagttcggaagtagaactagaaatgttgtgtttgatattttcacaggcaaaggtcaagatatggtttttaccgtatacagcgagcactggaggaaaatgagaagaataatgactgtcccattctttacaaacaaagtggttcaacagtataggttcggatgggaggacgaagccgctagagtagtcgaggatgttaaggcaaatcctgaagccgctaccaacggtattgtgttgaggaatagattacaacttttgatgtacaacaatatgtatagaataatgtttgacaggagatttgaatctgttgatgatccattattcctaaaacttaaggcattgaatggcgagagatcaaggttagctcaatcctttgaatacaacttcggtgacttcattcctatattgaggccattcttgagaggatatcttaagttgtgtcaggaaatcaaggacaaaaggttaaagctattcaaggactacttcgtcgacgagagaaaaaagttggagagtatcaagagcgtaggtaataactccttaaagtgcgccatagatcatattatcgaggcacaagaaaaaggcgagataaacgaggataacgtgttatacatcgtcgagaatatcaacgtggctgccattgaaactacactttggtctattgaatggggtatagcagaactagtgaataaccctgaaatccagaaaaaattgagacacgaattagacaccgtacttggagctggtgttcaaatttgtgaaccagatgttcaaaaattgccttatctacaggccgtgataaaagagactttaaggtacaggatggcaattccattgttagtcccacatatgaatcttcacgaagccaaattggccggctatgatatccctgcagagagcaaaattttggtaaacgcttggtggttagccaataatccagcacattggaacaaacctgatgagtttagaccagaaagatttttggaggaagaatccaaggtcgaggctaatggaaacgactttaagtacatccctttcggtgttggcagaagatcttgcccaggtataattcttgctttaccaatccttggaatagtaattggtaggttggttcaaaacttcgagttacttccacctccaggccaaagcaaaatagatacagccgaaaaaggtggacagttttcattgcaaatcctaaagcattccactattgtgtgtaaacctagaagttcttaa

SEQ ID NO: 6 - protein, 506 aa, Ammi majus
MMDFVLLEKALLGLFIATIVAITISKLRGKKLKLPPGPIPVPVFGNWLQVGDDLNQRNLVEYAKKFGDLFLLRMGQRNLVVVSSPDLAKDVLHTQGVEFGSRTRNVVFDIFTGKGQDMVFTVYSEHWRKMRRIMTVPFFTNKVVQQYRFGWEDEAARVVEDVKANPEAATNGIVLRNRLQLLMYNNMYRIMFDRRFESVDDPLFLKLKALNGERSRLAQSFEYNFGDFIPILRPFLRGYLKLCQEIKDKRLKLFKDYFVDERKKLESIKSVGNNSLKCAIDHIIEAQEKGEINEDNVLYIVENINVAAIETTLWSIEWGIAELVNNPEIQKKLRHELDTVLGAGVQICEPDVQKLPYLQAVIKETLRYRMAIPLLVPHMNLHEAKLAGYDIPAESKILVNAWWLANNPAHWNKPDEFRPERFLEEESKVEANGNDFKYIPFGVGRRSCPGIILALPILGIVIGRLVQNFELLPPPGQSKIDTAEKGGQFSLQILKHSTIVCKPRSS

SEQ ID NO: 7 - DNA, 1671 bp, Arabidopsis thaliana
  source 1..1671: mol_type = genomic DNA
atgacgacacaagatgtgatagtcaatgatcagaatgatcagaaacagtgtagtaatgacgtcattttccgatcgagattgcctgatatatacatccctaaccacctcccactccacgactacatcttcgaaaatatctcagagttcgccgctaagccatgcttgatcaacggtcccaccggcgaagtatacacctacgccgatgtccacgtaacatctcggaaactcgccgccggtcttcataacctcggcgtgaagcaacacgacgttgtaatgatcctcctcccgaactctcctgaagtagtcctcactttccttgccgcctccttcatcggcgcaatcaccacctccgcgaacccgttcttcactccggcggagatttctaaacaagccaaagcctccgctgcgaaactcatcgtcactcaatcccgttacgtcgataaaatcaagaacctccaaaacgacggcgttttgatcgtcaccaccgactccgacgccatccccgaaaactgcctccgtttctccgagttaactcagtccgaagaaccacgagtggactcaataccggagaagatttcgccagaagacgtcgtggcgcttcctttctcatccggcacgacgggtctccccaaaggagtgatgctaacacacaaaggtctagtcacgagcgtggcgcagcaagtcgacggcgagaatccgaatctttacttcaacagagacgacgtgatcctctgtgtcttgcctatgttccatatatacgctctcaactccatcatgctctgtagtctcagagttggtgccacgatcttgataatgcctaagttcgaaatcactctcttgttagagcagatacaaaggtgtaaagtcacggtggctatggtcgtgccaccgatcgttttagctatcgcgaagtcgccggagacggagaagtatgatctgagctcggttaggatggttaagtctggagcagctcctcttggtaaggagcttgaagatgctattagtgctaagtttcctaacgccaagctaggtcagggctatgggatgacagaagcaggtccggtgctagcaatgtcgttagggtttgctaaagagccgtttccagtgaagtcaggagcatgtggtacggtggtgaggaacgccgagatgaagatacttgatccagacacaggagattctttgcctaggaacaaacccggcgaaatatgcatccgtggcaaccaaatcatgaaaggctatctcaatgaccccttggccacggcatcgacgatcgataaagatggttggcttcacactggagacgtcggatttatcgatgatgacgacgagcttttcattgtggatagattgaaagaactcatcaagtacaaaggatttcaagtggctccagctgagctagagtctctcctcataggtcatccagaaatcaatgatgttgctgtcgtcgccatgaaggaagaagatgctggtgaggttcctgttgcgtttgtggtgagatcgaaagattcaaatatatccgaagatgaaatcaagcaattcgtgtcaaaacaggttgtgttttataagagaatcaacaaagtgttcttcactgactctattcctaaagctccatcagggaagatattgaggaaggatctaagagcaagactagcaaatggattaatgaactag

SEQ ID NO: 8 - protein, 556 aa, Arabidopsis thaliana
MTTQDVIVNDQNDQKQCSNDVIFRSRLPDIYIPNHLPLHDYIFENISEFAAKPCLINGPTGEVYTYADVHVTSRKLAAGLHNLGVKQHDVVMILLPNSPEVVLTFLAASFIGAITTSANPFFTPAEISKQAKASAAKLIVTQSRYVDKIKNLQNDGVLIVTTDSDAIPENCLRFSELTQSEEPRVDSIPEKISPEDVVALPFSSGTTGLPKGVMLTHKGLVTSVAQQVDGENPNLYFNRDDVILCVLPMFHIYALNSIMLCSLRVGATILIMPKFEITLLLEQIQRCKVTVAMVVPPIVLAIAKSPETEKYDLSSVRMVKSGAAPLGKELEDAISAKFPNAKLGQGYGMTEAGPVLAMSLGFAKEPFPVKSGACGTVVRNAEMKILDPDTGDSLPRNKPGEICIRGNQIMKGYLNDPLATASTIDKDGWLHTGDVGFIDDDDELFIVDRLKELIKYKGFQVAPAELESLLIGHPEINDVAVVAMKEEDAGEVPVAFVVRSKDSNISEDEIKQFVSKQVVFYKRINKVFFTDSIPKAPSGKILRKDLRARLANGLMN

SEQ ID NO: 9 - DNA, 1173 bp, Hypericum androsaemum
  source 1..1173: mol_type = genomic DNA
atggttactgttgaagaagttagaaaagctcaaagggcagaaggtccagccacagtgatggctattggaaccgcagttcctccaaattgtgtagatcaggccacttatcctgactactactttagaataacaaactctgagcataaggctgaattgaaagaaaagttccaaaggatgtgcgacaaatcacagatcaagaaaagatacatgtaccttaatgaggaagtcctaaaggaaaacccaaatatgtgtgcatacatggccccttcccttgacgctagacaagatattgtggttgtagaggtcccaaaattgggcaaggaagcagctgttaaagccataaaggaatggggtcaacctaagagcaaaatcacccaccttgtgttttgcactacaagcggagttgacatgccaggcgcagattatcagctaaccaaacttttgggtttaaggccttctgtaaaaagattgatgatgtaccaacaaggttgtttcgctggaggcactgtcttaagactagccaaggatcttgcagagaacaacaaaggtgctagggtgttggttgtatgctcagaaattacagccgtcacctttagaggaccaactgacactcacttagattccctagttggtcaggcattgtttggcgacggtgctgccgcaataatcattggaagtgatcctattccagaggtggaaaagcctctttttgaacttgttagcgctgcccaaactatattgccagattctgagggtgcaatcgacggccacttaagggaagtaggtctaaccttccatcttttgaaagatgtccctggtttaatttcaaagaacgtggaaaaatccctaacagaggcttttaaaccattgggtataagtgactggaatagcttattctggatcgctcacccaggcggccctgccatacttgaccaggttgaagcaaaattgagcttaaagccagaaaaactaagagctactagacatgtattgtcagagtatggtaacatgtccagtgcctgtgtccttttcattttggatgaaatgaggagaaaaagcaaggaggacggcctaaaaaccacaggtgagggaatcgaatggggtgttctattcggctttggtccaggccttactgtggagacagttgtacttcattcagtcgcaattaattag

SEQ ID NO: 10 - protein, 390 aa, Hypericum androsaemum
MVTVEEVRKAQRAEGPATVMAIGTAVPPNCVDQATYPDYYFRITNSEHKAELKEKFQRMCDKSQIKKRYMYLNEEVLKENPNMCAYMAPSLDARQDIVVVEVPKLGKEAAVKAIKEWGQPKSKITHLVFCTTSGVDMPGADYQLTKLLGLRPSVKRLMMYQQGCFAGGTVLRLAKDLAENNKGARVLVVCSEITAVTFRGPTDTHLDSLVGQALFGDGAAAIIIGSDPIPEVEKPLFELVSAAQTILPDSEGAIDGHLREVGLTFHLLKDVPGLISKNVEKSLTEAFKPLGISDWNSLFWIAHPGGPAILDQVEAKLSLKPEKLRATRHVLSEYGNMSSACVLFILDEMRRKSKEDGLKTTGEGIEWGVLFGFGPGLTVETVVLHSVAIN

SEQ ID NO: 11 - DNA, 669 bp, Medicago sativa
  source 1..669: mol_type = genomic DNA
atggctgcttccattaccgctattaccgttgaaaatttggaatacccagctgttgttacttctccagttactggtaagtcttactttttgggtggtgctggtgaaagaggtttgactattgaaggtaacttcattaagttcaccgccatcggtgtttacttggaagatattgctgttgcttctttggctgctaaatggaagggtaaatcctccgaagaattattggaaaccttggacttctacagagacattatttctggtccattcgaaaagttgatcagaggttccaagatcagagaattgtctggtccagaatactccagaaaggttatggaaaattgcgttgcccatttgaagtctgttggtacttatggtgatgctgaagctgaagctatgcaaaaatttgctgaagcctttaagccagttaattttccaccaggtgcttccgttttttacagacaatctccagatggtatcttgggtttgtctttttcaccagatacctccatcccagaaaaagaagctgctttgattgaaaacaaggctgtttcttctgctgtcttggaaactatgattggtgaacatgctgtttccccagatttgaaaagatgtttagctgctagattgcctgccttgttgaatgaaggtgcttttaagattggtaactaa

SEQ ID NO: 12 - protein, 222 aa, Medicago sativa
MAASITAITVENLEYPAVVTSPVTGKSYFLGGAGERGLTIEGNFIKFTAIGVYLEDIAVASLAAKWKGKSSEELLETLDFYRDIISGPFEKLIRGSKIRELSGPEYSRKVMENCVAHLKSVGTYGDAEAEAMQKFAEAFKPVNFPPGASVFYRQSPDGILGLSFSPDTSIPEKEAALIENKAVSSAVLETMIGEHAVSPDLKRCLAARLPALLNEGAFKIGN

SEQ ID NO: 13 - DNA, 633 bp, Petunia x hybrida
  source 1..633: mol_type = genomic DNA
atgggtaagaacgaagttatggttgacgaaattccattcccatctcaattcatgatgactactaagccattgccattgatgggtcacggtattactgacattgaaattcacttcttgcaaattaagttcactgctattggtgtttacttggacccagaaattgttactcacttgcaacaatggaagggtaagtctggtgctgaattgattgaaaacgacgaattcttcgaagctattgttaacgctccagttgacaagttcttgagagttgttgttattaaggaaattaagggttctcaatacggtgttcaattggaatctgctgttagagacagattggctgaagttgacaagtacgaagaagaagaagaagaagctttggaaaagattgttgaattcttccaatctaagtacttcaagaaggactctgttgttacttactctttcccagctacttctggtaacgttaagatttctttcgctactgaaggtaaggaagactctgaaattgaagttcaaaacgctaacgttgctggtgaaattaagaagtggtacttgggtggttctagaggtttgtctccaactactatttcttctttggctaacactttgtctgctgaattgtctaagtaa

SEQ ID NO: 14 - protein, 210 aa, Petunia x hybrida
MGKNEVMVDEIPFPSQFMMTTKPLPLMGHGITDIEIHFLQIKFTAIGVYLDPEIVTHLQQWKGKSGAELIENDEFFEAIVNAPVDKFLRVVVIKEIKGSQYGVQLESAVRDRLAEVDKYEEEEEEALEKIVEFFQSKYFKKDSVVTYSFPATSGNVKISFATEGKEDSEIEVQNANVAGEIKKWYLGGSRGLSPTTISSLANTLSAELSK

SEQ ID NO: 15 - DNA, 630 bp, Arabidopsis thaliana
  source 1..630: mol_type = genomic DNA
atgggtactgaaatggttatggttcacgaagttccattcccaccacaaattattacttctaagccattgtctttgttgggtcaaggtattactgacattgaaattcacttcttgcaagttaagttcactgctattggtgtttacttggacccatctgacgttaagactcacttggacaactggaagggtaagactggtaaggaattggctggtgacgacgacttcttcgacgctttggcttctgctgaaatggaaaaggttattagagttgttgttattaaggaaattaagggtgctcaatacggtgttcaattggaaaacactgttagagacagattggctgaagaagacaagtacgaagaagaagaagaaactgaattggaaaaggttgttggtttcttccaatctaagtacttcaaggctaactctgttattacttaccacttctctgctaaggacggtatttgtgaaattggtttcgaaactgaaggtaaggaagaagaaaagttgaaggttgaaaacgctaacgttgttggtatgatgcaaagatggtacttgtctggttctagaggtgtttctccatctactattgtttctattgctgactctatttctgctgttttgacttag

SEQ ID NO: 16 - protein, 209 aa, Arabidopsis thaliana
MGTEMVMVHEVPFPPQIITSKPLSLLGQGITDIEIHFLQVKFTAIGVYLDPSDVKTHLDNWKGKTGKELAGDDDFFDALASAEMEKVIRVVVIKEIKGAQYGVQLENTVRDRLAEEDKYEEEEETELEKVVGFFQSKYFKANSVITYHFSAKDGICEIGFETEGKEEEKLKVENANVVGMMQRWYLSGSRGVSPSTIVSIADSISAVLT

SEQ ID NO: 17 - DNA, 621 bp, Ipomoea nil
  source 1..621: mol_type = genomic DNA
atgggtactgaaatggttatggttgacgaaattccattcccaccacaagttaacttggacaacaagttgttgtctttgatgggtcacggtattactgacgttgaaattcacttcttgcaaattaagtacactgctattggtgtttacttggacccagaaattgtttctcacttgcaaaagtggaagggtaagactccagttgacttggctcaagacgacgacttcttcgaagctattattaacgctccagttgacaaggttttgagagttgttgttattaaggaaattaagggttctcaatacggtgttcaattggaaaactctgttagagacttgttggctgaagttgacaagtacgaagaagaagaagaagctgctttggaaaaggttgttgacttcttccaatctaagtacttcaagaagtcttctgttattactttctctttcccagctaacactgctactgctaagattgttttcgctactgaaggtaaggaagactcttctattgaagttgaaaacgctaacgttggtggtatgattaagaagtggtacttgggtggttctagagctgtttctccatctactatttcttctttggctaacattttgccagactag

SEQ ID NO: 18 - protein, 206 aa, Ipomoea nil
MGTEMVMVDEIPFPPQVNLDNKLLSLMGHGITDVEIHFLQIKYTAIGVYLDPEIVSHLQKWKGKTPVDLAQDDDFFEAIINAPVDKVLRVVVIKEIKGSQYGVQLENSVRDLLAEVDKYEEEEEAALEKVVDFFQSKYFKKSSVITFSFPANTATAKIVFATEGKEDSSIEVENANVGGMIKKWYLGGSRAVSPSTISSLANILPD

SEQ ID NO: 19 - DNA, 639 bp, Antirrhinum majus
  source 1..639: mol_type = genomic DNA
atgggttctgaagttgttgacgttgttatggttgacgaagttccattcccatctaagattactttcgctaagccattgtctttgttgggtcacggtttgactgacattgaaattcacttcttgcaaattaagttcactgctattggtgtttacttggacccagaaattgttgctcacttgttgaagtggaagggtaagccagctactgaattggctgaagacgacgacttgttcgacgctattatttctgctccagttgacaagattgttagagttgttgttattaaggaaattaagggttctcaatacggtgttcaattggaatctgctgttagagacagattggctgacgaagacaagtacgaagacgaagaagaagaagctttggaacaaattattgacttcttgcaatctaagtacttcaagaagaactctgttttgacttactacttcccagctacttctgctactgctgaaattattttcgctactgaaggtaaggaagactctaagattgaagttaagaacgctaacgttgttgacatgttgaagaagtggtacttgggtggtactagaggtgtttctccaactactattgcttctttggctactggtttgtctactgaattgtctaagtag

SEQ ID NO: 20 - protein, 212 aa, Antirrhinum majus
MGSEVVDVVMVDEVPFPSKITFAKPLSLLGHGLTDIEIHFLQIKFTAIGVYLDPEIVAHLLKWKGKPATELAEDDDLFDAIISAPVDKIVRVVVIKEIKGSQYGVQLESAVRDRLADEDKYEDEEEEALEQIIDFLQSKYFKKNSVLTYYFPATSATAEIIFATEGKEDSKIEVKNANVVDMLKKWYLGGTRGVSPTTIASLATGLSTELSK

SEQ ID NO: 21 - DNA, 630 bp, Glycine max
  source 1..630: mol_type = genomic DNA
atggctactgaagaagttttggttgacgaaattacttacccaactaagattactactactaagccattgtctttgttgggtcacggtattactgacatggaaattcacttcattcacgttaagttctactctattggtgtttacttggaaccagaagttgttggtcacttggaccaattcaagggtaagtctgctaaggaattggaagacaacgaagaattcttcaacgctttgatttctgctccagttgaaaagttcattagattggttgttattaaggaaattaagggtgctcaatacggtgttcaaattgaaactgctgttagagacagattggctgctgaagacaagtacgaagaagaagaagaagaagctttggaaaaggttattgaattcttccaatctaagtacttcaagaagttgtctgttattacttaccacttcccagctaactctgctactgctgaaattgttgtttctttggaaggtaaggaagactctaagtacgttattgaaaacgctaacgttgttgaagctattaagaagtggtacttgggtggttcttctgctgtttcttcttctactattcaatctttggcttctactttctctcaagaattgtctaagtag

SEQ ID NO: 22 - protein, 209 aa, Glycine max
MATEEVLVDEITYPTKITTTKPLSLLGHGITDMEIHFIHVKFYSIGVYLEPEVVGHLDQFKGKSAKELEDNEEFFNALISAPVEKFIRLVVIKEIKGAQYGVQIETAVRDRLAAEDKYEEEEEEALEKVIEFFQSKYFKKLSVITYHFPANSATAEIVVSLEGKEDSKYVIENANVVEAIKKWYLGGSSAVSSSTIQSLASTFSQELSK

SEQ ID NO: 23 - DNA, 2076 bp, Saccharomyces cerevisiae
  source 1..2076: mol_type = genomic DNA
atgccgtttggaatagacaacaccgacttcactgtcctggcggggctagtgcttgccgtgctactgtacgtaaagagaaactccatcaaggaactgctgatgtccgatgacggagatatcacagctgtcagctcgggcaacagagacattgctcaggtggtgaccgaaaacaacaagaactacttggtgttgtatgcgtcgcagactgggactgccgaggattacgccaaaaagttttccaaggagctggtggccaagttcaacctaaacgtgatgtgcgcagatgttgagaactacgactttgagtcgctaaacgatgtgcccgtcatagtctcgatttttatctctacatatggtgaaggagacttccccgacggggcggtcaactttgaagactttatttgtaatgcggaagcgggtgcactatcgaacctgaggtataatatgtttggtctgggaaattctacttatgaattctttaatggtgccgccaagaaggccgagaagcatctctccgctgcgggcgctatcagactaggcaagctcggtgaagctgatgatggtgcaggaactacagacgaagattacatggcctggaaggactccatcctggaggttttgaaagacgaactgcatttggacgaacaggaagccaagttcacctctcaattccagtacactgtgttgaacgaaatcactgactccatgtcgcttggtgaaccctctgctcactatttgccctcgcatcagttgaaccgcaacgcagacggcatccaattgggtcccttcgatttgtctcaaccgtatattgcacccatcgtgaaatctcgcgaactgttctcttccaatgaccgtaattgcatccactctgaatttgacttgtccggctctaacatcaagtactccactggtgaccatcttgctgtttggccttccaacccattggaaaaggtcgaacagttcttatccatattcaacctggaccctgaaaccatttttgacttgaagcccctggatcccaccgtcaaagtgcccttcccaacgccaactactattggcgctgctattaaacactatttggaaattacaggacctgtctccagacaattgttttcatctttgattcagttcgcccccaacgctgacgtcaaggaaaaattgactctgctttcgaaagacaaggaccaattcgccgtcgagataacctccaaatatttcaacatcgcagatgctctgaaatatttgtctgatggcgccaaatgggacaccgtacccatgcaattcttggtcgaatcagttccccaaatgactcctcgttactactctatctcttcctcttctctgtctgaaaagcaaaccgtccatgtcacctccattgtggaaaactttcctaacccagaattgcctgatgctcctccagttgttggtgttacgactaacttgttaagaaacattcaattggctcaaaacaatgttaacattgccgaaactaacctacctgttcactacgatttaaatggcccacgtaaacttttcgccaattacaaattgcccgtccacgttcgtcgttctaacttcagattgccttccaacccttccaccccagttatcatgatcggtccaggtaccggtgttgccccattccgtgggtttatcagagagcgtgtcgcgttcctcgaatcacaaaagaagggcggtaacaacgtttcgctaggtaagcatatactgttttatggatcccgtaacactgatgatttcttgtaccaggacgaatggccagaatacgccaaaaaattggatggttcgttcgaaatggtcgtggcccattccaggttgccaaacaccaaaaaagtttatgttcaagataaattaaaggattacgaagaccaagtatttgaaatgattaacaacggtgcatttatctacgtctgtggtgatgcaaagggtatggccaagggtgtgtcaaccgcattggttggcatcttatcccgtggtaaatccattaccactgatgaagcaacagagctaatcaagatgctcaagacttcaggtagataccaagaagatgtctggtaa

SEQ ID NO: 24 - protein, 691 aa, Saccharomyces cerevisiae
MPFGIDNTDFTVLAGLVLAVLLYVKRNSIKELLMSDDGDITAVSSGNRDIAQVVTENNKNYLVLYASQTGTAEDYAKKFSKELVAKFNLNVMCADVENYDFESLNDVPVIVSIFISTYGEGDFPDGAVNFEDFICNAEAGALSNLRYNMFGLGNSTYEFFNGAAKKAEKHLSAAGAIRLGKLGEADDGAGTTDEDYMAWKDSILEVLKDELHLDEQEAKFTSQFQYTVLNEITDSMSLGEPSAHYLPSHQLNRNADGIQLGPFDLSQPYIAPIVKSRELFSSNDRNCIHSEFDLSGSNIKYSTGDHLAVWPSNPLEKVEQFLSIFNLDPETIFDLKPLDPTVKVPFPTPTTIGAAIKHYLEITGPVSRQLFSSLIQFAPNADVKEKLTLLSKDKDQFAVEITSKYFNIADALKYLSDGAKWDTVPMQFLVESVPQMTPRYYSISSSSLSEKQTVHVTSIVENFPNPELPDAPPVVGVTTNLLRNIQLAQNNVNIAETNLPVHYDLNGPRKLFANYKLPVHVRRSNFRLPSNPSTPVIMIGPGTGVAPFRGFIRERVAFLESQKKGGNNVSLGKHILFYGSRNTDDFLYQDEWPEYAKKLDGSFEMVVAHSRLPNTKKVYVQDKLKDYEDQVFEMINNGAFIYVCGDAKGMAKGVSTALVGILSRGKSITTDEATELIKMLKTSGRYQEDVW

SEQ ID NO: 25 - DNA, 2079 bp, Arabidopsis thaliana
  source 1..2079: mol_type = genomic DNA
atgacttctgcactttatgcctccgatcttttcaaacaattgaaaagtatcatgggaacggattctttgtccgatgatgttgtattagttattgctacaacttctctggcactggttgctggtttcgttgtcttattgtggaaaaagaccacggcagatcgttccggcgagctaaagccactaatgatccctaagtctctgatggcgaaagatgaggatgatgacttagatctaggttctggaaaaacgagagtctctatcttcttcggcacacaaaccggaacagccgaaggattcgctaaagcactttcagaagagatcaaagcaagatacgaaaaggcggctgtaaaagtaatcgatttggatgattacgctgccgatgatgaccaatatgaggaaaagttgaaaaaggaaacattggctttcttttgtgtagccacgtatggtgatggtgaaccaaccgataacgccgcaagattctacaagtggtttactgaagagaacgaaagagatatcaagttgcagcaacttgcttacggcgtttttgccttaggtaacagacaatacgagcactttaacaagataggtattgtcttagatgaagagttatgcaaaaagggtgcgaagagattgattgaagtcggtttaggagatgatgatcaatctatcgaggatgactttaatgcatggaaggaatctttgtggtctgaattagataagttacttaaggacgaagatgataaatccgttgccactccatacacagccgtcattccagaatatagagtagttactcatgatccaagattcacaacacagaaatcaatggaaagtaatgtggctaatggtaatactaccatcgatattcatcatccatgtagagtagacgttgcagttcaaaaggaattgcacactcatgaatcagacagatcttgcatacatcttgaatttgatatatcacgtactggtatcacttacgaaacaggtgatcacgtgggtgtctacgctgaaaaccatgttgaaattgtagaggaagctggaaagttgttgggccatagtttagatcttgttttctcaattcatgccgataaagaggatggctcaccactagaaagtgcagtgcctccaccatttccaggaccatgcaccctaggtaccggtttagctcgttacgcggatctgttaaatcctccacgtaaatcagctctagtggccttggctgcgtacgccacagaaccttctgaggcagaaaaactgaaacatctaacttcaccagatggtaaggatgaatactcacaatggatagtagctagtcaacgttctttactagaagttatggctgctttcccatccgctaaacctcctttgggtgttttcttcgccgcaatagcgcctagactgcaaccaagatactattcaatttcatcctcacctagactggcaccatcaagagttcatgtcacatccgctttagtgtacggtccaactcctactggtagaatccataagggcgtttgttcaacatggatgaaaaacgcggttccagcagagaagtctcacgaatgttctggtgctccaatctttatcagagcctccaacttcaaactgccttccaatccttctactcctattgtcatggtcggtcctggtacaggtcttgctccattcagaggtttcttacaagagagaatggccttaaaggaggatggtgaagagttgggatcttctttgttgtttttcggctgtagaaacagacaaatggatttcatctacgaagatgaactgaataactttgtagatcaaggagttatttcagagttgataatggctttttctagagaaggtgctcagaaggagtacgtccaacacaaaatgatggaaaaggccgcacaagtttgggacttaatcaaagaggaaggctatctatatgtctgtggtgatgcaaagggtatggcaagagatgttcacagaacacttcatactatagtccaggaacaggaaggcgttagttcttctgaagcggaagcaattgtgaaaaagttacaaacagagggaagatacttgagagatgtgtggtaa

SEQ ID NO: 26 - protein, 692 aa, Arabidopsis thaliana [sequence omitted]

SEQ ID NO: 27 - DNA, 1539 bp, Petunia x hybrida
  source 1..1539: mol_type = genomic DNA
atggagattttaagtttaattttgtatacagttatcttcagtttcttattgcaatttattttgagatctttctttaggaaaagatatccattaccattacctccaggtccaaaaccatggccaataataggcaacttagtacacttgggacccaaaccacaccagtctaccgccgctatggcccaaacatatggtccattgatgtacttaaagatgggcttcgtagacgtcgttgtcgctgcatctgcaagtgttgctgcacaattcttgaagactcacgatgctaacttctcttctagacctccaaatagtggcgctgagcatatggcctataattaccaagacttggttttcgccccatacggccctaggtggagaatgttaaggaaaatatgttctgtgcacttgttctctacaaaagcattggatgatttcagacatgtcagacaagacgaagtaaagactttaaccagagcattagcttcagcaggtcagaagcccgtgaagttaggccaattattaaacgtctgtactactaatgctttagccagagtaatgttaggtaaaagagtcttcgctgacggttcaggcgatgttgacccacaagccgcagaattcaaatctatggtagttgagatgatggtcgtcgccggtgtatttaacataggagatttcattcctcaattaaattggttggacattcaaggtgtggccgctaaaatgaagaagttacatgctagattcgatgctttcttgacagacatattggaagaacataaaggtaaaatctttggtgaaatgaaggatttattaagtaccttaatctccttgaagaatgatgatgccgacaatgatggtggaaaattgacagatacagagattaaagcattattattaaacttgtttgttgcaggaactgatacttcatcctcaactgttgaatgggcaattgccgaattgatcagaaatccaaagattttggctcaggctcaacaagagatcgacaaagtggtaggtagagacaggttggtgggcgaattagatttagcacaattaacctacttggaagcaattgttaaggaaacctttagattgcatccctccactccattatcattgccaagaatagcatcagaatcatgtgaaatcaacggttactttatcccaaaaggatccactttattattgaatgtttgggctatagccagggatcctaatgcttgggccgatcctttagaatttagacctgaaagattcttgcctggtggtgaaaagcctaaggtggatgtaaggggaaatgattttgaggtgattccctttggagcaggtaggaggatttgcgctggaatgaatttgggtattaggatggttcagttaatgatcgcaacattgatacatgcatttaactgggatttggtttccggtcagttgcctgaaatgttgaacatggaagaggcttatggtttgacattgcagagagctgatcctttggttgttcatcccagacccagattggaagctcaggcttatatcggttga

SEQ ID NO: 28 - protein, 512 aa, Petunia x hybrida
MEILSLILYTVIFSFLLQFILRSFFRKRYPLPLPPGPKPWPIIGNLVHLGPKPHQSTAAMAQTYGPLMYLKMGFVDVVVAASASVAAQFLKTHDANFSSRPPNSGAEHMAYNYQDLVFAPYGPRWRMLRKICSVHLFSTKALDDFRHVRQDEVKTLTRALASAGQKPVKLGQLLNVCTTNALARVMLGKRVFADGSGDVDPQAAEFKSMVVEMMVVAGVFNIGDFIPQLNWLDIQGVAAKMKKLHARFDAFLTDILEEHKGKIFGEMKDLLSTLISLKNDDADNDGGKLTDTEIKALLLNLFVAGTDTSSSTVEWAIAELIRNPKILAQAQQEIDKVVGRDRLVGELDLAQLTYLEAIVKETFRLHPSTPLSLPRIASESCEINGYFIPKGSTLLLNVWAIARDPNAWADPLEFRPERFLPGGEKPKVDVRGNDFEVIPFGAGRRICAGMNLGIRMVQLMIATLIHAFNWDLVSGQLPEMLNMEEAYGLTLQRADPLVVHPRPRLEAQAYIG

SEQ ID NO: 29 - DNA, 1521 bp, Petunia x hybrida
  source 1..1521: mol_type = genomic DNA
atgatgttgttgaccgaattgggtgctgctacttccatttttttgattgcccatatcatcatctccaccttgatttctaaaaccaccggtagacatttgccaccaggtccaagaggttggccagttattggtgctttgccattattgggtgcaatgccacatgtttctttggctaaaatggctaagaaatacggtgccatcatgtacttgaaagttggtacttgtggtatggctgttgcttctactccagatgctgctaaggcttttttgaaaaccttggacatcaacttctctaacagaccaccaaatgctggtgctactcatttggcttataatgcccaagatatggttttcgctcattatggtccaagatggaagttgttgagaaagttgtctaacttgcatatgttgggtggtaaggctttggaaaattgggctaatgttagagccaatgaattgggtcatatgttgaagtccatgtccgatatgtctagagaaggtcaaagagttgttgtcgctgaaatgttgacttttgctatggctaacatgatcggtcaagtcatgttgtctaagagagttttcgttgataagggtgtcgaagtcaacgaattcaaggatatggttgttgaattgatgaccattgccggttactttaacatcggtgatttcattccatgtttggcttggatggacttgcaaggtattgaaaagagaatgaagagattgcacaagaagttcgacgctttgttgactaagatgttcgatgaacataaggctaccacctacgaaagaaaaggtaagccagatttcttggatgtcgttatggaaaacggtgataactctgaaggtgaaagattgtctaccaccaatattaaggccttgttgttgaacttgttcaccgctggtactgatacttcttcttctgctattgaatgggctttggcagaaatgatgaagaacccagctattttgaaaaaggctcaagccgaaatggatcaagttatcggtagaaacagaagattattggaatccgacattccaaacttgccatacttgagagctatctgcaaagaaacttttagaaagcacccatctaccccattgaacttgccaagaatttctaacgaaccatgcatcgttgatggttactacatcccaaaaaacaccagattgtccgttaacatttgggccattggtagagatccacaagtttgggaaaatccattggaattcaacccagaaagattcttgtctggtagaaactccaagattgacccaagaggtaatgacttcgaattgattccatttggtgccggtagaagaatttgtgctggtactagaatgggtatcgttatggtcgaatatatcttaggtactttggtccactctttcgattggaaattgccatccgaagttatcgaattgaatatggaagaagcctttggtttggccttgcaaaaagctgttccattagaagctatggttaccccaagattgcaattggatgtttacgttccataa

SEQ ID NO: 30 - protein, 506 aa, Petunia x hybrida
MMLLTELGAATSIFLIAHIIISTLISKTTGRHLPPGPRGWPVIGALPLLGAMPHVSLAKMAKKYGAIMYLKVGTCGMAVASTPDAAKAFLKTLDINFSNRPPNAGATHLAYNAQDMVFAHYGPRWKLLRKLSNLHMLGGKALENWANVRANELGHMLKSMSDMSREGQRVVVAEMLTFAMANMIGQVMLSKRVFVDKGVEVNEFKDMVVELMTIAGYFNIGDFIPCLAWMDLQGIEKRMKRLHKKFDALLTKMFDEHKATTYERKGKPDFLDVVMENGDNSEGERLSTTNIKALLLNLFTAGTDTSSSAIEWALAEMMKNPAILKKAQAEMDQVIGRNRRLLESDIPNLPYLRAICKETFRKHPSTPLNLPRISNEPCIVDGYYIPKNTRLSVNIWAIGRDPQVWENPLEFNPERFLSGRNSKIDPRGNDFELIPFGAGRRICAGTRMGIVMVEYILGTLVHSFDWKLPSEVIELNMEEAFGLALQKAVPLEAMVTPRLQLDVYVP

SEQ ID NO: 31 - DNA, 1098 bp, Petroselinum crispum
  source 1..1098: mol_type = genomic DNA
atggctccaactactattactgctttggctaaggaaaagactttgaacttggacttcgttagagacgaagacgaaagaccaaaggttgcttacaaccaattctctaacgaaattccaattatttctttggctggtttggacgacgactctgacggtagaagaccagaaatttgtagaaagattgttaaggcttgtgaagactggggtattttccaagttgttgaccacggtattgactctggtttgatttctgaaatgactagattgtctagagaattcttcgctttgccagctgaagaaaagttggaatacgacactactggtggtaagagaggtggtttcactatttctactgttttgcaaggtgacgacgctatggactggagagaattcgttacttacttctcttacccaattaacgctagagactactctagatggccaaagaagccagaaggttggagatctactactgaagtttactctgaaaagttgatggttttgggtgctaagttgttggaagttttgtctgaagctatgggtttggaaaagggtgacttgactaaggcttgtgttgacatggaacaaaaggttttgattaactactacccaacttgtccacaaccagacttgactttgggtgttagaagacacactgacccaggtactattactattttgttgcaagacatggttggtggtttgcaagctactagagacggtggtaagacttggattactgttcaaccagttgaaggtgctttcgttgttaacttgggtgaccacggtcactacttgtctaacggtagattcagaaacgctgaccaccaagctgttgttaactctacttcttctagattgtctattgctactttccaaaacccagctcaaaacgctattgtttacccattgaagattagagaaggtgaaaaggctattttggacgaagctattacttacgctgaaatgtacaagaagtgtatgactaagcacattgaagttgctactagaaagaagttggctaaggaaaagagattgcaagacgaaaaggctaagttggaaatgaagtctaagtctgctgacgaaaacttggcttag

SEQ ID NO: 32 - protein, 365 aa, Petroselinum crispum
MAPTTITALAKEKTLNLDFVRDEDERPKVAYNQFSNEIPIISLAGLDDDSDGRRPEICRKIVKACEDWGIFQVVDHGIDSGLISEMTRLSREFFALPAEEKLEYDTTGGKRGGFTISTVLQGDDAMDWREFVTYFSYPINARDYSRWPKKPEGWRSTTEVYSEKLMVLGAKLLEVLSEAMGLEKGDLTKACVDMEQKVLINYYPTCPQPDLTLGVRRHTDPGTITILLQDMVGGLQATRDGGKTWITVQPVEGAFVVNLGDHGHYLSNGRFRNADHQAVVNSTSSRLSIATFQNPAQNAIVYPLKIREGEKAILDEAITYAEMYKKCMTKHIEVATRKKLAKEKRLQDEKAKLEMKSKSADENLA

SEQ ID NO: 33 - DNA, 1521 bp, Antirrhinum majus
  source 1..1521: mol_type = genomic DNA
atgtctactttggtttactctactttgttcattttgtctactttgttgttgactttgttgactagaactagaagaaagactagaccaccaggtccattggctttgccattgattggtcacttgcacttgttgggtccaaagttgcaccacactttccaccaattctctcaaagatacggtccattgattcaattgtacttgggttctgttccatgtgttgttgcttctactccagaattggctagagaattcttgaagactcacgaattggacttctcttctagaaagcactctactgctattgacattgttacttacgactcttctttcgctttcgctccatacggtccatactggaagttcattaagaagttgtgtacttacgaattgttgggtgctagaaacttgtctcacttccaaccaattagagctttggaagttaactctttcttgagaattttgtacgaaaagactgaacaaaagcaatctgttaacgttactgaagaattggttaagttgacttctaacgttatttctaacatgatgttgggtattagatgttctggtactgaaggtgaagctgaagttgctagaactgttattagagaagttactcaaattttcggtgaattcgacgtttctgaaattgtttggttctgtaagaacttggacttgcaaggtattagaaagagatctgaagacattagaagaagatacgacgctttgttggaaaagattatttctgacagagaaagattgagattgagaggtggtggtggtggtggtggtggtgaagttaaggacttcttggacatgttgttggacgttatggaatctgaaaagtctgaagttgaattcactagagaacacttgaaggctttgattttggacttcttcactgctggtactgacactactgctattactactgaatgggctattgctgaattgatttctaacccaaacgttttgaagaaggctcaagaagaaatggacaaggttattggttctcaaagattgttgcaagaatctgacgctccaaacttgccatacttgaacgctattattaaggaaactttcagattgcacccaccaattccaatgttgactagaaagtctatttctgacgttgttgttaacggttacactattccagctaagactttgttgttcgttaacttgtggtctatgggtagaaacccaaactactgggaaaacccaatggaattcagaccagaaagattcttggaaaagggtactggttctattgacgttaagggtcaacacttcgaattgttgccattcggtactggtagaagaggttgtccaggtatgttgttgggtatgcaagaattgttctctattattggtgctatggttcaatgtttcgactggaagttgccagacggtgttaagtctgttgacatgactgaaagaccaggtttgactgctccaagagctaacgacttggtttgtcaattggttccaagaattgacccagttgttgtttctggtccataa

SEQ ID NO: 34 - protein, 506 aa, Antirrhinum majus
MSTLVYSTLFILSTLLLTLLTRTRRKTRPPGPLALPLIGHLHLLGPKLHHTFHQFSQRYGPLIQLYLGSVPCVVASTPELAREFLKTHELDFSSRKHSTAIDIVTYDSSFAFAPYGPYWKFIKKLCTYELLGARNLSHFQPIRALEVNSFLRILYEKTEQKQSVNVTEELVKLTSNVISNMMLGIRCSGTEGEAEVARTVIREVTQIFGEFDVSEIVWFCKNLDLQGIRKRSEDIRRRYDALLEKIISDRERLRLRGGGGGGGGEVKDFLDMLLDVMESEKSEVEFTREHLKALILDFFTAGTDTTAITTEWAIAELISNPNVLKKAQEEMDKVIGSQRLLQESDAPNLPYLNAIIKETFRLHPPIPMLTRKSISDVVVNGYTIPAKTLLFVNLWSMGRNPNYWENPMEFRPERFLEKGTGSIDVKGQHFELLPFGTGRRGCPGMLLGMQELFSIIGAMVQCFDWKLPDGVKSVDMTERPGLTAPRANDLVCQLVPRIDPVVVSGP

SEQ ID NO: 35 - DNA, 1566 bp, Glycine max
  source 1..1566: mol_type = genomic DNA
atgttgttggaattggctttgggtttgttcgttttggctttgttcttgcacttgagaccaactccatctgctaagtctaaggctttgagacacttgccaaacccaccatctccaaagccaagattgccattcattggtcacttgcacttgttgaaggacaagttgttgcactacgctttgattgacttgtctaagaagcacggtccattgttctctttgtctttcggttctatgccaactgttgttgcttctactccagaattgttcaagttgttcttgcaaactcacgaagctacttctttcaacactagattccaaacttctgctattagaagattgacttacgacaactctgttgctatggttccattcggtccatactggaagttcgttagaaagttgattatgaacgacttgttgaacgctactactgttaacaagttgagaccattgagaactcaacaaattagaaagttcttgagagttatggctcaatctgctgaagctcaaaagccattggacgttactgaagaattgttgaagtggactaactctactatttctatgatgatgttgggtgaagctgaagaaattagagacattgctagagaagttttgaagattttcggtgaatactctttgactgacttcatttggccattgaagtacttgaaggttggtaagtacgaaaagagaattgacgacattttgaacaagttcgacccagttgttgaaagagttattaagaagagaagagaaattgttagaagaagaaagaacggtgaagttgttgaaggtgaagcttctggtgttttcttggacactttgttggaattcgctgaagacgaaactatggaaattaagattactaaggaacaaattaagggtttggttgttgacttcttctctgctggtactgactctactgctgttgctactgaatgggctttggctgaattgattaacaacccaagagttttgcaaaaggctagagaagaagtttactctgttgttggtaaggacagattggttgacgaagttgacactcaaaacttgccatacattagagctattgttaaggaaactttcagaatgcacccaccattgccagttgttaagagaaagtgtactgaagaatgtgaaattaacggttacgttattccagaaggtgctttggttttgttcaacgtttggcaagttggtagagacccaaagtactgggacagaccatctgaattcagaccagaaagattcttggaaactggtgctgaaggtgaagctggtccattggacttgagaggtcaacacttccaattgttgccattcggttctggtagaagaatgtgtccaggtgttaacttggctacttctggtatggctactttgttggcttctttgattcaatgtttcgacttgcaagttttgggtccacaaggtcaaattttgaagggtgacgacgctaaggtttctatggaagaaagagctggtttgactgttccaagagctcactctttggtttgtgttccattggctagaattggtgttgcttctaagttgttgtcttaa

SEQ ID NO: 36 - protein, 521 aa, Glycine max
MLLELALGLFVLALFLHLRPTPSAKSKALRHLPNPPSPKPRLPFIGHLHLLKDKLLHYALIDLSKKHGPLFSLSFGSMPTVVASTPELFKLFLQTHEATSFNTRFQTSAIRRLTYDNSVAMVPFGPYWKFVRKLIMNDLLNATTVNKLRPLRTQQIRKFLRVMAQSAEAQKPLDVTEELLKWTNSTISMMMLGEAEEIRDIAREVLKIFGEYSLTDFIWPLKYLKVGKYEKRIDDILNKFDPVVERVIKKRREIVRRRKNGEVVEGEASGVFLDTLLEFAEDETMEIKITKEQIKGLVVDFFSAGTDSTAVATEWALAELINNPRVLQKAREEVYSVVGKDRLVDEVDTQNLPYIRAIVKETFRMHPPLPVVKRKCTEECEINGYVIPEGALVLFNVWQVGRDPKYWDRPSEFRPERFLETGAEGEAGPLDLRGQHFQLLPFGSGRRMCPGVNLATSGMATLLASLIQCFDLQVLGPQGQILKGDDAKVSMEERAGLTVPRAHSLVCVPLARIGVASKLLS

SEQ ID NO: 37 - DNA, 960 bp, Glycine max
  source 1..960: mol_type = genomic DNA
atggctaaggaaattgttaaggaattgttgccattgattagagtttacaaggacggttctgttgaaagattgttgtcttctgaaaacgttgctgcttctccagaagacccacaaactggtgtttcttctaaggacattgttattgctgacaacccatacgtttctgctagaattttcttgccaaagtctcaccacactaacaacaagttgccaattttcttgtacttccacggtggtgctttctgtgttgaatctgctttctctttcttcgttcacagatacttgaacattttggcttctgaagctaacattattgctatttctgttgacttcagattgttgccacaccacccaattccagctgcttacgaagacggttggactactttgaagtggattgcttctcacgctaacaacactaacactactaacccagaaccatggttgttgaaccacgctgacttcactaaggtttacgttggtggtgaaacttctggtgctaacattgctcacaacttgttgttgagagctggtaacgaatctttgccaggtgacttgaagattttgggtggtttgttgtgttgtccattcttctggggttctaagccaattggttctgaagctgttgaaggtcacgaacaatctttggctatgaaggtttggaacttcgcttgtccagacgctccaggtggtattgacaacccatggattaacccatgtgttccaggtgctccatctttggctactttggcttgttctaagttgttggttactattactggtaaggacgaattcagagacagagacattttgtaccaccacactgttgaacaatctggttggcaaggtgaattgcaattgttcgacgctggtgacgaagaacacgctttccaattgttcaagccagaaactcacttggctaaggctatgattaagagattggcttctttcttggtttaa

SEQ ID NO: 38 - protein, 319 aa, Glycine max
MAKEIVKELLPLIRVYKDGSVERLLSSENVAASPEDPQTGVSSKDIVIADNPYVSARIFLPKSHHTNNKLPIFLYFHGGAFCVESAFSFFVHRYLNILASEANIIAISVDFRLLPHHPIPAAYEDGWTTLKWIASHANNTNTTNPEPWLLNHADFTKVYVGGETSGANIAHNLLLRAGNESLPGDLKILGGLLCCPFFWGSKPIGSEAVEGHEQSLAMKVWNFACPDAPGGIDNPWINPCVPGAPSLATLACSKLLVTITGKDEFRDRDILYHHTVEQSGWQGELQLFDAGDEEHAFQLFKPETHLAKAMIKRLASFLV

SEQ ID NO: 39 - DNA, 1095 bp, Malus domestica
  source 1..1095: mol_type = genomic DNA
atggctccagccactaccttaacctctattgcacatgaaaagacattacagcagaagttcgttagagatgaggatgaaaggcctaaggttgcctataacgacttttctaatgaaattccaataatctctttggctggtatagacgaagtagaaggtagaaggggagaaatatgtaagaagattgttgcagcttgcgaagattggggcattttccagatcgtagaccatggtgtagatgccgaattgatatcagaaatgacaggtttggctagagaattcttcgcattgccttcagaagagaagttaaggtttgatatgtccggtggtaagaaaggtggttttatagtctctagtcatttacagggtgaagccgttcaagattggagagaaatcgtaacatatttctcatacccaattagacacagagattactccaggtggcctgataagccagaagcctggagggaagttactaagaaatactcagatgagttgatgggattagcttgtaaattgttgggcgtgttgtcagaagccatgggattggatacagaggccttgaccaaagcatgtgttgatatggaccaaaaggtagttgtcaacttctaccctaaatgccctcaaccagacttgacattaggcttgaaaagacataccgaccccggcactatcactttattattacaagaccaagtcggtggtttgcaggctactagagacgacggtaaaacctggatcactgttcaacccgttgaaggagcattcgtcgttaatttgggcgatcatggacacttattgtccaatggtagatttaagaatgctgatcaccaagctgtggtcaactctaatagtagtagattatccattgctacatttcagaacccagcacaagaagcaattgtttatcctttatctgtgagagaaggagagaagcctattttagaggcaccaattacatatactgagatgtataagaagaagatgtctaaagatttggagttagcaagattgaagaaattagctaaagagcaacaaagtcaagatttagagaaggctaaagtggatactaaaccagtggatgatatcttcgcttaa

SEQ ID NO: 40 - protein, 364 aa, Malus domestica
MAPATTLTSIAHEKTLQQKFVRDEDERPKVAYNDFSNEIPIISLAGIDEVEGRRGEICKKIVAACEDWGIFQIVDHGVDAELISEMTGLAREFFALPSEEKLRFDMSGGKKGGFIVSSHLQGEAVQDWREIVTYFSYPIRHRDYSRWPDKPEAWREVTKKYSDELMGLACKLLGVLSEAMGLDTEALTKACVDMDQKVVVNFYPKCPQPDLTLGLKRHTDPGTITLLLQDQVGGLQATRDDGKTWITVQPVEGAFVVNLGDHGHLLSNGRFKNADHQAVVNSNSSRLSIATFQNPAQEAIVYPLSVREGEKPILEAPITYTEMYKKKMSKDLELARLKKLAKEQQSQDLEKAKVDTKPVDDIFA

SEQ ID NO: 41 - DNA, 1008 bp, Citrus unshiu
  source 1..1008: mol_type = genomic DNA
atggaagttgaaagagttcaagctattgcttctttgtctcactctaacggtactattccagctgaattcattagaccagaaaaggaacaaccagcttctactacttaccacggtccagctccagaaattccaactattgacttggacgacccagttcaagacagattggttagatctattgctgaagcttctagagaatggggtattttccaagttactaaccacggtattccatctgacttgatttgtaagttgcaagctgttggtaaggaattcttcgaattgccacaagaagaaaaggaagtttactctagaccagctgacgctaaggacgttcaaggttacggtactaagttgcaaaaggaagttgaaggtaagaagtcttgggttgaccacttgttccacagagtttggccaccatcttctattaactacagattctggccaaagaacccaccatcttacagagctgttaacgaagaatacgctaagtacatgagagaagttgttgacaagttgttcacttacttgtctttgggtttgggtgttgaaggtggtgttttgaaggaagctgctggtggtgacgacattgaatacatgttgaagattaactactacccaccatgtccaagaccagacttggctttgggtgttgttgctcacactgacttgtctgctttgactgttttggttccaaacgaagttccaggtttgcaagttttcaaggacgacagatggattgacgctaagtacattccaaacgctttggttattcacattggtgaccaaattgaaattttgtctaacggtaagtacaaggctgttttgcacagaactactgttaacaaggacaagactagaatgtcttggccagttttcttggaaccaccagctgacactgttgttggtccattgccacaattggttgacgacgaaaacccaccaaagtacaaggctaagaagttcaaggactactcttactgtaagttgaacaagttgccacaataa

SEQ ID NO: 42 - protein, 335 aa, Citrus unshiu
MEVERVQAIASLSHSNGTIPAEFIRPEKEQPASTTYHGPAPEIPTIDLDDPVQDRLVRSIAEASREWGIFQVTNHGIPSDLICKLQAVGKEFFELPQEEKEVYSRPADAKDVQGYGTKLQKEVEGKKSWVDHLFHRVWPPSSINYRFWPKNPPSYRAVNEEYAKYMREVVDKLFTYLSLGLGVEGGVLKEAAGGDDIEYMLKINYYPPCPRPDLALGVVAHTDLSALTVLVPNEVPGLQVFKDDRWIDAKYIPNALVIHIGDQIEILSNGKYKAVLHRTTVNKDKTRMSWPVFLEPPADTVVGPLPQLVDDENPPKYKAKKFKDYSYCKLNKLPQ

SEQ ID NO: 43 - DNA, 1044 bp, Anthurium andraeanum
  source 1..1044: mol_type = genomic DNA
atgatgcacaaaggtacagtttgtgttactggtgctgccggcttcgtaggtagttggttaatcatgaggttattagaacaaggttactccgttaaggctacagtgagagatccttctaacatgaagaaagttaagcatttgttggatttacccggagcagcaaataggttgactttgtggaaggcagatttagttgatgaaggttcctttgatgaacctattcaaggttgcacaggtgtattccatgtcgcaactccaatggatttcgagtctaaagatcctgagagtgagatgattaaacctacaatcgagggcatgttaaacgttttgaggtcatgtgcaagagcatccagtactgtcagaagggtagttttcacttcctctgccggtactgttagtatccatgaaggcagaagacacttatacgatgaaaccagttggtcagacgtcgatttctgcagggccaagaagatgacaggttggatgtatttcgtctctaaaaccttagcagaaaaggccgcctgggatttcgcagaaaagaataacattgacttcatttctattatacccactttagtcaatggtccctttgttatgccaactatgccaccatcaatgttgtcagctttggctttaattaccagaaatgaacctcattactcaattttgaaccctgtgcaatttgtacatttggatgatttatgcaatgctcatattttcttgtttgaatgtccagatgctaagggtagatacatctgttcttcacacgatgtaacaatcgccggtttagctcaaatattgagacaaagatatccagagtttgacgtgccaacagaatttggagaaatggaggtgtttgacattatatcatattcttctaagaagttaactgacttgggatttgaatttaaatattctttagaggacatgtttgacggcgctatacagtcttgtagagaaaagggcttgttgcctccagctacaaaagaaccatcctatgctaccgaacaattgatagctaccggacaggacaatggacactaa

SEQ ID NO: 44 - protein, 347 aa, Anthurium andraeanum
MMHKGTVCVTGAAGFVGSWLIMRLLEQGYSVKATVRDPSNMKKVKHLLDLPGAANRLTLWKADLVDEGSFDEPIQGCTGVFHVATPMDFESKDPESEMIKPTIEGMLNVLRSCARASSTVRRVVFTSSAGTVSIHEGRRHLYDETSWSDVDFCRAKKMTGWMYFVSKTLAEKAAWDFAEKNNIDFISIIPTLVNGPFVMPTMPPSMLSALALITRNEPHYSILNPVQFVHLDDLCNAHIFLFECPDAKGRYICSSHDVTIAGLAQILRQRYPEFDVPTEFGEMEVFDIISYSSKKLTDLGFEFKYSLEDMFDGAIQSCREKGLLPPATKEPSYATEQLIATGQDNGH

SEQ ID NO: 45 - DNA, 1041 bp, Populus trichocarpa
  source 1..1041: mol_type = genomic DNA
atgggtactgaagctgaaaccgtttgtgttactggtgcttctggttttattggttcctggttgatcatgagattattggaaaaaggttacgctgttagagccactgttagagatccagataatatgaagaaggtcacccacttgttggaattgccaaaggcttctactcatttgactttgtggaaagccgatttgtctgttgaaggttcttacgatgaagctattcaaggttgtactggtgttttccatgttgctactccaatggatttcgaatctaaggatccagaaaacgaagttatcaagccaaccattaacggtgttttggatattatgagagcttgcgctaactctaagaccgttagaaagatcgttttcacttcttctgctggtactgttgatgtcgaagaaaaaagaaagccagtctacgatgaatcttgctggtctgatttggatttcgtccaatctattaagatgaccggttggatgtacttcgtttctaaaactttggctgaacaagctgcttggaagttcgctaaagaaaacaacttggacttcatctccattatcccaactttggttgttggtccattcatcatgcaatctatgccaccatctttgttgactgccttgtctttgattactggtaacgaagctcattacggtatcttgaaacaaggtcattacgttcacttggatgacttgtgtatgtcccatatcttcttgtacgaaaacccaaaagctgaaggtagatatatctgcaactctgatgatgccaacattcatgatttggctaagttgttgagagaaaagtacccagaatacaacgttccagctaagttcaaggatatcgacgaaaatttggcttgcgttgctttctcatctaagaagttgacagatttgggtttcgaattcaagtactccttggaagatatgtttgctggtgcagttgaaacctgtagagaaaagggtttgattccattgtcccacagaaaacaagtcgtcgaagaatgcaaagaaaatgaagttgttccagcttcttaa

SEQ ID NO: 46 - protein, 346 aa, Populus trichocarpa
MGTEAETVCVTGASGFIGSWLIMRLLEKGYAVRATVRDPDNMKKVTHLLELPKASTHLTLWKADLSVEGSYDEAIQGCTGVFHVATPMDFESKDPENEVIKPTINGVLDIMRACANSKTVRKIVFTSSAGTVDVEEKRKPVYDESCWSDLDFVQSIKMTGWMYFVSKTLAEQAAWKFAKENNLDFISIIPTLVVGPFIMQSMPPSLLTALSLITGNEAHYGILKQGHYVHLDDLCMSHIFLYENPKAEGRYICNSDDANIHDLAKLLREKYPEYNVPAKFKDIDENLACVAFSSKKLTDLGFEFKYSLEDMFAGAVETCREKGLIPLSHRKQVVEECKENEVVPAS

SEQ ID NO: 47 - DNA, 1086 bp, Irisx hollandica
  source 1..1086: mol_type = genomic DNA
atgatgtccccagttgttgttactggtgcttctggttatgttggttcttggttggttatgaagttgttgagagatggttatgctgttagagctactgttagagatccaactaatgtcgaaaagaccaagcctttgttggatttgccaggtgctgatgctttgttgactatttggaaagctgatttgggtcaagatggttcttttgataaggctgttgctggttgtactgctgtttttcatgttgctactccaatggacttcgaatctaaggatccagaaaacgaagttatcaagccaaccattaacggtgtcttgtccattatgagatcctgtaaaaaagccggtactgttaagagagttgttttcacttcttctgctggtactgtcgatgtcaaagaacatcaacaaaccgaatacgacgaatcctcttggtctgatgttgatttctgtagaagagttaagatgaccggttggatgtacttcgtttctaaaactttggctgaaagagctgcttgggaatttgctagagaaaacggtattgacttcatctccattatcccaactttggttgttggtccattcattactactactatgccaccatctatggttaccgctttgtcttttatgactggtaacgaagctcattaccacattattaagcacgcccaattggttcacttggatgatttgtgtgctgctcatatctacttgttgaatagacctgaagctaacggtagatatatctgctcatctcatgaagccaccattcatgatttggctagaatggttagagaaagacatccatggtgtggttctattccagaaaagttcgatggtatcgaaaaggatgttagaaccgttcacttctcatccaagagattattggatttgggtttcgaattcaagtacaccgtcgaagaaatgttcgacgaagctattagatcctgcgttgaaaagaagttgattccattgccagaaaatggtaacgttgatgctgctgctggtgctaaagatatggttcatggtgctgaagaacatgctagaattgctatggaattggaacctaagaagaaggttaagtaa

SEQ ID NO: 48 - protein, 361 aa, Irisx hollandica
MMSPVVVTGASGYVGSWLVMKLLRDGYAVRATVRDPTNVEKTKPLLDLPGADALLTIWKADLGQDGSFDKAVAGCTAVFHVATPMDFESKDPENEVIKPTINGVLSIMRSCKKAGTVKRVVFTSSAGTVDVKEHQQTEYDESSWSDVDFCRRVKMTGWMYFVSKTLAERAAWEFARENGIDFISIIPTLVVGPFITTTMPPSMVTALSFMTGNEAHYHIIKHAQLVHLDDLCAAHIYLLNRPEANGRYICSSHEATIHDLARMVRERHPWCGSIPEKFDGIEKDVRTVHFSSKRLLDLGFEFKYTVEEMFDEAIRSCVEKKLIPLPENGNVDAAAGAKDMVHGAEEHARIAMELEPKKKVK

SEQ ID NO: 49 - DNA, 1041 bp, Vitis vinifera
  source 1..1041: mol_type = genomic DNA
atgactgtttctccagttccatctccaaagggtagagttttgattgctggtgctactggtttcattggtcaattcgttgctgctgcttctttggacgctcacagaccaacttacattttggctagaccaggtccaagatctccatctaaggctaacattttcaaggctttggaagacaagggtgctattattgtttacggtttgattaacgaacaagaagctatggaaaagattttgaaggaacacgaaattgacattgttgtttctactgttggtggtgaatctattttggaccaaattgctttggttaaggctatgaaggctgttggtactattaagagattcttgccatctgaattcggtcacgacgttaacagagctgacccagttgaaccaggtttgaacatgtacagagaaaagagaagagttagacaattggttgaagaatctggtattccattcacttacatttgttgtaactctattgcttcttggccatactacaacaacattcacccatctgaagttttgccaccaactgacttcttccaaatttacggtgacggtaacgttaaggcttacttcgttgctggtactgacattggtaagttcactatgaagactgttgacgacgttagaactttgaacaagtctgttcacttcagaccatcttgtaactgtttgaacattaacgaattggcttctgtttgggaaaagaagattggtagaactttgccaagagttactgttactgaagacgacttgttggctgctgctggtgaaaacattattccacaatctgttgttgctgctttcactcacgacattttcattaagggttgtcaagttaacttctctattgacggtccagaagacgttgaagttactactttgtacccagaagactctttcagaactgttgaagaatgtttcggtgaatacattgttaagattgaagaaaagcaaccaactgctgactctgctattgctaacactggtccagttgttggtatgagacaagttactgctacttgtgcttaa

SEQ ID NO: 50 - protein, 346 aa, Vitis vinifera
MTVSPVPSPKGRVLIAGATGFIGQFVAAASLDAHRPTYILARPGPRSPSKANIFKALEDKGAIIVYGLINEQEAMEKILKEHEIDIVVSTVGGESILDQIALVKAMKAVGTIKRFLPSEFGHDVNRADPVEPGLNMYREKRRVRQLVEESGIPFTYICCNSIASWPYYNNIHPSEVLPPTDFFQIYGDGNVKAYFVAGTDIGKFTMKTVDDVRTLNKSVHFRPSCNCLNINELASVWEKKIGRTLPRVTVTEDDLLAAAGENIIPQSVVAAFTHDIFIKGCQVNFSIDGPEDVEVTTLYPEDSFRTVEECFGEYIVKIEEKQPTADSAIANTGPVVGMRQVTATCA

SEQ ID NO: 51 - DNA, 1017 bp, Lotus corniculatus
  source 1..1017: mol_type = genomic DNA
atggcttctattaagccaatttacgaaaagaagaaggcttgtgttattggtggtactggtttcgttgcttctttgttgattaagcaattgttggaaaagggttacgctgttaacactactgttagagacccagacaaccacaagaagatttctcacttgttggttttgcaatctttgggtgaattgaacattttcggtggtgaattgactgttgaaaacgacttcgacactccaattgaaggttctgaattggttttccaattggctactccagttaacttcgcttctgaagacccagaaaacgacatgattaagccagctattaagggtgttttgaacgttttgaagtcttgtgctagagctaaggttaagagagttattttgacttcttctgctgcttctgttactattggtgaattgaagggtactgacttggttatggacgaatctaactggactgacgttgaattcttgtctaacgctaagccaccaacttggggttacccagcttctaagactttggctgaaaaggctgcttggaagttcgctgaagaaaaccacattgacttgattactgttattccatctttgattactggtccatctttgactccagacattccatcttctgttggtttggctacttctttgattactggtaacgacttcttgattaacgctatgaagggtatgcaattgttgtctggttctatttctattactcacgttgaagacgtttgtagagctcacattttcgttgctgaaaagcaatctgcttctggtagatacatttgttgtgctcacaacacttctgttccagaattggctaagttcttgaacaagagatacccacaatacaaggtttctactgaattcaacgacttcccagctaaggctaagttgattatttctccagaaaagttgattaaggaaggtttctctttcaagtacggtgttgaagaaattttcgaccaaactttggaatacttgaagactaagggtgctttgaagaactaa

SEQ ID NO: 52 - protein, 338 aa, Lotus corniculatus
MASIKPIYEKKKACVIGGTGFVASLLIKQLLEKGYAVNTTVRDPDNHKKISHLLVLQSLGELNIFGGELTVENDFDTPIEGSELVFQLATPVNFASEDPENDMIKPAIKGVLNVLKSCARAKVKRVILTSSAASVTIGELKGTDLVMDESNWTDVEFLSNAKPPTWGYPASKTLAEKAAWKFAEENHIDLITVIPSLITGPSLTPDIPSSVGLATSLITGNDFLINAMKGMQLLSGSISITHVEDVCRAHIFVAEKQSASGRYICCAHNTSVPELAKFLNKRYPQYKVSTEFNDFPAKAKLIISPEKLIKEGFSFKYGVEEIFDQTLEYLKTKGALKN

SEQ ID NO: 53 - DNA, 1293 bp, Petunia x hybrida
  source 1..1293: mol_type = genomic DNA
atggttaacgccgttgttactaccccatctagagttgaatctttggctaagtctggtattcaagccatcccaaaagaatacgttagaccacaagaagaattgaacggtatcggtaacattttcgaagaagaaaagaaagacgaaggtccacaagttccaaccatcgatttgaaagaaatcgactccgaagacaaagaaatcagagaaaagtgccaccaattgaaaaaggctgctatggaatggggtgttatgcatttggttaatcacggtatctccgacgaattgatcaacagagttaaggttgctggtgaaacctttttcgatcaaccagtcgaagaaaaagaaaagtacgctaacgatcaagccaacggtaatgttcaaggttacggttctaaattggctaactctgcttgtggtcaattggaatgggaagattactttttccattgcgctttcccagaagataagagagatttgtctatctggccaaagaacccaactgattatactccagctacttctgaatacgccaagcaaattagagctttggctactaagattttgaccgtcttgtctattggtttgggtttggaagaaggtagattggaaaaagaagttggtggtatggaagatttgttgttgcaaatgaagatcaactactacccaaagtgtccacaaccagaattggctttgggtgttgaagctcatactgatgtttctgctttgaccttcatcttgcataatatggtcccaggtttacaattattctacgaaggtcaatgggttaccgctaagtgtgttccaaattccattatcatgcatatcggtgacaccatcgaaatcttgtctaacggtaaatacaagtccatcttgcacagaggtgttgtcaacaaagaaaaggttagattctcctgggctattttctgtgaaccacctaaagaaaagatcatcttgaagccattgccagaaactgttactgaagctgaaccaccaagatttccaccaagaacttttgctcaacatatggcccataagttgttcagaaaggatgataaggatgctgccgttgaacataaggttttcaacgaagatgaattggatactgctgctgaacacaaagtcttgaagaaggataatcaagacgctgttgctgaaaacaaggacatcaaagaagatgaacaatgtggtccagcagaacacaaagatatcaaagaagatggtcaaggtgctgctgcagaaaacaaggttttcaaagaaaacaatcaagatgtcgccgccgaagaatctaagtaa

SEQ ID NO: 54 - protein, 430 aa, Petunia x hybrida
MVNAVVTTPSRVESLAKSGIQAIPKEYVRPQEELNGIGNIFEEEKKDEGPQVPTIDLKEIDSEDKEIREKCHQLKKAAMEWGVMHLVNHGISDELINRVKVAGETFFDQPVEEKEKYANDQANGNVQGYGSKLANSACGQLEWEDYFFHCAFPEDKRDLSIWPKNPTDYTPATSEYAKQIRALATKILTVLSIGLGLEEGRLEKEVGGMEDLLLQMKINYYPKCPQPELALGVEAHTDVSALTFILHNMVPGLQLFYEGQWVTAKCVPNSIIMHIGDTIEILSNGKYKSILHRGVVNKEKVRFSWAIFCEPPKEKIILKPLPETVTEAEPPRFPPRTFAQHMAHKLFRKDDKDAAVEHKVFNEDELDTAAEHKVLKKDNQDAVAENKDIKEDEQCGPAEHKDIKEDGQGAAAENKVFKENNQDVAAEESK

SEQ ID NO: 55 - DNA, 699 bp, Petunia x hybrida
  source 1..699: mol_type = genomic DNA
atggttgttaaggtccacggttctgctatggctgcttgtccacaaagagttatggtttgcttgattgaattgggtgtggacttcgaattgatccacgttgatttggattccttggaacaaaagaagccagagtttttggtcttgcaaccatttggtcaagttccagttattgaagatggtgactttaggttgttcgaatccagagctattattaggtactacgctgctaagtacgaagtcaaaggttctaaattgactggtactaccttggaagaaaaggctttggttgatcaatggttggaagtcgaatctaacaactacaacgatctggtttacaacatggtcttgcagttgttggtttttccaaagatgggtcaaacttccgatttgaccttggttacaaaatgcgctaacaagttggaaaacgtgttcgacatctacgaacagagattgtctaagtctaaatacttggctggtgagttcttctctttggctgatttgtctcatttgccatccttgagattcttgatgaatgaaggtggtttctcccacttggttactaagagaaagtgtttacacgaatggtacttggacatctcctcaagagattcttggaaaaaggttctggacctgatgatgaagaagatttccgaaattgaagccgtttccattccagctaaagaagaagctaaggtctga

SEQ ID NO: 56 - protein, 232 aa, Petunia x hybrida
MVVKVHGSAMAACPQRVMVCLIELGVDFELIHVDLDSLEQKKPEFLVLQPFGQVPVIEDGDFRLFESRAIIRYYAAKYEVKGSKLTGTTLEEKALVDQWLEVESNNYNDLVYNMVLQLLVFPKMGQTSDLTLVTKCANKLENVFDIYEQRLSKSKYLAGEFFSLADLSHLPSLRFLMNEGGFSHLVTKRKCLHEWYLDISSRDSWKKVLDLMMKKISEIEAVSIPAKEEAKV

SEQ ID NO: 57 - DNA, 1380 bp, Dianthus caryophyllus
  source 1..1380: mol_type = genomic DNA
atgtccgccaactccaactacatgaacaagtctagattgcatgttgccgtttttccatttccatttggtactcatgctacccctttgttcaacattactcaaaagttggcttccttcatgccagatgttgttttctccttcttcaacatcccacaatccaacgctaagatttcctctgattttaagaacgacaccatcaacatgtacgatgtttgggatggtgttccagaaggttacgtttttaaaggtaagccacaagaagatatcgaattattcatgttggctgctccaccaactttgactgaagccttggctaaagctgaagttgaaactggtacaaaggtttcctgtattttgggtgatgctttcttgtggttcttggaagaattggctcaacaaaaacaagttccatggattaccacctacatgtccgaagaacattctttgttggctcatatctgcaccgatttgattagacaaaccattggtattcacgaaaaggccgaagaaagaaaggatgaagaattagatttcatcccaggtttgtccaagatcagagttcaagatttgcctgaaggtatcgttatgggtaacttggattcttacttcgccagaatgttgcatcaaatgggtagagctttgccaagagcatctgctgtatgtatttctagttgccaagaattggatccagttgctaccaatgaattgaacagaaagttgaacaagttgattaacgttggtccattgtccttgattacccaatcaaattctttgccatccggtacaaacaaatctttgggttggttggataagcaagaatccgaaaattctgttgcctacgtttcttttggttcagttgctagaccagatgctactgaaattactgctttggctcaagccttagaagctagtcaagttaagttcatctggtccatcagagataacttgaaggttcatttgccaggtggtttcattgaaaacactaaggataagggtatggttgtttcttgggttccacaaactgctgttttagctcataaggctgttggtgttttcattacccatttcggtcataactccattatggaatccattgcttctgaagttccaatgattggtagaccattcatcggtgaacaaaaattgaacggtagaatcgttgaagccaagtggtgtattggtttggttgttgaaggtggtgtttttactaaggatggtgtcttgagatccttgaacaagattttgggttctacccaaggtgaagaaatgagaagaaacatcagagacttgagattgatggttgataaggctttgtctccagatggttcttgtaacaccaacttgaaacacttggttgatatgatcgtcacctccaactaa

SEQ ID NO: 58 - protein, 459 aa, Dianthus caryophyllus
MSANSNYMNKSRLHVAVFPFPFGTHATPLFNITQKLASFMPDVVFSFFNIPQSNAKISSDFKNDTINMYDVWDGVPEGYVFKGKPQEDIELFMLAAPPTLTEALAKAEVETGTKVSCILGDAFLWFLEELAQQKQVPWITTYMSEEHSLLAHICTDLIRQTIGIHEKAEERKDEELDFIPGLSKIRVQDLPEGIVMGNLDSYFARMLHQMGRALPRASAVCISSCQELDPVATNELNRKLNKLINVGPLSLITQSNSLPSGTNKSLGWLDKQESENSVAYVSFGSVARPDATEITALAQALEASQVKFIWSIRDNLKVHLPGGFIENTKDKGMVVSWVPQTAVLAHKAVGVFITHFGHNSIMESIASEVPMIGRPFIGEQKLNGRIVEAKWCIGLVVEGGVFTKDGVLRSLNKILGSTQGEEMRRNIRDLRLMVDKALSPDGSCNTNLKHLVDMIVTSN

SEQ ID NO: 59 - DNA, 1392 bp, Fragaria x ananassa
  source 1..1392: mol_type = genomic DNA
atggcctctaatcaagctggtggtcatgttgctgttttggcttttccattttctactcatgctgcccctttgttgaatattgtctgtagattggctgctgctgctccatctactttgttttcttttttcaacaccaaacaatccaactcctccattttggcttcagatacttctgttttgagatacaccaacgtttgcgtttgtgaagttgctgatggtgttccagaaggttacgtttttgttggtaaaccacaagaagatatcgaattattcatgaaggctgccccagataacttcagaaaatgtttggaagctagtgttgccgaatctggtagagaagtttcttgtttggttaccgatgcttttttttggttcggtgctcatatggctgatgatatgggtggtgttccttgggttcctttttggactgctggtccagcttctttgtctgctcatgttcatactgatttgatcagaaacactacctctggtgattgccatgacgaaaaagaaaccattactgttattgccggtatgtccaaagttagaccacaagatttgccagaaggtatcattttcggtaacttggaatccttgttctccagaatgttgcatcaaatgggtttgatgttgccattagctactgccgtttttatcaactccttcgaagaattggatccagttatcaccaacgacttgaagtctaagttcaagagatttttgaacgtcggtccattggatttgttggaacctactgcttctgctgctactactacaccacaaactgctgaagctgttgctggtgatggttgtttgtcttggttggataagcaaaaagctgcctctgttgtttacgtttctttcggttctgttactagaccatctcctgaagaattgatggctttggctgaagccttggaagcctctagagttccatttttgtggtctttgagagacaacttgaagaacccacaattggacgaatttttgtccaagggtaaattgaacggtatggttgttccatgggctccacaaccacaagttttggctcatggttctgttggtgcttttgttactcattgtggttggaactctgttttggaatctgttgccggtggtgtaccattgatatgtagaccatttttcggtgaccaaaagttgaacgctagaatggttgaagatgtctggaagattggtttgagattggaaggtggtgttttcaccaagaatggtatgttgaagtccttggacatgttgttgtctcaagataagggtactaagatgaagaacaagatccacaccttgaagcaattggctcaacaagctgttgaacctaaaggttcttctaccagaaacttcgaatctttgttagaaatggctaccaccaactaa

SEQ ID NO: 60 - protein, 463 aa, Fragaria x ananassa
MASNQAGGHVAVLAFPFSTHAAPLLNIVCRLAAAAPSTLFSFFNTKQSNSSILASDTSVLRYTNVCVCEVADGVPEGYVFVGKPQEDIELFMKAAPDNFRKCLEASVAESGREVSCLVTDAFFWFGAHMADDMGGVPWVPFWTAGPASLSAHVHTDLIRNTTSGDCHDEKETITVIAGMSKVRPQDLPEGIIFGNLESLFSRMLHQMGLMLPLATAVFINSFEELDPVITNDLKSKFKRFLNVGPLDLLEPTASAATTTPQTAEAVAGDGCLSWLDKQKAASVVYVSFGSVTRPSPEELMALAEALEASRVPFLWSLRDNLKNPQLDEFLSKGKLNGMVVPWAPQPQVLAHGSVGAFVTHCGWNSVLESVAGGVPLICRPFFGDQKLNARMVEDVWKIGLRLEGGVFTKNGMLKSLDMLLSQDKGTKMKNKIHTLKQLAQQAVEPKGSSTRNFESLLEMATTN

SEQ ID NO: 61 - DNA, 1098 bp, Medicago sativa
  source 1..1098: mol_type = genomic DNA
atgggttcaacaggtgaaactcaaataacaccaacccacatatcagatgaagaagcaaacctcttcgccatgcaactagcaagtgcttcagttcttcccatgattttgaaatcagctcttgaacttgatctcttagaaatcattgctaaagctggacctggtgctcaaatttcacctattgaaattgcttctcagctaccaacaactaaccctgatgcaccagttatgttggaccgaatgttgcgtctcttggcttgttacataatcctcacatgttcagttcgtactcaacaagatggaaaggttcagagactttatggtttggctactgttgctaagtatttggttaagaatgaagatggtgtatccatttctgctcttaatctcatgaatcaggataaagtgctcatggaaagctggtaccacctaaaagatgcagtccttgatgggggcattccattcaacaaggcttatggaatgacagcctttgaataccatggaacagatccaaggtttaacaaggttttcaacaaggggatgtctgatcactctaccatcacaatgaagaaaattcttgagacctacacaggttttgaaggccttaaatctcttgttgatgtaggtggtggtactggagctgtaattaacacgattgtctcaaaatatcccactataaagggtataaattttgatttaccccatgtcattgaagatgctccatcttatccaggagttgagcatgttggtggagacatgtttgtcagtattccaaaggctgatgctgtttttatgaagtggatttgtcatgactggagtgatgagcactgcttgaaatttttgaagaactgctatgaggcactgccagacaatggaaaagtgattgtggcagaatgcatacttccagtggctccagattcaagcctggccacaaaaggtgtggttcacattgatgtgatcatgttggctcataatcctggtgggaaagagagaacacaaaaagagtttgaggatcttgccaaaggtgctggattccaaggtttcaaagtccattgtaatgctttcaacacatacatcatggagtttcttaagaaggtttaa

SEQ ID NO: 62 - protein, 365 aa, Medicago sativa
MGSTGETQITPTHISDEEANLFAMQLASASVLPMILKSALELDLLEIIAKAGPGAQISPIEIASQLPTTNPDAPVMLDRMLRLLACYIILTCSVRTQQDGKVQRLYGLATVAKYLVKNEDGVSISALNLMNQDKVLMESWYHLKDAVLDGGIPFNKAYGMTAFEYHGTDPRFNKVFNKGMSDHSTITMKKILETYTGFEGLKSLVDVGGGTGAVINTIVSKYPTIKGINFDLPHVIEDAPSYPGVEHVGGDMFVSIPKADAVFMKWICHDWSDEHCLKFLKNCYEALPDNGKVIVAECILPVAPDSSLATKGVVHIDVIMLAHNPGGKERTQKEFEDLAKGAGFQGFKVHCNAFNTYIMEFLKKV

SEQ ID NO: 63 - DNA, 699 bp, Arabidopsis thaliana
  source 1..699: mol_type = genomic DNA
atggctaaggatgaagccaagggattgctgaaaagtgaagagctctataaatatatactggagactagtgtgtacccacgcgagccagaggttctcagggagcttaggaacattactcataaccaccctcaagctggtatggctactgcaccagacgctggccagttgatggggatgctgctgaatctggtaaatgcaagaaagactatcgaggttggggttttcactggatactctcttctcctcactgctcttacgctaccagaagatggcaaggttatcgccattgacatgaatagagactcgtatgaaataggattgccggttataaagaaagctggtgttgaacacaaaattgatttcaaagagtctgaagctcttccagccctcgacgagcttttaaacaataaagtgaatgagggtggattcgacttcgcatttgtggatgcagacaagctgaattattggaactatcatgagaggcttattagattgatcaaggttggtgggatcatagtgtatgataacactctctggggaggatcagttgctgaaccggactcgtccacgcccgagtggaggatagaagtcaagaaagcaaccctcgagctgaacaagaaactctcggctgatcagcgtgtgcagatctcgcaagctgcgcttggcgatggtatcactatttgcaggaggttatattga

SEQ ID NO: 64 - protein, 232 aa, Arabidopsis thaliana
MAKDEAKGLLKSEELYKYILETSVYPREPEVLRELRNITHNHPQAGMATAPDAGQLMGMLLNLVNARKTIEVGVFTGYSLLLTALTLPEDGKVIAIDMNRDSYEIGLPVIKKAGVEHKIDFKESEALPALDELLNNKVNEGGFDFAFVDADKLNYWNYHERLIRLIKVGGIIVYDNTLWGGSVAEPDSSTPEWRIEVKKATLELNKKLSADQRVQISQAALGDGITICRRLY

SEQ ID NO: 65 - DNA, 1527 bp, Arabidopsis thaliana
  source 1..1527: mol_type = genomic DNA
atgtcgtggtttctaatagcggtggcgacaatcgccgccgtcgtatcctacaagctaatccaacggctaagatacaagttcccaccaggcccaagccccaagccgatcgtcggtaacctctacgacataaaaccggtccggttcagatgttactacgagtgggctcaatcttatggaccaatcatatcggtctggatcggttcaattctaaacgtggtcgtatctagcgccgagctagcaaaagaagttctgaaagaacacgaccagaaactcgccgaccggcaccggaacagatcgacggaagcatttagccgcaacggtcaggatcttatatgggccgattatgggcctcattacgtgaaggtgagaaaagtttgcacgcttgagctcttcacaccgaaacgactcgagtctctcagacctatccgtgaagatgaagtcaccgccatggttgaatccgtcttcagagactgtaaccttcctgaaaacagagcaaaaggtttacaactgaggaagtacttaggagcggttgcgttcaacaacataacgcggctagcctttgggaagcgttttatgaacgctgaaggtgttgtggacgagcaagggcttgagttcaaggccatagtatccaacggtctgaagctaggtgcttcactgtcaatagctgaacacatcccgtggctcaggtggatgtttccggctgatgagaaggcgtttgctgagcacggggctcgtcgtgaccgcctcactcgagctatcatggaggagcatactttggcccgtcaaaagtctagtggagcgaaacagcatttcgttgatgcgttgctaacgttgaaggatcagtatgatcttagtgaggatactatcattggtcttctatgggatatgatcacggcagggatggacacgacagcgataacagcggaatgggcgatggcggaaatgatcaagaatccaagagtgcaacaaaaagtgcaagaagagttcgacagagtggttggacttgaccggatcttaaccgaggcagatttctcccgcttaccttacttgcaatgcgtggtgaaagagtcattcaggctgcatcctccaacgcctctaatgctacctcaccgaagcaacgcagatgtcaagatcggaggctatgatattcccaaaggatcaaacgttcatgtgaatgtgtgggctgtggctagagacccggctgtatggaaaaatccatttgagtttagaccagagagattcttggaagaagatgttgacatgaagggtcatgattttaggctgcttccgtttggagctggaagacgggtttgtcccggtgcacaacttggtatcaatttggtaacttcgatgatgagtcatttgcttcaccattttgtttggacacctcctcaagggactaaaccggaggagattgacatgtctgaaaaccctggactcgttacttacatgcgtacccctgtgcaagcggttgcaacgcctcggttgccttcggatctgtacaaacgcgtgccttacgatatgtaa

SEQ ID NO: 66 - protein, 508 aa, Arabidopsis thaliana
MSWFLIAVATIAAVVSYKLIQRLRYKFPPGPSPKPIVGNLYDIKPVRFRCYYEWAQSYGPIISVWIGSILNVVVSSAELAKEVLKEHDQKLADRHRNRSTEAFSRNGQDLIWADYGPHYVKVRKVCTLELFTPKRLESLRPIREDEVTAMVESVFRDCNLPENRAKGLQLRKYLGAVAFNNITRLAFGKRFMNAEGVVDEQGLEFKAIVSNGLKLGASLSIAEHIPWLRWMFPADEKAFAEHGARRDRLTRAIMEEHTLARQKSSGAKQHFVDALLTLKDQYDLSEDTIIGLLWDMITAGMDTTAITAEWAMAEMIKNPRVQQKVQEEFDRVVGLDRILTEADFSRLPYLQCVVKESFRLHPPTPLMLPHRSNADVKIGGYDIPKGSNVHVNVWAVARDPAVWKNPFEFRPERFLEEDVDMKGHDFRLLPFGAGRRVCPGAQLGINLVTSMMSHLLHHFVWTPPQGTKPEEIDMSENPGLVTYMRTPVQAVATPRLPSDLYKRVPYDM

SEQ ID NO: 67 - DNA, 1302 bp, Arabidopsis thaliana
  source 1..1302: mol_type = genomic DNA
atgaaaattaacatcagagattccaccatggtccggcctgccaccgagacaccaatcactaatctttggaactccaacgtcgaccttgtcatccccagattccatacccctagtgtctacttctacagacccaccggcgcttccaatttctttgaccctcaggtcatgaaggaagctctttccaaagcccttgtccctttttaccctatggctggtcgcttgaagagagacgatgatggtcgtattgagatcgattgtaacggtgctggtgttctcttcgttgtggctgatactccttctgttatcgatgattttggtgattttgctcctacccttaatctccgtcagcttattcccgaagttgatcactccgctggcattcactctttcccgcttctcgttttgcaggtgactttctttaaatgtgggggagcttcacttggggttgggatgcaacatcacgcggcagatggtttctctggtcttcattttatcaacacatggtctgatatggctcgtggtcttgacctaaccattccacctttcattgatcgaacactcctccgagctagggacccgccacagcctgcttttcatcatgttgaatatcagcctgcaccaagtatgaagatacctcttgatccgtctaaatcaggacctgagaataccactgtctctatattcaaattaacacgagaccagcttgttgctcttaaggcgaaatccaaggaggatgggaacactgtcagctacagctcatacgagatgttggcagggcatgtgtggagatcagtgggaaaggcgcgagggcttccaaacgaccaagagacgaaactgtacattgcaactgatggaaggtctagactacgtccgcagctgcctcctggttactttgggaatgtgatattcactgcaacaccattggctgttgcaggggatttgttatctaagccaacatggtatgctgcaggacagattcatgatttcttggttcgtatggatgataactatctgaggtcagctcttgactacctggagatgcagcctgatctgtcagcccttgtccgcggtgcacatacctacaagtgcccaaatttgggaatcacaagctgggttagattacctatttatgatgcagactttggttggggtcgtcctatctttatgggacctggtggaattccatacgagggtttgtcttttgtgctaccaagtcctactaatgatggcagcttatccgttgccattgccctccaatctgaacacatgaaactgtttgagaagtttttgtttgagatatga

SEQ ID NO: 68 - protein, 433 aa, Arabidopsis thaliana
MKINIRDSTMVRPATETPITNLWNSNVDLVIPRFHTPSVYFYRPTGASNFFDPQVMKEALSKALVPFYPMAGRLKRDDDGRIEIDCNGAGVLFVVADTPSVIDDFGDFAPTLNLRQLIPEVDHSAGIHSFPLLVLQVTFFKCGGASLGVGMQHHAADGFSGLHFINTWSDMARGLDLTIPPFIDRTLLRARDPPQPAFHHVEYQPAPSMKIPLDPSKSGPENTTVSIFKLTRDQLVALKAKSKEDGNTVSYSSYEMLAGHVWRSVGKARGLPNDQETKLYIATDGRSRLRPQLPPGYFGNVIFTATPLAVAGDLLSKPTWYAAGQIHDFLVRMDDNYLRSALDYLEMQPDLSALVRGAHTYKCPNLGITSWVRLPIYDADFGWGRPIFMGPGGIPYEGLSFVLPSPTNDGSLSVAIALQSEHMKLFEKFLFEI

SEQ ID NO: 69 - DNA, 1518 bp, Delphinium grandiflorum
  source 1..1518: mol_type = genomic DNA
atgtgcccctcttttctagtgactcttcttcttctccagcttagtagtctagtggtggtactagttgtttgggcagaacaattgcccgaatttaatgtcagaagagatgatttcccatctaactttgtgttcggcgctggtacttcagctcttcaggttgaaggggccattgcagaagatggaaaaacacctaatatctgggacgtcgacagtcatatggggcatatgccggacaagagcaccacagatatagcttgcgattcataccacagatataaggaagatgtgaagataatgagtgatataggactcgaagcttatcgattttccattgcatggaccagaattcttccatatgggagaggattcatcaatccaaaaggggtcgagtattataacaatctcatcgacacactgttggaacatggaattcaaccacatgctacaatataccatatagatcaccctcagatacttgaagatgaatacggaggatggttaagcccgagaatgatcgaggacttcaccacctacgcagatgtatgttttagagagtttggcgacagggtttcgcactggacaaccatcaacgaacctaacataataagcttgggggcttacgacagtggtcagattccaccccatcgatgtacgcccccaggtgcctacaactgtacagctggtaactcatccgtcgaaccttacaaagcaatgcaccatttcttgctagcccatgcgtctgctgtgcaaatctacaggacaaaataccaggctaagcagaaaggtttgatagggctcaatgtgtacggtttctggtgtgcacctcagacaaactcgagggcagatattgaagcaaccaaaagagccacagctttctacactggctgggctgctgatcccctcgttttcggggactatccaataatcatgaaagagaatgttggctccagacttccatcgtttaccaagaacgagtccgagcttgtaaaagggtcatttgactttattgggctgaaccattatttcgtattttacatacaagatgaccctgaagagattacaacaccaattagcctgagaaatttcgatagtgacatgcgtgtcaaagcttcagttaagccaggggactctggtgatccatctggtttaaagaacctgcttcgatatttcaaggacaactatggaaatcctcctgtctatgttcatgagaatggatttgggtcgccgcaaaatgagacacttgatgacgacatgggcaggatccggtacataagcggctacataggaagcatgcttgaagcaatcaagaatggatcagacacaagaggatattttgtgtggtcgttcatggacgcttttgagattctaagtggataccaaacccggtatgggattgttcacgtcgactttgatgataagagtttgaagagacagctcaagccatcagcccagtggtattccaacttcatcaagaagaagaacacaacagaggatgagatatcgtactcttcacagtag

SEQ ID NO: 70 - protein, 505 aa, Delphinium grandiflorum
MCPSFLVTLLLLQLSSLVVVLVVWAEQLPEFNVRRDDFPSNFVFGAGTSALQVEGAIAEDGKTPNIWDVDSHMGHMPDKSTTDIACDSYHRYKEDVKIMSDIGLEAYRFSIAWTRILPYGRGFINPKGVEYYNNLIDTLLEHGIQPHATIYHIDHPQILEDEYGGWLSPRMIEDFTTYADVCFREFGDRVSHWTTINEPNIISLGAYDSGQIPPHRCTPPGAYNCTAGNSSVEPYKAMHHFLLAHASAVQIYRTKYQAKQKGLIGLNVYGFWCAPQTNSRADIEATKRATAFYTGWAADPLVFGDYPIIMKENVGSRLPSFTKNESELVKGSFDFIGLNHYFVFYIQDDPEEITTPISLRNFDSDMRVKASVKPGDSGDPSGLKNLLRYFKDNYGNPPVYVHENGFGSPQNETLDDDMGRIRYISGYIGSMLEAIKNGSDTRGYFVWSFMDAFEILSGYQTRYGIVHVDFDDKSLKRQLKPSAQWYSNFIKKKNTTEDEISYSSQ

SEQ ID NO: 71 - DNA, 1182 bp, Pinus densiflora
  source 1..1182: mol_type = genomic DNA
atgggtggtgttgacttcgaaggtttcagaaagttgcaaagagctgacggtttcgcttctattttggctattggtactgctaacccaccaaacgctgttgaccaatctacttacccagactactacttcagaattactggtaacgaacacaacactgaattgaaggacaagttcaagagaatttgtgaaagatctgctattaagcaaagatacatgtacttgactgaagaaattttgaagaagaacccagacgtttgtgctttcgttgaagttccatctttggacgctagacaagctatgttggctatggaagttccaagattggctaaggaagctgctgaaaaggctattcacgaatggggtcaatctaagtctggtattactcacttgattttctgttctactactactccagacttgccaggtgctgacttcgaagttgctaagttgttgggtttgcacccatctgttaagagagttggtgttttccaacacggttgtttcgctggtggtactgttttgagattggctaaggacttggctgaaaacaacagaggtgctagagttttggttatttgttctgaaactactgctgttactttcagaggtccatctgaaactcacttggactctttggttggtcaagctttgttcggtgacggtgcttctgctttgattgttggtgctgacccaattccacaagttgaaaaggcttgtttcgaaattgttagaacttctcaaactgttgttccaaactctgacggtgctattggtggtaaggttagagaagttggtttgactttccaattgaagggtgctgttccagacttgatttctgctaacattgaaaactgtttggttgaagctttctctcaattcaagatttgtgactggaacaagttgttctgggttgttcacccaggtggtagagctattttggacagagttgaagctaagttgaacttggacccaactaagttgattccaactagacacgttatgtctgaatacggtaacatgtcttctgcttgtgttcacttcattttggacgaaactagaaaggcttctttgagaaacggttgttctactactggtgaaggtttggaaatgggtgttttgttcggtttcggtccaggtttgactattgaaactgttgttttgaagtctgttccattgcaatag

SEQ ID NO: 72 - protein, 393 aa, Pinus densiflora
MGGVDFEGFRKLQRADGFASILAIGTANPPNAVDQSTYPDYYFRITGNEHNTELKDKFKRICERSAIKQRYMYLTEEILKKNPDVCAFVEVPSLDARQAMLAMEVPRLAKEAAEKAIHEWGQSKSGITHLIFCSTTTPDLPGADFEVAKLLGLHPSVKRVGVFQHGCFAGGTVLRLAKDLAENNRGARVLVICSETTAVTFRGPSETHLDSLVGQALFGDGASALIVGADPIPQVEKACFEIVRTSQTVVPNSDGAIGGKVREVGLTFQLKGAVPDLISANIENCLVEAFSQFKICDWNKLFWVVHPGGRAILDRVEAKLNLDPTKLIPTRHVMSEYGNMSSACVHFILDETRKASLRNGCSTTGEGLEMGVLFGFGPGLTIETVVLKSVPLQ

SEQ ID NO: 73 - DNA, 1179 bp, Vitis vinifera
  source 1..1179: mol_type = genomic DNA
atggcttctgttgaagaaattagaaacgctcaaagagctaagggtccagctactattttggctattggtactgctactccagaccactgtgtttaccaatctgactacgctgactactacttcagagttactaagtctgaacacatgactgctttgaagaagaagttcaacagaatttgtgacaagtctatgattaagaagagatacattcacttgactgaagaaatgttggaagaacacccaaacattggtgcttacatggctccatctttgaacattagacaagaaattattactgctgaagttccaaagttgggtaaggaagctgctttgaaggctttgaaggaatggggtcaaccaaagtctaagattactcacttggttttctgtactacttctggtgttgaaatgccaggtgctgactacaagttggctaacttgttgggtttggaaccatctgttagaagagttatgttggaccaacaaggttgttacgctggtggtactgttttgagaactgctaaggacttggctgaaaacaacgctggtgctagagttttggttgtttgttctgaatctactgttgttactttcagaggtccatctgaagacgctttggactctttggttggtcaagctttgttcggtgacggttctgctgctgttattgttggttctgacccagacatttctattgaaagaccattgttccaattggtttctgctgctcaaactttcattccaaactctgctggtgctattgctggtaacttgagagaagttggtttgactttccacttgtggccaaacgttccaactttgatttctgaaaacattgaaaagtgtttgactcaagctttcgacccattgggtatttctgactggaactctttgttctggattgctcacccaggtggtccagctattttggacgctgttgaagctaagttgaacttggacaagaagaagttggaagctactagacacgttttgtctgaatacggtaacatgtcttctgcttgtgttttgttcattttggacgaaatgagaaagaagtctttgaagggtgaaagagctactactggtgaaggtttggactggggtgttttgttcggtttcggtccaggtttgactattgaaactgttgttttgcactctattccaatggttactaactaa

SEQ ID NO: 74 - protein, 392 aa, Vitis vinifera
MASVEEIRNAQRAKGPATILAIGTATPDHCVYQSDYADYYFRVTKSEHMTALKKKFNRICDKSMIKKRYIHLTEEMLEEHPNIGAYMAPSLNIRQEIITAEVPKLGKEAALKALKEWGQPKSKITHLVFCTTSGVEMPGADYKLANLLGLEPSVRRVMLDQQGCYAGGTVLRTAKDLAENNAGARVLVVCSESTVVTFRGPSEDALDSLVGQALFGDGSAAVIVGSDPDISIERPLFQLVSAAQTFIPNSAGAIAGNLREVGLTFHLWPNVPTLISENIEKCLTQAFDPLGISDWNSLFWIAHPGGPAILDAVEAKLNLDKKKLEATRHVLSEYGNMSSACVLFILDEMRKKSLKGERATTGEGLDWGVLFGFGPGLTIETVVLHSIPMVTN

SEQ ID NO: 75 - DNA, 1125 bp, Sorghum bicolor
  source 1..1125: mol_type = genomic DNA
atggtactcatcagcgaggacagtagggagttgctccaagcccacgtcgagctatggaaccagacctacagctttatgaagtcggtggcgctcgccgttgctttagacctccgcatcgctgatgccatccaccgctgcggtggcgccgccaccctctcccagatactcggagagattggtgtccgcccatgtaagcttcccgccctccaccgcctaatgcgtgttctgaccgtctcaggaaccttcaccatcgtccagccatcagcggcaaccatgtcattggtgtcggacgggaatgagcttgtctataagctgacaacagcgtcccgcctcctcgtcagcagcgaaagctcggcgacggcgagcttgtctcctatgctgaaccacgtgcttagccccttccgtgactcgcccctcagcatggggctcactgcgtggttccggcacgatgaagatgaacaggcgcctggcccatgcccgttcaccctgatgtacggcacaaccttgtgggaggtgtgcagtcgtgacgacgcaatcaacgcgttgttcaacaacgccatggccgcagacagcaacttcctgatgcagattgtgttgagggagttcggcaaggtcttccacgggatagactcgctggtcgacgtcggcggtggggttgggggagccaccatggccattgccacggcgttcccgtctttgaagtgtaccgtactagacctccctcacgttgtcgccaaggctccgtccagttctattggcaacgtgcagtttgttgggggtgacatgtttgagagcattccaccagcgaatgttgtcttcctcaagtggattttgcatgactggagcaatgacgagtgtatcaagatattaaagaactgcaagcaagctatcccttctagagatgcaggaggaaagataataatcattgatgttgtggttgggtctgagtcatcagacaccaagcttctggagacacaagtaatgtatgatctccatctcatgaaaattggtggggttgaacgagatgagcaagagtggaagaaaatattcctcgaagctggatttaaggactacaatattataccagttttaggcctccgatcgatcattgagctatatccatga

SEQ ID NO: 76 - protein, 374 aa, Sorghum bicolor
MVLISEDSRELLQAHVELWNQTYSFMKSVALAVALDLRIADAIHRCGGAATLSQILGEIGVRPCKLPALHRLMRVLTVSGTFTIVQPSAATMSLVSDGNELVYKLTTASRLLVSSESSATASLSPMLNHVLSPFRDSPLSMGLTAWFRHDEDEQAPGPCPFTLMYGTTLWEVCSRDDAINALFNNAMAADSNFLMQIVLREFGKVFHGIDSLVDVGGGVGGATMAIATAFPSLKCTVLDLPHVVAKAPSSSIGNVQFVGGDMFESIPPANVVFLKWILHDWSNDECIKILKNCKQAIPSRDAGGKIIIIDVVVGSESSDTKLLETQVMYDLHLMKIGGVERDEQEWKKIFLEAGFKDYNIIPVLGLRSIIELYP

SEQ ID NO: 77 - DNA, 1383 bp, Dahlia variabilis
  source 1..1383: mol_type = genomic DNA
atggacaacatcccaaacttgaccatcttggaacactctagaatttctccaccaccatctactattggtcatagatcattgccattgactttcttcgatattgcctggttgttgtttccaccagttcatcacttgtacttctaccattttccatactccaagtcccatttcaccgaaactgttatcccaaatttgaagcactccttgtccattaccttgcaacattactttccattcgtcggtaagttgatcgtttacccaaatccacatgattctaccagaaagccagaaatcagacatgttgaaggtgattctgttgctttgactttcgctgaaactaccttggatttcaacgatttgtctgctaaccatccaagaaagtgcgaaaatttctatccattggttccaccattgggtaacgctgtaaaagaatctgattacgttaccttgccagttttctctgttcaagttacttacttcccaaactccggtatttctattggtttgaccaaccatcactctttgtctgatgctaacactagattcggttttttgaaggcttgggcttctgtttgtgaaactggtgaagatcaaccattcttgaaaaatggttccccaccagttttcgatagagttgttgttaacccacaattatacgaaaacagattgaatcaaaccagattgggtactttctaccaagctccttctttggttggttcttcatctgatagagttagagctactttcgttttggccagaactcatatttccggtttgaagaagcaagtcttgactcaattgccaatgttggaatacacttcttctttcactgttacctgcggttacatttggtcttgtatcgttaagtccttggtcaacatgggtgaaaaaaagggtgaagatgaattggaacaattcatcgtttctgtcggttgcagatcaagattagatccaccattaccagaaaactacttcggtaactgttctgctccatgtattgtcactattaagaacggtgtcttgaagggtgaaaacggttttgttatggctgctaagttgattggtgaaggtatctctaagatggttaacaagaagggtggtattttggaatacgctgatagatggtacgatggttttaagattccagctagaaagatgggtatttctggtactccaaagttgaacttctacgatattgatttcggttggggtaaggctatgaagtacgaagttgtttctattgactactccgcctctgtttctttgtcagcttgtaaagaatccgcccaagattttgaaattggtgtttgtttcccatccatgcaaatggaagcctttggtaaaatctttaacgacggtttggaatccgctattgcttcttaa

SEQ ID NO: 78 - protein, 460 aa, Dahlia variabilis
MDNIPNLTILEHSRISPPPSTIGHRSLPLTFFDIAWLLFPPVHHLYFYHFPYSKSHFTETVIPNLKHSLSITLQHYFPFVGKLIVYPNPHDSTRKPEIRHVEGDSVALTFAETTLDFNDLSANHPRKCENFYPLVPPLGNAVKESDYVTLPVFSVQVTYFPNSGISIGLTNHHSLSDANTRFGFLKAWASVCETGEDQPFLKNGSPPVFDRVVVNPQLYENRLNQTRLGTFYQAPSLVGSSSDRVRATFVLARTHISGLKKQVLTQLPMLEYTSSFTVTCGYIWSCIVKSLVNMGEKKGEDELEQFIVSVGCRSRLDPPLPENYFGNCSAPCIVTIKNGVLKGENGFVMAAKLIGEGISKMVNKKGGILEYADRWYDGFKIPARKMGISGTPKLNFYDIDFGWGKAMKYEVVSIDYSASVSLSACKESAQDFEIGVCFPSMQMEAFGKIFNDGLESAIAS

SEQ ID NO: 79 - DNA, 1407 bp, Arabidopsis thaliana
  source 1..1407: mol_type = genomic DNA
atggttgctcacttgcaaccaccaaagattattgaaacttgtcacatttctccaccaaagggtactgttccatctactactttgccattgactttcttcgacgctccatggttgtctttgccattggctgactctttgttcttcttctcttaccaaaactctactgaatctttcttgcaagacttcgttccaaacttgaagcactctttgtctattactttgcaacacttcttcccatacgctggtaagttgattattccaccaagaccagacccaccatacttgcactacaacgacggtcaagactctttggttttcactgttgctgaatctactgaaactgacttcgaccaattgaagtctgactctccaaaggacatttctgttttgcacggtgttttgccaaagttgccaccaccacacgtttctccagaaggtattcaaatgagaccaattatggctatgcaagttactattttcccaggtgctggtatttgtattggtaactctgctactcacgttgttgctgacggtgttactttctctcacttcatgaagtactggatgtctttgactaagtcttctggtaaggacccagctactgttttgttgccatctttgccaattcactcttgtagaaacatgattaaggacccaggtgaagttggtgctggtcacttggaaagattctggtctcaaaactctgctaagcactcttctcacgttactccagaaaacatggttagagctactttcactttgtctagaaagcaaattgacaacttgaagtcttgggttactgaacaatctgaaaaccaatctccagtttctactttcgttgttactttggctttcatttgggtttctttgattaagactttggttcaagactctgaaactaaggctaacgaagaagacaaggacgaagttttccacttgatgattaacgttgactgtagaaacagattgaagtacactcaaccaattccacaaacttacttcggtaactgtatggctccaggtattgtttctgttaagaagcacgacttgttgggtgaaaagtgtgttttggctgcttctgacgctattactgctagaattaaggacatgttgtcttctgacttgttgaagactgctccaagatggggtcaaggtgttagaaagtgggttatgtctcactacccaacttctattgctggtgctccaaagttgggtttgtacgacatggacttcggtttgggtaagccatgtaagatggaaattgttcacattgaaactggtggttctattgctttctctgaatctagagacggttctaacggtgttgaaattggtattgctttggaaaagaagaagatggacgttttcgactctattttgcaacaaggtattaagaagttcgctact

SEQ ID NO: 80 - protein, 469 aa, Arabidopsis thaliana
MVAHLQPPKIIETCHISPPKGTVPSTTLPLTFFDAPWLSLPLADSLFFFSYQNSTESFLQDFVPNLKHSLSITLQHFFPYAGKLIIPPRPDPPYLHYNDGQDSLVFTVAESTETDFDQLKSDSPKDISVLHGVLPKLPPPHVSPEGIQMRPIMAMQVTIFPGAGICIGNSATHVVADGVTFSHFMKYWMSLTKSSGKDPATVLLPSLPIHSCRNMIKDPGEVGAGHLERFWSQNSAKHSSHVTPENMVRATFTLSRKQIDNLKSWVTEQSENQSPVSTFVVTLAFIWVSLIKTLVQDSETKANEEDKDEVFHLMINVDCRNRLKYTQPIPQTYFGNCMAPGIVSVKKHDLLGEKCVLAASDAITARIKDMLSSDLLKTAPRWGQGVRKWVMSHYPTSIAGAPKLGLYDMDFGLGKPCKMEIVHIETGGSIAFSESRDGSNGVEIGIALEKKKMDVFDSILQQGIKKFAT

SEQ ID NO: 81 - DNA, 1395 bp, Vitis amurensis
  source 1..1395: mol_type = genomic DNA
atggctaacccacacccacatttcttgattattacttttccagcccaaggtcatattaacccagctttggaattggccaaaagattgattggtgttggtgctgatgttactttcgctactactattcatgccaagtccagattggttaagaacccaactgttgatggtttgagattctctactttctccgatggtcaagaagaaggtgttaagagaggtccaaacgaattgccagtttttcaaagattggcctccgaaaacttgtccgaattgattatggcttctgctaatgaaggtagaccaatctcttgtttgatctactccattttgattccaggtgctgctgaattggctagatcattcaatattccatctgctttcttgtggattcaaccagctactgttttggacatctattactactacttcaacggtttcggtgacttgatcagatccaaatcttctgatccatccttctccattgaattaccaggtttgccatctttgtccagacaagatttgccatcctttttcgttggttccgaccaaaatcaagaaaaccatgctttggctgcctttcaaaagcacttggaaattttggaacaagaagaaaacccaaaggtcttggttaacactttcgatgctttagaaccagaagccttgagagctgttgaaaagttgaaattgactgctgttggtccattggttccatctggtttttctgatggtaaagatgcttctgatacaccatctggtggtgatttgtctgatggttctagagattatatggaatggttgaagtccaagccagaatctactgttgtttacgtttccttcggttccatcagtatgttctctatgcaacaaatggaagaaatcgccagaggtttgttggaatctggtagaccatttttgtgggttatcagagctaaagaaaacggtgaagaaaacaaagaagaagataagttgtcctgccaagaagaattggaaaagcaaggtatgttgatccaatggtgctctcaaatggaagttttgtctcatccatctttgggttgtttcgttactcattgtggttggaactcctctattgaatctttagcttctggtgttccaatgattgcatttccacaatgggctgatcaaggtactaataccaagttgattaaggacgtttggaaaaccggtgttagattgatggttaacgaagaagaaattgtcacctccgacgaattgagaagatgcttggaattagttatgggtgatggtgaaaagggtcaagaaatgagaaagaatgctaagaagtggaagattttggctaaagaagccttaaaagaaggtggttcctctcacaagaatttgaagaacttcgttgacgaagtcatccaaggttactga

SEQ ID NO: 82 - protein, 464 aa, Vitis amurensis
MANPHPHFLIITFPAQGHINPALELAKRLIGVGADVTFATTIHAKSRLVKNPTVDGLRFSTFSDGQEEGVKRGPNELPVFQRLASENLSELIMASANEGRPISCLIYSILIPGAAELARSFNIPSAFLWIQPATVLDIYYYYFNGFGDLIRSKSSDPSFSIELPGLPSLSRQDLPSFFVGSDQNQENHALAAFQKHLEILEQEENPKVLVNTFDALEPEALRAVEKLKLTAVGPLVPSGFSDGKDASDTPSGGDLSDGSRDYMEWLKSKPESTVVYVSFGSISMFSMQQMEEIARGLLESGRPFLWVIRAKENGEENKEEDKLSCQEELEKQGMLIQWCSQMEVLSHPSLGCFVTHCGWNSSIESLASGVPMIAFPQWADQGTNTKLIKDVWKTGVRLMVNEEEIVTSDELRRCLELVMGDGEKGQEMRKNAKKWKILAKEALKEGGSSHKNLKNFVDEVIQGY

SEQ ID NO: 83 - DNA, 1398 bp, Nierembergia sp. NB17
  source 1..1398: mol_type = genomic DNA
atggaaaacgaaaaggtcttgcacgttgtcatgtttccattctttgctttcggtcatatttccccatttgctcaattggctaacaagttgtcatctcacggtgttaaggtttctttctttactgcttctggtaacgcctctagattgagatcaatgttgaattctgctccaaccaccacccatattgatatagttccattgactttgccacacgttgaaggtttgccaccaggttctgaatctactgctgaattgactccagttaccgctgaattattgaaggttgctttggatttgatgcaaccacaaatcaagaccttgttgtctcatttgaagccacacttcgttttgttcgattttgctcaagaatggttgccaaagatggctgatgaattgggtattaagaccgttttctactccgttttcgttgctttgtctactgcttttttgacttgtccagctagagttactgaacctaaaaagtacccaaccttggaagatatgaagaaaccaccattgggtttcccacatacttctattacatctgttaagaccttcgaagcccaagacttcttgtacattttcaagtcctttaacaacagaccaaccgtttacgatagagtcttgtctggtttgaaaggttgctctgctattttggctaagacctgttctcaaatggaaggtccttatatcgaatacgtcaagtcccaattcaagaagccagttttgttggttggtccagttgttccagatccaccatctggtaaattggaagaaaaatgggatgcttggttgaacaagtttgaagctggtactgttatctactgcagtttcggttctgaaaccttcttgaaggatgatcaaatcaaagaattggccttgggtttggaacaaactggtttgccttttttcttggtcttgaatttcccagctaacgttgatgcttcagccgaattgaatagaggtttgcctgaaggttttagagaaagagtcaaagaaaagggtgttatccattctggttgggttcaacaacaacatattttggcacatacctctgttggttgttacgtttgtcatgctggtttctcatctgttattgaagccttcatgaacgattgccaagttgttatgttgccacaaaagggtgaccaattattgaacgctaaattggtttccggtgatatgaaggctggtgttgaagttaatagaagagatgaagatggttacttctccaaggatgatattgaagaagccgttgaaaaggtcatggtcgaaaaagtcatcagagaaaatcaaaagaagtggaaagaatttttgttgaacaaagacacccactccaagttcgttgaagatttggttcatgatatgatggctatggctaagttgtctactacctga

SEQ ID NO: 84 - protein, 465 aa, Nierembergia sp. NB17
MENEKVLHVVMFPFFAFGHISPFAQLANKLSSHGVKVSFFTASGNASRLRSMLNSAPTTTHIDIVPLTLPHVEGLPPGSESTAELTPVTAELLKVALDLMQPQIKTLLSHLKPHFVLFDFAQEWLPKMADELGIKTVFYSVFVALSTAFLTCPARVTEPKKYPTLEDMKKPPLGFPHTSITSVKTFEAQDFLYIFKSFNNRPTVYDRVLSGLKGCSAILAKTCSQMEGPYIEYVKSQFKKPVLLVGPVVPDPPSGKLEEKWDAWLNKFEAGTVIYCSFGSETFLKDDQIKELALGLEQTGLPFFLVLNFPANVDASAELNRGLPEGFRERVKEKGVIHSGWVQQQHILAHTSVGCYVCHAGFSSVIEAFMNDCQVVMLPQKGDQLLNAKLVSGDMKAGVEVNRRDEDGYFSKDDIEEAVEKVMVEKVIRENQKKWKEFLLNKDTHSKFVEDLVHDMMAMAKLSTT

SEQ ID NO: 85 - DNA, 1344 bp, Clitoria ternatea
  source 1..1344: mol_type = genomic DNA
atggaaaacaacaagcacgtcgtcattttcccatttccatttggttctcatttgccacctttgttgaacttggttttcaaattggctcatgctgctccaaatacctctttctcttttattggtactcatacctccaacgctttcttgttcacaaaaagacatgttccagacaacatcagagttttcaccatttctgatggtatcccagaaggttacgttccaggtaacaatccaattgtcaagttggacttcttcttgtctactggtccagataatttgtgcaagggtattgaattggctgttgccgaaacaaagcaatctgttacctgtattattgccgatgctttcgttacctcttctttgttggttgctcaaactttgaatgttccatggatcgttttctggccaaatgtttcatgttccttgtccttgtacttctccatcgatttgattagagataagtgcaccaacgatgctaagaacgcttctttggattttttgccaggtttgtccaagttgagagttgaagatgttccaagaccacaagctatagttttggatggtaaagaaaccttgttcgccagaactttgaactctttgggtactgttttgccacaagcaaaagctgttgttgttaacttcttcgctgaattggatccaccattattcgttaaggacatgagatccaagttgcaatccttgttgttcgttgatccattgccatgtccacaattgttgttgccagaaactgattctaacggttgtatgtcttggttggactccaaatcttctagatcagttgcttacgtttgcttcggtactgctgtttctttgccaccacaagaagttgttgaagttgctgaagccttggaagaatctggttttccttttttgttggccttgtccgaatctttgattggtgttttaccaaagggtttggtcgaaagaactatgactagaggtaaagttgtttcttgggctccacaatctttggttttgtctcatggttctgttggtgtctttgttactcattgtggtgctaactctgttaccgaatctatttctaatggtgtccctatgatctgcagaccattttttggtgatcaaggtattgctgccagagttattcaagatatttgggaaatcggtgtcatcttggaaggtagaattttcactaagaacggtttcgtcaagaacttgaacttgatcttggtccaagaagaaggtaaaaagatcagagataacgccttgaaggttaagcaaatcgttcaagatgctgctggtccacatggtcaagctgctgaagattttaacactttggttgaaatgatctcctcttcttaa

SEQ ID NO: 86 - protein, 447 aa, Clitoria ternatea
MENNKHVVIFPFPFGSHLPPLLNLVFKLAHAAPNTSFSFIGTHTSNAFLFTKRHVPDNIRVFTISDGIPEGYVPGNNPIVKLDFFLSTGPDNLCKGIELAVAETKQSVTCIIADAFVTSSLLVAQTLNVPWIVFWPNVSCSLSLYFSIDLIRDKCTNDAKNASLDFLPGLSKLRVEDVPRPQAIVLDGKETLFARTLNSLGTVLPQAKAVVVNFFAELDPPLFVKDMRSKLQSLLFVDPLPCPQLLLPETDSNGCMSWLDSKSSRSVAYVCFGTAVSLPPQEVVEVAEALEESGFPFLLALSESLIGVLPKGLVERTMTRGKVVSWAPQSLVLSHGSVGVFVTHCGANSVTESISNGVPMICRPFFGDQGIAARVIQDIWEIGVILEGRIFTKNGFVKNLNLILVQEEGKKIRDNALKVKQIVQDAAGPHGQAAEDFNTLVEMISSS

SEQ ID NO: 87 - DNA, 1449 bp, Gentiana triflora
  source 1..1449: mol_type = genomic DNA
atggaccaattgcacgttttcttcttcccattcttggctaacggtcacattttgccaactattgacatggctaagttgttctcttctagaggtgttaaggctactttgattactactcacaacaactctgctattttcttgaaggctattaacagatctaagattttgggtttcgacatttctgttttgactattaagttcccatctgctgaattcggtttgccagaaggttacgaaactgctgaccaagctagatctattgacatgatggacgaattcttcagagcttgtattttgttgcaagaaccattggaagaattgttgaaggaacacagaccacaagctttggttgctgacttgttcttctactgggctaacgacgctgctgctaagttcggtattccaagattgttgttccacggttcttcttctttcgctatgattgctgctgaatctgttagaagaaacaagccatacaagaacttgtcttctgactctgacccattcgttgttccagacattccagacaagattattttgactaagtctcaagttccaactccagacgaaactgaagaaaacaacactcacattactgaaatgtggaagaacatttctgaatctgaaaacgactgttacggtgttattgttaactctttctacgaattggaaccagactacgttgactactgtaagaacgttttgggtagaagagcttggcacattggtccattgtctttgtgtaacaacgaaggtgaagacgttgctgaaagaggtaagaagtctgacattgacgctcacgaatgtttgaactggttggactctaagaacccagactctgttgtttacgtttgtttcggttctatggctaacttcaacgctgctcaattgcacgaattggctatgggtttggaagaatctggtcaagaattcatttgggttgttagaacttgtgttgacgaagaagacgaatctaagtggttcccagacggtttcgaaaagagagttcaagaaaacaacaagggtttgattattaagggttgggctccacaagttttgattttggaacacgaagctgttggtgctttcgtttctcactgtggttggaactctactttggaaggtatttgtggtggtgttgctatggttacttggccattgttcgctgaacaattctacaacgaaaagttgatgactgacattttgagaactggtgtttctgttggttctttgcaatggtctagagttactacttctgctgttgttgttaagagagaatctatttctaaggctgttagaagattgatggctgaagaagaaggtgttgacattagaaacagagctaaggctttgaaggaaaaggctaagaaggctgttgaaggtggtggttcttcttactctgacttgtctgctttgttggttgaattgtcttcttacccacacaactag

SEQ ID NO: 88 - protein, 482 aa, Gentiana triflora
MDQLHVFFFPFLANGHILPTIDMAKLFSSRGVKATLITTHNNSAIFLKAINRSKILGFDISVLTIKFPSAEFGLPEGYETADQARSIDMMDEFFRACILLQEPLEELLKEHRPQALVADLFFYWANDAAAKFGIPRLLFHGSSSFAMIAAESVRRNKPYKNLSSDSDPFVVPDIPDKIILTKSQVPTPDETEENNTHITEMWKNISESENDCYGVIVNSFYELEPDYVDYCKNVLGRRAWHIGPLSLCNNEGEDVAERGKKSDIDAHECLNWLDSKNPDSVVYVCFGSMANFNAAQLHELAMGLEESGQEFIWVVRTCVDEEDESKWFPDGFEKRVQENNKGLIIKGWAPQVLILEHEAVGAFVSHCGWNSTLEGICGGVAMVTWPLFAEQFYNEKLMTDILRTGVSVGSLQWSRVTTSAVVVKRESISKAVRRLMAEEEGVDIRNRAKALKEKAKKAVEGGGSSYSDLSALLVELSSYPHN

SEQ ID NO: 89 - DNA, 1410 bp, Gentiana triflora
  source 1..1410: mol_type = genomic DNA
atggaacaaattcaaatggttaaggttttggaaaagtgtcaagttactccaccatctgacactactgacgttgaattgtctttgccagttactttcttcgacattccatggttgcacttgaacaagatgcaatctttgttgttctacgacttcccatacccaagaactcacttcttggacactgttattccaaacttgaaggcttctttgtctttgactttgaagcactacgttccattgtctggtaacttgttgatgccaattaagtctggtgaaatgccaaagttccaatactctagagacgaaggtgactctattactttgattgttgctgaatctgaccaagacttcgactacttgaagggtcaccaattggttgactctaacgacttgcacggtttgttctacgttatgccaagagttattagaactatgcaagactacaaggttattccattggttgctgttcaagttactgttttcccaaacagaggtattgctgttgctttgactgctcaccactctattgctgacgctaagtctttcgttatgttcattaacgcttgggcttacattaacaagttcggtaaggacgctgacttgttgtctgctaacttgttgccatctttcgacagatctattattaaggacttgtacggtttggaagaaactttctggaacgaaatgcaagacgttttggaaatgttctctagattcggttctaagccaccaagattcaacaaggttagagctacttacgttttgtctttggctgaaattcaaaagttgaagaacaaggttttgaacttgagaggttctgaaccaactattagagttactactttcactatgacttgtggttacgtttggacttgtatggttaagtctaaggacgacgttgtttctgaagaatcttctaacgacgaaaacgaattggaatacttctctttcactgctgactgtagaggtttgttgactccaccatgtccaccaaactacttcggtaactgtttggcttcttgtgttgctaaggctactcacaaggaattggttggtgacaagggtttgttggttgctgttgctgctattggtgaagctattgaaaagagattgcacaacgaaaagggtgttttggctgacgctaagacttggttgtctgaatctaacggtattccatctaagagattcttgggtattactggttctccaaagttcgactcttacggtgttgacttcggttggggtaagccagctaagttcgacattacttctgttgactacgctgaattgatttacgttattcaatctagagacttcgaaaagggtgttgaaattggtgtttctttgccaaagattcacatggacgctttcgctaagattttcgaagaaggtttctgttctttgtcttag

SEQ ID NO: 90 - protein, 469 aa, Gentiana triflora
MEQIQMVKVLEKCQVTPPSDTTDVELSLPVTFFDIPWLHLNKMQSLLFYDFPYPRTHFLDTVIPNLKASLSLTLKHYVPLSGNLLMPIKSGEMPKFQYSRDEGDSITLIVAESDQDFDYLKGHQLVDSNDLHGLFYVMPRVIRTMQDYKVIPLVAVQVTVFPNRGIAVALTAHHSIADAKSFVMFINAWAYINKFGKDADLLSANLLPSFDRSIIKDLYGLEETFWNEMQDVLEMFSRFGSKPPRFNKVRATYVLSLAEIQKLKNKVLNLRGSEPTIRVTTFTMTCGYVWTCMVKSKDDVVSEESSNDENELEYFSFTADCRGLLTPPCPPNYFGNCLASCVAKATHKELVGDKGLLVAVAAIGEAIEKRLHNEKGVLADAKTWLSESNGIPSKRFLGITGSPKFDSYGVDFGWGKPAKFDITSVDYAELIYVIQSRDFEKGVEIGVSLPKIHMDAFAKIFEEGFCSLS

SEQ ID NO: 91 - DNA, 933 bp, Saccharomyces cerevisiae
  source 1..933: mol_type = genomic DNA
atgcctatcaccataaaaagccgctctaaagggttaagggacactgaaattgacttatccaaaaagcctactttagatgatgttttgaaaaaaatctctgctaataaccacaatatcagcaagtacaggataagattaacctacaaaaaggaatctaaacaagttccggttatttcagaatcgttttttcaagaagaggctgatgactcaatggaattcttcatcaaagatttgggtccccaaatttcatggagattagtcttcttttgtgagtatttgggtccagtcttggttcactcccttttttattatctatctaccattcccacagttgttgatagatggcacagtgctagctccgactataatccatttttaaacagggttgcatattttttaattttaggacattatggaaagagattatttgaaaccttatttgttcaccaattctctttagctactatgccaattttcaacctgttcaaaaattgtttccattactgggttctaagcggtctcatttcattcggttactttggctacggcttcccctttgggaatgctaagttattcaaatactattcatatttgaaattggatgacttgagtacattaattggtcttttcgtgctttcagaactatggaacttttattgccacattaaattgcgcctatggggtgactatcaaaagaagcatggtaacgctaagatccgtgtcccattgaatcaaggtattttcaatctttttgttgctcccaactatacttttgaagtttggtcttggatttggtttacttttgtgttcaagttcaatttatttgccgttttatttttgactgtttcaacagctcaaatgtacgcatgggctcaaaagaaaaacaaaaagtatcataccagaagagcattcttgattccatttgtattttga

SEQ ID NO: 92 - protein, 310 aa, Saccharomyces cerevisiae
MPITIKSRSKGLRDTEIDLSKKPTLDDVLKKISANNHNISKYRIRLTYKKESKQVPVISESFFQEEADDSMEFFIKDLGPQISWRLVFFCEYLGPVLVHSLFYYLSTIPTVVDRWHSASSDYNPFLNRVAYFLILGHYGKRLFETLFVHQFSLATMPIFNLFKNCFHYWVLSGLISFGYFGYGFPFGNAKLFKYYSYLKLDDLSTLIGLFVLSELWNFYCHIKLRLWGDYQKKHGNAKIRVPLNQGIFNLFVAPNYTFEVWSWIWFTFVFKFNLFAVLFLTVSTAQMYAWAQKKNKKYHTRRAFLIPFVF

SEQ ID NO: 93 - DNA, 1452 bp, Pyrus communis
  source 1..1452: mol_type = genomic DNA
atgggtgacgttattgttttgtacgcttctccaggtatgggtcacattgttgctatggttgaattgggtaagttcattgttcacagatacggtccacacaagttctctattactattttgtacacttgtggttctattgttgacactgcttctattccagtttacattagaagaatttctcactctcacccattcatttctttcagacaattcccaagagttactaacaacattactagaaacatttctgttccagctattactttcgacttcattagacaaaacgacccacacgttagatctgctttgcaagaaatttctaagtctgctactgttagagctttcattattgacttgttctgtacttctgctttgccaattggtaaggaattcaacattccaacttactacttccacacttctggtgctgctgttttggctgctttcttgtacttgccaaagattgacgaacaaactaagactactgaatctttcaaggacttgagagacactgttttcgaattcccaggttggaagtctccattgaaggctactcacatggttcaattggttttggacagaaacgacccagcttactctgacatgatttacttctgttctcacttgccaaagtctaacggtattattgttaacactttcgaagaattggaaccaccatctgttttgcaagctattgctggtggtttgtgtgttccagacggtccaactccaccagtttactacgttggtccattgattgaagaagaaaaggaattgtctaaggacgctgacgctgctgaaaaggaagactgtttgtcttggttggacaagcaaccatctagatctgttttgttcttgtgtttcggttctatgggttctttcccagctgctcaattgaaggaaattgctaacggtttggaagcttctggtcaaagattcttgtgggttgttaagaagccaccagttgaagaaaagtctaagcaagttcacggtgttgacgacttcgacttgaagggtgttttgccagaaggtttcttggaaagaactgctgacagaggtatggttgttaagtcttgggctccacaagttgttgttttgaagaaggaatctgttggtggtttcgttactcactgtggttggaactctgttttggaagctgttgttgctggtgttccaatgattgcttggccattgtacgctgaacaacacatgaacagaaacgttttggttactgacatggaaattgctattggtgttgaacaaagagacgaagaaggtggtttcgtttctggtgaagaagttgaaagaagagttagagaattgatggaatctgaaggtggtagagctttgagagaaagatgtaagaagttgggtgaaatggcttctgctgctttgggtgaaactggttcttctactagaaacttggttaacttcgtttcttctattacttaa

SEQ ID NO: 94 - protein, 483 aa, Pyrus communis
MGDVIVLYASPGMGHIVAMVELGKFIVHRYGPHKFSITILYTCGSIVDTASIPVYIRRISHSHPFISFRQFPRVTNNITRNISVPAITFDFIRQNDPHVRSALQEISKSATVRAFIIDLFCTSALPIGKEFNIPTYYFHTSGAAVLAAFLYLPKIDEQTKTTESFKDLRDTVFEFPGWKSPLKATHMVQLVLDRNDPAYSDMIYFCSHLPKSNGIIVNTFEELEPPSVLQAIAGGLCVPDGPTPPVYYVGPLIEEEKELSKDADAAEKEDCLSWLDKQPSRSVLFLCFGSMGSFPAAQLKEIANGLEASGQRFLWVVKKPPVEEKSKQVHGVDDFDLKGVLPEGFLERTADRGMVVKSWAPQVVVLKKESVGGFVTHCGWNSVLEAVVAGVPMIAWPLYAEQHMNRNVLVTDMEIAIGVEQRDEEGGFVSGEEVERRVRELMESEGGRALRERCKKLGEMASAALGETGSSTRNLVNFVSSIT

SEQ ID NO: 95 - DNA, 1416 bp, Oryza sativa
  source 1..1416: mol_type = genomic DNA
atgccgagctctggcgacgcggcgggcaggcggccgcatgtggtgctcatcccgagcgccggcatgggccacctcgtccccttcggccgcctcgccgtggcgctgtcctccggccacggctgcgacgtctccctcgtcacggtgctccccaccgtgtccaccgcggagtcgaagcacctcgacgcgctgttcgacgcgttcccggcggtgcggcggctcgacttcgagctcgcgccgttcgacgcgtccgagttccccggcgccgacccgttcttcctccggttcgaggccatgcggcggtcggcgccgctcctcggcccgctcctcaccggcgccggcgcgtcggcgctcgccacggacatcgcgctgacatccgtcgtcatacccgtggcgaaggagcagggcctcccgtgccacatcctcttcaccgcctccgccgcgatgctctccctctgcgcctacttccccacatacctcgacgccaacgctggcggcggcggcggcgtcggcgacgtcgacatccccggcgtgtaccgcatccccaaggcctccatcccgcaggcgctgcacgaccccaaccacctcttcacccgccagttcgtcgccaacggccggagcctcacgagcgccgccggcatcctcgtcaacacgttcgacgccttggagccggaggccgtcgcggccctgcagcagggcaaggtcgcctccggcttcccgccggtgttcgccgtggggccacttctcccggcgagcaaccaggcgaaagatccgcaggcgaactacatggagtggctcgacgcgcagccggcgcggtcggtggtgtacgtgagcttcggcagccggaaggccatctcaagggagcagctcagggagctcgccgccgggctggagggcagcggccaccggttcctgtgggtggtgaagagcaccgtcgtggacagggacgacgccgccgagctcggcgagctgctcgacgaggggttcttggagcgggtggagaagcgaggcctcgtcaccaaggcatgggtggatcaggaagaggtcctgaagcacgagtccgtggcgctgttcgtgagccactgcggctggaactcggtgacggaggcggcggcgagcggcgtcccggtgctggcgctgccgaggttcggcgaccagcgggtgaactccggcgtggtggcgcgcgccgggctcggcgtgtgggcggacacctggagctgggagggggaggccggggtgatcggcgcggaggagatatcggagaaggtgaaggcggcgatggcggacgaggcgttgcggatgaaggcggcgagcctcgccgaggccgccgcgaaggccgtcgccggcggtggatcgagccaccgttgtctggccgagttcgcgcggctgtgccaagggggaacatgtcgcactaattga

SEQ ID NO: 96 - protein, 471 aa, Oryza sativa
MPSSGDAAGRRPHVVLIPSAGMGHLVPFGRLAVALSSGHGCDVSLVTVLPTVSTAESKHLDALFDAFPAVRRLDFELAPFDASEFPGADPFFLRFEAMRRSAPLLGPLLTGAGASALATDIALTSVVIPVAKEQGLPCHILFTASAAMLSLCAYFPTYLDANAGGGGGVGDVDIPGVYRIPKASIPQALHDPNHLFTRQFVANGRSLTSAAGILVNTFDALEPEAVAALQQGKVASGFPPVFAVGPLLPASNQAKDPQANYMEWLDAQPARSVVYVSFGSRKAISREQLRELAAGLEGSGHRFLWVVKSTVVDRDDAAELGELLDEGFLERVEKRGLVTKAWVDQEEVLKHESVALFVSHCGWNSVTEAAASGVPVLALPRFGDQRVNSGVVARAGLGVWADTWSWEGEAGVIGAEEISEKVKAAMADEALRMKAASLAEAAAKAVAGGGSSHRCLAEFARLCQGGTCRTN

SEQ ID NO: 97 - DNA, 708 bp, Vitis vinifera
  source 1..708: mol_type = genomic DNA
atgtcttcttcttctcacagaggtattttgaagactgaagctttgactaagtacttgttggaaacttctgcttacccaagagaacacgaacaattgaagggtttgagagaagctactgttgaaaagtacaagtactggtctttgatgaacgttccagttgacgaaggtttgttcatttctatgttgttgaagattatgaacgctaagaagactattgaattgggtgttttcactggttactctttgttggctactgctttggctttgccacaagacggtaagattattgctgttgacccagacaaggaagcttaccaaactggtgttccattcattaagaaggctggtgttgaacacaagattaacttcattcaatctgacgctatgactgttttgaacgacttgattgctgacggtaaggaagaaggtactttggacttcgctatggttgacgctgacaaggaaaactacttgaactaccacgaattgttgttgaagttggttagagttggtggtattattgcttacgacaacactttgtggttcggttctgttgctagatctgaagaagaagaaatgatggacttcgaaagagctggtagagttcacttgatgaagttgaacaagttcttggcttctgacccaagagttgaattgtctcacttgtctattggtgacggtgttgctttgtgtagaagattgtactag

SEQ ID NO: 98 - protein, 235 aa, Vitis vinifera
MSSSSHRGILKTEALTKYLLETSAYPREHEQLKGLREATVEKYKYWSLMNVPVDEGLFISMLLKIMNAKKTIELGVFTGYSLLATALALPQDGKIIAVDPDKEAYQTGVPFIKKAGVEHKINFIQSDAMTVLNDLIADGKEEGTLDFAMVDADKENYLNYHELLLKLVRVGGIIAYDNTLWFGSVARSEEEEMMDFERAGRVHLMKLNKFLASDPRVELSHLSIGDGVALCRRLY

SEQ ID NO: 99 - DNA, 2004 bp, Arabidopsis thaliana
  source 1..2004: mol_type = genomic DNA
atggatgatactacgtataagccaaagaacattctcattactggagctgctggatttattgcttctcatgttgccaacagattaatccgtaactatcctgattacaagatcgttgttcttgacaagctagattactgttcagatctgaagaatcttgatccttctttttcttcaccaaatttcaagtttgtcaaaggagatatcgcgagtgatgatctcgttaactaccttctcatcactgaaaacattgatacgataatgcattttgctgctcaaactcatgttgataactcttttggtaatagctttgagtttaccaagaacaatatttatggtactcatgttcttttggaagcctgtaaagttacaggacagatcaggaggtttatccatgtgagtaccgatgaagtctatggagaaaccgatgaggatgctgctgtaggaaaccatgaagcctctcagctgttaccgacgaatccttactctgcaactaaggctggtgctgagatgcttgtgatggcttatggtagatcatatggattgcctgttattacgactcgcgggaacaatgtttatgggcctaaccagtttcctgaaaaaatgattcctaagttcatcttgttggctatgagtgggaagccgcttcccatccatggagatggatctaatgtccggagttacttgtactgcgaagacgttgctgaggcttttgaggttgttcttcacaaaggagaaatcggtcatgtctacaatgtcggcacaaaaagagaaaggagagtgatcgatgtggctagagacatctgcaaacttttcgggaaagaccctgagtcaagcattcagtttgtggagaaccggccctttaatgatcaaaggtacttccttgatgatcagaagctgaagaaattggggtggcaagagcgaacaaattgggaagatggattgaagaagacaatggactggtacactcagaatcctgagtggtggggtgatgtttctggagctttgcttcctcatccgagaatgcttatgatgcccggtggaagactttctgatggatctagtgagaagaaagacgtttcaagcaacacggtccagacatttacggttgtaacacctaagaatggtgattctggtgacaaggcttcgttgaagtttttgatctatggtaagactggttggcttggtggtcttctagggaaactatgtgagaagcaagggattacatatgagtatgggaaaggacgtctggaggatagagcttctcttgtggcggatattcgtagcatcaaacctactcatgtgtttaatgctgctggtttaactggcagacccaacgttgactggtgtgaatctcacaaaccagagaccattcgtgtaaatgtcgcaggtactttgactctagctgatgtttgcagagagaatgatctcttgatgatgaacttcgccaccggttgcatctttgagtatgacgctacacatcctgagggttcgggtataggtttcaaggaagaagacaagccaaatttctttggttctttctactcgaaaaccaaagccatggttgaggagctcttgagagaatttgacaatgtatgtaccttgagagtccggatgccaatctcctcagacctaaacaacccgagaaacttcatcacgaagatctcgcgctacaacaaagtggtggacatcccgaacagcatgaccgtactagacgagcttctcccaatctctatcgagatggcgaagagaaacctaagaggcatatggaatttcaccaacccaggggtggtgagccacaacgagatattggagatgtacaagaattacatcgagccaggttttaaatggtccaacttcacagtggaagaacaagcaaaggtcattgttgctgctcgaagcaacaacgaaatggatggatctaaactaagcaaggagttcccagagatgctctccatcaaagagtcactgctcaaatacgtctttgaaccaaacaagagaacctaa

SEQ ID NO: 100 - protein, 667 aa, Arabidopsis thaliana
MDDTTYKPKNILITGAAGFIASHVANRLIRNYPDYKIVVLDKLDYCSDLKNLDPSFSSPNFKFVKGDIASDDLVNYLLITENIDTIMHFAAQTHVDNSFGNSFEFTKNNIYGTHVLLEACKVTGQIRRFIHVSTDEVYGETDEDAAVGNHEASQLLPTNPYSATKAGAEMLVMAYGRSYGLPVITTRGNNVYGPNQFPEKMIPKFILLAMSGKPLPIHGDGSNVRSYLYCEDVAEAFEVVLHKGEIGHVYNVGTKRERRVIDVARDICKLFGKDPESSIQFVENRPFNDQRYFLDDQKLKKLGWQERTNWEDGLKKTMDWYTQNPEWWGDVSGALLPHPRMLMMPGGRLSDGSSEKKDVSSNTVQTFTVVTPKNGDSGDKASLKFLIYGKTGWLGGLLGKLCEKQGITYEYGKGRLEDRASLVADIRSIKPTHVFNAAGLTGRPNVDWCESHKPETIRVNVAGTLTLADVCRENDLLMMNFATGCIFEYDATHPEGSGIGFKEEDKPNFFGSFYSKTKAMVEELLREFDNVCTLRVRMPISSDLNNPRNFITKISRYNKVVDIPNSMTVLDELLPISIEMAKRNLRGIWNFTNPGVVSHNEILEMYKNYIEPGFKWSNFTVEEQAKVIVAARSNNEMDGSKLSKEFPEMLSIKESLLKYVFEPNKRT

SEQ ID NO: 101 - DNA, 827 bp, Saccharomyces cerevisiae
  source 1..827: mol_type = genomic DNA
gatcttccaactggcaccgctggcttgaacaacaataccagccttccaacttctgtaaataacggcggtacgccagtgccaccagtaccgttacctttcggtatacctcctttccccatgtttccaatgcccttcatgcctccaacggctactatcacaaatcctcatcaagctgacgcaagccctaagaaatgaataacaatactgacagtactaaataattgcctacttggcttcacatacgttgcatacgtcgatatagataataatgataatgacagcaggattatcgtaatacgtaatagttgaaaatctcaaaaatgtgtgggtcattacgtaaataatgataggaatgggattcttctatttttcctttttccattctagcagccgtcgggaaaacgtggcatcctctctttcgggctcaattggagtcacgctgccgtgagcatcctctctttccatatctaacaactgagcacgtaaccaatggaaaagcatgagcttagcgttgctccaaaaaagtattggatggttaataccatttgtctgttctcttctgactttgactcctcaaaaaaaaaaaatctacaatcaacagatcgcttcaattacgccctcacaaaaacttttttccttcttcttcgcccacgttaaattttatccctcatgttgtctaacggatttctgcacttgatttattataaaaaagacaaagacataatacttctctatcaatttcagttattgttcttccttgcgttattcttctgttcttctttttcttttgtcatatataaccataaccaagtaatacatattcaaaa

SEQ ID NO: 102 - DNA, 686 bp, Saccharomyces cerevisiae
  source 1..686: mol_type = genomic DNA
gatctcagttcgagtttatcattatcaatactgccatttcaaagaatacgtaaataattaatagtagtgattttcctaactttatttagtcaaaaaattagccttttaattctgctgtaacccgtacatgcccaaaatagggggcgggttacacagaatatataacatcgtaggtgtctgggtgaacagtttattcctggcatccactaaatataatggagcccgctttttaagctggcatccagaaaaaaaaagaatcccagcaccaaaatattgttttcttcaccaaccatcagttcataggtccattctcttagcgcaactacagagaacaggggcacaaacaggcaaaaaacggacacaacctcaatggagtgatgcaacctgcctggagtaaatgatgacacaaggcaattgacccacgcatgtatctatctcattttcttacaccttctattaccttctgctctctctgatttggaaaaagctgaaaaaaaaggttgaaaccagttccctgaaattattcccctacttgactaataagtatataaagacggtaggtattgattgtaattctgtaaatctatttcttaaacttcttaaattctacttttatagttagtcttttttttagttttaaaacaccaagaacttagtttcgaataaacacacataaacaaacaaaa

SEQ ID NO: 103 - DNA, 756 bp, Saccharomyces cerevisiae
  source 1..756: mol_type = genomic DNA
gatctacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaa

SEQ ID NO: 104 - DNA, 805 bp, Saccharomyces cerevisiae
  source 1..805: mol_type = genomic DNA
gatctatgcgactgggtgagcatatgttccgctgatgtgatgtgcaagataaacaagcaaggcagaaactaacttcttcttcatgtaataaacacaccccgcgtttatttacctatctctaaacttcaacaccttatatcataactaatatttcttgagataagcacactgcacccataccttccttaaaaacgtagcttccagtttttggtggttccggcttccttcccgattccgcccgctaaacgcatatttttgttgcctggtggcatttgcaaaatgcataacctatgcatttaaaagattatgtatgctcttctgacttttcgtgtgatgaggctcgtggaaaaaatgaataatttatgaatttgagaacaattttgtgttgttacggtattttactatggaataatcaatcaattgaggattttatgcaaatatcgtttgaatatttttccgaccctttgagtacttttcttcataattgcataatattgtccgctgcccctttttctgttagacggtgtcttgatctacttgctatcgttcaacaccaccttattttctaactattttttttttagctcatttgaatcagcttatggtgatggcacatttttgcataaacctagctgtcctcgttgaacataggaaaaaaaaatatataaacaaggctctttcactctccttgcaatcagatttgggtttgttccctttattttcatatttcttgtcatattcctttctcaattattattttctactcataacctcacgcaaaataacacagtcaaatctatcaaaa

SEQ ID NO: 105 - DNA, 1003 bp, Saccharomyces cerevisiae
  source 1..1003: mol_type = genomic DNA
gatctaatgctactattttggagatcaatctcagtacaaaacaatattaaaaagaggtgaattatttttccccccttattttttttttgttagaattgatccaaatgtaaataaacaatcacaaggaaaaaaaaaaaaaaaaaaaatagccgccatgaccccggatcgtcggttgtgatacggtcagggtagcgccctggtcaaacttcagaactaaaaaaataataaggaagaaaaaaatagctaatttttccggcagaaagattttcgctacccgaaagtttttccggcaagctaaatggaaaaaggaaagattattgaaagagaaagaaagaaaaaaaaaaaatgtacacccagacatcgggcttccacaatttcggctctattgttttccatctctcgcaacggcgggattcctctatggcgtgtgatgtctgtatctgttacttaatccagaaactggcacttgacccaactctgccacgtgggtcgttttgccatcgacagattgggagattttcatagtagaattcagcatgatagctacgtaaatgtgttccgcaccgtcacaaagtgttttctactgttctttcttctttcgttcattcagttgagttgagtgagtgctttgttcaatggatcttagctaaatgcatattttttctcttggtaaatgaatgcttgtgatgtcttccaagtgatttcctttccttcccatatgatgctaggtacctttagtgtcttcctaaaaaaaaaaaaaggctcgccatcaaaacgatattcgttggcttttttttctgaattataaatactctttggtaacttttcatttccaagaacctcttttttccagttatatcatggtcccctttcaaagttattctctactctttttcatattcattctttttcatcctttggttttttattcttaacttgtttattattctctcttgtttctatttacaagacaccaatcaaaacaaataaaacatcatcacaa

SEQ ID NO: 106 - DNA, 417 bp, Saccharomyces cerevisiae
  source 1..417: mol_type = genomic DNA
gatcttagcttcaaaatgtttctactccttttttactcttccagattttctcggactccgcgcatcgccgtaccacttcaaaacacccaagcacagcatactaaatttcccctctttcttcctctagggtgtcgttaattacccgtactaaaggtttggaaaagaaaaaagagaccgcctcgtttctttttcttcgtcgaaaaaggcaataaaaatttttatcacgtttctttttcttgaaaatttttttttttgatttttttctctttcgatgacctcccattgatatttaagttaataaacggtcttcaatttctcaagtttcagtttcatttttcttgttctattacaactttttttacttcttgctcattagaaagaaagcatagcaatctaatctaagttttaattacaaaa

SEQ ID NO: 107 - DNA, 565 bp, Saccharomyces cerevisiae
  source 1..565: mol_type = genomic DNA
gatctgggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaaca

SEQ ID NO: 108 - DNA, 538 bp, Saccharomyces cerevisiae
  source 1..538: mol_type = genomic DNA
gatctagttataataatcctacgttagtgtgagcgggatttaaactgtgaggaccttaatacattcagacacttctgcggtatcaccctacttattcccttcgagattatatctaggaacccatcaggttggtggaagattacccgttctaagacttttcagcttcctctattgatgttacacctggacaccccttttctggcatccagtttttaatcttcagtggcatgtgagattctccgaaattaattaaagcaatcacacaattctctcggataccacctcggttgaaactgacaggtggtttgttacacatgctaatgcaaaggagcctatatacctttggctcggctgctgtaacagggaatataaagggcagcataatttaggagtttagtgaacttgcaacatttactattttcccttcttacgtaaatatttttctttttaattctaaatcaatctttttcaattttttgtttgtattcttttcttgcttaaatctataactacaaaaaacacatacataaactaaaaa

SEQ ID NO: 109 - DNA, 1269 bp, synthetic construct
  misc_feature 1..1269: note = Synthetic
  source 1..1269: mol_type = other DNA
ggcgcgccagttacttgctctatgcgtttgcgcatcctctttttactttttttttttcagtaaagcctaagcataaatcgttttatacgtacgacacgttcaacttttcttggttagtagtggcaatctctgcaatacatacagggagtcatggtctatcatcttgtccaatcaaagaagcatcggttcagatcgagcaaactgtagggagaaaggaaagtagaaatgcagagtgtgctatatgtccaatctcggttttgtagtttggatgtcattagagatctaccacccaaccggctgctttcatgtggaacagaaaagaaatcggggcgcttcctcttctgtattcctttaattaacgtttttattcagccatctaaccatcatacccccatacggtaacaaaacctcttctaagaaaagaagtctctgctcctccgccatcttatttttattcgctgcgcgcgtttattgtcgcatcgctagccagcaaaaagttggttgcctttttttacctaaaaaagacacatctaactgattagttttccgttttaggatattgacgccaagcgtgcgtctgattcccgggtcatcgtccacctccggagaacaggccaccatcacgcatctgtgtctgaatttcatcacgaggcgcgccttttcccgtctttcagtgccttgttcagttcttcctgacgggcggtatatttctccagcttactagtttacgtggattgagccagcaatacagatcattattaaactgttttgtacatgatgttagtatataatcgtaaagcttttctaatatgtataccttatacatggaactccacagaacttgcaaacataccaaaaatcctttattcttgttcactcattttacatcaaaaaataatatttcagttattaaggaaaataaaaaaatagattagagaagcattttgaagaaatagtatattcttttattgaacctaagagcgtgatatttttactcgaaataaaatacgaaaaatctatacactcatctttccgactactattggctcctgctcaaaaaaagagggaaaaaaagctccaaaattctatcttttcctatcgctcctgtcctatccttattacgttcattactattttaatactatccattcttttattttcagtctaaaaaaaacatttctcataacgggaaaagcaaaaaaatgtcaagcttatacatcaaaacaccactgcatgcattatctgctggtccggattctcaggcgcgcc

SEQ ID NO: 110 - DNA, 1288 bp, synthetic construct
  misc_feature 1..1288: note = Synthetic
  source 1..1288: mol_type = other DNA
ggcgcgccgaggattttcgatggagcaggatgaggagaaatagtaccacatgtatatatccattacaaaaaggtttatatacaattacaatagacccttgttggggtttctgaaaaaagaagtagtcgatgccatcggcaataatacggaattacgagaaacacaatcccgatccttttttgggtaattacttcaccgattctaccgatttatcatgccaaaaaaaattcaccgtgggttctagaagtgccctttgaggattgtagccactctaacccacacggcctccttactagctgactaaggtgacaaaaccgcaaggactggaaagtcgccactcatctgaaaattctcaagtttttcactactgagtttatgctttcgaatttttttgttcggtaatagcacggcggttcgattcaattccgccgctccgagcgatgctccgcaaaactcagtaataagctttctgatggttcaccccttttttagcacgcggggtgtaactcaacagaaaaatgtgccatagaacaagactaggcaaaagccaaggagcgtttgccatgaacttccacaaccgggtcatcgtccacctccggagaacaggccaccatcacgcatctgtgtctgaatttcatcacgaggcgcgccttttcccgtctttcagtgccttgttcagttcttcctgacgggcggtatatttctccagcttactagcacacgactagcgctttcagatattaaaaagtttagatgtaggttttagcggtaacagttatataaatcgtgtttcttctcttgatgaaacaaaaaaatgctagaaaaactttgtcgtttcttacttttggtgcgctttgcagttttcgtggctagacttagaatcatttctcctcagatttcttgattaaagtttggtgcgaagccctactctaacattggtgttcttcttttcattcacgcaagttaagtccaggaaggtgagcaaatgctcatccttctgttcatgcgtgacggctgaattatccttatctggcgtacccgtgcagccgtttccgtgcctcggttcctccgagatatccttagggaccgccagggaccatgattgcgtcaactgttgtcaccgctccagaggatcctctgtaaccttttcaaccataaaaatagaaagggcacagggttggggtatttgaattttttttttgggttttttcgtattacttattacttaccttcttttctatataattttgtttttccctggtagagcggaatcttcccacggcgcgcc

SEQ ID NO: 111 - DNA, 1710 bp, synthetic construct
  misc_feature 1..1710: note = Synthetic
  source 1..1710: mol_type = other DNA
ggcgcgcctcatcgtccacctccggagaacaggccaccatcacgcatctgtgtctgaatttcatcacgacgcgccgctgcaggtcgacaacccttaatataacttcgtataatgtatgctatacgaagttattaggtctagagatcccaatacaacagatcacgtgatcttttgtaagatgaagttgaagtgagtgttgcaccgtgccaatgcaggtggctattagattaaatatgtgatttgttctattaagtttcctgtataattaatggggagcgctgattctcttttggtacgcttcccatccagcatttctgtatctttcaccttcaaccttaggatctctacccttggcgaaaagtcctctgccaacaatgatgatatctgatccaccacttacaacttcgtcgacggttctgtactgctgacccaatgcatcgcctttgtcgtctaaacctacacctggggtcatgattagccaatcaaacccttcttctcttcctcccatatcgttctgagcaatgaacccaataacgaaatctttatcactctttgcaatatcaacggtacccttagtatattcaccgtgtgctagagaacccttggaagacaattcagcaagcatcaataatccccttggttctttggtgacctcttgcgcaccttgtttcaagccagcaacaataccagcaccagtaaccccgtgggcgttggtgatatcagaccattctgcgatacggtaaacgcccgatgtatattgtaatttgactgtgttaccgatatcggcgaattttctgtcctcaaatatcaagaacttgtatttctctgccaatgctttcaatggaacgacagtaccctcataactgaaatcatccaagatatcaacgtgtgttttcaaaaggcaaatgtatggacccaacgtttcaacaagtttcaatagctcatcagtcgaacgaacgtcaagagaagcacacaaattggtcttcttttcatccattaaacgtaaaagtttcgatgcaaccggacttgcatgagtctcagctctactggtatatgattttgtggacatggtgcaactaattgacgggagtgtattgacgctggcgtactggctttcacaaaatggcccaatcacaaccacatcttagatagttgaaatgactttagataacatcaattgagatgagcttaatcatgtcaaagctaaaagtgtcaccatgaacgacaattcttaagcaaatcacgtgatatagatccacgaataaccaccatttgatgctcgaggcaagtaatgtgtgtaaaaaaatgcgttaccaccatccaatgcagaccgatcttctacccagaatcacatatatttatgtaccgagtaccttttttctatcttccaattgcttctcccatatgattgtctccgtaagctcgaaatttctaagttggattttaatcttcacgcaggatgacagttcgatgagcttctgaggagtgtttagaacataatcagtttatccatggtctatctcttcttgtcgctttttctcctcgatagaacctaaataaaacgagctctcgagaacccttaatataacttcgtataatgtatgctatacgaagttattaggtgatatcagatccggcgcgtggcacccttgcgggccatgtcatacaccgccttcagagcagccggacctatctgcccgttggcgcgcc

SEQ ID NO: 112 - DNA, 4427 bp, synthetic construct
  misc_feature 1..4427: note = Synthetic
  source 1..4427: mol_type = other DNA
ggcgcgcctttcccgtctttcagtgccttgttcagttcttcctgacgggcggtatatttctccagcttggcctatgcggccctgtcagaccaagtttacgagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacgcggccgctcgactacgtcgtaaggccgtttctgacagagtaaaattcttgagggaactttcaccattatgggaaatggttcaagaaggtattgacttaaactccatcaaatggtcaggtcattgagtgttttttatttgttgtatttttttttttttagagaaaatcctccaatatataaattaggaatcatagtttcatgattttctgttacacctaactttttgtgtggtgccctcctccttgtcaatattaatgttaaagtgcaattctttttccttatcacgttgagccattagtatcaatttgcttacctgtattcctttacatcctcctttttctccttcttgataaatgtatgtagattgcgtatatagtttcgtctaccctatgaacatattccattttgtaatttcgtgtcgtttctattatgaatttcatttataaagtttatgtacaaatatcataaaaaaagagaatctttttaagcaaggattttcttaacttcttcggcgacagcatcactgacttcggtggtactgttggaaccacctaaatcaccagttctgatacctgcatccaaaacctttttaactgcatcttcaatggccttaccttcttcaggcaagttcaatgacaatttcaacatcattgcagcagacaagatagtggcgatagggttgaccttattctttggcaaatctggagcagaaccgtggcatggttcgtacaaaccaaatgcggtgttcttgtctggcaaagaggccaaggacgcagatggcaacaaacccaaggaacctgggataacggaggcttcatcggagatgatatcaccaaacatgttgctggtgattataataccatttaggtgggttgggttcttaactaggatcatggcggcagaatcaatcaattgatgttgaaccttcaatgtagggaattcgttcttgatggtttcctccacagtttttctccataatcttgaagaggccaaaacattagctttatccaaggaccaaataggcaatggtggctcatgttgtagggccatgaaagcggccattcttgtgattctttgcacttctggaacggtgtattgttcactatcccaagcgacaccatcaccatcgtcttcctttctcttaccaaagtaaatacctcccactaattctctgacaacaacgaagtcagtacctttagcaaattgtggcttgattggagataagtctaaaagagagtcggatgcaaagttacatggtcttaagttggcgtacaattgaagttctttacggatttttagtaaaccttgttcaggtctaacactaccggtaccccatttaggaccacccacagcacctaacaaaacggcatcagccttcttggaggcttccagcgcctcatctggaagtggaacacctgtagcatcgatagcagcaccaccaattaaatgattttcgaaatcgaacttgacattggaacgaacatcagaaatagctttaagaaccttaatggcttcggctgtgatttcttgaccaacgtggtcacctggcaaaacgacgatcttcttaggggcagacattagaatggtatatccttgaaatatatatatatatattgctgaaatgtaaaaggtaagaaaagttagaaagtaagacgattgctaaccacctattggaaaaaacaataggtccttaaataatattgtcaacttcaagtattgtgatgcaagcatttagtcatgaacgcttctctattctatatgaaaagccggttccggcgctctcacctttcctttttctcccaatttttcagttgaaaaaggtatatgcgtcaggcgacctctgaaattaacaaaaaatttccagtcatcgaatttgattctgtgcgatagcgcccctgtgtgttctcgttatgttgaggaaaaaaataatggttgctaagagattcgaactcttgcatcttacgatacctgagtattcccacagttaactgcggtcaagatatttcttgaatcaggcgccttagaccgctcggccaaacaaccaattacttgttgagaaatagagtataattatcctataaatataacgtttttgaacacacatgaacaaggaagtacaggacaattgattttgaagagaatgtggattttgatgtaattgttgggattccatttttaataaggcaataatattaggtatgtagatatactagaagttctcctcgagcggccgcacgcgttcatcgtccacctccggagaacaggccaccatcacgcatctgtgtctgaatttcatcacgggcgcgcc

SEQ ID NO: 113 - DNA, 3967 bp, synthetic construct
  misc_feature 1..3967: note = Synthetic
  source 1..3967: mol_type = other DNA
ggcgcgcctttcccgtctttcagtgccttgttcagttcttcctgacgggcggtatatttctccagcttggcctatgcggccctgtcagaccaagtttacgagctcgcttggactcctgttgatagatccagtaatgacctcagaactccatctggatttgttcagaacgctcggttgccgccgggcgttttttattggtgagaatccaagcactagggacagtaagacgggtaagcctgttgatgataccgctgccttactgggtgcattagccagtctgaatgacctgtcacgggataatccgaagtggtcagactggaaaatcagagggcaggaactgctgaacagcaaaaagtcagatagcaccacatagcagacccgccataaaacgccctgagaagcccgtgacgggcttttcttgtattatgggtagtttccttgcatgaatccataaaaggcgcctgtagtgccatttacccccattcactgccagagccgtgagcgcagcgaactgaatgtcacgaaaaagacagcgactcaggtgcctgatggtcggagacaaaaggaatattcagcgatttgcccgagcttgcgagggtgctacttaagcctttagggttttaaggtctgttttgtagaggagcaaacagcgtttgcgacatccttttgtaatactgcggaactgactaaagtagtgagttatacacagggctgggatctattctttttatctttttttattctttctttattctataaattataaccacttgaatataaacaaaaaaaacacacaaaggtctagcggaatttacagagggtctagcagaatttacaagttttccagcaaaggtctagcagaatttacagatacccacaactcaaaggaaaaggacatgtaattatcattgactagcccatctcaattggtatagtgattaaaatcacctagaccaattgagatgtatgtctgaattagttgttttcaaagcaaatgaactagcgattagtcgctatgacttaacggagcatgaaaccaagctaattttatgctgtgtggcactactcaaccccacgattgaaaaccctacaaggaaagaacggacggtatcgttcacttataaccaatacgctcagatgatgaacatcagtagggaaaatgcttatggtgtattagctaaagcaaccagagagctgatgacgagaactgtggaaatcaggaatcctttggttaaaggctttgagattttccagtggacaaactatgccaagttctcaagcgaaaaattagaattagtttttagtgaagagatattgccttatcttttccagttaaaaaaattcataaaatataatctggaacatgttaagtcttttgaaaacaaatactctatgaggatttatgagtggttattaaaagaactaacacaaaagaaaactcacaaggcaaatatagagattagccttgatgaatttaagttcatgttaatgcttgaaaataactaccatgagtttaaaaggcttaaccaatgggttttgaaaccaataagtaaagatttaaacacttacagcaatatgaaattggtggttgataagcgaggccgcccgactgatacgttgattttccaagttgaactagatagacaaatggatctcgtaaccgaacttgagaacaaccagataaaaatgaatggtgacaaaataccaacaaccattacatcagattcctacctacataacggactaagaaaaacactacacgatgctttaactgcaaaaattcagctcaccagttttgaggcaaaatttttgagtgacatgcaaagtaagtatgatctcaatggttcgttctcatggctcacgcaaaaacaacgaaccacactagagaacatactggctaaatacggaaggatctgaggttcttatggctcttgtatctatcagtgaagcatcaagactaacaaacaaaagtagaacaactgttcaccgttacatatcaaagggaaaactgtccatatgcacagatgaaaacggtgtaaaaaagatagatacatcagagcttttacgagtttttggtgcattcaaagctgttcaccatgaacagatcgacaatgtaacgcggccgcctgcacggtcctgttccctagcatgtacgtgagcgtatttccttttaaaccacgacgctttgtcttcattcaacgtttcccattgtttttttctactattgctttgctgtgggaaaaacttatcgaaagatgacgactttttcttaattctcgttttaagagcttggtgagcgctaggagtcactgccaggtatcgtttgaacacggcattagtcagggaagtcataacacagtcctttcccgcaattttctttttctattactcttggcctcctctagtacactctatatttttttatgcctcggtaatgattttcatttttttttttccacctagcggatgactctttttttttcttagcgattggcattatcacataatgaattatacattatataaagtaatgtgatttcttcgaagaatatactaaaaaatgagcaggcaagataaacgaaggcaaagatgacagagcagaaagccctagtaaagcgtattacaaatgaaaccaagattcagattgcgatctctttaaagggtggtcccctagcgatagagcactcgatcttcccagaaaaagaggcagaagcagtagcagaacaggccacacaatcgcaagtgattaacgtccacacaggtatagggtttctggaccatatgatacatgctctggccaagcattccggctggtcgctaatcgttgagtgcattggtgacttacacatagacgaccatcacaccactgaagactgcgggattgctctcggtcaagcttttaaagaggccctaggggccgtgcgtggagtaaaaaggtttggatcaggatttgcgcctttggatgaggcactttccagagcggtggtagatctttcgaacaggccgtacgcagttgtcgaacttggtttgcaaagggagaaagtaggagatctctcttgcgagatgatcccgcattttcttgaaagctttgcagaggctagcagaattaccctccacgttgattgtctgcgaggcaagaatgatcatcaccgtagtgagagtgcgttcaaggctcttgcggttgccataagagaagccacctcgcccaatggtaccaacgatgttccctccaccaaaggtgttcttatgtagtgacaccgattatttaaagctgcagcatacgatatatatacatgtgtatatatgtatacctatgaatgtcagtaagtatgtatacgaacagtatgatactgaagatgacaaggtaatgcatcattctatacgtgtcattctgaacgaggcgcgctttccttttttctttttgctttttctttttttttctcttgaactcgatcgagaaaaaaaatataaaagagatggaggaacgggaaaaagttagttgtggtgataggtggcaagtggtattccgtaagaacaacaagaaaagcatttcatattatggctgaactgagcgaacaagtgcaaaatttaagcatcaacgacaacaacgagaatggttatgttcctcctcacttaagaggaaaaccaagaagtgccagaaataacagtagcaactacaataacaacaacggcggctacaacggtggccgtggcggtggcagcttctttagcaacaaccgtcgtggtggttacggcaacggtggtttcttcggtggaaacaacggtggcagcagatctaacggccgttctggtggtagatggatcgatggcaaacatgtcccagctccaagaaacgaaaaggccgagatcgccatatttggtgtggcggccgcacgcgttcatcgtccacctccggagaacaggccaccatcacgcatctgtgtctgaatttcatcacgggcgcgcc

SEQ ID NO: 114 - DNA, 1871 bp, synthetic construct
  misc_feature 1..1871: note = Synthetic
  source 1..1871: mol_type = other DNA
ggcgcgcctcatcgtccacctccggagaacaggccaccatcacgcatctgtgtctgaatttcatcacgacgcgccttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtattggaccaccctgtgggtttataagcgcgctgctggcgtgtaaggcggtgacggcgaaggaagggtccttttcatcacgtgctataaaaataattataatttaaattttttaatataaatatataaattaaaaatagaaagtaaaaaaagaaattaaagaaaaaatagtttttgttttccgaagatgtaaaagactctagggggatcgccaacaaatactaccttttatcttgctcttcctgctctcaggtattaatgccgaattgtttcatcttgtctgtgtagaagaccacacacgaaaatcctgtgattttacattttacttatcgttaatcgaatgtatatctatttaatctgcttttcttgtctaataaatatatatgtaaagtacgctttttgttgaaattttttaaacctttgtttatttttttttcttcattccgtaactcttctaccttctttatttactttctaaaatccaaatacaaaacataaaaataaataaacacagagtaaattcccaaattattccatcattaaaagatacgaggcgcgtgtaagttacaggcaagcgatccgtcctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccacggcgcgtggcacccttgcgggccatgtcatacaccgccttcagagcagccggacctatctgcccgttggcgcgcc

SEQ ID NO: 115 - DNA, 748 bp, synthetic construct
  misc_feature 1..748: note = Synthetic
  source 1..748: mol_type = other DNA
ggcgcgccccggctgtctgccatgctgcccggtgtaccgacataaccgccggtggcatagccgcgcatacgcgtctccagcgtgttttatctctgcgagcataatgcctgcgtcatccgccagcaggagctggactttactgatgcccgttatatctgcgaaaagaccgggatctggacccgtgatggcattctctggttttcgtcatccggtgaagagattgagccacctgacagtgtgacctttcacatctggacagcgtacagcccgttcaccacctgggtgcagattgtcaaagactggatgaaaacgaaaggggatacgggaaaacgtaaaaccttcgtaaacaccacgctcggtgagacgtgggaggcgaaaattggcgaacgtccggatgctgaagtgatggcagagcggaaagagcattattcagcgcccgttcctgaccgtgtggcttacctgaccgccggtatcgactcccagctggaccgctacgaaatgcgcgtatggggatgggggccgggtgaggaaagctggctgattgaccggcagattattatgggccgccacgacgatgaacagacgctgctgcgtgtggatgaggccatcaataaaacctatacccgccggaatggtgcagaaatgtcgatatcccgtatctgctgggatactggacgcgttttcccgtctttcagtgccttgttcagttcttcctgacgggcggtatatttctccagcttggcgcgcc

SEQ ID NO: 116 - DNA, 748 bp, synthetic construct
  misc_feature 1..748: note = Synthetic
  source 1..748: mol_type = other DNA
ggcgcgcccagccggtaaagattccccacgccaatccggctggttgcctccttcgtgaagacaaactcacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgttttcccgtctttcagtgccttgttcagttcttcctgacgggcggtatatttctccagcttggcgcgcc

SEQ ID NO: 117 - DNA, 748 bp, synthetic construct
  misc_feature 1..748: note = Synthetic
  source 1..748: mol_type = other DNA
ggcgcgccaccacggtgaacaatccccgctggctcatatttgccgccggttcccgtaaatcctccggtacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgttttcccgtctttcagtgccttgttcagttcttcctgacgggcggtatatttctccagcttggcgcgcc

SEQ ID NO: 118 - DNA, 748 bp, synthetic construct
  misc_feature 1..748: note = Synthetic
  source 1..748: mol_type = other DNA
ggcgcgccaagctggagaaatataccgcccgtcaggaagaactgaacaaggcactgaaagacgggaaaacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgtggcgcatccgcgtcaggcggtacagccattcaggccgctgcggcgaaattccattttgcaggcgcgcc

SEQ ID NO: 119 - DNA, 748 bp, synthetic construct
  misc_feature 1..748: note = Synthetic
  source 1..748: mol_type = other DNA
ggcgcgccgccaccaacagccccgccaatggcgctgccgatactcccgacaatccccaccattgcctgacgcgtccagtatcccagcagatacgggatatcgacatttctgcaccattccggcgggtataggttttattgatggcctcatccacacgcagcagcgtctgttcatcgtcgtggcggcccataataatctgccggtcaatcagccagctttcctcacccggcccccatccccatacgcgcatttcgtagcggtccagctgggagtcgataccggcggtcaggtaagccacacggtcaggaacgggcgctgaataatgctctttccgctctgccatcacttcagcatccggacgttcgccaattttcgcctcccacgtctcaccgagcgtggtgtttacgaaggttttacgttttcccgtatcccctttcgttttcatccagtctttgacaatctgcacccaggtggtgaacgggctgtacgctgtccagatgtgaaaggtcacactgtcaggtggctcaatctcttcaccggatgacgaaaaccagagaatgccatcacgggtccagatcccggtcttttcgcagatataacgggcatcagtaaagtccagctcctgctggcggatgacgcaggcattatgctcgcagagataaaacacgctggagacgcgttttcccgtctttcagtgccttgttcagttcttcctgacgggcggtatatttctccagcttggcgcgcc

SEQ ID NO: 120 - DNA, 1822 bp, synthetic construct
  misc_feature 1..1822: note = Synthetic
  source 1..1822: mol_type = other DNA
cgcgccggcacccttgcgggccatgtcatacaccgccttcagagcagccggacctatctgcccgttacgcgccagcttgcaaattaaagccttcgagcgtcccaaaaccttctcaagcaaggttttcagtataatgttacatgcgtacacgcgtctgtacagaaaaaaaagaaaaatttgaaatataaataacgttcttaatactaacataactataaaaaaataaatagggacctagacttcaggttgtctaactccttccttttcggttagagcggatgtggggggagggcgtgaatgtaagcgtgacataactaattacatgatatcgacaaaggaaaagggggacggatctccgaggcctcggacccgtcgggccgccgtcggacgtgccgcggatccccgggtcgagcctgaacggcctcgaggcctgaacggcctcgacgaattcattatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagctggccctgcaggccaagctttttgtttgtttatgtgtgtttattcgaaactaagttcttggtgttttaaaactaaaaaaaagactaactataaaagtagaatttaagaagtttaagaaatagatttacagaattacaatcaatacctaccgtctttatatacttattagtcaagtaggggaataatttcagggaactggtttcaaccttttttttcagctttttccaaatcagagagagcagaaggtaatagaaggtgtaagaaaatgagatagatacatgcgtgggtcaattgccttgtgtcatcatttactccaggcaggttgcatcactccattgaggttgtgtccgttttttgcctgtttgtgcccctgttctctgtagttgcgctaagagaatggacctatgaactgatggttggtgaagaaaacaatattttggtgctgggattctttttttttctggatgccagcttaaaaagcgggctccattatatttagtggatgccaggaataaactgttcacccagacacctacgatgttatatattctgtgtaacccgccccctattttgggcatgtacgggttacagcagaattaaaaggctaattttttgactaaataaagttaggaaaatcactactattaattatttacgtattctttgaaatggcagtattgataatgataaactcgaactgggcgcgtcgtgccgtcgttgttaatcaccacatggttattctgctcaaacgtcccggacgcctgcgaggcgcgcc

SEQ ID NO: 121 - DNA, 1879 bp, synthetic construct
  misc_feature 1..1879: note = Synthetic
  source 1..1879: mol_type = other DNA
ggcgcgccatgcgcggctatgccaccggcggttatgtcggtacaccgggcagcatggcagacagccggacgcgccacgcacagatattataacatctgcataataggcatttgcaagaattactcgtgagtaaggaaagagtgaggaactatcgcatacctgcatttaaagatgccgatttgggcgcgaatcctttattttggcttcaccctcatactattatcagggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaaagcttggcctgcagggccagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgtcgaggccgttcaggcctcgaggccgttcaggctcgacccggggatccgcggatctcttatgtctttacgatttatagttttcattatcaagtatgcctatattagtatatagcatctttagatgacagtgttcgaagtttcacgaataaaagataatattctactttttgctcccaccgcgtttgctagcacgagtgaacaccatccctcgcctgtgagttgtacccattcctctaaactgtagacatggtagcttcagcagtgttcgttatgtacggcatcctccaacaaacagtcggttatagtttgtcctgctcctctgaatcgtctccctcgatatttctcattttccttcggcgcgttcgcaggcgtccgggacgtttgagcagaataaccatgtggtgattaacaacgacggcacgggcgcgcc

SEQ ID NO: 122 - DNA, 1587 bp, synthetic construct
  misc_feature 1..1587: note = Synthetic
  source 1..1587: mol_type = other DNA
ggcgcgccccggctgtctgccatgctgcccggtgtaccgacataaccgccggtggcatagccgcgcatacgcgccatttccttccatcttgtgattcatgctatccatcttttttgagtatccaattaacgaagacgttaccagctgattgaaggttctcaaagtgactgtactccatgttttcttatcatccatgtagttatttttcaaactgcaaattcaagaaaaagccacgcgtgtgcaccttttttttccccttccagtgcattatgcaatagacagcacgagtctttgaaaaagtaacttataaaactgtatcaatttttaaacctaaatagattcataaactattcgttaatataaagtgttctaaactatgatgaaaaaataagcagaaaagactaataattcttagttaaaagcactccgcggatccccgggtcgagcctgaacggcctcgaggcctgaacggcctcgacgaattcattatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagctggccctgcaggccaagctttttgtaattaaaacttagattagattgctatgctttctttctaatgagcaagaagtaaaaaaagttgtaatagaacaagaaaaatgaaactgaaacttgagaaattgaagaccgtttattaacttaaatatcaatgggaggtcatcgaaagagaaaaaaatcaaaaaaaaaaattttcaagaaaaagaaacgtgataaaaatttttattgcctttttcgacgaagaaaaagaaacgaggcggtctcttttttcttttccaaacctttagtacgggtaattaacgacaccctagaggaagaaagaggggaaatttagtatgctgtgcttgggtgttttgaagtggtacggcgatgcgcggagtccgagaaaatctggaagagtaaaaaaggagtagaaacattttgaagctaggcgcgtcagccggtaaagattccccacgccaatccggctggttgcctccttcgtgaagacaaactcggcgcgcc

SEQ ID NO: 123 - DNA, 1928 bp, synthetic construct
  misc_feature 1..1928: note = Synthetic
  source 1..1928: mol_type = other DNA
ggcgcgcccagccggtaaagattccccacgccaatccggctggttgcctccttcgtgaagacaaactcacgcgcctccaaaatgagctatcaaaaacgatagatcgattaggatgactttgaaatgactccgcagtggactggccgttaatttcaagcgtgagtaaaatagtgcatgacaaaagatgagctaggcttttgtaaaaatatcttacgttgtaaaattttagaaatcattatttccttcatatcattttgtcattgaccttcagaagaaaagagccgaccaataatataaataaataaataaaaataatattccattatttctaaacagattcaatactcattaaaaaactatatcaattaatttgaattaaccgcggatccccgggtcgagcctgaacggcctcgaggcctgaacggcctcgacgaattcattatttgtagagctcatccatgccatgtgtaatcccagcagcagttacaaactcaagaaggaccatgtggtcacgcttttcgttgggatctttcgaaagggcagattgtgtcgacaggtaatggttgtctggtaaaaggacagggccatcgccaattggagtattttgttgataatggtctgctagttgaacggatccatcttcaatgttgtggcgaattttgaagttagctttgattccattcttttgtttgtctgccgtgatgtatacattgtgtgagttatagttgtactcgagtttgtgtccgagaatgtttccatcttctttaaaatcaataccttttaactcgatacgattaacaagggtatcaccttcaaacttgacttcagcacgcgtcttgtagttcccgtcatctttgaaagatatagtgcgttcctgtacataaccttcgggcatggcactcttgaaaaagtcatgccgtttcatatgatccggataacgggaaaagcattgaacaccataagagaaagtagtgacaagtgttggccatggaacaggtagttttccagtagtgcaaataaatttaagggtaagctggccctgcaggccaagctttttgatagatttgactgtgttattttgcgtgaggttatgagtagaaaataataattgagaaaggaatatgacaagaaatatgaaaataaagggaacaaacccaaatctgattgcaaggagagtgaaagagccttgtttatatatttttttttcctatgttcaacgaggacagctaggtttatgcaaaaatgtgccatcaccataagctgattcaaatgagctaaaaaaaaaatagttagaaaataaggtggtgttgaacgatagcaagtagatcaagacaccgtctaacagaaaaaggggcagcggacaatattatgcaattatgaagaaaagtactcaaagggtcggaaaaatattcaaacgatatttgcataaaatcctcaattgattgattattccatagtaaaataccgtaacaacacaaaattgttctcaaattcataaattattcattttttccacgagcctcatcacacgaaaagtcagaagagcatacataatcttttaaatgcataggttatgcattttgcaaatgccaccaggcaacaaaaatatgcgtttagcgggcggaatcgggaaggaagccggaaccaccaaaaactggaagctacgtttttaaggaaggtatgggtgcagtgtgcttatctcaagaaatattagttatgatataaggtgttgaagtttagagataggtaaataaacgcggggtgtgtttattacatgaagaagaagttagtttctgccttgcttgtttatcttgcacatcacatcagcggaacatatgctcacccagtcgcatggcgcgtaccacggtgaacaatccccgctggctcatatttgccgccggttcccgtaaatcctccggtggcgcgcc

SEQ ID NO: 124 - DNA, 1736 bp, synthetic construct
  misc_feature 1..1736: note = Synthetic
  source 1..1736: mol_type = other DNA
ggcgcgccaccacggtgaacaatccccgctggctcatatttgccgccggttcccgtaaatcctccggtacgcgccgggccgtatacttacatatagtagatgtcaagcgtaggcgcttcccctgccggctgtgagggcgccataaccaaggtatctatagaccgccaatcagcaaactacctccgtacattcatgttgcacccacacatttatacacccagaccgcgacaaattacccataaggttgtttgtgacggcgtcgtacaagagaacgtgggaactttttaggctcaccaaaaaagaaagaaaaaatacgagttgctgacagaagcctcaagaaaaaaaaaattcttcttcgactatgctggaggcagagatgatcgagccggtagttaactatatatagctaaattggttccatcaccttcttttctggtgtcgctccttctagtgctatttctggcttttcctatttttttttttccatttttctttctctctttctaatatataaattctcttgcattttctatttttctctctatctattctacttgtttattcccttcaaggtttttttttaaggagtacttgtttttagaatatacggtcaacgaactataattaactaaacaagcttggcctgcagggccagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgtcgaggccgttcaggcctcgaggccgttcaggctcgacccggggatccgcggacaaatcgctcttaaatatatacctaaagaacattaaagctatattataagcaaagatacgtaaattttgcttatattattatacacatatcatatttctatatttttaagatttggttatataatgtacgtaatgcaaaggaaataaattttatacattattgaacagcgtccaagtaactacattatgtgcactaatagtttagcgtcgtgaagactttattgtgtcgcgaaaagtaaaaattttaaaaattagagcaccttgaacttgcgaaaaaggttctcatcaactgtttaaaaggaggatatcaggtcctatttctgacaaacaatatacaaatttagtttcaaaggcgcgttgcaaaatggaatttcgccgcagcggcctgaatggctgtaccgcctgacgcggatgcgccggcgcgcc

SEQ ID NO: 125 - DNA, 2175 bp, synthetic construct
  misc_feature 1..2175: note = Synthetic
  source 1..2175: mol_type = other DNA
ggcgcgcccaggcaatggtggggattgtcgggagtatcggcagcgccattggcggggctgttggtggcacgcgccaatgctactattttggagatcaatctcagtacaaaacaatattaaaaagaggtgaattatttttccccccttattttttttttgttagaattgatccaaatgtaaataaacaatcacaaggaaaaaaaaaaaaaaaaaaaaatagccgccatgaccccggatcgtcggttgtgatacggtcagggtagcgccctggtcaaacttcagaactaaaaaaataataaggaagaaaaaaatagctaatttttccggcagaaagattttcgctacccgaaagtttttccggcaagctaaatggaaaaaggaaagattattgaaagagaaagaaagaaaaaaaaaaaatgtacacccagacatcgggcttccacaatttcggctctattgttttccatctctcgcaacggcgggattcctctatggcgtgtgatgtctgtatctgttacttaatccagaaactggcacttgacccaactctgccacgtgggtcgttttgccatcgacagattgggagattttcatagtagaattcagcatgatagctacgtaaatgtgttccgcaccgtcacaaagtgttttctactgttctttcttctttcgttcattcagttgagttgagtgagtgctttgttcaatggatcttagctaaaatgcatattttttctcttggtaaatgaatgcttgtgatgtcttccaagtgatttcctttccttcccatatgatgctaggtacctttagtgtcttcctaaaaaaaaaaaaaggctcgccatcaaaacgatattcgttggcttttttttctgaattataaatactctttggtaacttttcatttccaagaacctcttttttccagttatatcatggtcccctttcaaagttattctctactctttttcatattcattctttttcatcctttggttttttattcttaacttgtttattattctctcttgtttctatttacaagacaccaatcaaaacaaataaaacatcatcacaaagcttggcctgcagggccagcttacccttaaatttatttgcactactggaaaactacctgttccatggccaacacttgtcactactttctcttatggtgttcaatgcttttcccgttatccggatcatatgaaacggcatgactttttcaagagtgccatgcccgaaggttatgtacaggaacgcactatatctttcaaagatgacgggaactacaagacgcgtgctgaagtcaagtttgaaggtgatacccttgttaatcgtatcgagttaaaaggtattgattttaaagaagatggaaacattctcggacacaaactcgagtacaactataactcacacaatgtatacatcacggcagacaaacaaaagaatggaatcaaagctaacttcaaaattcgccacaacattgaagatggatccgttcaactagcagaccattatcaacaaaatactccaattggcgatggccctgtccttttaccagacaaccattacctgtcgacacaatctgccctttcgaaagatcccaacgaaaagcgtgaccacatggtccttcttgagtttgtaactgctgctgggattacacatggcatggatgagctctacaaataatgaattcgtcgaggccgttcaggcctcgaggccgttcaggctcgacccggggatccgcggccgcaggcctaaattgatctagagctttggacttcttcgccagaggtttggtcaagtctccaatcaaggttgtcggcttgtctaccttgccagaaatttacgaaaagatggaaaagggtcaaatcgttggtagatacgttgttgacacttctaaataagcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgcggcgcgtggcgcatccgcgtcaggcggtacagccattcaggccgctgcggcgaaattccattttgcaggcgcgcc

(19) United States
(12) Patent Application Publication
(10) Pub. No.: US 2026/0250728 A1
(43) Pub. Date: Aug. 27, 2026

(54) RECOMBINANT HOST CELLS WITH IMPROVED PRODUCTION OF TETRAKETIDE DERIVATIVES

(71) Applicants: Michael NAESBY, Huningue (FR); IPTECTOR ASSETS APS, Lynge (DK)

(72) Inventor: Michael NAESBY, Huningue (FR)

(73) Assignee: IPTECTOR ASSETS APS, Lynge (DK)

(21) Appl. No.: 19/538,807

(22) Filed: Feb. 12, 2026

Related U.S. Application Data
(63) Continuation of application No. 17/420,990, filed on Jan. 6, 2020.

(30) Foreign Application Priority Data
Jan. 6, 2019 (EP) 19150443.0
Apr. 5, 2019 (EP) 19167702.0
Jun. 27, 2019 (EP) 19182812.8

(51) Int. Cl. C12P 17/06; C12N 9/00; C12N 9/02; C12N 9/04; C12N 9/10; C12N 9/88; C12N 15/52

(52) U.S. Cl. CPC C12P 17/06; C12N 9/0006; C12N 9/001; C12N 9/0042; C12N 9/0071; C12N 9/0093; C12N 9/1088; C12N 9/88; C12N 9/93; C12N 15/52; C12Y 101/01219; C12Y 103/01074; C12Y 106/02004; C12Y 114/11022; C12Y 114/11023; C12Y 117/01003; C12Y 205/01018; C12Y 402/01105; C12Y 403/01024; C12Y 403/01025; C12Y 602/01012

(57) ABSTRACT

The present invention relates to arecombinant microbial host cell producing a tetraketide or derivatives thereof from one or more substrates selected from cinnamoyl-CoA, p-Coumaroyl-CoA, Caffeoyl-CoA, Feruloyl-CoA, malonyl-CoA, sinapoyl-CoA and dihydro derivatives thereof, comprising an operative biosynthetic metabolic pathway for the tetraketide or derivatives thereof comprising a chalcone isomerase-like (CHIL) polypeptide heterologous to the host cell and a Type 3 polyketide synthase (PKS).

### CROSS-REFERENCE TO RELATED APPLICATIONS

This application is a continuation of U.S. patent application Ser. No. 17/420,990 filed 6 Jul. 2021, which is a national phase application of International Application No. PCT/EP2020/050150, filed 6 Jan. 2020, which claims the benefit of European Patent Application No. 19150443.0, filed 6 Jan. 2019, European Patent Application No. 19167702.0 filed 5 Apr. 2019 and European Patent Application No. 19182812.8 filed 27 Jun. 2019, the disclosures of which are incorporated, in their entireties, by this reference.

### FIELD OF THE INVENTION

The present invention relates to recombinant host cells producing tetraketide derivatives; to recombinant polynucleotides comprising a sequence encoding pathway enzymes and polypeptides, operably linked to promotor nucleotide sequences facilitating expression of the pathway enzymes. Further, the invention relates to cell cultures comprising the host cell of the invention, to methods of producing the tetraketide derivatives; to fermentation liquids resulting from such methods, to compositions comprising the fermentation liquid and to the use of such compositions.

### BACKGROUND OF THE INVENTION

Tetraketide derivatives comprise a large and versatile group of compounds including chalcones, dihydrochalcones, stilbenes and dihydrostilbenes and further derivatives thereof.

Dihydrochalcones include subgroups of useful compounds such as phlorizin, nothofagin, and aspalatin, with reported human health benefits and other uses (Ibdah, 2018, J. Agric. Food Chem., 66 (10): 2273-2280; Eichenberger, 2017, Met. Eng. 39:80-89)

Stilbenes include subgroups of compounds such as pinosylvin, resveratrol, piceatannol and pterostilbene with reported benefits in human health (Tsai, 2017, J. Food and Drug Analysis 25 (1): 134-147).

Dihydrostilbenes and their acids, dihydrostilbene carboxylates, include subgroups of useful compounds such as the amorfrutins, which has recently attracted attention due to their potential benefits in human health, including reported activities in the areas of diabetes and cancer (Weidner, 2012, Proc. Natl. Acad. Sci USA. 109 (19):7257-62; Weidner, 2016, J. Nat. Prod. 79(1):2-12).

Chalcone derivatives encompasses a significant group of compounds, such as flavonoids. Flavonoids derives in natural biological systems from various CoA activated phenylpropanoid precursors, most commonly cinnamoyl-CoA and p-coumaroyl-CoA, extended in successive rounds with multiple molecules of malonyl-CoA, to form the basic tetraketide backbone, which is then cyclized into the basic chalcone structure. The final ring closure is then isomerized to form the central flavanone structure, from which most other flavonoids, including anthocyanins, are derived. Anthocyanins is a flavonoid derivative derived from naringenin, which are found naturally in flowers, where they provide bright red and purple colors. Anthocyanins are also found in vegetables and fruits, mostly in the outer parts, such as stem and peel. Anthocyanins are useful as dyes or coloring agents, and furthermore, anthocyanins have caught attention for their human health promoting properties. Other useful flavonoids are epicatechins, which are used as dietary supplements.

Some efforts have been done in developing heterologous production of tetraketide derivatives using unicellular hosts, particularly Escherichia coli and Saccharomyces cerevisiae.

Regarding the chalcone derivatives various flavanones, flavones, and flavonols have been produced in E. coli from phenyl propanoid precursors (see e.g. Yan Y et al., Appl Environ Microbiol. 2005, 71(9):5610-3; Jiang H et al., Appl Environ Microbiol. 2005, 71(6):2962-9; and Leonard E et al., Appl Environ Microbiol. 2007, 73(12):3877-86). Production of other flavonoids have been demonstrated by exogenously feeding suitable precursors, for example isoflavonoids made by feeding liquiritigenin; flavan-3-ols and flavan-4-ols made from feeding flavanones; and anthocyanins made from feeding flavanones or (+)-catechin. In yeast some flavanones, flavones, and flavonols have been made from phenyl propanoids, while few reports have been made on producing flavonoids from sugar e.g. naringenin (Koopman et al. 2012, Microb Cell Fact. 11:155) or various flavanones and flavonols (Naesby et al. 2009, Microb Cell Fact. 8:45). WO2017/050853 discloses assembly of a pathway designed for production of anthocyanin in yeast.

One challenge in designing heterologous pathways and producing tetraketide derivatives in host cells is to achieve industrially and commercially relevant yields. One heterologous biosynthetic pathway for the central flavanone naringenin expressed in yeast lead to massive accumulation of p-coumaric acid and/or its degradation product phloretic acid (Lehka et al. FEMS Yeast Res. 2017, 17(1)). An effort to increase CHS activity, in order to balance the pathway, did not prevent the accumulation of phloretic acid, the degradation product of p-coumaric acid. (Koopman et al., Microb Cell Fact 2012, 11:155).

Morita et al. The Plant Journal, 2014, 78: 294-304 discloses that mutations in a chalcone isomerase-like protein (CHIL), a non-enzymatic type IV chalcone isomerase, seem to have effect on the production of flavonoids in plants, and suggests that in plants the wild type (wt) CHIL is involved in CHS activity though an unknown mechanism. Another study (Jiang et al., J. Exp. Bot. 2015, 66, 22:7165-7179), shows that expression of Arabidopsis thaliana CHIL seemingly affects the levels of flavonols and proanthocyanidins, but not that of anthocyanins. A further study (Fujino et al., Plant Biotechnol 2014, 31:105-114) showed that in snapdragon, CHIL was abundantly expressed irrespective of flower color, a trait closely linked to the production of anthocyanins. A recent study in hops (Ban et al., PNAS, 2018, 115(22):E5223-E5232) shows involvement of two CHILs in a biosynthetic pathway towards the prenylated chalcone xanthohumol. One CHIL (CHIL2), seemed to affect chalcone synthase and prenyl transferase through protein-protein interactions, whereas the other CHIL (CHIL1), was suggested to stabilize the ring-open conformation of the chalcone precursor. Based on these contradictory studies, no clear role can be assigned to CHILs in plants. CHIL is not known in unicellular microbial hosts and functions and effects in heterologous biosynthetic pathways in such hosts have not yet been explored.

Another obstacle challenging unicellular heterologous production of tetraketide derivatives such as anthocyanins (ACN) and epicatechins (EPC) is that several enzymes in the pathways are promiscuous in regards of both substrate and catalyzed reaction, which leads to poor efficiency for specific desired products.

Anthocyanin synthase (ANS), which is an enzyme known from pathways producing anthocyanins, has been shown to produce more flavonol than anthocyanidin in vitro (Turnbull et al., Chem. Commun., 2000, 2473-74; Turnbull et al., Bioorg. Med. Chem. Lett., 2003, 13:3853-57; Turnbull et al., J. Biol. Chem. 2004, 279:1206-16; Welford et al., Chem. Commun., 2001, 1828-29). In PhD thesis work of Michael Eisenberger: Biosynthesis of plant polyketides in yeast; Technical University, Darmstadt, Germany; published Apr. 3, 2019, production of anthocyanin in yeast employing a pathway including glutathione-S-transferase was tested.

In view of this art there is a need to modify and to improve pathways for industrial production of tetraketide derivatives in heterologous unicellular host cells, in order to increase the yield of such products to commercially sustainable levels.

### SUMMARY OF THE INVENTION

The present inventors contemplate that the accumulation of p-coumaric acid and/or its degradation product phloretic acid reported by Lehka et al. FEMS Yeast Res. 2017, 17(1)), together with the failure to prevent the phloretic acid accumulation reported by Koopman et al., Microb Cell Fact 2012, 11:155, indicates a slow rate of conversion of the precursors phenylpropanoyl-CoA and malonyl-CoA into tetraketides, a process that is reported to rely on the activity of polyketide synthases (PKS) such as chalcone synthase (CHS) or stilbene synthase (STS). In search of solutions to improve the effectiveness of tetraketide derivative production by fermentation of simple carbon sources, e.g. sugars, and using biosynthetic pathways expressed in microbial hosts, the inventors have found that production is affected by non-structural genes such as CHIL's and surprisingly, it has been found that the co-expression of CHILs has a dramatically positive effect on production of tetraketide derivatives such as flavonoids in strains co-expressing PKS's in the biosynthetic pathway. The effect is evident as an improved activity is observed at the early committed steps of CHS and STS, leading to formation of tetraketide derivatives such as flavonoids, dihydrochalcones and stilbenes. It is contemplated that via the increase in upstream efficiency at PKS level increased production can be achieved of basically any downstream tetraketide derivative.

The present inventors have also found substrate and product promiscuity of pathway enzymes to impede the efficient heterologous production of end products. This is particularly noticeable for the anthocyanidin synthase (ANS), an enzyme belonging to the group of 2-oxoglutarate dependent dioxygenases (2ODDs). ANS has very high similarity to flavonol synthase (FLS), and in yeast and bacteria ANS has been found to catalyze many of the same reactions normally associated with FLS and flavonol synthesis. The inventors have found that expression of biosynthetic pathways directed to anthocyanin (ACN) production, can result in high amounts of undesired flavonols (both aglycones and their 3-O-glycosides) instead of the desired ACNs. Similarly, pathways directed towards epicatechins (EPC) is contemplated to result in flavonol accumulation at the expense of EPC's. Without being bound to the theory, the accumulation of flavonols is contemplated to be associated with an unwanted substrate/product promiscuity of ANS and the present inventors have observed that expressing the full length, structural ACN pathway in yeast can result in accumulation of up to a hundred fold more flavonols than ACNs—and that unwanted flavonol production was a general feature of all ANS enzymes observed. The present invention employing CHIL to further increase downstream metabolite levels in e.g. flavonoid pathways such as the pathway to ACN or EPC, is contemplated to further amplify the undesired formation of flavonols. In search of ways to improve biosynthetic pathways for producing ACN's and/or EPC's co-expression of a glutathione-S-transferase (GST) can direct ANS product specificity towards anthocyanidin and in particular in combination with the increased levels of precursor metabolites caused by employing the CHIL and PKS of the present invention, dramatically shift the preference of ANS towards anthocyanidins. The co-expression of GST, together with structural genes such as ANS, solves the inherent problem of unwanted flavonol side-product formation by the ANS enzyme. Hence, when ANS, GST, and an anthocyanin glycosyl transferase (such as A3GT) is co-expressed, this results predominantly in ACN production, and when ANS, GST, and an anthocyanidin reductase (ANR) is co-expressed the production is predominantly of EPCs.

Accordingly, the present invention provides in a first aspect a recombinant microbial host cell producing a tetraketide or derivatives thereof from one or more substrates selected from cinnamoyl-CoA, p-Coumaroyl-CoA, Caffeoyl-CoA, Feruloyl-CoA, malonyl-CoA, sinapoyl-CoA, and dihydro derivatives thereof, comprising an operative biosynthetic metabolic pathway for the tetraketide or derivatives thereof comprising a chalcone isomerase-like (CHIL) polypeptide heterologous to the host cell and a Type 3 polyketide synthase (PKS).

In a further aspect the invention provides a recombinant polynucleotide construct comprising a nucleotide encoding the chalcone isomerase-like (CHIL) polypeptide and/or the Type 3 polyketide synthase (PKS) of the invention, operably linked to a promotor which is heterologous to the CHIL and/or type 3 PKS encoding polynucleotide.

In a further aspect the invention provides an expression vector comprising the polynucleotide construct of the invention.

In a further aspect the invention provides a cell culture, comprising host cells of the invention and a growth medium.

In a further aspect the invention provides a method for producing a tetraketide derivative comprising
a) culturing the cell culture of the invention at conditions allowing the host cell to convert the one or more substrates into the tetraketide derivative; and
b) optionally recovering and/or isolating the tetraketide derivative.

In a further aspect the invention provides a fermentation liquid comprising the cell culture of the invention and its contents of tetraketide or derivative thereof.

In a further aspect the invention provides a composition comprising the fermentation liquid of the invention and one or more agents, additives and/or excipients.

In a further aspect the invention provides a use of the composition of the invention, wherein the tetraketide derivative is an anthocyanin as a dye, colorant or non-therapeutic bioactive compound.

In a further aspect the invention provides a method of preparing a pharmaceutical preparation comprising subjecting the composition of the invention to one or more steps transforming the composition and its contents of tetraketide or derivative thereof into a therapeutically relevant mixture comprising one or more pharmaceutical grade additives and/or adjuvants.

In a further aspect the invention provides a pharmaceutical preparation obtainable from the pharmaceutical preparation method of the invention.

In a further aspect the invention provides the pharmaceutical preparation of the invention for use as a medicament.

### DESCRIPTION OF DRAWINGS AND FIGURES

FIG. 1 depicts the early flavonoid biosynthetic pathway, from the amino acids phenylalanine or tyrosine to the common flavanones, shown with the various hydroxylation patterns of the B-ring (ring names indicated for naringenin): pinocembrin with no hydroxylation of B-ring, naringenin with one hydroxylation (4′), eriodictyol with two hydroxylations (3′,4′), and pentahydroxyflavanone with three (3′4′5′), respectively. All flavanones further have hydroxylations at the A-ring (3,5). The number of hydroxylations on the B-ring depends on the starter molecule, cinnamic acid or p-coumaric acid, and the P450 enzymes F3′H and F3′5′H. Bold arrows indicate the most common plant pathway to naringenin, the precursor of most other flavonoids. Names of enzymes are indicated as abbreviations, with corresponding full names as provided in list of SEQ IDs.

FIG. 2 depicts examples of flavonoid biosynthetic pathways derived from the common flavanone precursor. For simplicity, only the most common flavanone, naringenin, with a single B-ring hydroxylation is shown. Abbreviated enzyme names correspond to full names as provided in the list of SEQ IDs.

FIG. 3 depicts examples of stilbene biosynthetic pathways derived from cinnamic acid and p-coumaric acid, respectively. Abbreviated enzyme names correspond to full names as provided in the list of SEQ IDs.

FIG. 4 depicts examples of dihydrochalcone and dihydrostilbene biosynthetic pathways derived from p-coumaric acid by including a DBR. Similar types of compounds can be derived from cinnamoyl-CoA (not shown). Abbreviated enzyme names correspond to full names as provided in the list of SEQ IDs.

FIG. 5 depicts the biosynthetic pathway from flavanones to flavanols (aka flavan-3-ols) and anthocyanins. For simplicity, only derivatives with a single B-ring hydroxylation is shown. In planta, the pathway from naringenin normally leads to derivatives such as (−)-epiafzelechin and pelargonidin-3-O-glucoside. However, ex planta a number of side activities of ANS have been reported, as indicated by dashed lined arrows. Abbreviated enzyme names correspond to full names as provided in the list of SEQ IDs.

FIG. 6 depicts a graphic representation of the integration constructs used to integrate multiple genes into the genome of the host, S. cerevisiae, via in vivo homologous recombination (see Eichenberger et al., Met. Eng., 2017, 39:80-89 for details). Each DNA fragment, comprising a gene expression cassette (promoter-gene-terminator), or a specific helper fragment (see Example no. 2) is nested between homologous recombination tags (HRT), i.e 60 base pair sequences indicated with a letter, in which the preceding fragment has a sequence that is identical to the next fragment and so forth. All fragments have been excised from the backbone of the plasmid, in which they are maintained and amplified, by a restriction enzyme digest, using the Asc I recognition sites flanking each fragment. One fragment comprises two parts, separated by Asc I, which has homology to the intended genomic target sequence, allowing concomitant in vivo integration, by homologous recombination, of all fragments after a single host transformation event (see Shao 2009, Nucl. Acids Res., 37 (2) e16 for details).

FIG. 7 depicts a schematic representation of the HRT in vivo plasmid assembly. Shown is an example of the fragments needed for in vivo assembly, in S. cerevisae, of a multi gene expression plasmid. Each fragment comprises either a gene expression cassette, or the elements required for stable maintenance and replication in yeast. The gene expression cassettes comprise a yeast promoter, a gene encoding sequence, and a yeast terminator sequence. Each fragment is flanked by homologous recombination tags (HRTs) as described in FIG. 6, and by restriction enzyme recognition sites for Asc I. This restriction enzyme is used to release each fragment form the backbone of the plasmid used to maintain and amplify the fragment (typically a bacterial pUC based plasmid). After a single transformation of yeast with a mixture of the relevant fragments, these are in vivo assembled into a single copy plasmid, by way of the host's DNA repair system, allowing stable expression of the heterologous genes comprised in the expression cassettes.

### INCORPORATION BY REFERENCE

All publications, patents, and patent applications referred to herein are incorporated by reference to the same extent as if each individual publication, patent, or patent application was specifically and individually indicated to be incorporated by reference. In the event of a conflict between a term herein and a term in an incorporated reference, the term herein prevails and controls.

### DETAILED DESCRIPTION OF THE INVENTION

In this section the invention is described in further detailed embodiments.

### Definitions

The terms “polypeptide” and “protein” are used herein interchangeably. Proteins with catalytic function are referred to as “enzymes”.

The term “PAL” as used herein refers to phenylalanine ammonia lyase, an EC4.3.1.24 enzyme capable of catalyzing conversion of phenylalanine to cinnamic acid.

The term “C4H” as used herein refers to the enzyme trans-cinnamate 4-monooxygenase also known as cinnamate 4-hydroxylase, an EC1.14.14.91 CYP450 enzyme capable of catalyzing conversion of cinnamic acid to p-coumaric acid.

The term “CYP450 as used herein” refers to an enzyme of the Cytochrome P450 family, capable of oxidizing a range of substrates. Upon acting on a substrate, CYP450 must be reduced by its cognate reductase (CPR) to regain catalytic capacity.

The term “CPR” as used herein refers to cytochrome P450 reductase, an EC1.6.2.4 enzyme catalyzing the reduction of CYP450 enzymes.

The term “TAL” as used herein refers to tyrosine ammonia lyase or a PAL enzyme with TAL activity, an EC4.3.1.25 enzyme capable of catalyzing conversion of tyrosine to p-coumaric acid.

The term “C3H” as used herein refers to coumarate 3-hydroxylase, an EC1.14.13 enzyme catalyzing conversion of p-coumaric acid to caffeic acid.

The term “4CL” as used herein refers to 4-coumarate-CoA-ligase, an EC6.2.1.12 enzyme capable of catalyzing conversion of the ligation of CoA to various phenylpropanoic acids.

The term “COMT” as used herein refers to caffeic acid 3-O-methyltransferase, an EC2.1.1.68 enzyme capable of catalyzing conversion of caffeic acid to ferulic acid.

The term “CCOMT” as used herein refers to caffeoyl-CoA-O-methyltransferase, an EC2.1.1.104 enzyme capable of catalyzing conversion of caffeoyl-CoA to feruloyl-CoA.

The term “HCT” as used herein refers to shikimate O-hydroxycinnamoyltransferase, an EC2.3.1.133 enzyme capable of ligating shikimate to various CoA-activated phenylpropanoic acids.

The term “C3′H” as used herein refers to 5-O-(4-coumaroyl)-D-quinate 3′-monooxygenase, an EC1.14.14.96 enzyme capable of catalyzing conversion of trans-5-O-(4-coumaroyl)-D-quinate or trans-5-O-(4-coumaroyl)-shikimate to trans-5-O-caffeoyl-D-quinate or trans-5-O-caffeoyl-D-shikimate, respectively.

The term “Type 3 PKS” or “type 3 polyketide synthase” as used herein refers to polyketide synthase, an enzyme capable of catalyzing the condensation of a CoA-activated substrate with one or more malonyl-CoA units.

The term “CHIL” as used herein refers to chalcone isomerase-like protein, a polypeptide also known as the non-catalytic type IV CHI.

The term “CHS” as used herein refers to chalcone synthase, a type 3 polyketide synthase enzyme capable of synthesizing a chalcone by condensing 3 molecules of malonyl-CoA with a phenylpropanoyl CoA (aka (hydroxy)-cinnamoyl-CoA), such as a naringenin chalcone from one molecule of p-coumaroyl CoA and three molecules of malonyl CoA.

The term “CHI” as used herein refers to chalcone isomerase, an enzyme capable of stereospecifically isomerizing naringenin chalcone to (2S)-naringenin.

The term “STS” as used herein refers to a stilbene synthase, a type 3 polyketide synthase enzyme capable of catalyzing the formation of a stilbene or dihydrostilbene from one molecule of cinnamoyl CoA or p-coumaroyl CoA and three molecules of malonyl CoA.

The term “FH” as used herein refers to flavonoid hydroxylase, an enzyme capable of hydroxylating flavonoids.

The term “F3H” as used herein refers to the enzyme flavanone 3-hydroxylase, an enzyme capable of hydroxylating (2S)-Naringenin at the 3-position to (2R,3R)-dihydrokaempferol, a dihydroflavonol. F3H belongs to the 2-oxoglutarate-dependent dioxygenase (20DD) family.

The terms “F3′H” and “F3′5′H” as used herein refers to flavonoid 3′-hydroxylase and flavonoid 3′,5′-hydroxylase respectively, which are CYP450 enzymes capable of catalyzing hydroxylation of naringenin to form eriodictyol and 5,7,3′4′5′-pentahydroxyflavanone, respectively, or of catalyzing hydroxylation of dihydrokaempferol (DHK) to form (2R,3R)-dihydroquercetin and dihydromyricetin, respectively.

The term “DFR” as used herein refers to dihydroflavonol 4-reductase, an enzyme capable of reducing dihydroflavonols to the corresponding 3,4-cis leucoanthocyanidins.

The term “LAR” as used herein refers to leucoanthocyanidin reductase, an enzyme capable of catalyzing conversion of leucoanthocyanidins into flavan-3-ols, such as catechins.

The term “ANS as used herein” refers to anthocyanidin synthase (also known as leucoanthocyanidin dioxygenase or LDOX), an enzyme capable of synthesizing anthocyanidins from 3,4-cis leucoanthocyanidin. ANS belongs to the 2ODD family

The term “GST” as used herein refers to glutathione-S-transferase, a class of enzymes best known for their ability to catalyze the conjugation of the reduced form of glutathione (GSH) to xenobiotic substrates for the purpose of detoxificationan. It should be noted, however, that GSTs may have a range of other functions, some of which might have not yet been fully elucidated.

The term “ANR” as used herein refers to anthocyanidin reductase, an enzyme capable of catalyzing conversion of anthocyanidin into epicatechins, such as (−)-epicatechin (2R,3R).

The term “UGT” as used herein refers to UDP-dependent glycosyltransferase, an enzyme capable of catalyzing conversion of anthocyanidins into anthocyanins.

The term “UDP-glucose” as used herein refers to uridine diphosphate glucose.

The term “A3GT” as used herein refers to anthocyanidin 3-O-glycosyltransferase, an enzyme capable of catalyzing glycosylation of anthocyanidins and/or other flavonoids at its 3-0 position using UDP-glucose.

The term “A3′GT” as used herein refers to anthocyanin 3′-O-glycosyl transferase, an enzyme capable of catalyzing glycosylation of anthocyanidins and/or other flavonoids at its 3′-O position using an UDP-sugar, such as UDP-glucose.

The term “A5GT” as used herein refers to anthocyanin-5-O-glycosyl transferase, an enzyme capable of catalyzing glycosylation of anthocyanidins and/or other flavonoids at its 5-0 position using an UDP-sugar, such as UDP-glucose.

The term “A7GT” as used herein refers to anthocyanin-7-O-glycosyl transferase, an enzyme capable of catalyzing glycosylation of anthocyanidins and/or other flavonoids at its 7-0 position using an UDP-sugar, such as UDP-glucose.

The term “A3′5′GT” as used herein refers to anthocyanin 3′5′-di-O-glycosyl transferase, an enzyme capable of catalyzing glycosylation of anthocyanidins and/or other flavonoids at its 3′-O and its 5′-O position using an UDP-sugar, such as UDP-glucose.

The term “IFS” as used herein refers to an enzyme capable of catalyzing conversion of flavanones into isoflavones, a conversion normally assisted by a dehydratase, such as the 2-hydroxy-isoflavanone dehydratase (EC4.2.1.105).

The term “HIFD” as used herein refers to Hydroxy isoflavanone dehydratase (EC4.2.1.105) an enzyme capable of catalyzing XXcapable of catalyzing the loss of a hydroxy group during the conversion of 2-hydroxy-isoflavones to isoflavones

The term “FNS” as used herein refers to, an enzyme capable of catalyzing conversion of a flavanone to a flavone, whether as the sole catalyst or assisted by a dehydratase.

The term “FLS” as used herein refers to, an enzyme capable of catalyzing conversion of dihydroflavonols (flavanonols) to flavonols.

The term “AOMT” as used herein refers to, an enzyme capable of methylating anthocyanins at one or more of the B-ring hydroxyl groups.

The term “AAT” as used herein refers to, an enzyme capable of transferring an acyl group to an anthocyanin, in particular to sugar moieties of anthocyanins.

The term “AAroAT” as used herein refers to anthocyanin aromatic acyl transferase, an enzyme capable of transferring an aromatic acyl group to an anthocyanin, in particular to sugar moieties of anthocyanins.

The term “AAliAT” as used herein refers to anthocyanin aliphatic acyl transferase, an enzyme capable of transferring an aliphatic acyl group to an anthocyanin, in particular to sugar moieties of anthocyanins.

The term “functional homolog” refers to a polypeptide that has sequence similarity to a reference polypeptide, and that carries out one or more of the biochemical or physiological function(s) of the reference polypeptide. A functional homolog and the reference polypeptide can be a natural occurring polypeptide, and the sequence similarity can be due to convergent or divergent evolutionary events. As such, functional homologs may be designated in the art as homologs, or orthologs, or paralogs. Variants of a naturally occurring functional homologues, such as polypeptides encoded by mutants of a wild type coding sequence, can themselves be functional homologs. Functional homologs can also be created via site-directed mutagenesis of the coding sequence for a polypeptide, or by combining domains from the coding sequences for different naturally-occurring polypeptides (“domain swapping”). Techniques for modifying genes encoding functional polypeptides described herein are known and include, inter alia, directed evolution techniques, site-directed mutagenesis techniques and random mutagenesis techniques, and can be useful to increase specific activity of a polypeptide, alter substrate specificity, alter expression levels, alter subcellular location, or modify polypeptide-polypeptide interactions in a desired manner. Such modified polypeptides are considered functional homologs. Functional homolog is sometimes applied to the polynucleotide that encodes a functionally homologous polypeptide.

The term “substrate” or “precursor”, as used herein refers to any compound that can be converted into a different compound. For example, XX can be a substrate for YY and can be converted into ZZ. For clarity, substrates and/or precursors include both compounds generated in situ by an enzymatic reaction in a cell or exogenously provided compounds, such as exogenously provided organic carbon molecules which the host cell can metabolize into a desired compound.

The term “Chalcone” as used herein refers to a molecule of the general formula:

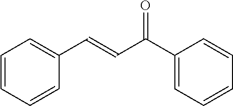

The term “flavonoid precursor” as used herein refers to intermediate compounds relevant for the pathways in a cell for producing flavonoids. These include the CHS starter molecules selected from acetyl-CoA, malonyl-CoA, cinnamic acid, cinnamoyl-CoA, p-coumaric acid, p-coumaroyl-CoA, benzoate, benzoyl-CoA, hydroxybenzoate, p-hydroxybenzoyl-CoA, sinapate, sinapoyl-CoA, ferulate, feruloyl-CoA, caffeate, caffeoyl-CoA, and intermediates from the groups of chalcones, flavanones, dihydroflavonols, leucoanthocyanidins, and anthocyanidins. Flavonoid precursors can be used to feed the recombinant host to achieve increased production of the end product. As will be known in the art, the same can be achieved by feeding the recombinant host with an excess of host molecules, such as aromatic amino acids.

The term “flavonoid” refers to molecules that have the general structure of a 15-carbon skeleton, which consists of two phenyl rings (A and B) and a heterocyclic ring (C). This carbon structure can be abbreviated C6-C3-C6. They are the result of sequential condensation reaction between phenylpropanoyl-CoA and 3 molecules of malonyl-CoA. The ring names as well as the conventional numbering of carbon atoms in flavonoids follows from:

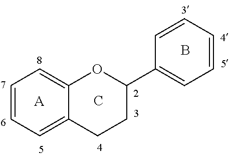

Flavonoids includes the non-ketone compounds which are more specifically termed flavanoids. Hence, the term “flavonoid” includes flavanones, flavones, isoflavones, flavanonols (dihydroflavonols), flavonols, flavans (flavan-3-ols, flavan-4-ols, and flavan-3,4-ols), and anthocyanidins, and refers to compounds of the formula I or II:

### (I)

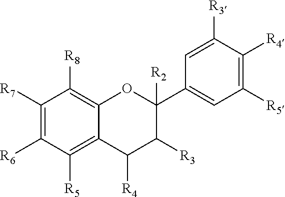

wherein
(a) the bond between C2 and C3 can be a single bond or a double bond,
(b) the B-ring phenyl group can be a C2-phenyl- or C3-phenyl-group,
(c) the configuration at C2 and C3 can be either R or S,
(d) R2, R3, R5, R6, R7, R8, R3′, R4′, and R5′ are selected from the group consisting of —H, and —OH; and
(e) R4 is selected from the group consisting of —H, ═O, and —OH;
(f) R6 and R8 are selected from the group consisting of —H, and —OH, and -glycosyl, and prenyl; and
(g) any of the hydroxyl groups (—OH) can be further modified by single or multiple additions of methyl groups, and/or one or more sugar groups, and/or acyl groups (aliphatic or aromatic), and/or prenyl groups, and/or any combination of said groups.

### (II)

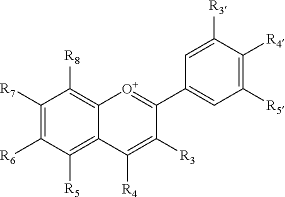

wherein
(a) R3, R5, R6, R7, R8, R3′, R4′, and R5′ are selected from the group consisting of —H, and —OH; and
(b) any of the hydroxyl groups (—OH) can be further modified by single or multiple additions of methyl groups, and/or one or more sugar groups, and/or acyl groups (aliphatic or aromatic), and/or prenyl groups, and/or any combination of said groups.

One or more hydroxyl groups of the flavonoid can be substituted with residues such as methyl, acyl, and glycosyl residues. The hydroxyl groups can be methylated at one or more positions. One or more hydroxyl groups can also be glycosylated with one or more sugar residues, the sugar residues being selected from the group consisting of glucose, rhamnose, xylose, galactose, and arabinose. The sugar residue can also be the sugar acid derivative, such as glucuronic acid. The residues consisting of one or more glycosides can be, for example, a monosaccharide, disaccharide, or a trisaccharide. The monosaccharide, disaccharide, and the trisaccharide can, for example, consist of sugar residues selected from the group consisting of glucose, rhamnose, xylose, galactose, and arabinose, and any combination thereof. One or more hydroxyl groups of the flavonoid can also be acylated, for example a flavonoid glycoside can be acylated, at one or more positions, on one or more of the sugar residues. Acyl residues can be selected from the group consisting of cinnamoyl, coumaroyl, caffeoyl, sinapoyl, feruloyl, malonyl, benzoyl, and hydroxybenzoyl. Further, acylated flavonoid glycoside, can be glycosylated on the acyl residue. Preferably, the flavonoid is glycosylated at one or more hydroxyl group, these primary glycosyl residues being further glycosylated and/or acylated. Optionally, these secondary glycosyl and/or acyl residues can be further substituted with sugar and/or acyl groups. It will be understood by a person skilled in the art, that glycosyl residues can be derivatized at one or more positions, whereas acyl groups are rarely derivatized at more than one position.

The term “anthocyanin” as used herein refers to any anthocyanidin comprising at least one glycosylation. Base anthocyanin structure is represented by molecules, including but not limited to those selected from pelargonidin, cyanidin, delphinidin, malvidin, peonidin, petunidin and their derivatives. It also includes chemical compounds belonging to the 3-deoxyanthocyanidins such as luteolinidin or diosmetinidin.

The term “Dihydrochalcone” as used herein refers to a molecule of the general formula:

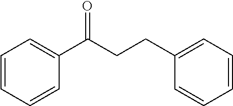

The term “Stilbene” as used herein refers to cis or trans molecules of the general formula:

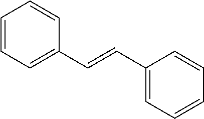

The term “Dihydrostilbene” as used herein refers to a molecule of the general formula:

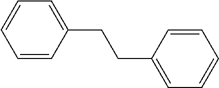

The term “microorganism” or “microbial cell” as used herein refers to a microscopic live organism, which may exist in its single-celled form, or in a colony of cells. Microbials comprise prokaryotes and some eukaryotes such as fungi, yeast, and molds. In one aspect microbials includes cells of higher organisms such as plants and animals unless the cells are isolated or cultured. In another aspect microbials excludes cells of higher organisms such as plants and animals unless the cells are isolated or cultured. Microorganism, host, host cell, recombinant host, and recombinant host cell are terms which are used interchangeably.

The term “host cell” refers to any cell type that is susceptible to transformation, transfection, transduction, or the like with a polynucleotide construct or expression vector comprising a polynucleotide of the present invention. The term “host cell” encompasses any progeny of a parent cell that is not identical to the parent cell due to mutations that occur during replication.

The term “recombinant host cell” is intended to refer to a host cell, wherein at least one DNA sequence has been modified, deleted from, or added to the genome, thereby augmenting or altering the genome. Such DNA sequences include but are not limited to genes that are not naturally present, DNA sequences that are not normally transcribed into RNA or translated into a protein (“expressed”), and other genes or DNA sequences which one desires to introduce into the non-recombinant host cell. Typically, the genome of a recombinant host cell described herein is augmented through stable introduction of one or more recombinant genes that may be inserted into the host cell genome and/or by way of an episomal vector (e.g., plasmid, YAC, etc.). Generally, introduced DNA is not originally resident in the host cell that is the recipient of the DNA, but it is within the scope of this disclosure to isolate a DNA segment from a given host cell, and to subsequently introduce one or more additional copies of that DNA into the same host cell, e.g., to enhance production of the product of a gene or alter the expression pattern of a gene. In some instances, the introduced DNA may modify or even replace an endogenous gene or DNA sequence by, e.g., homologous recombination or site-directed mutagenesis. In one embodiment the recombinant host cell is a host cell comprising and expressing heterologous or recombinant polynucleotide genes.

The term “in vivo”, as used herein refers to within a living cell, including, for example, a microorganism or a plant cell.

The term “in vitro”, as used herein refers to outside a living cell, including, without limitation, for example, in a microwell plate, a tube, a flask, a beaker, a tank, a reactor and the like.

The terms “polynucleotide,” “nucleotide,” “oligonucleotide,” and “nucleic acid” can be used interchangeably to refer to nucleotide sequence comprising DNA, RNA, derivatives thereof, or combinations thereof.

The term “polynucletide construct” refers to a polynucleotide molecule, either single- or double stranded, which is isolated from a naturally occurring gene or is modified to contain segments of polynucleotides in a manner that would not otherwise exist in nature or which is synthetic, and which comprises one or more control sequences.

The term “structural gene” as used herein refers to genes that encode enzymes that are directly involved in the conversion of a chemical substrate into the next chemical intermediate of a biosynthetic pathway, where “intermediate” can mean a chemical compound, which is the product of one catalytic conversion, or the substrate of the next catalytic conversion in the pathway.

The term “non-structural gene” as used herein refers to genes encoding a peptide or protein which is not itself catalytically active, but which can act, in a variety of ways, to support and enhance the function of enzymes encoded by the structural genes.

The term “gene” as used herein refers here to the expressible, polypeptide-encoding DNA sequence, corresponding to the RNA sequence found in the mature messenger RNA (mRNA) transcript. The DNA sequence corresponding to the RNA sequence is known as copyDNA or simply cDNA, and typically comprises at least the part of the mRNA that encodes a polypeptide, also known as the “coding sequence” or the “open reading frame” (ORF). Genes can be codon optimized for the intended host organism, using standard methods in the art, or can be prepared by reverse transcription of the mRNA, followed by PCR amplification to prepare cDNA. The polypeptide encoded by the ORF can be a functional protein, with or without catalytic function. The former are normally considered to be enzymes. Although the term “gene” normally refers to a DNA sequence encoding a known polypeptide, it is known from the art that DNA sequences of genes can be changed (mutated, truncated, extended, fused) while retaining the desired function of the polypeptide.

The term “cDNA” refers to a DNA molecule that can be prepared by reverse transcription from a mature, spliced, mRNA molecule obtained from a eukaryotic or prokaryotic cell. cDNA lacks intron sequences that may be present in the corresponding genomic DNA. The initial, primary RNA transcript is a precursor to mRNA that is processed through a series of steps, including splicing, before appearing as mature spliced mRNA.

The term “coding sequence” refers to a nucleotide sequence, which directly specifies the amino acid sequence of a polypeptide. The boundaries of the coding sequence are generally determined by an open reading frame, which begins with a start codon such as ATG, GTG, or TTG and ends with a stop codon such as TAA, TAG, or TGA. The coding sequence may be a genomic DNA, cDNA, synthetic DNA, or a combination thereof.

The term “control sequence”, “regulatory sequence” “control region” and “regulatory region” as used herein interchangeably (prokaryotic and eukaryotic) refers to nucleotide sequences (control sequence) that influence transcription or translation initiation rate, or the stability of a transcript or the resulting translation product. Regulatory regions include, without limitation, promoter sequences, enhancer sequences, response elements, protein recognition sites, inducible elements, protein binding sequences, 5′ and 3′ untranslated regions (UTRs), transcriptional start sites, termination sequences, polyadenylation sequences, introns, and combinations thereof. A regulatory region typically comprises at least a core (basal) promoter. A regulatory region also can include at least one control element, such as an enhancer sequence, an upstream enhancer element, or an upstream activation region (UAR). A regulatory region is operably linked to a coding sequence by positioning the regulatory region and the coding sequence so that the regulatory region is effective for regulating transcription or translation of the coding sequence. For example, to operably link a coding sequence and a promoter sequence, the translation initiation site of the translational reading frame of the coding sequence is typically positioned between one and about fifty nucleotides downstream of the promoter. A regulatory region can however be positioned as much as about 5,000 nucleotides upstream of the translation initiation site or about 2,000 nucleotides upstream of the transcription start site. The choice of regulatory regions to be included depends upon several factors, including, but not limited to, efficiency, selectability, inducibility, desired expression level, and preferential expression during certain culture stages. It is a routine matter for one of skill in the art to modulate the expression of a coding sequence by appropriately selecting and positioning regulatory regions relative to the coding sequence. It will be understood that more than one regulatory region can be present, e.g., introns, enhancers, upstream activation regions, transcription terminators, and inducible elements. One or more genes can be combined in a recombinant nucleic acid construct under the influence of common regulatory sequences. Combining a plurality of genes into such modules, particularly a polycistronic module, facilitates the expression of genes in a variety of species.

The term “expression vector” refers to a linear or circular DNA molecule that comprises a polynucleotide sequence encoding a polypeptide, and which is operably linked to control sequences that provide for its expression.

The term “operably linked” refers to a configuration in which a control sequence is placed at an appropriate position relative to the coding polynucleotide such that the control sequence directs expression of the coding polynucleotide.

The term “recombinant” as used herein, refers to any polynucleotide sequence that has been modified, mutated, truncated, or fused to another polynucleotide, in order to modify the function or expression of the sequence. The term “recombinant host” refers to any microorganisms comprising recombinant polynucleotide sequences, either by mutation or by intentional introduction of a polynucleotide sequence, or any other re-arrangement of its native polynucleotide sequence.

The term “recombinant gene” refers to a gene or DNA sequence that is introduced into a recipient host, regardless of whether the same or a similar gene or DNA sequence may already be present in such a host. “Introduced,” or “augmented” in this context, is known in the art to mean introduced or augmented by the hand of man. Thus, a recombinant gene can be a DNA sequence from another species, or can be a DNA sequence that originated from or is present in the same species, but has been incorporated into a host by recombinant methods to form a recombinant host. It will be appreciated that a recombinant gene that is introduced into a host can be identical to a DNA sequence that is normally present in the host being transformed. For any recombinant gene, one or more additional copies of the DNA can be introduced, to thereby permit overexpression or modified expression of the gene product of that DNA. A recombinant gene encoding a polypeptide described herein comprises the coding sequence for that polypeptide, operably linked in sense orientation to one or more control sequences suitable for expressing the polypeptide. Because many microorganisms are capable of expressing multiple gene products from a polycistronic mRNA, multiple polypeptides can be expressed under the control of a single regulatory sequence for those microorganisms, if desired. A coding sequence and a regulatory region are considered to be operably linked when control sequences within the regulatory region are positioned so that the regulatory region is effective for regulating transcription or translation of the sequence.

The terms “heterologous sequence,” “heterologous coding sequence,” and “heterologous gene” are used to describe a sequence or gene derived from a species other than the recombinant host. For example, if the recombinant host is an S. cerevisiae cell, then the cell would include a heterologous sequence derived from an organism other than S. cerevisiae. A heterologous coding sequence or gene, for example, can be from a prokaryotic microorganism, a eukaryotic microorganism, a plant, an animal, an insect, or from a fungus different to the recombinant host expressing the heterologous sequence.

In some aspects the coding sequence for a polypeptide described herein originates from a species other than the recombinant host, i.e., is a heterologous gene. In some of these aspects the coding sequence can be a chimaera, consisting of domains or regions from various organisms, or can be completely synthetic based on in silico modelling. Thus, if the recombinant host is a microorganism, the coding sequence can be from other prokaryotic or eukaryotic microorganisms, from plants or from animals. In many cases, the coding sequence for a polypeptide described herein is identified in a species other than the recombinant host, i.e., is a heterologous nucleic acid. In some cases, however, the coding sequence is a sequence that is native to the host and is being reintroduced into that organism. A native sequence can often be distinguished from the naturally occurring sequence by the presence of non-natural sequences linked to the exogenous nucleic acid, e.g., non-native regulatory sequences flanking a native sequence in a recombinant gene construct. Further, a native coding sequence can be operably linked to a native regulatory sequence, in a non-native manner. When such native regulatory sequences are used to control expression of a coding sequence other than its natural cognate coding sequence, the regulatory sequence is said to be heterologous to this native coding sequence. In addition, stably transformed exogenous genes typically are integrated at positions other than the position where the native sequence is found.

The terms “codon optimization” and “codon optimized” as used herein refer to a technique to maximize protein expression in fast-growing microorganisms such as E. coli or S. cerevisiae by increasing the translation efficiency of a particular gene. Codon optimization can be achieved, for example, by converting a nucleotide sequence of one species into a genetic sequence, which better reflects the translation machinery of a different, host species. Optimal codons help to achieve faster translation rates and high accuracy.

The term “metabolic pathway” as used herein is intended to mean two or more enzymes acting sequentially to convert chemical substrate(s) into chemical product(s). Enzymes are characterized by having catalytic activity, which can change the chemical structure of the substrate(s). An enzyme may have more than one substrate and produce more than one product. The enzyme may also depend on co-factors, which can be chemical compounds or can be a protein or enzyme. The CPR that reduces the Cytochrome P450 is an example of an enzymatic co-factor. The term “operative biosynthetic metabolic pathway” refers to a metabolic pathway that occurs in a live recombinant host, as described herein, and does not naturally occur in the host.

The term “engineered microorganism” refers to a recombinant host that contains an engineered biosynthetic pathway or operative metabolic pathway. Methods well known to those skilled in the art can be used to construct genetic expression constructs and recombinant cells according to this invention. These methods include in vitro recombinant DNA techniques, synthetic techniques, in vivo recombination techniques, and polymerase chain reaction (PCR) techniques. See, for example, techniques as described in Green & Sambrook, 2012, MOLECULAR CLONING: A LABORATORY MANUAL, Fourth Edition, Cold Spring Harbor Laboratory, New York; Ausubel et al., 1989, CURRENT PROTOCOLS IN MOLECULAR BIOLOGY, Greene Publishing Associates and Wiley Interscience, New York, and PCR Protocols: A Guide to Methods and Applications (Innis et al., 1990, Academic Press, San Diego, CA).

The term “expression” includes any step involved in the production of a polypeptide including, but not limited to, transcription, post-transcriptional modification, translation, post-translational modification, and secretion. “Functional expression” implies that a recombinant polynucleotide or its encoded polypeptide retains an activity similar to the expected and intended activity

The term “cell culture” as used herein refers to a culture medium comprising a plurality of recombinant host cells of the invention. A cell culture may comprise a single strain of recombinant host or may comprise two or more distinct host strains. The culture medium may be any medium that may comprise a recombinant host, e.g., a liquid medium (i.e., a culture broth) or a semi-solid medium, and may comprise additional components, e.g., a carbon source such as dextrose, sucrose, glycerol, or acetate; a nitrogen source such as ammonium sulfate, urea, or amino acids; a phosphate source; vitamins; trace elements; salts; amino acids; nucleobases; yeast extract; aminoglycoside antibiotics such as G418 and hygromycin B.

The term “conditions” as used herein for culturing cell cultures of the invention is intended to mean physico-chemical condition for the culturing of host cells allowing the culture to propagate and to express enzymes of the operative biosynthetic metabolic pathway in an active form and for these enzymes to operate effectively to produce a desired product of the pathway.

The terms “substantially” or “approximately” or “about”, as used herein refers to a reasonable deviation around a value or parameter such that the value or parameter is not significantly changed. These terms of deviation from a value should be construed as including a deviation of the value where the deviation would not negate the meaning of the value deviated from. For example, in relation to a reference numerical value the terms of degree can include a range of values plus or minus 10% from that value. For example, using these deviating terms can also include a range deviation plus or minus such as plus or minus 9%, 8%, 7%, 6%, 5%, 4%, 3%, 2%, or 1% from a specified value.

The term “and/or” as used herein is intended to represent an inclusive “or”. The wording X and/or Y is meant to mean both X or Y and X and Y. Further the wording X, Y and/or Z is intended to mean X, Y and Z alone or any combination of X, Y, and Z.

The term “isolated” as used herein about a compound, refers to any compound, which by means of human intervention, has been put in a form or environment that differs from the form or environment in which it is found in nature. Isolated compounds include but is not limited to compounds of the invention for which the ratio of the compounds relative to other constituents with which they are associated in nature is increased or decreased. In an important embodiment the amount of compound is increased relative to other constituents with which the compound is associated in nature. In an embodiment the compound of the invention may be isolated into a pure or substantially pure form. In this context a substantially pure compound means that the compound is separated from other extraneous or unwanted material present from the onset of producing the compound or generated in the manufacturing process. Such a substantially pure compound preparation contains less than 10%, such as less than 8%, such as less than 6%, such as less than 5%, such as less than 4%, such as less than 3%, such as less than 2%, such as less than 1%, such as less than 0.5% by weight of other extraneous or unwanted material usually associated with the compound when expressed natively or recombinantly. In an embodiment the isolated compound is at least 90% pure, such as at least 91% pure, such as at least 92% pure, such as at least 93% pure, such as at least 94% pure, such as at least 95% pure, such as at least 96% pure, such as at least 97% pure, such as at least 98% pure, such as at least 99% pure, such as at least 99.5% pure, such as 100% pure by weight.

The term “non-naturally occurring” as used herein about a substance, refers to any substance that is not normally found in nature or natural biological systems. In this context the term “found in nature or in natural biological systems” does not include the finding of a substance in nature resulting from releasing the substance to nature by deliberate or accidental human intervention. Non-naturally occurring substances may include substances completely or partially synthetized by human intervention and/or substances prepared by human modification of a natural substance.

The term “% identity” as used herein about polynucleotide or polypeptide sequences refers to the degree of identity in percent between two sequences. The term “% identity” for any polynucleotide or polypeptide relative to a reference polynucleotide or polypeptide is to be understood and determined as follows. A reference sequence (e.g. a polynucleotide or polypeptide sequence) is aligned to one or more candidate sequences using the computer program ClustalW (version 1.83, default parameters), which allows alignments of polynucleotide or polypeptide sequences to be carried out across their entire length (global alignment). See Chenna et al., Nucleic Acids Res., 31 (13): 3497-500 (2003). ClustalW calculates the best match between a reference and one or more candidate sequences, and aligns them so that identities, similarities, and differences can be determined. Gaps of one or more residues can be inserted into a reference sequence, a candidate sequence, or both, to maximize sequence alignments. For fast pairwise alignment of polynucleotide sequences, the following default parameters are used: word size: 2; window size: 4; scoring method: percentage; number of top diagonals: 4; and gap penalty: 5. For multiple alignment of polynucleotide sequences, the following parameters are used: gap opening penalty: 10.0; gap extension penalty: 5.0; and weight transitions: yes. For fast pairwise alignment of protein sequences, the following parameters are used: word size: 1; window size: 5; scoring method: percentage; number of top diagonals: 5; gap penalty: 3. For multiple alignment of protein sequences, the following parameters are used: weight matrix: blosum; gap opening penalty: 10.0; gap extension penalty: 0.05; hydrophilic gaps: on; hydrophilic residues: Gly, Pro, Ser, Asn, Asp, Gin, Glu, Arg, and Lys; residue-specific gap penalties: on. The ClustalW output is a sequence alignment that reflects the relationship between sequences. ClustalW can be run, for example, at the Baylor College of Medicine Search Launcher site on the World Wide Web (searchlauncher.bcm.tmc.edu/multi-align/multi-align.html) and at the European Bioinformatics Institute site on the World Wide Web (ebi.ac.uk/clustalw). To determine % identity of a candidate polynucleotide or amino acid sequence to a reference sequence, the sequences are aligned using ClustalW, the number of identical matches in the alignment is divided by the length of the reference sequence, and the result is multiplied by 100. It is noted that the % identity value can be rounded to the nearest tenth. For example, 78.11, 78.12, 78.13, and 78.14 are rounded down to 78.1, while 78.15, 78.16, 78.17, 78.18, and 78.19 are rounded up to 78.2. It will be appreciated that polynucleotides or polypeptides described herein can include additional nucleotides encoding additional amino acids that are not involved in polypeptide function (such as enzymatic activity), and thus such a polypeptide can be longer than would otherwise be the case. For example, a polypeptide can include a purification tag (e.g., HIS tag or GST tag), a chloroplast transit peptide, a mitochondrial transit peptide, an amyloplast peptide, signal peptide, or a secretion tag added to the amino or carboxy terminus. In some aspects, a polypeptide includes an amino acid sequence that functions as a reporter, e.g., a green fluorescent protein or yellow fluorescent protein. It will be appreciated that because of the degeneracy of the genetic code, a number of different polynucleotides can encode a particular polypeptide; i.e., for many amino acids, there is more than one nucleotide triplet that serves as the codon for the amino acid. Thus, codons in the coding sequence for a given polypeptide can be modified such that optimal expression in a particular host is obtained, using appropriate codon bias tables for that host (e.g., microorganism). As isolated polynucleotides, these modified sequences can exist as purified molecules and can be incorporated into a vector or a virus for use in constructing modules for recombinant polynucleotide constructs.

The term “comprise” and “include” as used throughout the specification and the accompanying claims as well as variations such as “comprises”, “comprising”, “includes” and “including” are to be interpreted inclusively. These words are intended to convey the possible inclusion of other elements or integers not specifically recited, where the context allows.

The articles “a” and “an” as used herein refers to one or to more than one (i.e. to one or at least one) of the grammatical object of the article. By way of example, “an element” may mean one element or more than one element.

Terms like “preferably”, “commonly”, “particularly”, and “typically” are not utilized herein to limit the scope of the claimed invention or to imply that certain features are critical, essential, or even important to the structure or function of the claimed invention. Rather, these terms are merely intended to highlight alternative or additional features that can or cannot be utilized in a particular embodiment of the present invention.

The term “product ratio” as used herein about enzymes refers to the ratio of products produced by an enzyme from a substrate. For example, ANS in the anthocyanin pathway will under different conditions covert leucoanthocyanidins into flavonol and anthocyanidin in various product ratios depending on the conditions.

The term “detectable concentration” refers to a level of tetraketides or derivatives thereof or other measured compounds measured in mg/L, nM, μM, or mM. Tetraketides or derivatives thereof can be detected and/or analyzed by techniques generally available to one skilled in the art, for example, but not limited to, thin layer chromatography (TLC), high-performance liquid chromatography (HPLC), ultraviolet-visible spectroscopy/spectrophotometry (UV-Vis), mass spectrometry (MS), nuclear magnetic resonance spectroscopy (NMR) or any combination thereof such as LC-MS.

Term “endogenous” or “native” as used herein refers to a gene or a polypeptide in a host cell which originates from the same host cell.

The term “deletion” as used herein refers to manipulation of a gene so that it is no longer expressed in a host cell.

The term “disruption” as used herein refers to manipulation of a gene or any of the machinery participating in the expression the gene, so that it is no longer expressed in a host cell.

The term “attenuation” as used herein refers to manipulation of a gene or any of the machinery participating in the expression the gene, so that it the expression of the gene is reduced as compared to expression without the manipulation.

### Recombinant Microbial Host Cells

The invention provides in a first aspect a recombinant microbial host cell producing a tetraketide or derivatives thereof from one or more substrates selected from cinnamoyl-CoA, p-Coumaroyl-CoA, Caffeoyl-CoA, Feruloyl-CoA, and dihydro derivatives thereof, comprising an operative biosynthetic metabolic pathway for the tetraketide or derivatives thereof comprising a chalcone isomerase-like (CHIL) polypeptide heterologous to the host cell and a Type 3 polyketide synthase (PKS). Further, the one or more substrates may also include malonyl-CoA and/or sinapoyl-CoA. It has been found that CHIL improves the function of PKS, so in a further embodiment the CHIL polypeptide increases PKS conversion of the one or more substrates into the respective tetraketide or derivatives thereof in the host cell of the invention compared to a recombinant host cell absent of the CHIL polypeptide. The CHIL enhance the catalytic efficiency of the PKS at least 1.25 fold, such as at least 1.5 fold, such as at least 1.75 fold, such as at least 2 fold, such as at least 3 fold, such as at least 5 fold, such as at least 10 fold, such as at least 50 fold, such as at least 100 fold, such as at least 500 fold, such as at least 1000 fold. In a still further embodiment the CHIL polypeptide of the invention has at least 80% identity to the CHIL polypeptide encoded by the sequence set forth in SEQ ID NO: 13, SEQ ID NO: 15, SEQ ID NO: 17, SEQ ID NO: 19, and/or SEQ ID NO: 21. More particularly in this embodiment the % sequence identity for the mentioned sequences may be at least 85%, such as at least 90%, such as at least 92%, such as at least 94%, such as at least 96%, such as at least 98%, such as at least 99%, such as at least 99.5%, such as at least 99.8%, such as at least 99.9%, such as 100% identity. The CHIL or the PKS or both the CHIL and the PKS are heterologous to the host cell of the invention.

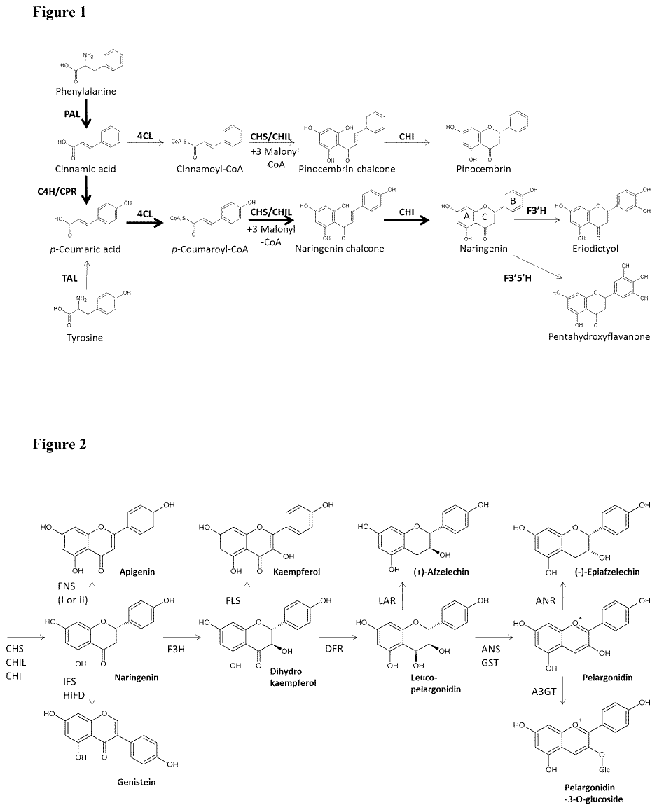

In the initial tetraketide operative biosynthetic metabolic pathway (see FIG. 1) intermediate p-coumaroyl-CoA is produced via the phenylpropanoid pathway. In plants, the starting molecule is typically phenylalanine, which is deaminated by the action of PAL type enzymes to form cinnamic acid. Cinnamic acid is then hydroxylated to p-coumaric acid (also called 4-coumaric acid) e.g. by C4H. Alternatively, p-coumaric acid is formed directly from tyrosine by the action of TAL type enzymes. Some enzymes have both PAL and TAL activity. The enzyme 4CL activates p-coumaric acid to p-coumaroyl-CoA by attachment of a CoA group.

Accordingly, the host cell of the invention comprises in a further embodiment an operative biosynthetic metabolic pathway capable of producing the one or more substrates of the invention comprising one or more polypeptides selected from:
a) phenylalanine ammonia lyase (PAL/EC 4.3.1.24);
b) tyrosine ammonia lyase (TAL/EC 4.3.1.25);
c) CYP450 reductase (CPR/EC 1.6.2.4);
d) cinnamate 4-hydroxylase (C4H/EC 1.14.14.91);
e) Coumarate 3-hydroxylase (C3H/EC 1.14.13);
f) 4-coumaric acid-CoA ligase (4CL/EC 6.2.1.12);
g) caffeic acid 3-O-methyltransferase (COMT/EC 2.1.1.68);
h) caffeoyl-CoA-O-methyltransferase (CCOMT/EC 2.1.1.104);
i) shikimate O-hydroxycinnamoyltransferase (HCT/EC 2.3.1.133);
j) 5-O-(4-coumaroyl)-D-quinate 3′-monooxygenase (C3′H/EC 1.14.14.96); and
k) double bond reductase (DBR).

Among these pathway enzymes the host cell of the invention can in one embodiment comprise:
a) one or more polypeptides selected from phenylalanine ammonia lyase (PAL); tyrosine ammonia lyase (TAL), cinnamate 4-hydroxylase (C4H), coumarate 3-hydroxylase (C3H), caffeic acid 3-O-methyltransferase (COMT); shikimate O-hydroxycinnamoyltransferase (HCT); and 5-O-(4-coumaroyl)-D-quinate 3′-monooxygenase (C3′H); capable of producing one or more compounds selected from cinnamic acid, p-coumaric acid, caffeic acid and ferulic acid;
b) 4-coumaric acid-CoA ligase (4CL); and caffeoyl-CoA O-methyltransferase (CCOMT); capable of converting the one or more compounds selected from cinnamic acid; cinnamoyl-CoA; p-coumaric acid; p-coumaroyl-CoA; caffeic acid; caffeoyl-CoA caffeic acid; and ferulic acid; into one or more compounds selected from cinnamoyl-CoA; p-Coumaroyl-CoA; Caffeoyl-CoA; and Feruloyl-CoA; and
c) optionally double bond reductase (DBR).

In one embodiment the CYP450 reductase (CPR) is native to the host cell. However, one or more heterologous CPRs may be co-expressed to regenerate the CYP450 enzymes. Further, in certain embodiments it may be advantageous to express or overexpress the Saccharomyces cerevisiae ICE2 gene (NM_001179438) or homologues thereof, which may improve the stability of the Cytochrome P450 reductase (CPR) and thereby the efficiency of the CYP450 enzyme reaction (see e.g. Emmerstorfer A et al., 2015, Biotechnol. J., 10:623-35). Similarly, it may be advantageous to express or overexpress a CytB5 enzyme, such as the CytB5 from Petunia x hybrida (acc. no. AF098510) or a homologue thereof, which may enhance the function of CYP450 enzymes (see e.g. De Vetten N et al., 1999, Proc. Natl. Acad. Sci. USA, 96:778-783).

For a host cell comprising an initial tetraketide operative biosynthetic metabolic pathway the corresponding:
a) phenylalanine ammonia lyase (PAL) has at least 80% identity to the phenylalanine ammonia lyase encoded by the sequence set forth in SEQ ID NO: 1;
b) tyrosine ammonia lyase (TAL) has at least 80% identity to the tyrosine ammonia lyase encoded by the sequence set forth in SEQ ID NO: 3;
c) CYP450 reductase (CPR) has at least 80% identity to a CYP450 reductases encoded by a sequence set forth in SEQ ID NO: 23 or SEQ ID NO: 25;
d) trans-cinnamate 4-monooxygenase (C4H) has at least 80% identity to the trans-cinnamate 4-monooxygenase encoded by the sequence set forth in SEQ ID NO: 5;
e) 4-coumaric acid-CoA ligase (4CL) has at least 80% identity to the 4-coumaric acid-CoA ligase encoded by the sequence set forth in SEQ ID NO: 7;
f) caffeic acid 3-O-methyltransferase (COMT) has at least 80% identity to the caffeic acid 3-O-methyltransferase encoded by the sequence set forth in SEQ ID NO: 61;
g) caffeoyl-CoA-O-methyltransferase (CCOMT) has at least 80% identity to the caffeoyl-CoA-O-methyltransferase (CCOMT) encoded by the sequence set forth in SEQ ID NO: 63;
h) shikimate O-hydroxycinnamoyltransferase (HCT) has at least 80% identity to the shikimate O-hydroxycinnamoyltransferase encoded by the sequence set forth in SEQ ID NO: 67;
i) 5-O-(4-coumaroyl)-D-quinate 3′-monooxygenase (C3′H) has at least 80% identity to the 5-O-(4-coumaroyl)-D-quinate 3′-monooxygenase encoded by the sequence set forth in SEQ ID NO: 65; and/or
j) double bond reductase (DBR) has at least 80% identity to the double bond reductase (DBR) encoded by the sequence set forth in SEQ ID NO: 91.

More particularly in this embodiment the % sequence identity for the mentioned sequences may be at least 85%, such as at least 90%, such as at least 92%, such as at least 94%, such as at least 96%, such as at least 98%, such as at least 99%, such as at least 99.5%, such as at least 99.8%, such as at least 99.9%, such as 100% identity.

The tetraketide derivative of the invention is preferably selected from chalcones, dihydrochalcones, stilbenes and dihydrostilbenes or derivatives thereof.

In a particular embodiment involving metabolome formation and/or other stabilizing effects, the initial tetraketide operative biosynthetic metabolic pathway of the invention may further benefit from also including CHI in addition to CHIL.

### Flavonoid Tetraketide Derivatives

In the flavonoid pathway (see FIG. 1), CHS is the first committed enzyme and, although CHS in some cases accepts a variety of cinnamoyl- and hydroxycinnamoyl-CoA substrates, it normally catalyzes synthesis of naringenin chalcone from one molecule of p-coumaroyl-CoA and three molecules of malonyl-CoA. Naringenin chalcone is stereospecifically isomerized to the colorless (2S)-naringenin, a flavanone, by CHI. (2S)-Naringenin is hydroxylated at the 3-position by F3H to yield (2R,3R)-dihydrokaempferol, a dihydroflavonol. Alternatively, the flavanones are turned into flavones by two different types of flavone synthase enzymes, either a 2-oxoglutarate-dependent dioxygenase (FNS I) or an NADPH-dependent cytochrome P450 monooxygenase (FNS II). The dihydroflavonols can be branched into flavonols by the action of flavonol synthase (FLS). F3′H and F3′5′H catalyze hydroxylation of naringenin or dihydrokaempferol (DHK) to form eriodictyol and pentahydroxyflavanone, or (2R,3R)-dihydroquercetin and dihydromyricetin, respectively. F3′H and F3′5′H determine the hydroxylation pattern of the B-ring of flavonoids and anthocyanins and are necessary for cyanidin and delphinidin production, respectively. They are key enzymes that determine the structures of flavonoids and anthocyanins. Dihydroflavonols are reduced to leucoanthocyanidins by DFR. Leucoanthocyanidins can be turned into catechins by the action of leucoanthocyanidin reductase (LAR). Alternatively, anthocyanidin synthase (ANS) catalyzes the synthesis of corresponding, colored anthocyanidins. Additionally, in order to form more stable anthocyanins, anthocyanidins can be 3-glucosylated by the action of A3GT. Alternatively, the anthocyanidins can be converted to epicatechins by the action of anthocyanidin reductase (ANR).

Accordingly, the polyketide synthase (PKS) of the invention may be a chalcone synthase (CHS) capable of converting the substrates into a chalcone and the operative biosynthetic metabolic pathway further comprises a chalcone isomerase (CHI) capable of converting the chalcone into a flavanone. Preferably the chalcone synthase (CHS) has at least 80% identity to the chalcone synthase (CHS) encoded by the sequence set forth in SEQ ID NO: 9 and/or the chalcone isomerase (CHI) has at least 80% identity to the chalcone isomerase encoded by the sequence set forth in SEQ ID NO: 11. More particularly in this embodiment the % sequence identity for the mentioned sequences may be at least 85%, such as at least 90%, such as at least 92%, such as at least 94%, such as at least 96%, such as at least 98%, such as at least 99%, such as at least 99.5%, such as at least 99.8%, such as at least 99.9%, such as 100% identity.

Further, the flavanone is preferably selected from one of naringenin, pinocembrin, and eriodictyol. Further the host cell of the invention can comprise polypeptides of an operative biosynthetic metabolic pathway capable of converting the flavanone into a flavonoid which is not a flavanone. Particular non-flavanone flavonoids include those selected from one or more of flavone, isoflavone, flavanonols, flavonol, flavans, anthocyanidins, and derivatives thereof.

In a particular embodiment the isoflavone is selected from genistein and daidzein and in another embodiment the flavonol is selected from quercetin, kaempferol and myricetin.

In particular, the non-flavanone flavonoid is selected from one or more of flavans, anthocyanidins and derivatives thereof. The anthocyanidin can be selected from anyone of pelargonidin, cyanidin, delphinidin, peonidin, petunidin, and malvidin.

The flavan can be a flavanol or derivatives thereof, such as a flavanol selected from one or more of flavan-3-ol, flavan-4-ol, flavan-3,4-diol and derivatives thereof. The flavan-3-ol can be selected from one or more of afzelechin, catechin, gallocatechin, catechin 3-gallate, Gallocatechin 3-gallate, epiafzelechin, Epicatechin, Epigallocatechin, Epicatechin 3-gallate, Epigallocatechin 3-gallate and any isomers thereof.

The host cell of the invention may comprise in the operative biosynthetic metabolic pathway one or more polypeptides of a flavonoid pathway selected from
a) flavonoid hydroxylase (FH);
b) dihydroflavonol-4-reductase (DFR);
c) leucoanthocyanidin reductase (LAR);
d) anthocyanidin synthase (ANS);
e) glutathione-S-transferase (GST);
f) anthocyanidin reductase (ANR);
g) UDP-dependent glycosyltransferase (UGT);
h) Isoflavone synthase (IFS);
i) Hydroxy isoflavanone dehydratase (HIFD);
j) Flavone synthase (FNS);
k) Flavonol synthase (FLS);
l) anthocyanin-O-methyl transferase (AOMT); and
m) anthocyanin acyl transferase (AAT).

The UDP-dependent glycosyltransferase (UGT) in this flavonoid pathway can be selected from one or more of:
a) anthocyanidin 3-O-glycosyltransferase (A3GT);
b) anthocyanin 3′-O-glycosyl transferase (A3′GT);
c) anthocyanin-5-O-glycosyl transferase (A5GT); and
d) anthocyanin 3′5′-di-O-glycosyl transferase (A3′5′GT).

In particular, the UDP-dependent glycosyltransferase (UGT) is anthocyanidin 3-O-glycosyltransferase (A3GT).

The flavonoid hydrolase (FH) in this flavonoid pathway can be selected from one or more of:
a) Flavonoid 3-hydroxylase (F3H);
b) flavonoid 3′-hydroxylase (F3′H); and
c) flavonoid 3′-5′-hydroxylase (F3′5′H).

The anthocyanin acyl transferase (AAT) in this flavonoid pathway can be selected from one or more of: anthocyanin aromatic acyl transferase (AAroAT); and anthocyanin aliphatic acyl transferase (AAliAT).

In a particular embodiment the flavanone is naringenin, the flavonoid is an anthocyanin and the operative biosynthetic metabolic pathway comprises, in addition to CHIL and CHS:
a) chalcone isomerase (CHI);
b) one or more polypeptides selected from flavanone 3-hydroxylase (F3H); flavonoid 3′-hydroxylase (F3′H) and Flavonoid 3′-5′-hydroxylase (F3′5′H);
c) dihydroflavonol-4-reductase (DFR);
d) anthocyanidin synthase (ANS); and
e) one or more polypeptides selected from anthocyanidin 3-O-glycosyltransferase (A3GT); anthocyanin 3′-O-glycosyl transferase (A3′GT); anthocyanin-5-O-glycosyl transferase (A5GT); anthocyanin-7-O-glycosyl transferase (A7GT); anthocyanin 3′5′-di-O-glycosyl transferase (A3′5′GT); anthocyanin acyl transferase (AAT); and anthocyanin-O-methyl transferase (AOMT).

In this particular embodiment the operative biosynthetic metabolic flavonoid pathway may also comprise a glutathione-S-transferase (GST); which modifies the product ratio of the anthocyanidin synthase (ANS) by decreasing ANS formation of flavanol or derivatives thereof and increasing ANS formation of anthocyanidin or derivatives thereof when converting leucoanthocyanidin. The product ratio is preferably modified by an at least 1.25 fold increase in formation of anthocyanidin or derivatives thereof, such as at least 1.5 fold, such as at least 1.75 fold, such as at least 2 fold, such as at least 3 fold, such as at least 5 fold, such as at least 10 fold, such as at least 50 fold, such as at least 100 fold, such as at least 500 fold, such as at least 1000 fold. Further, the glutathione-S-transferase (GST) may be expressed in the host cell of the invention as a fused polypeptide with the anthocyanidin synthase (ANS). Still further the glutathione-S-transferase (GST) may be co-expressed with the anthocyanidin synthase (ANS) in the host cell of the invention. In an embodiment the glutathione-S-transferase (GST) is heterologous to the host cell.

In certain embodiments of the invention, in which the flavonoid or anthocyanin is further derivatized by acylation, the host cell may comprise further enzymes that allow the production of CoA activated phenylpropanoid acyl-donor molecules. For example, the bacterial enzymes HpaB and HpaC, such as the HpaB from Pseudomonas aeruginosa (acc. no. PKG21040) or its homologues, and the HpaC from Salmonella enterica (acc. no. GAR62209) or ist homologues, may be included for the conversion of p-coumarate to caffeate. Any combination of HpaB and HpaC homologs may have the desired effect of hydroxylating the 3-position of p-coumarate (see e.g. Liu L et al., Engineering 2019, 5:287-295). Further, the caffeate, or the caffeoyl-CoA, may be further hydroxylated and/or methylated by expressing, in the host cell, an O-methyl transferase (OMT) such as the OMT1 from Arabidopsis thaliana (acc. no. AED96460 and with the function EC 2.1.1.68 or EC 2.1.1.42) or a homologue thereof, and/or a ferulate 5-hydroxylase (F5H) sauch as the Arabidopsis thaliana FAH1 (acc. no. NM_119790) or a homologue thereof. Combinations of these enzymes, together with the 4CL described above (with the function EC 6.2.1.12) will enable the production of the various CoA-activated phenylpropanoids, such as CoA esters of caffeate, ferulate, and sinapate. Similarly, the host cell may comprise heterologous nucleic acid sequences encoding one or more enzymes for the biosynthesis of benzoyl-CoA and/or p-hydroxybenzoyl-CoA. The anthocyanin is preferably selected from one or more of pelargonidin-3-O-glycoside (P3G), cyanidin-3-O-glycoside (C3G) delphinidin-3-O-glycoside (D3G); peonidin-3-O-glycoside, petunidin-3-O-glycoside, malvidin-3-O-glycoside; and derivatives thereof. A particularly interesting anthocyanin is selected from one or more of Petunidin 3-p-coumaroylrutinoside-5-glucoside; petunidin 3-feruloylrutinoside-5-glucoside; malvidin 3-p-coumaroylrutinoside-5-glucoside; pelargonidin 3-feruloylrutinoside-5-glucoside; pelargonidin rutinoside; pelargonidin 3-rutinoside-5-glucoside; pelargonidin 3-p-coumaroylrutinoside-5-glucoside; petunidin 3-rutinoside-5-glucoside; pelargonidin 3-rutinoside-5-glucoside; peonidin 3-rutinoside-5-glucoside; malvidin 3-rutinoside-5-glucoside; petunidin 3-rutinoside; pelargonidin 3-rutinoside; malvidin 3-rutinoside; petunidin 3-caffeoylrutinoside-5-glucoside; delphinidin 3-p-coumaroylrutinoside-5-glucoside; pelargonidin feruloyl-xylosyl-glucosylgalactoside; cyanidin 3-p-coumaroylrutinoside-5-glucoside; petunidin 3-p-coumaroylrutinoside-5-glucoside; petunidin 3-feruloylrutinoside-5-glucoside; pelargonidin 3-p-coumaroylrutinoside-5-glucoside; peonidin 3-p-coumaroylrutinoside-5-glucoside; malvidin 3-p-coumaroylrutinoside-5-glucoside; pelargonidin 3-feruloylrutinoside-5-glucoside; peonidin 3-feruloylrutinoside-5-glucoside; malvidin 3-feruloylrutinoside-5-glucoside; petunidin 3-p-coumaroylrutinoside; pelargonidin 3-p-coumaroylrutinoside; Cyanidin 3-O-glucoside; cyanidin 3;7-O-diglucoside; cyanidin 3-O-(3″;6″-O-dimalonyl)-glucoside; cyanidin 3-O-6″-O-malonylglucoside; delphinidin 3-O-glucoside; delphinidin 3;5;3′-O-triglucoside; delphinidin 3-O-(3″;6″-O-dimalonyl)-glucoside; delphinidin 3-O-6″-O-malonylglucoside; delphinidin 3-O-rutinoside; delphinidin 3-O-rutinoside 7-O-glucoside; delphinidin 3-O-rutinoside 7-O-(6″-O-p-hydroxybenzoyl)-glucoside; pelargonidin 3-O-glucoside; Cyanidin 3-(6″;6′″-di-p-coumarylsophoroside)-5-(6-malonylglucoside); Cyanidin 3-(6″;6′″-dicaffeylsophoroside)-5-glucoside; Cyanidin 3-(6″;6″-disinapylsophoroside)-5-glucoside; Cyanidin 3-(6″-caffeyl-6′″-ferulylsophoroside)-5-glucoside; Cyanidin 3-(6″-ferulyl-2″-sinapylsambubioside)-5-glucoside; Cyanidin 3-(6″-ferulyl-2″-sinapylsophoroside)-5-glucoside; Cyanidin 3-(6″-p-coumaryl-2″-sinapylsophoroside)-5-glucoside; Cyanidin 3-(6-malonylglucoside)-7;3′-di-(6-feruloylglucoside); Cyanidin 3-(6-malonylglucoside)-7-(6-feruloylglucoside)-3′-glucoside; Cyanidin 3-[2-(6-p-coumarylglucosyl)-6-caffeoylglucoside]-5-glucoside; Cyanidin 3-[2-(6-p-coumarylglucosyl)-6-p-coumarylglucoside)]-5-glucoside; Cyanidin 3-[6″-(4-glucosylcaffeyl isophoroside]-5-glucoside; Cyanidin 3-O-[2″-O-(2″-O-(sinapoyl) xylosyl) 6″-O-(p-coumaroyl) glucoside] 5-O-glucoside; Cyanidin 3-O-[beta-D-glucopyranoside]-7;3′-di-O-[6-O-(sinapyl)-beta-D-glucopyranoside]; Delphinidin 3-(6″;6″-di-p-coumarylsophoroside)-5-(6-malonylglucoside); Delphinidin 3-(6-malonylglucoside)-3′;5′-di-(6-p-coumaroylglucoside); Delphinidin 3-(diferuloyl) sophoroside-5-glucoside; Delphinidin 3-[2-(6-(feruloylglucoside)-6-feruloylglucoside]-5-(6-malonylglucoside); Delphinidin 3-[2-(6-feruloylglucoside)-6-p-coumaroylglucoside]-5-(6-malonylglucoside); Delphinidin 3-glucoside-5-(6-caffeoylglucoside)-3′-(6-(E)-p-coumaroylglucoside); Delphinidin 3-glucoside-7;3′-di-(6-(E)-sinapoylglucoside); Delphinidin 3-rutinoside-7-(6-p-coumaroylglucoside)-3′-glucoside; Cyanidin 3-glucoside-5;3′-di-(caffeoylglucoside); Malvidin 3-O-[6-O-(4-O-(4-O-(6-O-(trans-caffeoyl)-beta-D-glucopyranosyl)-trans-p-coumaroyl)-alpha-L-rhamnopyranosyl)-beta-D-glucopyranoside]; Pelargonidin 3-(6″;2′″-diferulylsambubioside)-5-(6-malonylglucoside); Pelargonidin 3-(6″-ferulyl-2″-sinapylsambubioside)-5-(6-malonylglucoside); Pelargonidin 3-(6″-ferulyl-2″-sinapylsambubioside)-5-glucoside; Pelargonidin 3-(6″-p-coumaryl-2″-sinapyl-sambubioside)-5-(6-malonylglucoside); Pelargonidin 3-(6″-p-coumaryl-2′″-sinapylsambubioside)-5-glucoside; Pelargonidin 3-[6′″-(3-glucosylcaffeyl) sophoroside]-5-glucoside; Pelargonidin 3-O-(6-O-malonyl-beta-D-glucopyranoside)-7-O-(6-O-(4-O-(trans-caffeyl)-beta-D-glucopyranosyl)-trans-caffeyl)-beta-D-glucopyranoside); Pelargonidin 3-O-[2-O-(2-(E)-feruloyl-beta-D-glucopyranosyl)-6-O-(E)-feruloyl-beta-D-glucopyranoside]-5-O-(beta-D-glucopyranoside); Pelargonidin 3-(2-(2-ferulylglucosyl)-6-ferulylglucoside)-5-glucoside; Pelargonidin 3-O-[2-O-(6-(E)-caffeoyl-beta-D-glucopyranosyl)-6-O-(E)-caffeoyl-beta-D-glucopyranoside]-5-O-(beta-D-glucopyranoside); Pelargonidin 3-(2-(6-caffeylglucosyl)-6-caffeylglucoside)-5-glucoside; Pelargonidin 3-O-[2-O-(6-(E)-caffeoyl-beta-D-glucopyranosyl)-6-O-(E)-feruloyl-beta-D-glucopyranoside]-5-O-(beta-D-glucopyranoside); Pelargonidin 3-(2-(6-caffeylglucosyl)-6-ferulylglucoside)-5-glucoside; Pelargonidin 3-O-[2-O-(6-(E)-caffeoyl-beta-D-glucopyranosyl)-6-O-(E)-p-coumaroyl-beta-D-glucopyranoside]-5-O-(beta-D-glucopyranoside); Pelargonidin 3-(2-(6-caffeylglucosyl)-6-p-coumarylglucoside)-5-glucoside; Pelargonidin 3-O-[2-O-(6-(E)-feruloyl-beta-D-glucopyranosyl)-6-O-(E)-caffeoyl-beta-D-glucopyranoside]-5-O-(beta-D-glucopyranoside); Pelargonidin 3-(2-(6-ferulylglucosyl)-6-caffeylglucoside)-5-glucoside; Pelargonidin 3-O-[2-O-(6-(E)-feruloyl-beta-D-glucopyranosyl)-6-O-(E)-feruloyl-beta-D-glucopyranoside]-5-O-(beta-D-glucopyranoside); Pelargonidin 3-(2-(6-ferulylglucosyl)-6-ferulylglucoside)-5-glucoside; Pelargonidin 3-O-[2-O-(6-(E)-feruloyl-beta-D-glucopyranosyl)-6-O-(E)-p-coumaroyl-beta-D-glucopyranoside]-5-O-(beta-D-glucopyranoside); Pelargonidin 3-(2-(6-ferulylglucosyl)-6-p-coumarylglucoside)-5-glucoside; Peonidin 3-[6″-(4-Petunidin 3-O-[6-O-(4-O-(4-O-(beta-D-glucosylcaffeyl) sophoroside]-5-glucoside; glucopyranosyl)-trans-p-coumaroyl)-alpha-L-rhamnopyranosyl)-beta-D-glucopyranoside]-5-O-[beta-D-glucopyranoside].

Alternatively, the selection of anthocyanin can be based on specific parameters, such as colour and/or stability of the molecule under various conditions, such as different pH, temperature, oxygen level, etc. In this embodiment, a host cell producing a basic anthocyanin scaffold, such as Pelargonidin-3-O-glucoside, Cyanidin-3-O-glucoside, or Delphinidin-3-O-glucoside, further expresses one or more randomly introduced genes encoding one or more of an O-methyl transferase, a glycosyl transferase, and an acyl transferase, plus any necessary accessory genes, e.g. those for producing various activated sugar- and acyl-donors. Assay conditions can thus be varied to allow selection of the molecules with desired properties, produced by the random combination of such modifying enzymes. In certain embodiments the assay conditions can be such as to include the presense of metal ions, or flavonoids which together with anthocyanins are able to result in co-pigmentation. In the case of flavonoids for co-pigmentation, these flavonoids may be added directly to the assay system, or they may be co-produced, together with anthocyanin, by the host itself or by co-cultivation with a secondary host.

In an alternative embodiment the flavonoid is a flavan-3-ol selected from (−)-epiafzelechin; (−)-epicatechin; and (−)-epigallocatechin and the operative biosynthetic metabolic pathway comprises:
a) chalcone isomerase (CHI);
b) one or more polypeptides selected from flavanone 3-hydroxylase (F3H); flavonoid 3′-hydroxylase (F3′H) and Flavonoid 3′-5′-hydroxylase (F3′5′H);
c) dihydroflavonol-4-reductase (DFR);
d) anthocyanidin synthase (ANS); and
e) anthocyanidin reductase (ANR).

Also, in this embodiment the operative biosynthetic metabolic flavonoid pathway may advantageously comprise glutathione-S-transferase (GST) which modifies the product ratio of the anthocyanidin synthase (ANS) by decreasing ANS formation of flavanol or derivatives thereof and increasing ANS formation of anthocyanidin or derivatives thereof when converting leucoanthocyanidin. The product ratio is preferably modified by an at least 1.25 fold increase in formation of anthocyanidin or derivatives thereof, such as at least 1.5 fold, such as at least 1.75 fold, such as at least 2 fold, such as at least 3 fold, such as at least 5 fold, such as at least 10 fold, such as at least 50 fold, such as at least 100 fold, such as at least 500 fold, such as at least 1000 fold. Further, the glutathione-S-transferase (GST) may be expressed in the host cell of the invention as a fused polypeptide with the anthocyanidin synthase (ANS). Still further the glutathione-S-transferase (GST) may be co-expressed with the anthocyanidin synthase (ANS) in the host cell of the invention. In an embodiment, the glutathione-S-transferase (GST) is heterologous to the host cell.

For a host cell comprising a flavonoid operative biosynthetic metabolic pathway the corresponding:
a) flavanone 3-hydroxylase (F3H) has at least 80% identity to the flavanone 3-hydroxylase encoded by the sequence set forth in SEQ ID NO: 39;
b) flavonoid 3′-hydroxylase (F3′H) has at least 80% identity to the flavonoid 3′-hydroxylase encoded by the sequence set forth in SEQ ID NO: 27;
c) flavonoid 3′,5′-hydroxylase (F3′5′H) has at least 80% identity to the flavonoid 3′,5′-hydroxylase encoded by the sequence set forth in SEQ ID NO: 29;
d) isoflavone synthase (IFS) has at least 80% identity to the isoflavone synthase encoded by the sequence set forth in SEQ ID NO: 35;
e) Hydroxy isoflavanone dehydratase (HIFD) has at least 80% identity to the isoflavone synthase encoded by the sequence set forth in SEQ ID NO: 37
f) flavone synthase (FNS) has at least 80% identity to the flavone synthase encoded by the sequence set forth in SEQ ID NO: 31, or SEQ ID NO:33
g) flavonol synthase (FLS) has at least 80% identity to the flavonol synthase encoded by the sequence set forth in SEQ ID NO: 41;
h) dihydroflavonol-4-reductase (DFR) has at least 80% identity to the dihydroflavonol-4-reductase encoded by the sequence set forth in SEQ ID NO: 43, SEQ ID NO: 45, or SEQ ID NO: 47;
i) anthocyanidin synthase (ANS) has at least 80% identity to an anthocyanidin synthase (ANS) encoded by the sequence set forth in SEQ ID NO: 53;
j) glutathione-S-transferase (GST) has at least 80% identity to the glutathione-S-transferase (GST) encoded by the sequence set forth in SEQ ID NO: 55;
k) leucoanthocyanidin reductase (LAR) has at least 80% identity to the leucoanthocyanidin reductase (LAR) encoded by the sequence set forth in SEQ ID NO: 49;
l) anthocyanidin reductase (ANR) has at least 80% identity to an anthocyanidin reductase (ANR) encoded by the sequence set forth in SEQ ID NO: 51;
m) anthocyanidin 3-O-glycosyltransferase (A3GT) has at least 80% identity to an anthocyanidin 3-O-glycosyltransferase encoded by the sequence set forth in SEQ ID NO: 57 or SEQ ID NO: 59;
n) anthocyanidin 3′-O-glycosyltransferase (A3′GT) has at least 80% identity to an anthocyanidin 3-O-glycosyltransferase encoded by the sequence set forth in SEQ ID NO: 87;
o) anthocyanin-5-O-glycosyl transferase (A5GT) has at least 80% identity to the anthocyanin-5-O-glycosyl transferase encoded by the sequence set forth in SEQ ID NO: 81;
p) anthocyanin-7-O-glycosyl transferase (A7GT) has at least 80% identity to the anthocyanin-7-O-glycosyl transferase encoded by the sequence set forth in SEQ ID NO: 69;
q) anthocyanin 3′5′-O-di-glycosyl transferase (A3′5′GT) has at least 80% identity to the anthocyanin 3′5′-di-O-glycosyl transferase (A3′5′GT) encoded by the sequence set forth in SEQ ID NO: 85;
r) anthocyanin aromatic acyl transferase (AAroAT) has at least 80% identity to the anthocyanin aromatic acyl transferase encoded by the sequence set forth in SEQ ID NO: 79;
s) anthocyanin aliphatic acyl transferase (AAliAT) has at least 80% identity to the anthocyanin aliphatic acyl transferase encoded by the sequence set forth in SEQ ID NO: 77; and/or
t) anthocyanidin O-methyl transferase (AOMT) has at least 80% identity to the anthocyanin O-methyl transferase encoded by the sequence set forth in SEQ ID NO: 97.

More particularly in this embodiment the % sequence identity for the mentioned sequences may be at least 85%, such as at least 90%, such as at least 92%, such as at least 94%, such as at least 96%, such as at least 98%, such as at least 99%, such as at least 99.5%, such as at least 99.8%, such as at least 99.9%, such as 100% identity.

### Stilbenes and Derivatives

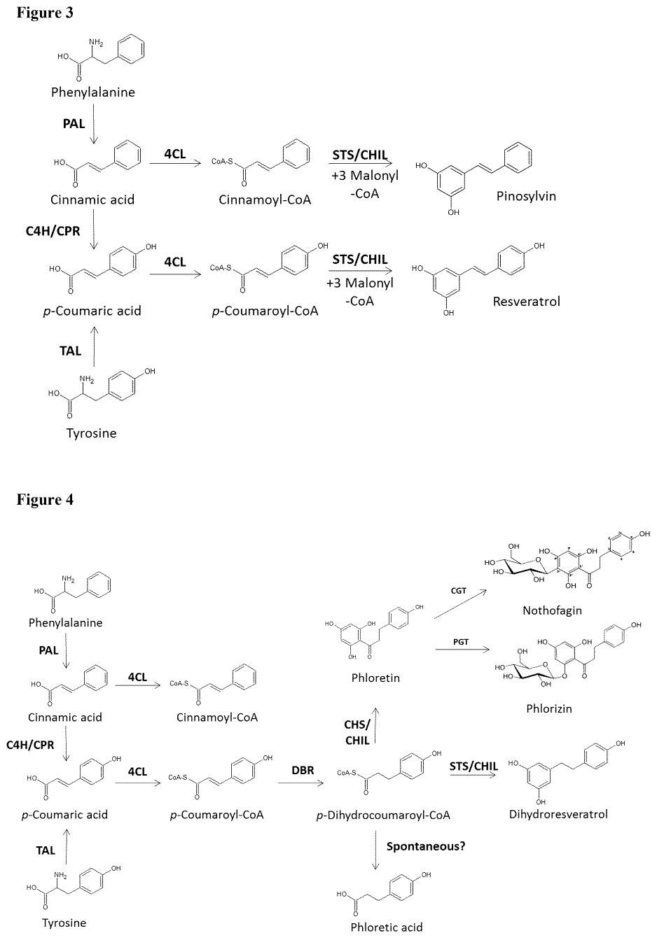

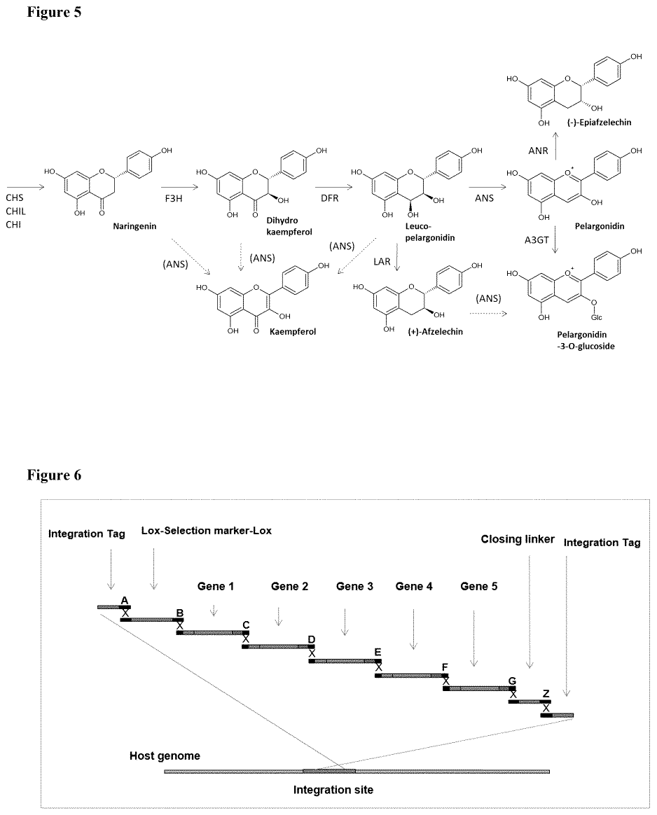

In the Stilbene pathway (see FIG. 3), Stilbene Synthase (STS) is the first committed enzyme and catalyzes synthesis of pinosylvin from cinnamoyl-CoA, and resveratrol from p-coumaroyl-CoA. These products may be further modified by hydroxylations, methylations, and/or prenylations.

Accordingly, the polyketide synthase (PKS) may also be a stilbene synthase (STS), capable of converting the substrates into a stilbene or a dihydrostilbene derivative. Preferably the stilbene synthase (STS) has at least 80% identity to the stilbene synthase (STS) encoded by the sequence set forth in SEQ ID NO: 71 or SEQ ID NO: 73. Further, the operative biosynthetic metabolic pathway may comprise trans-resveratrol di-O-methyltransferase (ROMT/EC 2.1.1.240).

The stilbene may be selected from one or more of 3,5-dihydroxystilbene (pinosylvin); 3,4′,5-trihydroxystilbene (resveratrol); 3,3′,4′,5-tetrahydroxy stilbene (piceatannol); and 3,5-dimethoxy-4′-hydroxystilbene (pterostilbene); and the operative biosynthetic metabolic pathway may comprise one or more polypeptides capable of hydroxylating and/or methylating stilbenes and/or dihydrostilbenes.

### Dihydrochalcones, Dihydrostilbenes, and Derivatives

For making tetraketide derivatives, such as dihydrochalcone and dihydrostilbene and further derivatives thereof the host cell of the invention may also comprises a Double Bond Reductase (DBR) capable of converting the one or more substrates selected from cinnamoyl-CoA, p-Coumaroyl-CoA, Caffeoyl-CoA, Feruoyl-CoA into the respective dihydrocinnamoyl-CoA, p-dihydrocoumaroyl-CoA, dihydrocaffeoyl-CoA and dihydroferuloyl-CoA which can then be converted to the further dihydro-derivatives by the Type 3 Polyketide Synthase (PKS). If the PKS is CHS the dihydro-substrates may be converted into dihydrochalcones, while if the PKS is STS the dihydro-substrates may be converted into dihydro-stilbenes. The dihydrochalcones and/or dihydrostilbenes may then be further derivatized upon including into the host cell further enzymes of the flavonoid and/or the stilbene operative biosynthetic metabolic pathway. The Double Bond Reductase (DBR) is in one embodiment native to the host cell, while in another embodiment it is heterologous to the host cell. The Double Bond Reductase (DBR) may be TSC13. Preferably, Double Bond Reductase (DBR) has at least 80% identity to the Double Bond Reductase (DBR) encoded by the sequence set forth in SEQ ID NO: 91; the chalcone synthase (CHS) has at least 80% identity to the chalcone synthase (CHS) encoded by the sequence set forth in SEQ ID NO: 9 and/or the stilbene synthase (STS) has at least 80% identity to the stilbene synthase (STS) encoded by the sequence set forth in SEQ ID NO: 71 or SEQ ID NO: 73. More particularly in this embodiment the % sequence identity for the mentioned sequences may be at least 85%, such as at least 90%, such as at least 92%, such as at least 94%, such as at least 96%, such as at least 98%, such as at least 99%, such as at least 99.5%, such as at least 99.8%, such as at least 99.9%, such as 100% identity.

The dihydrochalcone can be pinocembrin dihydrochalcone or naringenin dihydrochalcone (phloretin) or derivatives thereof, most preferably phloretin. The derivatives are preferably Phlorizin or Nothofagin.

For the operative biosynthetic metabolic pathway of the invention a singularity or a plurality of polypeptides, i.e. at least one, are heterologous to the host cell, i.e. they are encoded by genes that are heterologous to the host cell, and particularly a plurality of polypeptides are heterologous such as 2, 3, 4, 5, 6, 7, 8, 9, 10, 11, 12, 13, 14, 15 or all of the pathway polypeptides are heterologous to the host cell.

Recombinant Host Cells with Improved ANS Function

The invention provides in a further aspect a separate microbial recombinant host cell, comprising a glutathione-S-transferase (GST) and an operative biosynthetic metabolic pathway capable of producing an anthocyanidin comprising an anthocyanidin synthase (ANS) capable of converting a leucoanthocyanidin and/or a flavanol substrate into an anthocyanidin. The glutathione-S-transferase (GST) can modify the product specificity of the anthocyanidin synthase (ANS) by lowering formation of flavonol or derivatives thereof and increasing formation of anthocyanidin or derivatives thereof in the second host cell compared to a host cell wherein the is no glutathione-S-transferase (GST) present.

The glutathione-S-transferase (GST) preferably increases the anthocyanidin synthase (ANS) product ratio towards formation of anthocyanidin or derivatives thereof by at least 1.25 fold, such as at least 1.5 fold, such as at least 1.75 fold, such as at least 2 fold, such as at least 3 fold, such as at least 5 fold, such as at least 10 fold, such as at least 50 fold, such as at least 100 fold, such as at least 500 fold, such as at least 1000 fold.

The glutathione-S-transferase (GST) may in the second host cell be expressed as fused polypeptides with the anthocyanidin synthase (ANS) or it may be co-expressed with the anthocyanidin synthase (ANS).

The second host cell of the invention may further comprise a second operative biosynthetic metabolic pathway comprising one or more polypeptides of a flavonoid pathway selected from:
a) phenylalanine ammonia lyase (PAL);
b) tyrosine ammonia lyase (TAL);
c) CYP450 reductase (CPR);
d) cinnamate 4-hydroxylase (C4H);
e) cinnamate 3-hydroxylase (C3H);
f) 4-coumaric acid-CoA ligase (4CL);
g) Caffeic acid-O-methyl transferase (COMT);
h) chalcone isomerase-like (CHIL):
i) chalcone synthase (CHS);
j) chalcone isomerase (CHI);
k) flavonoid hydroxylase (FH);
l) dihydroflavonol-4-reductase (DFR);
m) leucoanthocyanidin reductase (LAR);
n) anthocyanidin reductase (ANR);
o) UDP-dependent glycosyltransferase (UGT);
p) anthocyanin-O-methyl transferase (AOMT); and
q) anthocyanin acyl transferase (AAT).
   The UDP-dependent glycosyltransferase (UGT) in this flavonoid pathway can be selected from one or more of:
e) anthocyanidin 3-O-glycosyltransferase (A3GT);
f) anthocyanin 3′-O-glycosyl transferase (A3′GT);
g) anthocyanin-5-O-glycosyl transferase (A5GT); and
h) anthocyanin 3′5′-di-O-glycosyl transferase (A3′5′GT).

The flavonoid hydroxylase (FH) in this flavonoid pathway can be selected from one or more of:
d) Flavonoid 3-hydroxylase (F3H);
e) flavonoid 3′-hydroxylase (F3′H); and
f) flavonoid 3′-5′-hydroxylase (F3′5′H).
   The anthocyanin acyl transferase (AAT) in this flavonoid pathway can be selected from one or more of: anthocyanin aromatic acyl transferase (AAroAT); and anthocyanin aliphatic acyl transferase (AAliAT).
   In a particular embodiment the second operative biosynthetic metabolic pathway comprises:
a) chalcone isomerase (CHI);
b) one or more polypeptides selected from flavanone 3-hydroxylase (F3H); flavonoid 3′-hydroxylase (F3′H) and Flavonoid 3′-5′-hydroxylase (F3′5′H); and
c) dihydroflavonol-4-reductase (DFR).
   In particular, the anthocyanidin can be selected from anyone of pelargonidin, cyanidin, delphinidin, peonidin, petunidin, and malvidin.

The second operative biosynthetic metabolic pathway comprises in an embodiment one or more polypeptides capable of further modifying the anthocyanidin into an anthocyanin, said polypeptides selected from:
a) anthocyanidin 3-O-glycosyltransferase (A3GT);
b) anthocyanin 3′-O-glycosyl transferase (A3′GT);
c) anthocyanin-5-O-glycosyl transferase (A5GT);
d) anthocyanin-7-O-glycosyl transferase (A7GT);
e) anthocyanin 3′5′-di-O-glycosyl transferase (A3′5′GT);
f) anthocyanin acyl transferase (AAT);
g) anthocyanin-O-methyl transferase (AOMT).
   Particularly, the second operative biosynthetic metabolic pathway comprises anthocyanidin 3-O-glycosyltransferase (A3GT).

The anthocyanin is particularly selected from one or more of pelargonidin-3-O-glucoside (P3G), cyanidin-3-O-glucoside (C3G), or delphinidin-3-O-glucoside (D3G) or derivatives thereof. The anthocyanin can more specifically be one or more of Petunidin 3-p-coumaroylrutinoside-5-glucoside; petunidin 3-feruloylrutinoside-5-glucoside; malvidin 3-p-coumaroylrutinoside-5-glucoside; pelargonidin 3-feruloylrutinoside-5-glucoside; pelargonidin rutinoside; pelargonidin 3-rutinoside-5-glucoside; pelargonidin 3-p-coumaroylrutinoside-5-glucoside; petunidin 3-rutinoside-5-glucoside; pelargonidin 3-rutinoside-5-glucoside; peonidin 3-rutinoside-5-glucoside; malvidin 3-rutinoside-5-glucoside; petunidin 3-rutinoside; pelargonidin 3-rutinoside; malvidin 3-rutinoside; petunidin 3-caffeoylrutinoside-5-glucoside; delphinidin 3-p-coumaroylrutinoside-5-glucoside; pelargonidin feruloyl-xylosyl-glucosylgalactoside; cyanidin 3-p-coumaroylrutinoside-5-glucoside; petunidin 3-p-coumaroylrutinoside-5-glucoside; petunidin 3-feruloylrutinoside-5-glucoside; pelargonidin 3-p-coumaroylrutinoside-5-glucoside; peonidin 3-p-coumaroylrutinoside-5-glucoside; malvidin 3-p-coumaroylrutinoside-5-glucoside; pelargonidin 3-feruloylrutinoside-5-glucoside; peonidin 3-feruloylrutinoside-5-glucoside; malvidin 3-feruloylrutinoside-5-glucoside; petunidin 3-p-coumaroylrutinoside; pelargonidin 3-p-coumaroylrutinoside; Cyanidin 3-O-glucoside; cyanidin 3;7-O-diglucoside; cyanidin 3-O-(3″;6″-O-dimalonyl)-glucoside; cyanidin 3-O-6″-O-malonylglucoside; delphinidin 3-O-glucoside; delphinidin 3;5;3′-O-triglucoside; delphinidin 3-O-(3″;6″-O-dimalonyl)-glucoside; delphinidin 3-O-6″-O-malonylglucoside; delphinidin 3-O-rutinoside; delphinidin 3-O-rutinoside 7-O-glucoside; delphinidin 3-O-rutinoside 7-O-(6″-O-p-hydroxybenzoyl)-glucoside; pelargonidin 3-O-glucoside; Cyanidin 3-(6″;6″-di-p-coumarylsophoroside)-5-(6-malonylglucoside); Cyanidin 3-(6″;6″-dicaffeylsophoroside)-5-glucoside; Cyanidin 3-(6″;6″-disinapylsophoroside)-5-glucoside; Cyanidin 3-(6″-caffeyl-6′″-ferulylsophoroside)-5-glucoside; Cyanidin 3-(6″-ferulyl-2″-sinapylsambubioside)-5-glucoside; Cyanidin 3-(6″-ferulyl-2″-sinapylsophoroside)-5-glucoside; Cyanidin 3-(6″-p-coumaryl-2″-sinapylsophoroside)-5-glucoside; Cyanidin 3-(6-malonylglucoside)-7;3′-di-(6 -feruloylglucoside); Cyanidin 3-(6-malonylglucoside)-7-(6-feruloylglucoside)-3′-glucoside; Cyanidin 3-[2-(6-p-coumarylglucosyl)-6-caffeoylglucoside]-5-glucoside; Cyanidin 3-[2-(6-p-coumarylglucosyl)-6-p-coumarylglucoside)]-5-glucoside; Cyanidin 3-[6″-(4-glucosylcaffeyl isophoroside]-5-glucoside; Cyanidin 3-O-[2″-O-(2″-O-(sinapoyl) xylosyl) 6″-O-(p-coumaroyl) glucoside] 5-O-glucoside; Cyanidin 3-O-[beta-D-glucopyranoside]-7;3′-di-O-[6-O-(sinapyl)-beta-D-glucopyranoside]; Delphinidin 3-(6″;6″-di-p-coumarylsophoroside)-5-(6-malonylglucoside); Delphinidin 3-(6-malonylglucoside)-3′;5′-di-(6-p-coumaroylglucoside); Delphinidin 3-(diferuloyl) sophoroside-5-glucoside; Delphinidin 3-[2-(6-(feruloylglucoside)-6-feruloylglucoside]-5-(6-malonylglucoside); Delphinidin 3-[2-(6-feruloylglucoside)-6-p-coumaroylglucoside]-5-(6-malonylglucoside); Delphinidin 3-glucoside-5-(6-caffeoylglucoside)-3′-(6-(E)-p-coumaroylglucoside); Delphinidin 3-glucoside-7;3′-di-(6-(E)-sinapoylglucoside); Delphinidin 3-rutinoside-7-(6-p-coumaroylglucoside)-3′-glucoside; Cyanidin 3-glucoside-5;3′-di-(caffeoylglucoside); Malvidin 3-O-[6-O-(4-O-(4-O-(6-O-(trans-caffeoyl)-beta-D-glucopyranosyl)-trans-p-coumaroyl)-alpha-L-rhamnopyranosyl)-beta-D-glucopyranoside]; Pelargonidin 3-(6″;2′″-diferulylsambubioside)-5-(6-malonylglucoside); Pelargonidin 3-(6″-ferulyl-2′″-sinapylsambubioside)-5-(6-malonylglucoside); Pelargonidin 3-(6″-ferulyl-2″-sinapylsambubioside)-5-glucoside; Pelargonidin 3-(6″-p-coumaryl-2′″-sinapyl-sambubioside)-5-(6-malonylglucoside); Pelargonidin 3-(6″-p-coumaryl-2′″-sinapylsambubioside)-5-glucoside; Pelargonidin 3-[6′″-(3-glucosylcaffeyl) sophoroside]-5-glucoside; Pelargonidin 3-O-(6-O-malonyl-beta-D-glucopyranoside)-7-O-(6-O-(4-O-(trans-caffeyl)-beta-D-glucopyranosyl)-trans-caffeyl)-beta-D-glucopyranoside); Pelargonidin 3-O-[2-O-(2-(E)-feruloyl-beta-D-glucopyranosyl)-6-O-(E)-feruloyl-beta-D-glucopyranoside]-5-O-(beta-D-glucopyranoside); Pelargonidin 3-(2-(2-ferulylglucosyl)-6-ferulylglucoside)-5-glucoside; Pelargonidin 3-O-[2-O-(6-(E)-caffeoyl-beta-D-glucopyranosyl)-6-O-(E)-caffeoyl-beta-D-glucopyranoside]-5-O-(beta-3-(2-(6-caffeylglucosyl)-6-caffeylglucoside)-5-glucoside; D-glucopyranoside); Pelargonidin Pelargonidin 3-O-[2-O-(6-(E)-caffeoyl-beta-D-glucopyranosyl)-6-O-(E)-feruloyl-beta-D-glucopyranoside]-5-O-(beta-D-glucopyranoside); Pelargonidin 3-(2-(6-caffeylglucosyl)-6-ferulylglucoside)-5-glucoside; Pelargonidin 3-O-[2-O-(6-(E)-caffeoyl-beta-D-glucopyranosyl)-6-O-(E)-p-coumaroyl-beta-D-glucopyranoside]-5-O-(beta-D-glucopyranoside); Pelargonidin 3-(2-(6-caffeylglucosyl)-6-p-coumarylglucoside)-5-glucoside; Pelargonidin 3-O-[2-O-(6-(E)-feruloyl-beta-D-glucopyranosyl)-6-O-(E)-caffeoyl-beta-D-glucopyranoside]-5-O-(beta-D-glucopyranoside); Pelargonidin 3-(2-(6-ferulylglucosyl)-6-caffeylglucoside)-5-glucoside; Pelargonidin 3-O-[2-O-(6-(E)-feruloyl-beta-D-glucopyranosyl)-6-O-(E)-feruloyl-beta-D-glucopyranoside]-5-O-(beta-D-glucopyranoside); Pelargonidin 3-(2-(6-ferulylglucosyl)-6 -ferulylglucoside)-5-glucoside; Pelargonidin 3-O-[2-O-(6-(E)-feruloyl-beta-D-glucopyranosyl)-6-O-(E)-p-coumaroyl-beta-D-glucopyranoside]-5-O-(beta-D-glucopyranoside); Pelargonidin 3-(2-(6-ferulylglucosyl)-6-p-coumarylglucoside)-5-glucoside; Peonidin 3-[6″-(4-glucosylcaffeyl) sophoroside]-5-glucoside; Petunidin 3-O-[6-O-(4-O-(4-O-(beta-D-glucopyranosyl)-trans-p-coumaroyl)-alpha-L-rhamnopyranosyl)-beta-D-glucopyranoside]-5-O-[beta-D-glucopyranoside].

The second operative biosynthetic metabolic pathway may further comprise anthocyanidin reductase (ANR) capable of converting the anthocyanidin into one or more flavan-3-ols selected from (−)-epicatechin; (−)-epiafzelechin; and (−)-epigallocatechin.

For a host cell comprising the second operative biosynthetic metabolic pathway the corresponding:
a) phenylalanine ammonia lyase (PAL) has at least 80% identity to the phenylalanine ammonia lyase encoded by the sequence set forth in SEQ ID NO: 1;
b) tyrosine ammonia lyase (TAL) has at least 80% identity to the tyrosine ammonia lyase encoded by the sequence set forth in SEQ ID NO: 3;
c) CYP450 reductase (CPR) has at least 80% identity to a CYP450 reductases encoded by a sequence set forth in SEQ ID NO: 23 or SEQ ID NO: 25;
d) trans-cinnamate 4-monooxygenase (C4H) has at least 80% identity to the trans-cinnamate 4-monooxygenase encoded by the sequence set forth in SEQ ID NO: 5;
e) 4-coumaric acid-CoA ligase (4CL) has at least 80% identity to the 4-coumaric acid-CoA ligase encoded by the sequence set forth in SEQ ID NO: 7;
f) chalcone synthase (CHS) has at least 80% identity to the chalcone synthase (CHS) encoded by the sequence set forth in SEQ ID NO: 9;
g) caffeic acid 3-O-methyltransferase (COMT) has at least 80% identity to the caffeic acid 3-O-methyltransferase encoded by the sequence set forth in SEQ ID NO: 61;
h) chalcone isomerase-like (CHIL) polypeptide has at least 80% identity to the chalcone isomerase-like (CHIL) polypeptide encoded by the sequence set forth in SEQ ID NO: 13, SEQ ID NO: 15, SEQ ID NO: 17, SEQ ID NO: 19, and/or SEQ ID NO: 21;
i) the chalcone isomerase (CHI) has at least 80% identity to the chalcone isomerase encoded by the sequence set forth in SEQ ID NO: 11;
j) the flavanone 3-hydroxylase (F3H) has at least 80% identity to the flavanone 3-hydroxylase encoded by the sequence set forth in SEQ ID NO: 39;
k) the flavonoid 3′-hydroxylase (F3′H) has at least 80% identity to a flavonoid 3′-hydroxylase encoded by a sequence set forth in SEQ ID NO: 27;
l) the flavonoid 3′,5′-hydroxylase (F3′5′H) has at least 80% identity to a flavonoid 3′,5′-hydroxylase encoded by a sequence set forth in SEQ ID NO: 29;
m) the dihydroflavonol-4-reductase (DFR) has at least 80% identity to a dihydroflavonol-4-reductase encoded by a sequence set forth in SEQ ID NO: 43, SEQ ID NO: 45, or SEQ ID NO: 47;
n) the anthocyanidin synthase (ANS) has at least 80% identity to an anthocyanidin synthase (ANS) encoded by a sequence set forth in SEQ ID NO: 53;
o) glutathione-S-transferase (GST) has at least 80% identity to an glutathione-S-transferase (GST) encoded by a sequence set forth in SEQ ID NO: 55;
p) the leucoanthocyanidin reductase (LAR) has at least 80% identity to a leucoanthocyanidin reductase (LAR) encoded by a sequence set forth in SEQ ID NO: 49;
q) the anthocyanidin reductase (ANR) has at least 80% identity to an anthocyanidin reductase (ANR) encoded by a sequence set forth in SEQ ID NO: 51
r) the anthocyanidin 3-O-glycosyltransferase (A3GT) has at least 80% identity to an anthocyanidin 3-O-glycosyltransferase encoded by a sequence set forth in SEQ ID NO: 57 or SEQ ID NO: 59;
s) anthocyanidin 3′-O-glycosyltransferase (A3′GT) has at least 80% identity to an anthocyanidin 3-O-glycosyltransferase encoded by a sequence set forth in SEQ ID NO: 87;
t) the anthocyanin-5-O-glycosyl transferase (A5GT) has at least 80% identity to the anthocyanin-5-O-glycosyl transferase encoded by the sequence set forth in SEQ ID NO: 81;
u) anthocyanin-7-O-glycosyl transferase (A7GT) has at least 80% identity to the anthocyanin-7-O-glycosyl transferase encoded by the sequence set forth in SEQ ID NO: 69;
v) anthocyanin 3′5′-O-di-glycosyl transferase (A3′5′GT) has at least 80% identity to the anthocyanin 3′5′-di-O-glycosyl transferase (A3′5′GT) encoded by the sequence set forth in SEQ ID NO: 85;
w) anthocyanin aromatic acyl transferase (AAroAT) has at least 80% identity to the anthocyanin aromatic acyl transferase encoded by the sequence set forth in SEQ ID NO: 79;
x) anthocyanin aliphatic acyl transferase (AAliAT) has at least 80% identity to the anthocyanin aliphatic acyl transferase encoded by the sequence set forth in SEQ ID NO: 77; and/or
y) anthocyanidin O-methyl transferase (AOMT) has at least 80% identity to the anthocyanin O-methyl transferase encoded by the sequence set forth in SEQ ID NO: 97.

For the second operative biosynthetic metabolic pathway a singularity or a plurality of polypeptides, i.e. at least one, are heterologous to the second host cell, i.e. they are encoded by genes that are heterologous to the second host cell, and particularly a plurality of polypeptides are heterologous such as 2, 3, 4, 5, 6, 7, 8, 9, 10, 11, 12, 13, 14, 15 or all of the pathway polypeptides are heterologous to the second host cell. In an embodiment one or more of the glutathione-S-transferase (GST) and the anthocyanidin synthase (ANS) is heterologous to the second host cell.

### Sources of Pathway Enzymes

In the operative biosynthetic metabolic pathways of the invention a singularity or a plurality of polypeptides/enzymes (i.e. at least one) and genes encoding them are heterologous to the host cell. Depending on the host cell the heterologous enzymes/polypeptides may be sourced from various sources. For example, in the case of a yeast host, the genes may be derived from plants, archaea, bacteria, animals, and other fungi. In an embodiment, at least one heterologous gene is derived from a plant. In certain embodiments, the heterologous genes can be selected from any one or a combination of organisms. For example, organisms which can be the source of heterologous genes for use in the host cell of the invention include species from one or more of the following plant genera: Petunia, Malus, Anthurium, Zea, Arabidopsis, Ammi, Glycine, Hordeum, Medicago, Populus, Fragaria, Dianthus, and the like. Representative species from these genera that may be used include Petunia x hybrida, Malus domestica, Anthurium andraeanum, Arabidopsis thaliana, Ammi majus, Hordeum vulgare, Medicago sativa, Populus trichocarpa, Fragaria x ananassa, and Dianthus caryuphyllus. In a further embodiment, genes encoding orthogonal enzymes from other organisms may also be included. Hence, there may be many options for constructing operational biosynthetic metabolic pathways by identifying a set of genes/enzymes that will work well together in a given microorganism.

Host optimization to improve expression of the heterologous pathways described is also possible. This may for example be done in such a way as to improve the ability of the host to provide higher levels of precursor molecules, tolerate higher levels of product, or to eliminate unwanted host enzyme activity, which interferes with the heterologous flavonoid-producing pathway. It may be advantageous to include transporter polypeptides, to facilitate transport of intermediates or end products of the heterologous biosynthic pathway across cell membranes, such as cell or vacuolar membranes. Such transporters are well known in the art, and may facilitate uptake of precursor molecules or the secretion of end products, sometimes increasing the overall production of end product. As will be understood by a skilled person, any enzyme of the operative biosynthetic metabolic pathway can be a target for optimization by genetic modifications, such as specific deletions, insertions, alterations, e.g., by mutagenesis, to improve both the specificity and turn-over rate of that enzyme. It will also be understood that the operational biosynthetic metabolic pathway may be constructed in such a way that, for each biosynthetic step, one or more genes is included. The one or more gene may be orthologs, carrying out the same biosynthetic reaction, or they be multiple copies of the same gene. Moreover, while specific enzymes are disclosed herein, the skilled person will appreciate that each disclosed enzyme represents its enzymatic function not only the listed enzyme and the disclosure should not be considered to be limited to the particular enzyme exemplified herein by name or sequence.

### Host Cells

Host cells of the invention (including both host cells and second host cells) may be eukaryotic cells selected from the group consisting of mammalian, amphibian, insect, plant, or fungal cells. The host cells may be a fungal cell selected from phylas consisting of Ascomycota, Basidiomycota, Neocallimastigomycota, Glomeromycota, Blastocladiomycota, Chytridiomycota, Zygomycota, Oomycota and Microsporidia. In particular the host cell is a yeast cell selected from the group consisting of ascosporogenous yeast (Endomycetales), basidiosporogenous yeast, and Fungi Imperfecti yeast (Blastomycetes), particularly a yeast cell is selected from the genera consisting of Saccharomyces, Kluveromyces, Candida, Pichia, Debaromyces, Hansenula, Yarrowia, Zygosaccharomyces, Arxula, Cyberlindnera, Xanthophyllomyces and Schizosaccharomyces. For specific species the yeast host cell may be selected from the species consisting of Kluyveromyces lactis, Saccharomyces carlsbergensis, Saccharomyces cerevisiae, Saccharomyces diastaticus, Saccharomyces douglasii, Saccharomyces kluyveri, Saccharomyces norbensis, Saccharomyces oviformis, Schizosaccharomyces pombe, Candida glabrata, Cyberlindnera jadinii, Pichia pastoris, Kluyveromyces lactis, Hansenula polymorpha, Candida boidinii, Arxula adeninivorans, Xanthophyllomyces dendrorhous, Candida albicans, and Yarrowia lipolytica. In another embodiment the host cell is filamentous fungus. Suitable filamentous fungal host cell may be selected among the phylas consisting of Ascomycota, Eumycota and Oomycota, particularly selected from the genera of Acremonium, Aspergillus, Ashbya, Aureobasidium, Bjerkandera, Ceriporiopsis, Chrysosporium, Coprinus, Corio/us, Cryptococcus, Filibasidium, Fusarium, Gibberella, Humicola, Magnaporthe, Mucor, Myceliophthora, Neocallimastix, Neurospora, Paecilomyces, Penicillium, Phanerochaete, Phlebia, Piromyces, Pleurotus, Schizophyllum, Talaromyces, Thermoascus, Thielavia, Tolypocladium, Trametes, and Trichoderma.

More specially a filamentous fungal host cell may be selected among the species of Aspergillus awamori, Aspergillus foetidus, Aspergillus fumigatus, Aspergillus japonicus, Aspergillus nidulans, Aspergillus niger, Aspergillus oryzae, Ashbya gossypii, Bjerkandera adusta, Ceriporiopsis aneirina, Ceriporiopsis caregiea, Ceriporiopsis gilvescens, Ceriporiopsis pannocinta, Ceriporiopsis rivulosa, Ceriporiopsis subrufa, Ceriporiopsis subvermispora, Chrysosporiuminops, Chrysosporiumkeratinophilum, Chrysosporium lucknowense, Chrysosporium merdarium, Chrysosporium pannicola, Chrysosporium queenslandicum, Chrysosporium tropicum, Chrysosporium zonatum, Coprinus cinereus, Coriolus hirsutus, Fusarium bactridioides, Fusarium cerealis, Fusarium crookwellense, Fusarium culmorum, Fusarium graminearum, Fusarium graminum, Fusarium heterosporum, Fusarium negundi, Fusarium oxysporum, Fusarium reticulatum, Fusarium roseum, Fusarium sambucinum, Fusarium sarcochroum, Fusarium sporotrichioides, Fusarium sulphureum, Fusarium torulosum, Fusarium trichothecioides, Fusarium venenatum, Gibberella fujikuroi, Humicola insolens, Humicola lanuginosa, Mucor miehei, Myceliophthora thermophila, Neurospora crassa, Penicillium purpurogenum, Phanerochaete chrysosporium, Phlebia radiata, Pleurotus eryngii, Thielavia terrestris, Trametes villosa, Trametes versicolor, Trichoderma harzianum, Trichoderma koningii, Trichoderma longibrachiatum, Trichoderma reesei, and Trichoderma viride.

In a particular embodiment the host cell of the invention is a yeast, particularly a yeast which belongs to the genus Saccharomyces, Klyuveromyces, Candida, Pichia, Debaromyces, Hansenula, Yarrowia, Zygosaccharomyces, or Schizosaccharomyces; such as the yeast Saccharomyces cerevisiae.

Host cells of the invention (including both host cells and second host cells) may also be prokaryotic cells, such as bacteria. Accordingly, the host cell of the invention may be a bacterium of a genera selected from Escherichia, Lactobacillus, Lactococcus, Corynebacterium, Bacillus, Acetobacter, Acinetobacter, Pseudomonas or Rhodobacter. In particular the host cell may be selected from the species of Escherichia coli, Corynebacterium glutamicum, Bacillus subtilus, Rhodobacter sphaeroides, Rhodobacter capsulatus, or Rhodotorula toruloides. In one embodiment the bacterium is Escherichia coli.

In a further alternative embodiment, the host cell of the invention may be an algae such as algaes of the species Blakeslea trispora, Dunaliella salina, Haematococcus pluvialis, Chlorella sp., Undaria pinnatifida, Sargassum, Laminaria japonica, or Scenedesmus almeriensis species. In a further alternative embodiment, the host cell of the invention is a cyanobacterium such as cyanobacteria selected from the species Blakeslea trispora, Dunaliella salina, Haematococcus pluvialis, Chlorella sp., Undaria pinnatifida, Sargassum, Laminaria japonica, or Scenedesmus almeriensis.

Alternative host cells of the invention may be plant cells for example of the genus Physcomitrella. In addition to plant cells the invention also provides an isolated plant, e.g., a transgenic plant, plant part, or plant cell culture comprising the operative biosynthetic metabolic pathway of the invention and producing the tetraketide or derivatives thereof in useful quantities. The tetraketide or derivatives thereof may be recovered from the plant or plant part or cell. The transgenic plant can be dicotyledonous (a dicot) or monocotyledonous (a monocot). Examples of monocot plants are grasses, such as meadow grass (blue grass, Poa), forage grass such as Festuca, Lolium, temperate grass, such as Agrostis, and cereals, e.g., wheat, oats, rye, barley, rice, sorghum, and maize (corn). Examples of dicot plants are tobacco, legumes, such as lupins, potato, sugar beet, pea, bean and soybean, and cruciferous plants (family Brassicaceae), such as cauliflower, rape seed, and the closely related model organism Arabidopsis thaliana. Examples of plant parts are stem, callus, leaves, root, fruits, seeds, and tubers as well as the individual tissues comprising these parts, e.g., epidermis, mesophyll, parenchyme, vascular tissues, meristems. Specific plant cell compartments, such as chloroplasts, apoplasts, mitochondria, vacuoles, peroxisomes and cytoplasm are also considered to be a plant part. Furthermore, any plant cell, whatever the tissue origin, is considered to be a plant part. Likewise, plant parts such as specific tissues and cells isolated to facilitate the utilization of the invention are also considered plant parts, e.g., embryos, endosperms, aleurone and seed coats. Also included within the scope of the present invention are the progeny of such plants, plant parts, and plant cells. The transgenic plant or plant cells comprising the operative pathway of the invention and produce the tetraketide or derivatives thereof may be constructed in accordance with methods known in the art. In short, the plant or plant cell is constructed by incorporating one or more expression vectors of the invention into the plant host genome or chloroplast genome and propagating the resulting modified plant or plant cell into a transgenic plant or plant cell.

The host cell of the invention may further be modified to provide an increased amount of a substrate for at least one polypeptide of the operative biosynthetic metabolic pathway and/or the host cell of the invention may be even further genetically modified to exhibit increased tolerance towards one or more substrates, intermediates, or product molecules from the operative biosynthetic metabolic pathway. Moreover, the host cell of the invention may comprise one or more native genes which have been attenuated, disrupted and/or deleted using method known in the art.

### Nucleotide Constructs

The invention also provides a recombinant polynucleotide construct comprising a nucleotide encoding the chalcone isomerase-like (CHIL) polypeptide and/or the Type 3 polyketide synthase (PKS) of the invention, operably linked to one or more control sequences, such as a promotor which is heterologous to the CHIL and/or type 3 PKS encoding polynucleotide.

Polynucleotides may be manipulated in a variety of ways to enable or optimize expression. Manipulation of the polynucleotide prior to its insertion into an expression vector may be desirable or necessary depending on the expression vector. The techniques for modifying polynucleotides utilizing recombinant DNA methods are well known in the art.

The control sequence may be a promoter, which is a polynucleotide sequence that is recognized by a host cell for expression of a polynucleotide. The promoter contains transcriptional control sequences that mediate the expression of the polypeptide. The promoter may be any polynucleotide that shows transcriptional activity in the host cell including mutant, truncated, and hybrid promoters, and may be obtained from genes encoding extracellular or intracellular polypeptides either native or heterologous to the host cell. The promoter may be an inducible promoter.

Examples of suitable promoters for directing transcription of the polynucleotide construct of the invention in a yeast host are listed as SEQ ID NOS: 101-108. They also include promoters obtained from the genes for Saccharomyces cerevisiae enolase (EN0-1), Saccharomyces cerevisiae galactokinase (GAL1), Saccharomyces cerevisiae alcohol dehydrogenase/glyceraldehyde-3-phosphate dehydrogenase (ADH1, ADH2/GAP), Saccharomyces cerevisiae triose phosphate isomerase (TPI), Saccharomyces cerevisiae metallothionein (CUP1), and Saccharomyces cerevisiae 3-phosphoglycerate kinase. Other useful promoters for yeast host cells are described by Romanos et al., 1992, Yeast 8:423-488.

The control sequence may also be a transcription terminator, which is recognized by a host cell to terminate transcription. The terminator is operably linked to the 3′-terminus of the polynucleotide encoding the polypeptide. Any terminator that is functional in the host cell may be used.

The control sequence may also be an mRNA stabilizer region downstream of a promoter and upstream of the coding sequence of a gene which increases expression of the gene.

The control sequence may also be a leader, a non-translated region of an mRNA that is important for translation by the host cell. The leader is operably linked to the 5′-terminus of the polynucleotide encoding the polypeptide. Any leader that is functional in the host cell may be used.

The control sequence may also be a polyadenylation sequence; a sequence operably linked to the 3′-terminus of the polynucleotide and, when transcribed, is recognized by the host cell as a signal to add polyadenosine residues to transcribed mRNA. Any polyadenylation sequence that is functional in the host cell may be used.

It may also be desirable to add regulatory sequences that regulate expression of the polypeptide relative to the growth of the host cell. Examples of regulatory systems are those that cause expression of the gene to be turned on or off in response to a chemical or physical stimulus, including the presence of a regulatory compound.

A CHIL encoding polynucleotide in the polynucleotide construct of the invention has in one embodiment at least 70%, such at least 75%, such as at least 80%, such as at least 90%, such as at least 95%, such as at least 99%, such as at least 100% identity to SEQ ID NO: 13, SEQ ID NO: 15, SEQ ID NO: 17, SEQ ID NO: 19, and/or SEQ ID NO: 21. The PKS encoding polynucleotide in the polynucleotide construct of the invention encodes in another embodiment a chalcone synthase (CHS) which has at least 70%, such at least 75%, such as at least 80%, such as at least 90%, such as at least 95%, such as at least 99%, such as at least 100% identity to SEQ ID NO: 9. The PKS encoding polynucleotide in the polynucleotide construct of the invention encodes in a further embodiment a stilbene synthase (STS) which has at least 70%, such at least 75%, such as at least 80%, such as at least 90%, such as at least 95%, such as at least 99%, such as at least 100% identity to SEQ ID NO: 71 or SEQ ID NO: 73.

A GST encoding polynucleotide construct of the invention has in one embodiment at least 70%, such at least 75%, such as at least 80%, such as at least 90%, such as at least 95%, such as at least 99%, such as at least 100% identity to SEQ ID NO: 55.

The transcriptional control sequences of the regulatory region controls can thus be a promoter, transcription terminator, mRNA stabilizer region, a leader, and/or a polyadenylation sequence and it can be either native or heterologous to the host cell. In one embodiment the control sequence is a promoter which is native to S. cerevisiae and has at least 70%, such at least 75%, such as at least 80%, such as at least 90%, such as at least 95%, such as at least 99%, such as at least 100% identity to a sequence selected from the groups consisting of SEQ ID NO: 101, SEQ ID NO: 102; SEQ ID NO: 103; SEQ ID NO: 104; SEQ ID NO: 105; SEQ ID NO: 106; SEQ ID NO: 107 and SEQ ID NO: 108.

Further the invention provides an expression vector comprising a polynucleotide construct of the invention.

Accordingly, in a special embodiment the host cell of the invention comprises an integrated polynucleotide construct of the invention and/or the expression vector of the invention.

The host cell may be further manipulated to contain a plurality of copies of any native or heterologous genes in the operative biosynthetic metabolic pathway producing tertraketide or derivatives thereof. For example, the host cell of the invention may include at least 2 copies of genes encoding the CHIL and/or type 3 PKS and/or the GST.

In a further embodiment the host cell has been further modified by introducing one or more heterologous genes, or by modifying one or more native genes, encoding a transporter, e.g. of the multidrug and toxic compound extrusion (MATE)-type or the ATP-binding cassettes (ABC)-type (reviewed by Zhao, 2015). Although GST has previously been contemplated to be associated with transport, the current application demonstrates that GST is involved in anthocyanidin production, independent of transporters. However, it is contemplated herein that the combination of GST and transporters for metabolites of the pathway and/or target molecule would have a beneficial impact on the final compound yield. This includes the engineering of host native transporters, e.g. transporter overexpression or engineering of native transporter specificity in order to facilitate transport of flavonoids and anthocyanins.

Although certain flavonoids are known to be secreted from plant roots, most flavonoids and anthocyanins are predominantly stored in the plant vacuole, and hence, the transport into the vacuole is the normal function of most flavonoid and anthocyanin transporters. However, it is conceivable that engineering transporters to relocate them to the host plasma membrane, as opposed to the tonoplast of vacuoles, would increase transport out of the cell, thereby increasing overall yield. This could happen by relieving internal stress from accumulating compounds, relieving unknown feedback inhibition, or by creating a sink for the pool of product molecules. Such engineering of transporters, in order to relocate them to the plasma membrane, is known in the art and can be applied to both C-terminal localization signals (Wolfenstetter et al. 2012) or N-terminal localization signals (Wang 2014). Transporters that could possibly be re-engineered includes, but are not limited to, ABC type transporters and MATE-type transporters. Some examples of ABC-type transporters are the ABCCs or ABCGs from Zea mays (Goodman et al. 2004; Badone et al. 2010), Vitis vinifera (Francisco et al. 2013), Homo sapiens (Chowdhurry et al. 2014), Arabidopsis thaliana (Liu et al. 2001; Francisco et al. 2013), or Medicago truncatula (Banasiak et al. 2013). Examples of suitable MATE-type transporters are MATEs from Solanum lycopersicum (Mathews et al. 2003), Arabidopsis thaliana (Debeaujon et al. 2001; Marinova et al. 2007; Zhao & Dixon, 2009), Vitis viniferas (Gomez et al. 2009), Medicago truncatula (Zhao & Dixon 2009; Zhao et al. 2011), Malus domestica (Frank et al. 2011), Brassica napus (Chai et al. 2009), Gossypium hirsitum (Gao et al. 2016) and Vaccinium corymbosum (Chen et al. 2015).

In addition, the host may have both ABC-type and MATE-type transporters suitable for engineering towards enhanced transport out of the cell.

### Cultures

The invention further provides a cell culture, comprising the host cells of the invention and a growth medium. The invention also provides a second cell culture, comprising the second host cell of the invention and a growth medium.

### Methods of Producing Tetraketide or Derivatives Thereof.

In a separate aspect the invention provides a method for producing a tetraketide derivative comprising:
a) culturing the cell culture of the invention at conditions allowing the host cell to convert the one or more substrates into the tetraketide derivative; and
b) optionally recovering and/or isolating the tetraketide derivative.

In the method of the invention the recovering step may comprise separating a liquid phase of the host cell or cell culture from a solid phase of the host cell or cell culture to obtain a supernatant comprising the tetraketide derivative by one or more steps selected from:
a) contacting the supernatant with one or more adsorbent resins in order to obtain at least a portion of the produced tetraketide derivative;
b) contacting the supernatant with one or more ion exchange or reversed-phase chromatography columns in order to obtain at least a portion of the tetraketide derivative;
c) crystallizing or extracting the tetraketide derivative; and
d) evaporating the solvent of the liquid phase to concentrate the tetraketide derivative;
thereby recovering the tetraketide derivative.

In an embodiment the method of the invention further comprises one or more elements selected from:
a) culturing the cell culture in a nutrient medium comprising vitamins, trace elements, salts, amino acids, nitrogen, and a carbon source;
b) culturing the cell culture under aerobic or anaerobic conditions
c) culturing the cell culture under agitation;
d) culturing the cell culture at a temperature of between 20-50° C.;
e) culturing the cell culture at a pH of between 3-9; and
f) culturing the cell culture for between 20 to 120 hours.

The method of the invention may further comprise at least one step of producing the tetraketide derivative, which is performed in vitro.

In a further aspect, the invention provides a method for increasing the catalytic activity of CHS and/or STS by co-expression of CHIL and optionally CHI, thereby increasing overall productivity and, hence, production of tetraketide derivatives. The co-expression of CHIL, with CHS and/or STS and optionally CHI, allows in vivo association of the proteins to enhance the overall productivity over the method not employing CHIL. In this embodiment the relative production of tetraketide derivatives may be increased at least 1.25 fold, such as at least 1.5 fold, such as at least 1.75 fold, such as at least 2 fold, such as at least 3 fold, such as at least 5 fold, such as at least 10 fold, such as at least 50 fold, such as at least 100 fold, such as at least 500 fold, such as at least 1000 fold.

In a still further aspect the invention provides a method for producing epicatechin or anthocyanin or a derivative thereof comprising incorporating a heterologous glutathione-S-transferase (GST) in a recombinant host cell of the invention comprising an operative biosynthetic metabolic pathway capable of producing said epicatechin or anthocyanin or a derivative thereof and culturing said host cell to produce said epicatechin or anthocyanin or a derivative thereof.

In a still further aspect, the invention provides a method for converting a leucoanthocyanidin into an anthocyanidin comprising contacting in a host cell of the invention the leucoanthocyanidin with an anthocyanidin synthase (ANS) in combination with a glutathione-S-transferase (GST).

In a still further aspect, the invention provides a method of modifying the product ratio of an anthocyanidin synthase (ANS) by decreasing ANS formation of flavonol or derivatives thereof and increasing ANS formation of anthocyanidin or derivatives thereof when converting leucoanthocyanidin, comprising contacting in a host cell of the invention the ANS with a glutathione-S-transferase (GST) in a reaction medium. In this embodiment the product ratio is preferably modified by an at least 1.25 fold increase in formation of anthocyanidin or derivatives thereof, such as at least 1.5 fold, such as at least 1.75 fold, such as at least 2 fold, such as at least 3 fold, such as at least 5 fold, such as at least 10 fold, such as at least 50 fold, such as at least 100 fold, such as at least 500 fold, such as at least 1000 fold.

It is appreciated by a person skilled in the art, that host cells and cultures of the invention can be cultivated using conventional cell culture or fermentation processes, including, inter alia, chemostat, batch, fed-batch cultivations, continuous perfusion fermentation, and continuous perfusion cell culture.

The skilled person will also appreciate that after the host cells have been grown in culture for a desired period of time, the tertraketide derivatives can then be recovered from the culture or culture medium using standard techniques known in the art.

### Fermentation Liquids

In a still further aspect, the invention provides a fermentation liquid comprising the cell culture of the invention and its contents of tetraketide derivatives. In the fermentation liquid of the invention at least 50% of the host cells may be lysed such as at least 75%, such as at least 95%, such as at least 99%. Also, in the fermentation liquid of the invention at least 50% (w/w) of solid cellular material may have been removed such as at least 75% (w/w), such as at least 95% (w/w), such as at least 99% (w/w). In the fermentation liquid of the invention the tetraketide derivative is in an embodiment a flavan-3-ol selected from one or more of (−)-epiafzelechin; (−)-epicatechin; and (−)-epigallocatechin, while in another embodiment the tetraketide derivative is an anthocyanin selected from one or more of pelargonidin-3-O-glycoside (P3G), cyanidin-3-O-glycoside (C3G) delphinidin-3-O-glycoside (D3G); peonidin-3-O-glycoside, petunidin-3-O-glycoside, malvidin-3-O-glycoside, and derivatives thereof. Still further in the fermentation liquid of the invention the tetraketide derivative may be a stilbene selected from one or more of pinosylvin; resveratrol; piceatannol; and pterostilbene. In the fermentation liquid of the invention the tetraketide derivative may also be a dihydrochalcone selected from one or more of pinocembrin dihydrochalcone or naringenin dihydrochalcone (phloretin).

Preferably, the tetraketide derivative concentration in the fermentation liquid is at least 5 mg/L, such as at least 10 mg/L, such as at least 20 mg/l, such as at least 50 mg/L, such as at least 100 mg/L, such as at least 500 mg/L, such as at least 1000 mg/L, such as at least 5000 mg/L, such as at least 10000 mg/L, such as at least 50000 mg/L.

### Compositions

In a further aspect the invention provides a composition comprising the fermentation liquid of the invention and one or more agents, additives and/or excipients. Agents, additives and/or excipients includes formulation additives, stabilising agent and fillers.

The composition may contain one or more co-pigments, which can affect stability, color, and hue of the tetraketide derivatives such as anthocyanins. This can be an intramolecular interaction e.g. of the acyl group with the rest of the tetraketide derivative, but it can also be an intermolecular interaction with other molecules in the composition. For processing, formulation and storage of products containing tetraketide derivatives, stabilization of the intact tetraketide derivatives may be desired. However, e.g. in vivo therapeutic effects of tetraketide derivatives can also be due to one or more of native degradation products or metabolites, in which case certain instability of the tetraketide derivatives in the composition may be desired for some applications. Notably, the amount of for example native anthocyanin in plasma has been quoted as less than 1% of the consumed quantities. This has been due to limited intestinal absorption, high rates of cellular uptake, metabolism and excretion. Therefore, for therapeutic applications of tetraketide derivatives, it can be advantageous to use tetraketide derivatives with instability at the relevant stage of the digestive tract, or further derivatization for maximum adsorption at the relevant stage of the digestive tract. Colonic metabolism of tetraketide derivatives can also be considered. Therefore, in some instances “improved stability” of tetraketide derivatives may actually be a decrease in stability for delivery to a specific stage of the digestive tract or colon. The chemical forms of tetraketide derivatives ingested in the diet may not be the ones that reach a specific target in the body instead it may be their respective metabolites as they are formed through metaboli.sm

The composition of the invention may be formulated into a dry solid form by using methods known in the art. Further, the composition may be in dry form such as a spray dried, spray cooled, lyophilized, flash frozen, granular, microgranular, capsule or microcapsule form made using methods known in the art.

The composition of the invention may also be formulated into liquid stabilized form using methods known in the art. Further, the composition may be in liquid form such as a stabilized liquid comprising one or more stabilizers such as sugars and/or polyols (e.g. sugar alcohols) and/or organic acids (e.g. lactic acid).

The composition of the invention may further be characterised by being a food product, a dietary supplement, a pharmaceutical product, a cosmetic product.

### Pharmaceutical Preparations

The invention further provides a method for preparing a pharmaceutical preparation comprising subjecting the composition of the invention to one or more steps transforming the composition and its contents of tetraketide derivatives into a therapeutically relevant mixture further comprising one or more pharmaceutical grade additives and/or adjuvants.

The invention also provides a pharmaceutical preparation obtained or obtainable from the method for preparing the pharmaceutical preparation.

### Use of Tetraketide Derivatives of the Invention

A composition of the invention may be used for non-therapeutic purposes for example as a dye, colorant or pigment, as pH indicators, as food additives, as antioxidants, in cosmetics, or as non-therapeutic food and nutritional supplements.

Further, the pharmaceutical preparation of the invention can be used as a medicament. Tetraketide derivatives of the invention are known to be active in the prevention and/or treatment of a range of disorders linked to human metabolic syndrome, including obesity, diabetes, insulin resistance, hyperglycemia, and neuropathy. Further, tetraketide derivatives of the invention are known to be active in the prevention and/or treatment of cardiovascular diseases, including inflammations, atherosclerosis, hypercholesterolemia, and hypertension; in the prevention and/or treatment of neurodegenerative disorders, such as cognitive disorders, memory loss, alzheimer's disease, depressions, and anxiety; in the prevention and/or treatment of cancers, such as renal and colorectal cancers, or cancers of colon, liver, pancreas, or prostate cancer; in the prevention and/or treatment of skin disorders, including atopic dermatitis, eye care, venous diseases, fatigues, hormonal disruptions, and viral or bacterial infections; for the use in hormonal replacement therapy. Accordingly, the invention provides the pharmaceutical preparation of the invention for use in the prevention and/or treatment of a disease selected from:
a) metabolic syndrome, including obesity, diabetes, insulin resistance, hyperglycemia, and neuropathy;
b) cardiovascular diseases, including inflammations, atherosclerosis, hypercholesterolemia, and hypertension;
c) neurodegenerative disorders, such as cognitive disorders, memory loss, alzheimer's disease, depressions, and anxiety;
d) cancers, such as renal and colorectal cancers, or cancers of colon, liver, pancreas, or prostate;
e) skin disorders, including atopic dermatitis;
f) ophthalmic disorders;
g) venous diseases;
h) fatigue;
i) endocrine disorders, including hormonal disruptions; and
j) infections, including viral or bacterial infections.

Further, in an embodiment the pharmaceutical preparation of the invention can be used in a method of treating a disease in a mammal in need thereof comprising administering a therapeutically effective amount of the pharmaceutical preparation of the invention to the mammal.

### REFERENCE TO SEQUENCE LISTING

The present application includes a Sequence Listing for the sequences of table A, titled 17420990_sequence-listing_st26, and 233,781 bytes in size, with a creation date of Apr. 30, 2026, prepared in WIPO Sequence 2.3.0, which is submitted electronically in ST26 format which is hereby incorporated by reference in its entirety

TABLE A

| SEQ ID NO | | | | Species |
|---|---|---|---|---|
| SEQ ID NO: 1 | DNA coding sequence of | PAL | from | Arabidopsis thaliana |
| SEQ ID NO: 2 | Polypeptide sequence of | PAL | from | Arabidopsis thaliana |
| SEQ ID NO: 3 | DNA coding sequence of | TA | from | Zea mays |
| SEQ ID NO: 4 | Polypeptide sequence of | TA | from | Zea mays |
| SEQ ID NO: 5 | DNA coding sequence of | C4H | from | Ammi majus |
| SEQ ID NO: 6 | Polypeptide sequence of | C4H | from | Ammi majus |
| SEQ ID NO: 7 | DNA coding sequence of | 4CL | from | Arabidopsis thaliana |
| SEQ ID NO: 8 | Polypeptide sequence of | 4CL | from | Arabidopsis thaliana |
| SEQ ID NO: 9 | DNA coding sequence of | CHS | from | Hypericum androsaemum |
| SEQ ID NO: 10 | Polypeptide sequence of | CHS | from | Hypericum androsaemum |
| SEQ ID NO: 11 | DNA coding sequence of | CHI | from | Medicago sativa |
| SEQ ID NO: 12 | Polypeptide sequence of | CHI | from | Medicago sativa |
| SEQ ID NO: 13 | DNA coding sequence of | CHIL | from | Petunia × hybrida |
| SEQ ID NO: 14 | Polypeptide sequence of | CHIL | from | Petunia × hybrida |
| SEQ ID NO: 15 | DNA coding sequence of | CHIL | from | Arabidopsis thaliana |
| SEQ ID NO: 16 | Polypeptide sequence of | CHIL | from | Arabidopsis thaliana |
| SEQ ID NO: 17 | DNA coding sequence of | CHIL | from | Ipomoea nil |
| SEQ ID NO: 18 | Polypeptide sequence of | CHIL | from | Ipomoea nil |
| SEQ ID NO: 19 | DNA coding sequence of | CHIL | from | Antirrhinum majus |
| SEQ ID NO: 20 | Polypeptide sequence of | CHIL | from | Antirrhinum majus |
| SEQ ID NO: 21 | DNA coding sequence of | CHIL | from | Glycine max |
| SEQ ID NO: 22 | Polypeptide sequence of | CHIL | from | Glycine max |
| SEQ ID NO: 23 | DNA coding sequence of | CPR | from | Saccharomyces cerevisiae |
| SEQ ID NO: 24 | Polypeptide sequence of | CPR | from | Saccharomyces cerevisiae |
| SEQ ID NO: 25 | DNA coding sequence of | CPR | from | Arabidopsis thaliana |
| SEQ ID NO: 26 | Polypeptide sequence of | CPR | from | Arabidopsis thaliana |
| SEQ ID NO: 27 | DNA coding sequence of | F3′H | from | Petunia × hybrida |
| SEQ ID NO: 28 | Polypeptide sequence of | F3′H | from | Petunia × hybrida |
| SEQ ID NO: 29 | DNA coding sequence of | F3′5′H | from | Petunia × hybrida |
| SEQ ID NO: 30 | Polypeptide sequence of | F3′5′H | from | Petunia × hybrida |
| SEQ ID NO: 31 | DNA coding sequence of | FNS I | from | Petroselinum crispum |
| SEQ ID NO: 32 | Polypeptide sequence of | FNS I | from | Petroselinum crispum |
| SEQ ID NO: 33 | DNA coding sequence of | FNS II | from | Antirrhinum majus |
| SEQ ID NO: 34 | Polypeptide sequence of | FNS II | from | Antirrhinum majus |
| SEQ ID NO: 35 | DNA coding sequence of | IFS | from | Glycine max |
| SEQ ID NO: 36 | Polypeptide sequence of | IFS | from | Glycine max |
| SEQ ID NO: 37 | DNA coding sequence of | HIFD | from | Glycine max |
| SEQ ID NO: 38 | Polypeptide sequence of | HIFD | from | Glycine max |
| SEQ ID NO: 39 | DNA coding sequence of | F3H | from | Malus domestica |
| SEQ ID NO: 40 | Polypeptide sequence of | F3H | from | Malus domestica |
| SEQ ID NO: 41 | DNA coding sequence of | FLS | from | Citrus unshiu |
| SEQ ID NO: 42 | Polypeptide sequence of | FLS | from | Citrus unshiu |
| SEQ ID NO: 43 | DNA coding sequence of | DFR | from | Anthurium andraeanum |
| SEQ ID NO: 44 | Polypeptide sequence of | DFR | from | Anthurium andraeanum |
| SEQ ID NO: 45 | DNA coding sequence of | DFR | from | Populus trichocarpa |
| SEQ ID NO: 46 | Polypeptide sequence of | DFR | from | Populus trichocarpa |
| SEQ ID NO: 47 | DNA coding sequence of | DFR | from | Iris × hollandica |
| SEQ ID NO: 48 | Polypeptide sequence of | DFR | from | Iris × hollandica |
| SEQ ID NO: 49 | DNA coding sequence of | LAR | from | Vitis vinifera |
| SEQ ID NO: 50 | Polypeptide sequence of | LAR | from | Vitis vinifera |
| SEQ ID NO: 51 | DNA coding sequence of | ANR | from | Lotus corniculatus |
| SEQ ID NO: 52 | Polypeptide sequence of | ANR | from | Lotus corniculatus |
| SEQ ID NO: 53 | DNA coding sequence of | ANS | from | Petunia × hybrida |
| SEQ ID NO: 54 | Polypeptide sequence of | ANS | from | Petunia × hybrida |
| SEQ ID NO: 55 | DNA coding sequence of | GST | from | Petunia × hybrida |
| SEQ ID NO: 56 | Polypeptide sequence of | GST | from | Petunia × hybrida |
| SEQ ID NO: 57 | DNA coding sequence of | A3GT | from | Dianthus caryophyllus |
| SEQ ID NO: 58 | Polypeptide sequence of | A3GT | from | Dianthus caryophyllus |
| SEQ ID NO: 59 | DNA coding sequence of | A3GT | from | Fragaria × ananassa |
| SEQ ID NO: 60 | Polypeptide sequence of | A3GT | from | Fragaria × ananassa |
| SEQ ID NO: 61 | DNA coding sequence of | COMT | from | Medicago sativa |
| SEQ ID NO: 62 | Polypeptide sequence of | COMT | from | Medicago sativa |
| SEQ ID NO: 63 | DNA coding sequence of | CCOMT | from | Arabidopsis thaliana |
| SEQ ID NO: 64 | Polypeptide sequence of | CCOMT | from | Arabidopsis thaliana |
| SEQ ID NO: 65 | DNA coding sequence of | C3′H | from | Arabidopsis thaliana |
| SEQ ID NO: 66 | Polypeptide sequence of | C3′H | from | Arabidopsis thaliana |
| SEQ ID NO: 67 | DNA coding sequence of | HCT | from | Arabidopsis thaliana |
| SEQ ID NO: 68 | Polypeptide sequence of | HCT | from | Arabidopsis thaliana |
| SEQ ID NO: 69 | DNA coding sequence of | C3H | from | N/A |
| SEQ ID NO: 70 | Polypeptide sequence of | C3H | from | N/A |
| SEQ ID NO: 71 | DNA coding sequence of | STS | from | Pinus densiflora |
| SEQ ID NO: 72 | Polypeptide sequence of | STS | from | Pinus densiflora |
| SEQ ID NO: 73 | DNA coding sequence of | STS | from | Vitis vinifera |
| SEQ ID NO: 74 | Polypeptide sequence of | STS | from | Vitis vinifera |
| SEQ ID NO: 75 | DNA coding sequence of | ROMT | from | Sorghum bicolor |
| SEQ ID NO: 76 | Polypeptide sequence of | ROMT | from | Sorghum bicolor |
| SEQ ID NO: 77 | DNA coding sequence of | AAliAT | from | Dahlia variabilis |
| SEQ ID NO: 78 | Polypeptide sequence of | AAliAT | from | Dahlia variabilis |
| SEQ ID NO: 79 | DNA coding sequence of | AAroAT | from | Arabidopsis thaliana |
| SEQ ID NO: 80 | Polypeptide sequence of | AAroAT | from | Arabidopsis thaliana |
| SEQ ID NO: 81 | DNA coding sequence of | A5GT | from | Vitis amurensis |
| SEQ ID NO: 82 | Polypeptide sequence of | A5GT | from | Vitis amurensis |
| SEQ ID NO: 83 | DNA coding sequence of | A3GRhaT | from | Nierembergia sp. NB17 |
| SEQ ID NO: 84 | Polypeptide sequence of | A3GRhaT | from | Nierembergia sp. NB17 |
| SEQ ID NO: 85 | DNA coding sequence of | A3′5′GT | from | Clitoria ternatea |
| SEQ ID NO: 86 | Polypeptide sequence of | A3′5′GT | from | Clitoria ternatea |
| SEQ ID NO: 87 | DNA coding sequence of | A3′GT | from | Gentiana triflora |
| SEQ ID NO: 88 | Polypeptide sequence of | A3′GT | from | Gentiana triflora |
| SEQ ID NO: 89 | DNA coding sequence of | AAroAT | from | Gentiana triflora |
| SEQ ID NO: 90 | Polypeptide sequence of | AAroAT | from | Gentiana triflora |
| SEQ ID NO: 91 | DNA coding sequence of | DBR | from | Saccharomyces cerevisiae |
| SEQ ID NO: 92 | Polypeptide sequence of | DBR | from | Saccharomyces cerevisiae |
| SEQ ID NO: 93 | DNA coding sequence of | PGT | from | Pyrus communis |
| SEQ ID NO: 94 | Polypeptide sequence of | PGT | from | Pyrus communis |
| SEQ ID NO: 95 | DNA coding sequence of | CGT | from | Oryza sativa |
| SEQ ID NO: 96 | Polypeptide sequence of | CGT | from | Oryza sativa |
| SEQ ID NO: 97 | DNA coding sequence of | AOMT | from | Vitis vinifera |
| SEQ ID NO: 98 | Polypeptide sequence of | AOMT | from | Vitis vinifera |
| SEQ ID NO: 99 | DNA coding sequence of | RHM2 | from | Arabidopsis thaliana |
| SEQ ID NO: 100 | Polypeptide sequence of | RHM2 | from | Arabidopsis thaliana |
| SEQ ID NO: 101 | DNA promoter sequence | FBA1 | from | Saccharomyces cerevisiae |
| SEQ ID NO: 102 | DNA promoter sequence | GPD1 | from | Saccharomyces cerevisiae |
| SEQ ID NO: 103 | DNA promoter sequence | PGK1 | from | Saccharomyces cerevisiae |
| SEQ ID NO: 104 | DNA promoter sequence | PDC1 | from | Saccharomyces cerevisiae |
| SEQ ID NO: 105 | DNA promoter sequence | PYK1 | from | Saccharomyces cerevisiae |
| SEQ ID NO: 106 | DNA promoter sequence | TEF1 | from | Saccharomyces cerevisiae |
| SEQ ID NO: 107 | DNA promoter sequence | TEF2 | from | Saccharomyces cerevisiae |
| SEQ ID NO: 108 | DNA promoter sequence | TPI1 | from | Saccharomyces cerevisiae |

Further, the sequence listing include the following sequences
SEQ ID NO: 109 DNA ZA helper construct for integration into host genome locus XI-3
SEQ ID NO: 110 DNA ZA helper construct for integration into host genome locus XI-2
SEQ ID NO: 111 DNA AB helper construct, comprising the URA3 auxotrophic marker
SEQ ID NO: 112 DNA ZA helper construct for plasmids, comprising the LEU2 auxotrophic marker
SEQ ID NO: 113 DNA ZA helper construct for plasmids, comprising the HIS3 auxotrophic marker
SEQ ID NO: 114 DNA AB helper construct for plasmids, comprising ARS/CEN
SEQ ID NO: 115 DNA DZ linker fragment
SEQ ID NO: 116 DNA EZ linker fragment
SEQ ID NO: 117 DNA FZ linker fragment
SEQ ID NO: 118 DNA GZ linker fragment
SEQ ID NO: 119 DNA HZ linker fragment
SEQ ID NO: 120 DNA BC tags flanking promoter/terminator cassette Gpd1/Cyc1
SEQ ID NO: 121 DNA CD tags flanking promoter/terminator cassette Pgk1/Adh2
SEQ ID NO: 122 DNA DE tags flanking promoter/terminator cassette Tef1/Eno2
SEQ ID NO: 123 DNA EF tags flanking promoter/terminator cassette Pdc1/Fba1
SEQ ID NO: 124 DNA FG tags flanking promoter/terminator cassette Tef2/Pgi1
SEQ ID NO: 125 DNA GH tags flanking promoter/terminator cassette Pyk1/Adh1

### LIST OF REFERENCES

Akita et al., Planta 2011, 234 (6): 1127-36
Arend et al., Biotech. & Bioeng, 2001, 78:126-131
Ausubel et al., 1989, Current protocols in molecular biology, Greene Publishing Associates and Wiley Interscience, New York, USA
Ban et al., PNAS, 2018, 115(22): E5223-32.
Chenna et al., Nucleic Acids Res., 2003, 31 (13): 3497-500.
De Vetten N. Et al., 1999, Proc. Natl. Acad. Sci. USA, 96:778-783
Eichenberger et al., FEMS Yeast Res. 2018, 1: 18(4)
Eichenberger et al., Met. Eng., 2017, 39:80-89
Eichenberger, Michael; Biosynthesis of plant polyketides in yeast. Technische Universität, Darmstadt
[Ph.D. Thesis], (2019) Darmstadt University, 3 Apr. 2019 10:27
Emmerstorfer A et al., 2015, Biotechnol. J., 10:623-35
Fujino et al., Plant Biotechnol 2014, 31:105-14.
Gietz et al., Nat. Protoc. 2007, 2(1): 35-7
Green & Sambrook, 2012, Molecular cloning: A laboratory Manual, Fourth Edition, Cold Spring Harbor Laboratory, New York, USA
Hugueney et al., Plant Physiol. 2009, 150 (4): 2057-70
Innis et al.; 1990, PCR Protocols: A Guide to Methods and Applications, Acad. Press, San Diego, CA, USA.
Jiang et al., J. Exp. Bot. 2015, 66, 22:7165-79
Jiang H et al., Appl Environ Microbiol. 2005, 71(6):2962-9
Koopman et al., Microb Cell Fact 2012, 11:155.
Lehka et al. FEMS Yeast Res. 2017, 17(1).
Leonard E et al., Appl Environ Microbiol. 2007, 73(12): 3877-86
Liu L et al., Engineering 2019, 5:287-295
Mikkelsen et al., Met. Eng., 2012, 14: 104-11
Morita et al. The Plant Journal, 2014, 78: 294-304
Paquette et al., Phytochemistry, 2003, 62: 399-413
Roldan et al., Plant J. 2014, 80(4): 695-708
Sasaki et al., Molecules, 2014, 19: 18747-66
Shao et al., Nucl. Acids Res. 2009, 37(2):e16
Sikorski and Hieter 1989, Genetics 122: 19-27
Turnbull et al., Bioorg. Med. Chem. Lett., 2003, 13: 3853-57
Turnbull et al., Chem. Commun., 2000, 2473-74
Turnbull et al., J. Biol. Chem. 2004, 279: 1206-16
Welford et al., Chem. Commun., 2001, 1828-29.
Yan Y et al., Appl Environ Microbiol. 2005, 71(9): 5610-3
Yin et al., PLoS One, 2011, 6(11): e27995
Zhao J; Trends Plant Sci.; 2015; vol. 20(9); pp 576-85.
Wolfenstetter S et al.; Plant Cell; 2012; vol. 24(1); pp 215-32.
Wang X et al.; Plant Cell; 2014; vol. 26(10); pp 4102-18.
Goodman C D et al.; Plant Cell; 2004; vol. 16(7); pp 1812-26.
Badone F C et al.; Planta; 2010; vol. 231(5); pp 1189-99.
Francisco R M et al.; Plant Cell; 2013; vol. 25(5); pp 1840-54.
Chowdhury S et al.; J. Biol. Chem.; 2014; vol. 289(23); pp 16129-47.
Liu G et al.; J. Biol. Chem.; 2001; vol. 276(12); pp 8648-56.
Banasiak J et al.; J. Exp. Bot.; 2013; vol. 64(4); pp 1005-15.
Mathews H et al; Plant Cell; 2003; vol. 15(8); pp 1689-703.
Debeaujon I et al.; Plant Cell; 2001; vol. 13(4); pp 853-71.
Marinova K. et al.; Plant Cell; 2007; vol. 19(6); pp 2023-38.
Zhao J et al.; Plant Cell; 2009; vol. 21(8); pp 2323-40. Erratum in: Plant Cell; 2010; vol. 22 (3); p991.
Gomez Cet al.; Plant Physiol.; 2009; vol. 150(1); pp 402-15.
Zhao J et al.; Plant Cell; 2011; vol. 23(4); pp 1536-55.
Frank S et al.; Plant Biol. (Stuttg); 2011; vol. 13(1); pp 42-50.
Chai Y R et al.; Mol. Genet. Genomics; 2009; vol. 281(1); pp 109-23.
Gao J S et al.; Gene; 2016; vol. 576(2 Pt 2); pp 763-9.
Chen L et al.; PLoS One; 2015; vol. 10(3); e0118578; doi: 10.1371/journal.pone.0118578; eCollection 2015; PubMed PMID: 25781331; PubMed Central PMCID: PMC4363705.

### EXAMPLES

The invention will be further described in the following examples, which do not limit the scope of the invention described in the claims.

### Materials and Methods

### Materials

Chemicals used in the examples herein e.g. for buffers and substrates are commercial products of at least reagent grade.

### Strains and Genes

For demonstrating production of tetraketide derivatives a base S. cerevisiae strain, S288c, is engineered to integrate the genes of the pathways of the invention. The S. cerevisiae S288C, strain NCYC 3608, is obtained from the National Collection of Yeast Cultures (NCYC), Norwich, U.K.

All pathway genes/polynucleotides referred to and disclosed herein (SEQ ID NOS: 1-100) encoding the enzymes and proteins used, are manufactured synthetically by a commercial supplier using codons optimized for expression in yeast, S. cerevisiae, except for ScCPR1 (SEQ ID NO: 23), which is amplified by PCR from yeast genomic DNA. During synthesis, all genes are appended with the DNA sequence AAGCTTAAA at the 5′-end, including a Hind III restriction recognition site and a Kozak sequence, and with the DNA sequence CCGCGG at the 3′-end, including a Sac II recognition site.

### Assays and Reagents

Tetraketide derivatives are analyzed using liquid-chromatography coupled to mass spectrometry (LC/MS). An HSS T3 column (Waters AG, Baden-Dättwil, Switzerland), 130 Å, 1.7 μm, 2.1 mm×100 mm is employed using the conditions indicated in table 1 below.

The following solution are used:
Solution A=0.1% aqueous solution of formic acid,
Solution B=0.1% solution of formic acid in acetonitrile.

TABLE 1
Chromatographic gradient for LCMS analysis
of flavonoids and anthocyanins.

| Time | Flow | | |
|---|---|---|---|
| (min) | (mL/min) | % A | % B |
| Initial | 0.400 | 95.0 | 5.0 |
| 3.00 | 0.400 | 80.0 | 20.0 |
| 4.30 | 0.400 | 80.0 | 20.0 |
| 9.00 | 0.400 | 55.0 | 45.0 |
| 11.00 | 0.400 | 0.0 | 100.0 |
| 13.00 | 0.400 | 0.0 | 100.0 |
| 13.01 | 0.400 | 95.0 | 5.0 |
| 15.00 | 0.400 | 95.0 | 5.0 |

For mass spectrum (MS) analysis, full scan spectrum data are recorded using a Xevo® G2-XS Mass spectrometer (Waters Cooperation, Milford, US) with the parameters indicated in table 2, below.

TABLE 2
Mass spectrometry parameters.

| Source Parameter | Value | |
|---|---|---|
| Ion Source | Electrospray Positive Mode (ESI−) | |
| Capillary Voltage | 2.0 | kV |
| Sampling Cone | 40 | V |
| Source Offset | 80 | V |
| Source Temperature | 150° | C. |
| Desolvation Temperature | 500° | C. |
| Cone gas flow | 100 | L/h |
| Desolvation gas flow | 1000 | L/h |

| | |
|---|---|
| Mass Range | From 50 to 1200 m/z |
| Lock Mass | Leucin Enkephalin (ESI+) |

For each compound, an extracted ion chromatogram within a mass window of 0.01 Da is calculated. Peak areas and compound quantities are calculated according to the retention time and linear calibration curve of the respective standard compounds (obtained from Sigma-Aldrich, Switzerland and/or Extrasynthese, Genay, France) where ever available.

### Example 1—Culturing Recombinant Host Cells to Produce the Tetraketide Derivatives of the Invention

For demonstrating successful tetraketide derivative production in engineered yeast via heterologous full-length biosynthetic pathways, genes encoding highly efficient recombinant enzymes were integrated and expressed in the base yeast strain under near optimal conditions to achieve sufficient flow through the pathway to produce useful amounts of the tetraketide derivative.

For culturing the engineered yeast strain, cultures of the strain are grown in 96 well, deep well plates (DWP) at 30° C., using 5 cm shaking diameter, and 300 rpm. Pre-cultures are grown for 24 hours from single colonies in 300 μL SC dropout medium (Formedium, Hunstanton, UK), as required for auxotrophic selection. The SC dropout medium contained:
1.47 g/L Synthetic Complete (Kaiser) Drop Out: Leu, His, Ura (Formedium, Hunstanton, UK),
6.7 g/L Yeast Nitrogen Base without Amino Acids,
20 g/L D-(+)-Glucose,
76 mg/L histidine, and/or
380 mg/L leucine, and/or
76 mg/L uracil depending on the auxotrophies of the strains.
   pH of the medium is adjusted to 5.8 with hydrochloric acid.

Main cultures are inoculated in 300 μL of the same medium to a 1:100 dilution of the pre-culture and cultured for 72 hours at 30° C., in 96 well, 1.1 mL deep well plates (DWP) as described by Eichenberger et al. (FEMS Yeast Res. 2018, 1: 18(4)). After 72 hours all cultures had reached essentially the same final optical density (OD) at 600 nm. It is contemplated that all tetraketide derivatives of interest are located both intra- and extracellularly, and product titers are calculated based on extraction of total culture volumes.

For extraction and stabilization of tetraketide derivatives, 150 μL culture broth is mixed with 150 μL acidified methanol (1% hydrochloric acid) and incubated for 30 min in a 96 well DWP at 30° C., 5 cm shaking diameter, and 300 rpm and subsequently clarified by centrifugation at 4000 g for 5 min. The clarified lysates are analyzed by LC-MS.

### Example 2—Engineering of Base Yeast Strain to Produce Tetraketide Derivative Naringenin

The naringenin structural pathway is assembled by in vivo homologous recombination and simultaneous integration (Eichenberger et al., FEMS Yeast Res. 2018, 1: 18(4)) into the base S. cerevisiae strain to create a naringenin producing strain, marked BG1.

The genes listed in table 3) below are used for constructing the naringenin pathway. For generating the naringenin metabolic precursor, p-coumaric acid (see the pathway of FIG. 1), as alternative to or in addition to PAL and C4H a tyrosine ammonia lyase (TAL), such as the one specified by SEQ ID NO: 3 and 4 could have been used. The genes are cloned into expression cassettes, comprising native promoters and terminators, which by Asc I digestion are released as individual DNA fragments (Eichenberger et al., Met. Eng., 2017, 39:80-89). The expression cassettes, into which the genes are cloned, are constructed in such a way that the Gpd1/Cyc1 promoter/terminator pair is flanked by HRT tags B and C (SEQ ID NO: 120), the Pgk1/Adh2 pair by C and D (SEQ ID NO: 121), the Tef1/Eno2 pair by D and E (SEQ ID NO: 122), the Pdc1/Fba1 pair by E and F (SEQ ID NO: 123), the Tef2/Pgi1 pair by F and G (SEQ ID NO: 124), and the Pyk1/Adh1 pair by G and H (SEQ ID NO: 125), respectively. All cassettes comprise an approx. 650 bps spacer fragment, that is excised and replaced, during cloning, by the genes intended for expression. Hence, after excision of the cassettes, by Asc I digestion, these fragments are autonomously assembled by homologous recombination after introduction into yeast. Table 3) also lists some additional DNA fragments, which are used to assemble the integration construct. At both ends each DNA fragment comprised so-called Homologous Recombination Tags (HRTs) that allow in vivo assembly and integration into the genome in a single step process (see below or Eichenberger et al. FEMS Yeast Res. 2018 Jun. 1; 18(4))

TABLE 3
Naringenin pathway genes used in Example No. 2.

| Cassette | Content | SEQ ID NO | Species |
|---|---|---|---|
| ZA | Integration tag | 109 | N/A |
| | for XI-3 | | |
| AB | URA3 and LoxP | 111 | N/A |
| BC | AtPAL2 | 1 | Arabidopsis thaliana |
| CD | AmC4H | 5 | Ammi majus |
| DE | At4CL2 | 7 | Arabidopsis thaliana |
| EF | HaCHS | 9 | Hypericum androsaemum |
| FG | MsCHI | 11 | Medicago sativa |
| GH | ScCPR1 | 23 | Saccharomyces cerevisiae |
| HZ | Linker | 119 | N/A |

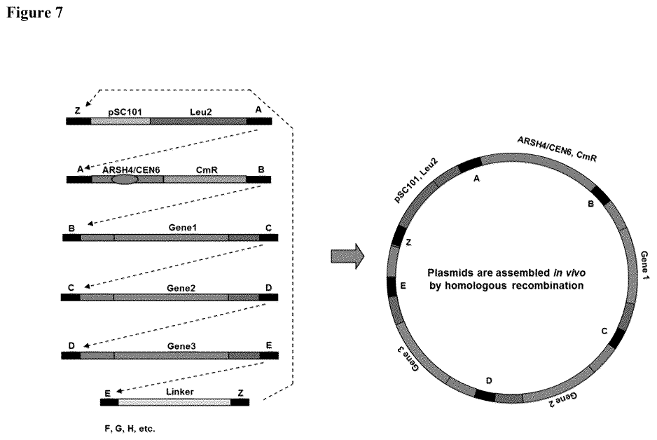

All genes are cloned into Hind III and Sac II of pUC18 based vectors containing yeast expression cassettes, said cassettes comprising the native yeast promoter and terminator sequences, separated by a spacer and multi-cloning sequence, comprising the Hind III and Sac II sites. Promoters (SEQ ID Nos: 101-108) and terminators, are previously described by Shao et al. (Nucl. Acids Res. 2009, 37(2):e16). Each expression cassette is flanked at both ends by 60 bp Homologous Recombination Tag (HRT) sequences, which are, in turn, flanked by Asc I restriction enzyme recognition sites (FIGS. 6 and 7). The HRTs, named A, B, C, D, E, F, G, H, and Z, are designed such that the 3′-end HRT of the first expression cassette fragment is identical to the 5′-end HRT of the second expression cassette fragment, and so forth. Three helper fragments are used to integrate multiple expression cassettes into the yeast genome by homologous recombination. One helper fragment (ZA, SEQ ID NO: 109), included the two recombination tags for integration into the site XI-3, described by Mikkelsen et al. (Met. Eng., 2012, 14:104-11), each tag having homology to sequences in the yeast genome. The integration tags are both flanked by HRTs and separated by an Asc I site. The second helper fragment (AB, SEQ ID NO: 111) included a yeast auxotrophic marker gene (URA3) flanked by LoxP sites. This fragment also had flanking HRTs. The third helper fragment (HZ, SEQ ID NO: 119) is designed only with HRTs, separated by a short 650 bp spacer sequence. All helper fragments had been cloned in the Asc I site of a pUC18 based plasmid backbone for amplification in E. coli. FIGS. 6 and 7 depicts how the DNA assembler technology, based on Shao et al. (Nucl. Acids Res. 2009, 37 (2): e16) is used to assemble biosynthetic pathways by homologous recombination, either for stable maintenance on a plasmid (FIG. 7) or after integration into the host genome (FIG. 6).

To integrate the naringenin pathway into the base yeast strain, plasmid DNA from the three helper plasmids (SEQ ID NOS: 109, 111, and 119) is mixed with plasmid DNA from each of the plasmids containing the expression cassettes. The mix of plasmid DNA is digested with Asc I. This treatment released all fragments from the plasmid backbone and created fragments with HRTs at the ends, these being sequentially overlapping with the HRT of the next fragment.

The base yeast strain is transformed with the digested mix, and the naringenin pathway is self-assembled and integrated by in vivo homologous recombination as described by Shao et al. 2009. Following integration the URA3 marker is excised by standard procedures, using the Cre recombinase.

Following integration, the genes are transcribed and translated into the enzymes of the naringenin biosynthetic pathway, plus the additional polypeptide ScCPR1, known to improve the activity of C4H. Successful naringenin production is confirmed by LC/MS.

### Example 3—Boosting Naringenin Production by Chalcone Isomerase-Like Protein (CHIL)

To further improve the production of naringenin, the gene encoding a P. hybrida chalcone isomerase-like protein (PhCHIL; SEQ ID NO. 13) is introduced into the naringenin producing strain, BG1 of example 2, on a single copy plasmid. This plasmid is based on the pRS413 (Sikorski R. S. and Hieter P. 1989, Genetics 122:19-27), and comprised the expression cassette of the GPD1 promoter (SEQ ID NO: 102) and the ADHI terminator. A control strain is also created, comprising the pRS413 plasmid with an empty expression cassette, introduced into the naringenin producing BG1 strain. This control strain is cultured alongside the strain comprising the PhCHIL gene, and the production of naringenin is analysed. Compared to the control strain, the strain comprising the PhCHIL gene exhibited a more than 1.5 fold increase in naringenin production.

### Example 4—Engineering of Base Yeast Strain to Produce Naringenin, Eriodictyol, and Pentahydroxyflavanone Using CHIL

Strains producing naringenin, eriodictyol, and 5,7,3′,4′,5′-pentahydroxyflavanone are created by assembly of HRT plasmids into the naringenin producing strain BG1 of example 2 according to Table 4 below. Five different CHIL enzymes are tested for their ability to increase flavanone production. Hence, the BC cassette is either empty, or carries one of the five CHIL enzymes: 1) Petunia x hybrida PhCHIL (SEQ ID NO: 13), 2) Arabidopsis thaliana AtCHIL (SEQ ID NO: 15), 3) Ipomoea nil InCHIL (SEQ ID NO: 17), 4) Antirrhinum majus AmCHIL (SEQ ID NO: 19), or 5) Glycine max GmCHIL (SEQ ID NO: 21). The CD cassette carried the Arabidopsis thaliana AtCPR (SEQ ID NO: 25), and the DE cassette is either empty, or comprised a flavonoid-3′-hydroxylase (PhF3′H; SEQ ID NO: 27) or a flavonoid-3′5′-hydroxylase (PhF3′5′H; SEQ ID NO: 29), both from Petunia X hybrida. FIG. 7 depicts pathway assembly on HRT plasmids.

The backbone of the HRT vectors is formed by the DNA fragments ZA (SEQ ID NO: 112), AB (SEQ ID NO: 114), and EZ (SEQ ID NO: 116), which contained a yeast selection marker gene (LEU2), an autonomously replicating sequence (ARSH4) and a yeast centromere (CEN6), and a 650 bp stuffer sequence, respectively (see Table 4 below). The ZA fragment further comprised a bacterial origin of replication (pSC101), and the AB fragment further comprised the chloramphenicol resistance marker gene (CmR), but none of these two functionalities are used in the current embodiment. Expression of each gene is driven by a yeast native promoter as described in example 2 above. The DNA helper fragments, as well as the gene expression cassettes, are flanked by 60 bp homologous recombination tags (HRT), where each terminal tag is identical to the first tag of the following cassette. Each HRT cassette included terminal Asc I restriction sites to allow excision from the donor plasmid backbone.

TABLE 4
Pathway gene cassettes for production of naringenin,
eriodictyol, and pentahydroxy-flavanone in BG1 strain

| Cassette | Content | SEQ ID NO | Species |
|---|---|---|---|
| ZA | LEU2, pSC101 | 112 | N/A |
| AB | ARSH4/CEN6, CmR | 114 | N/A |
| BC | Empty or CHIL | 13, 15, 17, 19, | Petunia × hybrida, |
| | | or 21 | Arabidopsis thaliana, |
| | | | Ipomoea nil, |
| | | | Antirrhinum majus, |
| | | | or Glycine max |
| CD | AtCPR | 25 | Arabidopsis thaliana |
| DE | Empty, PhF3′H, or | 27 or 29 | Petunia × hybrida |
| | PhF3′5′H | | |
| EZ | Linker | 116 | N/A |

The CHIL enzymes in the BC cassette are from various plant species as listed in the Table 4).

Plasmids containing the described helper fragments and gene expression cassettes of Table 4) are combined into 18 different reaction mixtures and digested with Asc I in a 20 μL reaction volume. The digest is performed for 2 h at 37° C. The entire volume of each reaction is used to transform yeast strain BG1, creating 18 new strains.

After transformation, using the LiAC/SS carrier DNA/PEG method (see e.g., Gietz et al., Nat Protoc. 2007; 2 (1): 35-7), cells are grown at 30° C. for 72 h. Next, four clones from each of the 18 resulting transformations are re-streaked onto fresh plates and grown for 48 h at 30° C.

All clones are then grown in 2 mL liquid cultures for 72 hours. Subsequently, 1 volume of acidified methanol is added, and after ½ hour of shaking at 30° C., cell debris are spun down by centrifugation and the cleared supernatants are collected for analysis by LC/MS. Naringenin, eriodictyol, and pentahydroxyflavanone production is markedly higher, approx. 30-70%, in strains co-expressing a CHIL protein, compared to control strains with no CHIL. The PhCHIL is chosen for further experiments.

### Example 5—Engineering of Base Yeast Strain to Produce Apigenin, Luteolin, and Genistein

Strains producing flavones and isoflavones are created by assembly of HRT plasmids into strain BG1 of example 2 according to table 5 below. The BC cassette is either empty, or carries the Petunia x hybrida PhCHIL (SEQ ID NO: 13), the CD cassette carried the AtCPR1 (SEQ ID NO: 25), the DE cassette is either empty (for apigenin), or carried the PhF3′H (SEQ ID NO: 27) for luteolin, or the 2-hydroxy-isoflavanone dehydratase (GmHIFD; SEQ ID NO: 37) from Glycine max for genistein, and the EF cassette carried the Antirrhinum majus flavone synthase (AmFNSII; SEQ ID NO: 33) or the Glycine max isoflavone synthase (GmIFS; SEQ ID NO: 35), for flavone and isoflavone synthesis, respectively. Hence, one set of fragments included the combination of FNS II with an empty cassette, or the combination of FNS II together with F3′H, in order to produce apigenin and luteolin, respectively. Another set included the combination of IFS and HIFD, for production of genistein.

The backbone of the HRT vectors is formed by the DNA fragments ZA (SEQ ID NO: 112), AB (SEQ ID NO: 114), and FZ (SEQ ID NO: 117), which contained a yeast selection marker gene (LEU2), an autonomously replicating sequence (ARSH4) and a yeast centromere (CEN6), and a 650 bp stuffer sequence, respectively (see table 5 below). Expression of each gene is driven by a yeast native promoter as described in example 2 above. The DNA helper fragments, as well as the gene expression cassettes, are flanked by 60 bp homologous recombination tags (HRT), where each terminal tag is identical to the first tag of the following cassette. Each HRT cassette included terminal Asc I restriction sites to allow excision from the donor plasmid backbone.

TABLE 5
Pathway gene cassettes for production of apigenin,
luteolin, and genistein in BG1 strain

| Cassette | Content | SEQ ID NO | Species |
|---|---|---|---|
| ZA | LEU2, pSC101 | 112 | N/A |
| AB | ARSH4/CEN6, CmR | 114 | N/A |
| BC | Empty or PhCHIL | 13 | Petunia × hybrida |
| CD | AtCPR | 25 | Arabidopsis thaliana |
| DE | Empty, PhF3′H or | 27 or 37 | Petunia × hybrida |
| | GmHIFD | | Glycine max |
| EF | AmFNSII or GmIFS | 33 or 35 | Antirrhinum majus |
| | | | Glycine max |
| FZ | Linker | 117 | N/A |

DNA fragments listed in table 5. are prepared as described above and used to transform strain BG1. The resulting strains are grown and analyzed, as described above in example 4 for production of flavones and isoflavones.

Production of apigenin and luteolin is markedly higher, more than 50%, in strains expressing the PhCHIL, compared to control strains with no PhCHIL. The production of genistein is approx. 60% higher in the strain expressing PhCHIL.

### Example 6—Engineering of Base Yeast Strain to Produce Dihydroflavonols (Flavanonols)

The three dihydroflavonols (DHF), dihydrokaempferol (DHK), dihydroquercetin (DHQ), and dihydromyricetin (DHM) are intermediates for flavonols, but also for the extended flavonoid pathway towards catechins and anthocyanins. In order to demonstrate the effects of chalcone isomerase-like protein on these extended pathways, a set of strains are first prepared, comprising the three DHF pathways, with or without co-expression of PhCHIL. These six strains are prepared by transforming the strain BG1 of example 2 with HRT plasmids comprising the genes listed in table 6. Hence, the BC cassette is either empty, or carried the Petunia x hybrida PhCHIL (SEQ ID NO: 13), the CD cassette carried the AtCPR1 (SEQ ID NO: 25), the DE cassette is either empty (for DHK), or carried the PhF3′H (SEQ ID NO: 27) for DHQ, or the PhF3′5′H (SEQ ID NO: 29) for DHM, and the EF cassette carried the Malus domestica flavonoid-3-O-hydroxylase (F3H; SEQ ID NO: 39).

The backbone of the HRT vectors is formed by the DNA fragments ZA (SEQ ID NO: 112), AB (SEQ ID NO: 114), and FZ (SEQ ID NO: 117), which contained a yeast selection marker gene (LEU2), an autonomously replicating sequence (ARSH4) and a yeast centromere (CEN6), and a 650 bp stuffer sequence, respectively (see table 6 below). Expression of each gene is driven by a yeast native promoter as described in example 2, above. The DNA helper fragments, as well as the gene expression cassettes, are flanked by 60 bp homologous recombination tags (HRT), where each terminal tag is identical to the first tag of the following cassette. Each HRT cassette included terminal Asc I restriction sites to allow excision from the donor plasmid backbone.

TABLE 6
Pathway gene cassettes for production
of dihydroflavonols in BG1 strain

| Cassette | Content | SEQ ID NO | Species |
|---|---|---|---|
| ZA | LEU2, pSC101 | 112 | N/A |
| AB | ARSH4/CEN6, CmR | 114 | N/A |
| BC | PhCHIL or empty | 13 | Petunia × hybrida |
| CD | AtCPR | 25 | Arabidopsis thaliana |
| DE | Empty, PhF3′H or | 27 or 29 | Petunia × hybrida |
| | PhF3′5′H | | |
| EF | MdF3H | 39 | Malus domestica |
| FZ | Linker | 117 | N/A |

DNA fragments listed in table 6 are prepared as described above and used to transform strain BG1. The resulting strains are grown and analyzed, as described above in example 4, confirming production of the expected DHFs. The strains producing dihydrokaempferol (DHK) are named BG5_DHK and BG5_DHKc, including or missing the PhCHIL, respectively. Similarly, the strains producing dihydroquercetin (DHQ) are named BG5_DHQ and BG5_DHQc, including or missing the PhCHIL, respectively, and the strains producing dihydromyricetin (DHM) are named BG5_DHM and BG5_DHMc, including or missing the PhCHIL, respectively. Production of dihydroflavonols is markedly higher, more than 50%, in strains expressing the PhCHIL compared to control strains with no PhCHIL.

### Example 7—Engineering of Base Yeast Strain to Produce Flavonols

In order to demonstrate the effect of co-expression of CHIL on production of flavonols, all six BG5 strains of example 6 are transformed with an additional plasmid, based on pRS413 (see example 2), comprising the Citrus unshiu flavonol synthase (CuFLS; SEQ ID NO: 41). The strains are cultured and analysed as described above, and production of kaempferol, quercetin, and myricetin, respectively, is confirmed. Production of flavonols is markedly higher, more than 50%, in strains expressing the PhCHIL compared to control strains with no PhCHIL.

### Example 8—Engineering of Base Yeast Strain to Produce Afzelechin and (+)-Catechin

In order to study the effect of CHIL expression on production of catechins, four BG5 strains, the BG5_DHK, BG5_DHKc, BG5_DHQ, and BG5DHQc (see example 6) are transformed with an additional HRT plasmid, comprising the dihydroflavonol reductase (DFR) and leucoanthocyanidin reductase (LAR). Hence, the second HRT plasmid comprised the Anthurium andraeanum DFR (AaDFR; SEQ ID NO: 43) in the BC cassette, and the Vitis vinifera LAR (VvLAR; SEQ ID NO: 49) in the CD cassette, as listed in Table No. 7 below. The backbone of the second HRT vectors is formed by the DNA fragments ZA (SEQ ID NO: 113), AB (SEQ ID NO: 114), and DZ (SEQ ID NO: 115), which contained a yeast selection marker gene (HIS3), an autonomously replicating sequence (ARSH4) and a yeast centromere (CEN6), and a 650 bp stuffer sequence, respectively (see table 7 below).

TABLE 7
Pathway gene cassettes for production of afzelechin and catechin

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | AaDFR | 43 | Anthurium andraeanum |
| CD | VvLAR | 49 | Vitis vinifera |
| DZ | Linker | 115 | N/A |

The strains are cultured and analyzed as described above and production of afzelechin and (+)-catechin, respectively, is confirmed. Production of catechins is markedly higher, more than 50%, in strains expressing the PhCHIL compared to control strains with no PhCHIL.

### Example 9—Engineering of Yeast Strain to Produce Epi-Afzelechin and (−)-Epicatechin

In order to study the effect of CHIL expression on production of epicatechins, four BG5 strains, the BG5_DHK, BG5_DHKc, BG5_DHQ, and BG5DHQc (see example 6) are transformed with a second HRT plasmid, comprising the dihydroflavonol reductase (DFR), the anthocyanidin synthase (ANS), and the anthocyanidin reductase (ANR). In addition, and in order to also study the effect of co-expressing a glutathione-S-reductase (GST) together with the ANS enzyme, the GST gene is introduced, together with DFR, ANS, and ANR, into the same four BG5 strains. The four strains BG5_DHK, BG5_DHKc, BG5_DHQ, and BG5DHQc are, thus, transformed with HRT plasmids, comprising the DFR, ANS, ANR, and either an empty cassette, or a cassette comprising the GST, resulting in a total of eight strains.

Specifically, the second HRT plasmid in those strains comprised the Anthurium andraeanum DFR (AaDFR; SEQ ID NO: 43) in the BC cassette, the Petunia x hybrida GST (PhGST; SEQ ID NO: 55) or an empty cassette in the CD cassette, the Petunia x hybrida ANS (PhANS; SEQ ID NO: 53) in the DE cassette, and the Lotus corniculatus ANR (LcANR; SEQ ID NO: 51) in the EF cassette, as listed in table 8, below.

The backbone of the second HRT vectors is formed by the DNA fragments ZA (SEQ ID NO: 113), AB (SEQ ID NO: 114), and FZ (SEQ ID NO: 117), which contained a yeast selection marker gene (HIS3), an autonomously replicating sequence (ARSH4) and a yeast centromere (CEN6), and a 650 bp stuffer sequence, respectively (see table 8, below).

TABLE 8
Pathway gene cassettes for production
of epi-afzelechin and (—)-epicatechin

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | AaDFR | 43 | Anthurium andraeanum |
| CD | Empty or PhGST | 55 | Petunia × hybrida |
| DE | PhANS | 53 | Petunia × hybrida |
| EF | LcANR | 51 | Lotus corniculatus |
| FZ | Linker | 117 | N/A |

The strains are cultured and analyzed as described above and production of epi-afzelechin and (−)-epicatechin, respectively, is confirmed. Production of both catechins is markedly higher, more than 50%, in strains expressing the PhCHIL compared to control strains with no PhCHIL. Remarkably, the production of catechins increased more than 20-fold after co-expression of PhGST. Moreover, the strains co-expressing the GST exhibited a significant reduction in the levels of flavonols. This is remarkable since, in the absence of GST, the ANS enzyme is reported to produce flavonols as an undesired side-product (see Eichenberger et al. 2018 above).

### Example 10—Engineering of Yeast Strain to Produce Pelargonidin-3-O-Glucoside, Cyanidin-3-O-Glucoside, and Delphinidin-3-O-Glucoside

In order to study the effect of CHIL expression on production of anthocyanins, all six BG5 strains (see example 6) are transformed with an additional HRT plasmid, comprising the dihydroflavonol reductase (DFR), the anthocyanidin synthase (ANS), and the anthocyanidin-3-O-glycosyl transferase (A3GT). In addition, and in order to also study the effect of co-expressing a glutathione-S-reductase (GST) together with the ANS enzyme, the GST is introduced, together with DFR, ANS, and A3GT, into the same six BG5 strains. The six BG5 strains are, thus, transformed with HRT plasmids, comprising the DFR, ANS, A3GT, and either an empty cassette, or a cassette comprising the GST, resulting in a total of twelve strains. Because DFRs and A3GTs exhibit specific substrate preferences, depending on the number of B-ring hydroxylations, various enzymes are used, reflecting those preferences (see Eichenberger et al. 2018 above).

Specifically, the second HRT plasmid in strains for production of pelargonidin-3-O-glucoside (P3G) comprised the Anthurium andraeanum DFR (AaDFR; SEQ ID NO: 43) in the BC cassette, the Petunia x hybrida GST (PhGST; SEQ ID NO: 55) or an empty cassette in the CD cassette, the Petunia x hybrida ANS (PhANS; SEQ ID NO: 53) in the DE cassette, and the Dianthus caryophyllus A3GT (DcA3GT; SEQ ID NO: 57) in the EF cassette, as listed in table 9, below.

Similarly, the second HRT plasmid in strains for cyanidin-3-O-glucoside (C3G) production comprised the Populus trichocarpa DFR (PtDFR; SEQ ID NO: 45) in the BC cassette, the Petunia x hybrida GST (PhGST; SEQ ID NO: 55) or an empty cassette in the CD cassette, the Petunia x hybrida ANS (PhANS; SEQ ID NO: 53) in the DE cassette, and the Fragaria x ananassa A3GT (FaA3GT; SEQ ID NO: 59) in the EF cassette, as listed in table 9, below.

Finally, the second HRT plasmid in strains for delphinidin-3-O-glucoside (D3G) production comprised the Iris x hollandica DFR (IhDFR; SEQ ID NO: 47) in the BC cassette, the Petunia x hybrida GST (PhGST; SEQ ID NO: 55) or an empty cassette in the CD cassette, the Petunia x hybrida ANS (PhANS; SEQ ID NO: 53) in the DE cassette, and the Fragaria x ananassa A3GT (FaA3GT; SEQ ID NO: 59) in the EF cassette, as listed in table 9, below.

The backbone of the second HRT vectors is formed by the DNA fragments ZA (SEQ ID NO: 113), AB (SEQ ID NO: 114), and FZ (SEQ ID NO: 117), which contained a yeast selection marker gene (HIS3), an autonomously replicating sequence (ARSH4) and a yeast centromere (CEN6), and a 650 bp stuffer sequence, respectively (see table 9, below).

TABLE 9
Pathway gene cassettes for production of P3G, C3G, and D3G

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | AaDFR (P3G), PtDFR | 43, 45, or 47 | Anthurium andraeanum, |
| | (C3G), or lhDFR | | Populus trichocarpa, or |
| | (D3G) | | Iris × hollandica |
| CD | Empty or PhGST | 55 | Petunia × hybrida |
| DE | PhANS | 53 | Petunia × hybrida |
| EF | DcA3GT (P3G) or | 57 or 59 | Dianthus caryophyllus |
| | FaA3GT (C3G and | | Fragaria × ananassa |
| | D3G) | | |
| FZ | Linker | 117 | N/A |

The strains are cultured and analysed as described above and production of P3G, C3G, and D3G, respectively, is confirmed. Production of anthocyanins is markedly higher, more than 50%, in strains expressing the PhCHIL compared to control strains with no PhCHIL. Remarkably, the production of anthocyanin increased more than 20-fold after co-expression of PhGST. Moreover, the strains co-expressing the PhGST exhibited a significant reduction in the levels of flavonols, including their glucosides. This is remarkable since, in the absence of GST, the ANS enzyme is reported to produce flavonols as an undesired side-product (see Eichenberger et al. 2018 above).

### Example 11—Engineering of Yeast Strain to Produce Anthocyanidins

The anthocyanidins pelargonidin (Pg), cyanidin (Cy), and delphinidin (Dp) are unstable intermediates in the biosynthetic pathways to anthocyanins and epicatechins. However, in order to extend the biosynthetic pathways beyond the 3 basic anthocyanins (see example 10) the pathway from naringenin to anthocyanidin is first integrated into strain BG1 of example 2.

Integration is done into the site XI-2, described by Mikkelsen et al. (Metabolic Engineering 2012, 14:104-11), as described in example 2 for integration into XI-3. The plasmids used are listed in table 10. Hence, for pelargonidin (Pg) production the second integration comprised the genes MdF3H (SEQ ID NO: 39), AtCPR1 (SEQ ID NO: 25), AaDFR (SEQ ID NO: 43), and PhANS (SEQ ID NO: 53). For cyanidin (Cy) production the second integration comprised the genes PhF3′H (SEQ ID NO: 27), AtCPR1 (SEQ ID NO: 25), MdF3H (SEQ ID NO: 39), PtDFR (SEQ ID NO: 45), and PhANS (SEQ ID NO: 53). For delphinidin (Dp) production the second integration comprised the genes PhF3′5′H (SEQ ID NO: 29), AtCPR1 (SEQ ID NO: 25), MdF3H (SEQ ID NO: 39), IhDFR (SEQ ID NO: 47), and PhANS (SEQ ID NO: 53) as listed in table 10, below.

As described in Example 2, integration is done using three helper fragments, the ZA (SEQ ID NO: 110), which included the two recombination tags for integration into the site XI-2, (described by Mikkelsen et al. Met. Eng., 2012, 14:104-11), the AB (SEQ ID NO: 111) which included a yeast auxotrophic marker gene (URA3) flanked by LoxP sites, and the linker fragment GZ (SEQ ID NO: 118).

TABLE 10
Plasmids used for integrating, into yeast, the pathways
to pelagonidin (Pg), cyanidin (Cy), and delphinidin (Dp).

| Cassette | Content | SEQ ID NO | Species |
|---|---|---|---|
| ZA | Integration tag for | 110 | N/A |
| | XI-2 | | |
| AB | URA3 and LoxP | 111 | N/A |
| BC | PhANS | 53 | Petunia × hybrida |
| CD | Empty (Pg) | N/A | N/A |
| | PhF3′H (Cy) | 27 | Petunia × hybrida |
| | PhF3′5′H (Dp) | 29 | Petunia × hybrida |
| DE | MdF3H | 39 | Malus domestica |
| EF | AaDFR (Pg) | 43 | Anthurium andraeanum |
| | PtDFR (Cy) | 45 | Populus trichocarpa |
| | lhDFR (Dp) | 47 | Iris × hollandica |
| FG | AtCPR1 | 25 | Arabidopsis thaliana |
| GZ | Linker | 118 | N/A |

Following transformation, single colonies are selected and cultured. Crude DNA preparations are prepared and used as template for PCR, using standard techniques, to confirm correct integrations into the XI-2 site. For each anthocyanidin a clone with the expected PCR profile is chosen, and the strains are named BG6_Pg (pelargonidin), BG6_Cy (cyanidin), and BG6_Dp (delphinidin), respectively. The URA3 marker gene is subsequently excised, by standard procedures, using the Cre recombinase.

### Example 12—Engineering of Yeast Strain to Produce Modified Anthocyanins

The three strains constructed in example 11, comprising the pathways to pelargonidin, cyanidin, and delphinidin, respectively, are used to test various secondary modifications of the central scaffold of anthocyanins. As described in example 11, each of these strains had the full-length pathway to the respective anthocyanidin integrated into the yeast genome via two integrations. Each strain is then provided with two HRT plasmids, assembled in vivo as described above, comprising various modifying genes.

A first HRT plasmid is assembled with PhCHIL (SEQ ID NO: 13), PhGST (SEQ ID NO: 55), and either DcA3GT (SEQ ID NO: 57) for pelargonidin-3-O-glucoside (P3G), or FaA3GT (SEQ ID NO: 59) for cyanidin-3-O-glucoside (C3G) and delphinidin-3-O-glucoside (D3G) production (table 11). Helper fragments ZA (SEQ ID NO: 112), AB (SEQ ID NO: 114), and FZ (SEQ ID NO: 117) are used, as described above, for the in vivo assembly of plasmids.

TABLE 11
Cassettes used to assemble HRT plasmid
for P3G, C3G, and D3G production

| Cassette | Content | SEQ ID NO | Species |
|---|---|---|---|
| ZA | LEU2, pSC101 | 112 | N/A |
| AB | ARSH4/CEN6, CmR | 114 | N/A |
| BC | PhCHIL | 13 | Petunia × hybrida |
| CD | PhGST | 55 | Petunia × hybrida |
| DE | Empty | N/A | N/A |
| EF | DcA3GT (P3G) | 57 | Dianthus caryophyllus |
| | FaA3GT(C3G and D3G) | 59 | Fragaria × ananassa |
| FZ | Linker | 117 | N/A |

Following transformation, single colonies are selected, grown, and analyzed as described above. Production of P3G, C3G, and D3G, respectively, is verified.

A second HRT plasmid comprised the anthocyanin aliphatic acyl transferase (AAliAT) from Dahlia variabilis, i.e. the anthocyanidin-3-O-glucoside-6″-malonyl-transferase gene (Dv3MAT, SEQ ID NO. 77) in combination with the Fragaria x ananassa FaA3GT (SEQ ID NO: 59) (table 12) to produce anthocyanidin-3-O-(6″-malonyl)-glucosides. The inclusion of an additional A3GT is assumed to improve the 3-O-glycosylation.

Helper fragments ZA (SEQ ID NO: 113), AB (SEQ ID NO: 114), and EZ (SEQ ID NO: 116) are used, as described above, for the in vivo assembly of plasmids.

TABLE NO. 12
Cassettes used to assemble HRT plasmid for
aliphatic acylation of P3G, C3G, and D3G.

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | Dv3MAT | 77 | Dahlia variabilis |
| CD | FaA3GT | 59 | Fragaria × ananassa |
| DE | Empty | N/A | N/A |
| EZ | Linker | 116 | N/A |

Strains BG6_Pg (pelargonidin), BG6_Cy (cyanidin), and BG6_Dp (delphinidin), described in example 11, are thus transformed with a first and a second HRT plasmid, according to tables 11 and 12, respectively.

Resulting strains are grown in appropriate culture medium and analyzed as described above. As predicted, strains derived from BG6_Pg are shown to produce pelargonidin-3-O-(6″-malonyl)-glucoside, whereas strains derived from BG6_Cy are shown to produce cyanidin-3-O-(6″-malonyl)-glucoside, and strains derived from BG6_Dp are shown to produce delphinidin-3-O-(6″-malonyl)-glucoside

Another second HRT plasmid comprised an anthocyanin aromatic acyl transferase (AAroAT) from Arabidopsis thaliana, i.e. the anthocyanin-3-O-glucoside-6″-O-p-coumaroyltransferase (At3AT1; SEQ ID NO. 79) in combination with the Fragaria x ananassa FaA3GT (SEQ ID NO: 59) (table 13) for production of anthocyanidin-3-O-(6″-O-coumarate)-glucosides.

Helper fragments ZA (SEQ ID NO: 113), AB (SEQ ID NO: 114), and EZ (SEQ ID NO: 116) are used, as described above, for the in vivo assembly of plasmids.

TABLE 13
Cassettes used to assemble HRT plasmid for
aromatic acylation of P3G, C3G, and D3G.

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | Empty | N/A | N/A |
| CD | FaA3GT | 59 | Fragaria × ananassa |
| DE | At3AT1 | 79 | Arabidopsis thaliana |
| EZ | Linker | 116 | N/A |

Strains BG6_Pg (pelargonidin), BG6_Cy (cyanidin), and BG6_Dp (delphinidin), described in example 11, are thus transformed with a first and a second HRT plasmid, according to tables 11 and 13, respectively.

Resulting strains are grown in appropriate culture medium and analyzed as described above. As predicted, strains derived from BG6_Pg are shown to produce pelargonidin-3-O-(6″-coumaroyl)-glucoside, whereas strains derived from BG6_Cy are shown to produce cyanidin-3-O-(6″-coumaroyl)-glucoside, and strains derived from BG6_Dp are shown to produce delphinidin-3-O-(6″-coumaroyl)-glucoside.

Another second HRT plasmid comprised the Vitis amurensis anthocyanidin-3-O-glucoside-5-O-glycosyltransferase (VaA5GT, SEQ ID NO. 81) in combination with the Fragaria x ananassa FaA3GT (SEQ ID NO: 59) (table 14) for production of anthocyanidin-3,5-di-O-glucosides.

Helper fragments ZA (SEQ ID NO: 113), AB (SEQ ID NO: 114), and EZ (SEQ ID NO: 116) are used, as described above, for the in vivo assembly of plasmids.

TABLE NO. 14
Cassettes used to assemble HRT plasmid for
5-O-glycosylation of P3G, C3G, and D3G.

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | Empty | N/A | N/A |
| CD | FaA3GT | 59 | Fragaria ananassa |
| DE | VaA5GT | 81 | Vitis amurensis |
| EZ | Linker | 116 | N/A |

Strains BG6_Pg (pelargonidin), BG6_Cy (cyanidin), and BG6_Dp (delphinidin), described in Example No. 11, are thus transformed with a first and a second HRT plasmid, according to Tables No. 11 and 14, respectively.

Resulting strains are grown in appropriate culture medium, and analysed as described above. As predicted, strains derived from BG6_Pg are shown to produce pelargonidin-3,5-di-O-glucoside, whereas strains derived from BG6_Cy are shown to produce cyanidin-3,5-di-O-glucoside, and strains derived from BG6_Dp are shown to produce delphinidin-3,5-di-O-glucoside

Another second HRT plasmid comprised the Nierenbergia ssp. anthocyanidin-3-O-glucoside-6″-O-rhamnosyltransferase (NsA3GRhaT, SEQ ID NO. 83) and the Arabidopsis thaliana NDP-rhamnose synthase (AtRHM2, SEQ ID NO. 99) in combination with the Fragaria x ananassa FaA3GT (SEQ ID NO: 59) (table 15) for production of anthocyanidin-3-O-rutinosides.

Helper fragments ZA (SEQ ID NO: 113), AB (SEQ ID NO: 114), and EZ (SEQ ID NO: 116) are used, as described above, for the in vivo assembly of plasmids.

TABLE 15
Cassettes used to assemble HRT plasmid for production
of anthocyanidin 3-O-rutinosides.

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | AtRHM2 | 99 | Arabidopsis thaliana |
| CD | FaA3GT | 59 | Fragaria ananassa |
| DE | NsA3GRhaT | 83 | Nierenbergia ssp |
| EZ | Linker | 116 | N/A |

Strains BG6_Pg (pelargonidin), BG6_Cy (cyanidin), and BG6_Dp (delphinidin), described in example 11, are thus transformed with a first and a second HRT plasmid, according to Tables No. 11 and 15, respectively.

Resulting strains are grown in appropriate culture medium, and analysed as described above. As predicted, strains derived from BG6_Pg are shown to produce pelargonidin-3-O-rutinoside, whereas strains derived from BG6_Cy are shown to produce cyanidin-3-O-rutinoside, and strains derived from BG6_Dp are shown to produce delphinidin-3-O-rutinoside.

Another second HRT plasmid comprised the Clitoria ternatea anthocyanidin-3-O-glucoside-3′,5′-O-glycosyltransferase (CtA3′5′GT, SEQ ID NO. 85) in combination with the Fragaria x ananassa FaA3GT (SEQ ID NO: 59) (table 16) for production of cyanidin-3,3′-di-O-glucoside and delphinidin-3,3′,5′-tri-O-glucoside.

Helper fragments ZA (SEQ ID NO: 113), AB (SEQ ID NO: 114), and EZ (SEQ ID NO: 116) are used, as described above, for the in vivo assembly of plasmids.

TABLE 16
Cassettes used to assemble HRT plasmid for production
of cyanidin-3,3′-di-O-glucoside and delphinidin-
3,3′,5′-tri-O-glucoside

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | Empty | N/A | N/A |
| CD | FaA3GT | 59 | Fragaria ananassa |
| DE | CtA3′5′GT | 85 | Clitoria ternatea |
| EZ | Linker | 116 | N/A |

Strains BG6_Pg (pelargonidin), BG6_Cy (cyanidin), and BG6_Dp (delphinidin), described in example 11, are thus transformed with a first and a second HRT plasmid, according to Tables No. 11 and 16, respectively.

Resulting strains are grown in appropriate culture medium and analyzed as described above. As predicted, strains derived from BG6_Pg produced no new compounds, whereas strains derived from BG6_Cy are shown to produce cyanidin-3,3′-di-O-glucoside, and strains derived from BG6_Dp are shown to produce delphinidin-3,3′,5′-tri-O-glucoside.

Another second HRT plasmid comprised the Clitoria ternatea anthocyanidin-3-O-glucoside-3′,5′-O-glycosyltransferase (CtA3′5′GT, SEQ ID NO. 85) in combination with the anthocyanin aliphatic acyl transferase (AAliAT) from Dahlia variabilis, i.e. the anthocyanidin-3-O-glucoside-6″-malonyl-transferase gene (Dv3MAT, SEQ ID NO. 77) and the Fragaria x ananassa FaA3GT (SEQ ID NO: 59) (table 17) for production of malonylated cyanidin-3,3′-di-O-glucoside and delphinidin-3,3′,5′-tri-O-glucoside, respectively.

Helper fragments ZA (SEQ ID NO: 113), AB (SEQ ID NO: 114), and EZ (SEQ ID NO: 116) are used, as described above, for the in vivo assembly of plasmids.

TABLE NO. 17
Cassettes used to assemble HRT plasmid for production
of malonylated cyanidin-3,3′-di-O-glucoside and delphinidin-
3,3′,5′-tri-O-glucoside.

| Cassette | Content | SEQ ID NO | Species |
|---|---|---|---|
| ZA | HIS3, pSC101 | 113 | N/A |
| AB | ARSH4/CEN6, CmR | 114 | N/A |
| BO | Dv3MAT | 77 | Dahlia variabilis |
| CD | FaA3GT | 59 | Fragaria ananassa |
| DE | CtA3′5′GT | 85 | Clitoria ternatea |
| EZ | Linker | 116 | N/A |

Strains BG6_Pg (pelargonidin), BG6_Cy (cyanidin), and BG6_Dp (delphinidin), described in example 11, are thus transformed with a first and a second HRT plasmid, according to Tables No. 11 and 17, respectively.

Resulting strains are grown in appropriate culture medium, and analysed as described above. As predicted, strains derived from BG6_Pg are shown to produce pelargonidin-3-O-(6″-malonyl)-glucoside, whereas strains derived from BG6_Cy are shown to produce cyanidin-3-O-(6″-malonyl)-glucosyl-3′-O-glucoside, and strains derived from BG6_Dp are shown to produce delphinidin-3-O-(6″-malonyl)-glucosyl-3′,5′-di-O-glucoside

Another second HRT plasmid comprised the Vitis amurensis anthocyanidin-3-O-glucoside-5-O-glycosyltransferase (VaA5GT, SEQ ID NO. 81) in combination with the Gentiana triflora anthocyanin 3′-glucosyltransferase (GtA3′GT, SEQ ID NO. 87) and the the Gentiana triflora anthocyanin 5-aromatic acyltransferase (GtGAT4, SEQ ID NO: 89), an enzyme reported to transfer caffeoyl to both the 5- and 3′-glucose (U.S. Pat. No. 8,053,648). The DNA fragments used for production of delphinidin 3-O-glucosyl-5-O-(6-O-caffeoyl-glucosyl)-3′-O-(6-O-caffeoyl-glucoside) (gentiodelphin) are shown in table 18.

Helper fragments ZA (SEQ ID NO: 113), AB (SEQ ID NO: 114), and EZ (SEQ ID NO: 116) are used, as described above, for the in vivo assembly of plasmids.

TABLE 18
Cassettes used to assemble HRT plasmid
for production of gentiodelphin

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | GtA3′GT | 87 | Gentiana triflora |
| CD | GtpGAT4 | 89 | Gentiana triflora |
| DE | VaA5GT | 81 | Vitis amurensis |
| EZ | Linker | 116 | N/A |

Strain BG6_Dp (delphinidin), described in Example No. 10, is thus transformed with a first and a second HRT plasmid, according to Tables No. 11 and 18, respectively.

A resulting strain is grown in medium as described in Example No 1, except that caffeic acid is added to the medium at a starting concentration of 20 mg/L. The strain is grown for 72 hours and analyzed as described above. Two compounds with molecular masses corresponding to delphinidin 3-O-glucosyl-5-O-(6″-caffeoyl)-glucoside-3′-O-glucoside, and delphinidin 3-O-glucosyl-5-O-(6-O-caffeoyl-glucosyl)-3′-O-(6-O-caffeoyl-glucoside) (gentiodelphin), respectively, are detected in the medium, and the extract showed a distinct blue colour corresponding to the blue colour associated with gentiodelphin.

In summary, all secondary HRT plasmids (tables 12-18) are assembled in vivo, in yeast strains already comprising the full length integrated pathways to pelargonidin, cyanidin, and delphinidin, respectively, as well as the first HRT plasmid (table 11) comprising the PhCHIL (SEQ ID NO: 13), PhGST (SEQ ID NO: 55), and either DcA3GT (SEQ ID NO: 57) for pelargonidin derivatives or FaA3GT (SEQ ID NO: 59) for cyanidin and delphinidin derivatives, as described above. It is noted that, as a result, several strains would have two copies of A3GTs. Resulting strains are grown in liquid cultures for 72 hours with the appropriate selection, and with sugar as the sole carbon source (except for caffeic acid in one experiment). Production of modified anthocyanins is confirmed by LC-MS as described above.

### Example 13—Engineering of Yeast Strain to Produce Dihydrochalcones

A basic yeast strain, Saccharomyces cerevisiae S288C as described in Eichenberger et al. (Met. Eng., 2017, 39:80-89), is used to construct the biosynthetic pathways to phlorizin and nothofagin (FIG. 4), either with or without co-expression of the Petunia x hybrida CHIL gene (SEQ ID NO: 13). The pathways are assembled, in vivo, on two HRT plasmids.

A first plasmid is assembled using the genes listed in table 19, to generate a strain producing phloretin. Hence, the plasmid comprised the pathway structural genes phenylalanine ammonia lyase (PAL) from A. thaliana (SEQ ID NO: 1), the cinnamate-4-hydroxylase (C4H) from Ammi majus (SEQ ID NO: 5), the 4-coumarate-CoA ligase (4CL) from A. thaliana (SEQ ID NO: 7), and the chalcone synthase (CHS) from Hypericum androsaemum (SEQ ID NO: 9). In addition, it comprised an additional copy of the native genes ScCPR1 (SEQ ID NO: 23), for regenerating the activity of C4H, and ScTSC13 (SEQ ID NO: 91), a double bond reductase (DBR) known to increase the amount of dihydrocoumaroyl-CoA needed for phloretin and phlorizin production (see Eichenberger et al., Met. Eng., 2017, 39:80-89).

Helper fragments ZA (SEQ ID NO: 112), AB (SEQ ID NO: 114), and HZ (SEQ ID NO: 119) are used, as described above, for the in vivo assembly of plasmids. After transformation of the yeast, production of phloretin is verified.

A second plasmid is assembled, using the genes listed in table 20, comprising the Pyrus communis UDP-dependent glycosyl transferase (PGT; SEQ ID NO: 93) or the UDP dependent C-glycosyl transferase (CGT) from Oryza sativa (SEQ ID NO: 95) in the CD cassette. The BC cassette is either empty (control) or comprised the gene encoding CHIL from Petunia x hybrida (SEQ ID NO: 13).

Helper fragments ZA (SEQ ID NO: 112), AB (SEQ ID NO: 114), and DZ (SEQ ID NO: 115) are used, as described above, for the in vivo assembly of plasmids.

The resulting strains are grown and analysed as described above. Surprisingly, the strains co-expressing PhCHIL produced more phlorizin and nothofagin, compared to the control strains without PhCHIL.

TABLE 19
Cassettes used to assemble HRT plasmid for phloretin production

| Cassette | Content | SEQ ID NO | Species |
|---|---|---|---|
| ZA | LEU2, pSC101 | 112 | N/A |
| AB | ARSH4/CEN6, | 114 | N/A |
| | CmR | | |
| BC | AtPAL | 1 | Arabidopsis thaliana |
| CD | AmC4H | 5 | Ammi majus |
| DE | At4CL | 7 | Arabidopsis thaliana |
| EF | HaCHS | 9 | Hypericum androsaemum |
| FG | ScTSC13 | 91 | Saccharomyces cerevisiae |
| GH | ScCPR1 | 23 | Saccharomyces cerevisiae |
| HZ | Linker | 119 | N/A |

TABLE 20
Cassettes used to assemble HRT plasmid
for phlorizin and nothofagin production

| Cassette | Content | SEQ ID NO | Species |
|---|---|---|---|
| ZA | HIS3, pSC101 | 113 | N/A |
| AB | ARSH4/CEN6, CmR | 114 | N/A |
| BC | Empty or PhCHIL | 13 | Petunia × hybrida |
| CD | PcPGT or OsCGT | 93 or 95 | Pyrus communis, |
| | | | Oryza sativa |
| DZ | Linker | 115 | N/A |

### Example 14—Engineering of Yeast Strain to Produce Resveratrol

A basic yeast strain Saccharomyces cerevisiae S288C as described in Eichenberger et al. (Met. Eng., 2017, 39:80-89), is used to construct the biosynthetic pathway to resveratrol, either with or without co-expression of the Petunia x hybrida CHIL gene (SEQ ID NO: 13). The pathway is assembled, in vivo, onto an HRT plasmid.

The HRT plasmid is assembled, using the genes listed in table 21. Hence, the plasmid comprised the pathway structural genes phenylalanine ammonia lyase (PAL) from A. thaliana (SEQ ID NO: 1), the cinnamate-4-hydroxylase (C4H) from Ammi majus (SEQ ID NO: 5), the 4-coumarate-CoA ligase (4CL) from A. thaliana (SEQ ID NO: 7), and the stilbene synthase (STS) from Pinus densiflora (SEQ ID NO: 71) or from Vitis vinifera (SEQ ID NO: 73). In addition, it comprised an additional copy of the native genes ScCPR1 (SEQ ID NO: 23), for regenerating the C4H, and either an empty gene cassette (control) or the Petunia x hybrida CHIL gene (SEQ ID NO: 13).

Helper fragments ZA (SEQ ID NO: 112), AB (SEQ ID NO: 114), and HZ (SEQ ID NO: 119) are used, as described above, for the in vivo assembly of plasmids.

After transformation of yeast, the resulting strains are grown and analyzed as described above. The strains co-expressing STS from Pinus densiflora (SEQ ID NO: 71) are shown to produce pinosylvin, and the strains co-expressing STS from Vitis vinifera (SEQ ID NO: 73) are shown to produce resveratrol. Surprisingly, all strains co-expressing PhCHIL produced more stilbenes, compared to the control strain without PhCHIL.

TABLE 21
Cassettes used to assemble HRT plasmid for resveratrol production

| Cassette | Content | SEQ ID NO | Species |
|---|---|---|---|
| ZA | LEU2, pSC101 | 112 | N/A |
| AB | ARSH4/CEN6, | 114 | N/A |
| | CmR | | |
| BC | AtPAL | 1 | Arabidopsis thaliana |
| CD | AmC4H | 5 | Ammi majus |
| DE | At4CL | 7 | Arabidopsis thaliana |
| EF | PdSTS, or | 71 | Pinus densiflora |
| | VvSTS | 73 | Vitis vinifera |
| FG | Empty or PhCHIL | 13 | Petunia × hybrida |
| GH | ScCPR1 | 82 | Saccharomyces cerevisiae |
| HZ | Linker | 119 | N/A |

### Example 15—Methylation to Produce Peonidin, Petunidin, and Malvidin

Several naturally occurring anthocyanins are methylated at one or more hydroxyl group on the B-ring of the basic scaffold, as found for example in the cyanin derivative peonidin, and the delphinidin derivatives petunidin and malvidin. A small number of O-methyl transferases (OMT) have been characterized, which exhibit specificity for anthocyanins, such as the VvAOMT from Vitis vinifera (Hugueney et al., Plant Physiol. 2009, 150 (4): 2057-70), the CkmAOMT2 from a cyclamen hybrid (Akita et al., Planta 2011, 234 (6): 1127-36), and the SIAOMT from Solanum lycopersicum (Roldan et al., Plant J. 2014, 80 (4): 695-708). In order to demonstrate production of methylated anthocyanins in yeast we chose the VvAOMT (SEQ ID NO: 97) and co-expressed this enzyme in strains comprising the full-length pathways to C3G or D3G, respectively. The strains are based on the cyanidin and delphinidin producing strains described above in example 11, and an HRT plasmid is assembled in these two strains according to table 22, below. Hence, this plasmid comprised the PhCHIL (SEQ ID NO: 13), the PhGST (SEQ ID NO: 55), The FaA3GT (SEQ ID NO: 59), and the VvAOMT (SEQ ID NO: 97).

Helper fragments ZA (SEQ ID NO: 112), AB (SEQ ID NO: 114), and FZ (SEQ ID NO: 117) are used, as described above, for the in vivo assembly of plasmids.

TABLE 22
Cassettes used to assemble HRT plasmid peonidin,
petunidin, and malvidin production

| Cassette | Content | SEQ ID NO | Species |
|---|---|---|---|
| ZA | LEU2, pSC101 | 112 | N/A |
| AB | ARSH4/CEN6, CmR | 114 | N/A |
| BC | PhCHIL | 13 | Petunia × hybrida |
| CD | PhGST | 55 | Petunia × hybrida |
| DE | VvAOMT | 97 | Vitis vinifera |
| EF | FaA3GT | 59 | Fragaria × ananassa |
| FZ | Linker | 117 | N/A |

After transformation of yeast, the resulting strains are grown and analyzed as described above. The strain originally producing cyanidin are shown to produce peonidin, the 3′-O-methylated derivative of C3G, and the strain originally producing delphinidin is shown to produce both petunidin, the 3′-O-methylated derivative of D3G, and malvidin, the 3′5′-O-di-methylated derivative of D3G.

1. A recombinant microbial host cell comprising metabolic pathway producing an anthocyanidin, said pathway comprising a glutathione-S-transferase (GST) and an anthocyanidin synthase (ANS) converting a leucoanthocyanidin and/or a flavanol substrate into an anthocyanidin.

2. The host cell of claim 1, wherein the GST modifies conversion of leucoanthocyanidin to anthocyanidin, by increasing the production ratio of anthocyanidin or derivatives thereof and decreasing the production ratio of flavonol or derivatives thereof by a factor selected from at least 1.25 fold, at least 1.5 fold, at least 1.75 fold, at least 2 fold, at least 3 fold, at least 5 fold, at least 10 fold, at least 50 fold, at least 100 fold, at least 500 fold, at least 1000 fold increase in formation of anthocyanidin.

3. The host cell of claim 1, wherein the glutathione-S-transferase (GST) is expressed as fused polypeptides with the anthocyanidin synthase (ANS) or wherein the glutathione-S-transferase (GST) is co-expressed with the anthocyanidin synthase (ANS).

4. The host cell of claim 1, wherein the glutathione-S-transferase (GST) has an amino acid sequence which is at least 80% identity to the glutathione-S-transferase (GST) encoded by a sequence set forth in SEQ ID NO: 55.

5. The host cell of claim 1, wherein the anthocyanidin synthase (ANS) has an amino acid sequence which is at least 80% identity to the anthocyanidin synthase (ANS) encoded by a sequence set forth in SEQ ID NO: 53.

6. The host cell of claim 1, wherein the metabolic pathway further comprises one or more polypeptides selected from:
a. a phenylalanine ammonia lyase (PAL/EC 4.3.1.24);
b. a tyrosine ammonia lyase (TAL/EC 4.3.1.25);
c. a CYP450 reductase (CPR/EC 1.6.2.4);
d. a cinnamate 4-hydroxylase (C4H/EC 1.14.14.91);
e. a Coumarate 3-hydroxylase (C3H/EC 1.14.13);
f. a 4-coumaric acid-CoA ligase (4CL/EC 6.2.1.12);
g. a chalcone synthase (CHS);
h. a chalcone isomerase (CHI);
i. a chalcone isomerase-like (CHIL) polypeptide heterologous;
j. a flavonoid hydroxylase (FH);
k. a flavonoid 3-hydroxylase (F3H);
l. a flavonoid 3′-hydroxylase (F3′H);
m. a flavonoid 3′-5′-hydroxylase (F3′5′H).
n. a dihydroflavonol-4-reductase (DFR); and
o. a leucoanthocyanidin reductase (LAR).

7. The host cell of claim 6, further comprising a UDP-dependent glycosyltransferase (UGT) converting the anthocyanidin into an anthocyanin, said UGT selected from selected from:
a. an anthocyanidin 3-O-glycosyltransferase (A3GT);
b. an anthocyanin 3′-O-glycosyl transferase (A3′GT);
c. an anthocyanin-5-O-glycosyl transferase (A5GT); and
d. an anthocyanin 3′5′-di-O-glycosyl transferase (A3′5′GT).

8. The host cell of claim 7, further comprising a polypeptide further modifying the anthocyanin, said polypeptide selected from:
a. an anthocyanin-O-methyl transferase (AOMT);
b. an anthocyanin acyl transferase (AAT);
c. an anthocyanin aromatic acyl transferase (AAroAT); and
d. an anthocyanin aliphatic acyl transferase (AAliAT).

9. The host cell of claim 6 further comprising an anthocyanidine reductase (ANR), converting the anthocyanidine into epi-afzelechin and/or (−)-epicatechin.

10. The host cell of claim 6, wherein the
a. phenylalanine ammonia lyase (PAL) has at least 80% identity to the phenylalanine ammonia lyase encoded by the sequence set forth in SEQ ID NO: 1;
b. tyrosine ammonia lyase (TAL) has at least 80% identity to the tyrosine ammonia lyase encoded by the sequence set forth in SEQ ID NO: 3;
c. CYP450 reductase (CPR) has at least 80% identity to the CYP450 reductases encoded by a sequence set forth in SEQ ID NO: 23 or SEQ ID NO: 25;
d. trans-cinnamate 4-monooxygenase (C4H) has at least 80% identity to the trans-cinnamate 4-monooxygenase encoded by the sequence set forth in SEQ ID NO: 5;
e. 4-coumaric acid-CoA ligase (4CL) has at least 80% identity to the 4-coumaric acid-CoA ligase encoded by the sequence set forth in SEQ ID NO: 7;
f. chalcone synthase (CHS) has at least 70% identity to the chalcone synthase (CHS) encoded by the sequence set forth in SEQ ID NO: 9;
g. chalcone isomerase (CHI) has at least 70% identity to the chalcone isomerase (CHI) encoded by the sequence set forth in SEQ ID NO: 11;
h. chalcone isomerase-like (CHIL) polypeptide heterologous has at least 70% identity to the chalcone isomerase-like (CHIL) polypeptide encoded by the sequence set forth in SEQ ID NO: 13;
i. flavanone 3-hydroxylase (F3H) has at least 80% identity to the flavanone 3-hydroxylase (F3H) encoded by the sequence set forth in SEQ ID NO: 39;
j. flavonoid 3′-hydroxylase (F3′H) has at least 80% identity to the flavonoid 3′-hydroxylase (F3′H) encoded by a sequence set forth in SEQ ID NO: 27;
k. flavonoid 3′,5′-hydroxylase (F3′5′H) has at least 80% identity to the flavonoid 3′,5′-hydroxylase (F3′5′H) encoded by a sequence set forth in SEQ ID NO: 29;
l. dihydroflavonol-4-reductase (DFR) has at least 80% identity to the dihydroflavonol-4-reductase (DFR) encoded by a sequence set forth in SEQ ID NO: 43, SEQ ID NO: 45, or SEQ ID NO: 47;
m. leucoanthocyanidin reductase (LAR) has at least 80% identity to the leucoanthocyanidin reductase (LAR) encoded by a sequence set forth in SEQ ID NO: 49;
n. anthocyanidin 3-O-glycosyltransferase (A3GT) has at least 80% identity to the anthocyanidin 3-O-glycosyltransferase (A3GT) encoded by a sequence set forth in SEQ ID NO: 57 or SEQ ID NO: 59;
o. anthocyanidin 3′-O-glycosyltransferase (A3′GT) has at least 80% identity to the anthocyanidin 3-O-glycosyltransferase (A3′GT) encoded by a sequence set forth in SEQ ID NO: 87;
p. anthocyanin-5-O-glycosyl transferase (A5GT) has at least 80% identity to the anthocyanin-5-O-glycosyl transferase (A5GT encoded by the sequence set forth in SEQ ID NO: 81;
q. anthocyanin-7-O-glycosyl transferase (A7GT) has at least 80% identity to the anthocyanin-7-O-glycosyl transferase (A7GT) encoded by the sequence set forth in SEQ ID NO: 69;
r. anthocyanin 3′5′-O-di-glycosyl transferase (A3′5′GT) has at least 80% identity to the anthocyanin 3′5′-di-O-glycosyl transferase (A3′5′GT) encoded by the sequence set forth in SEQ ID NO: 85;
s. anthocyanin aromatic acyl transferase (AAroAT) has at least 80% identity to the anthocyanin aromatic acyl transferase (AAroAT) encoded by the sequence set forth in SEQ ID NO: 79;
t. anthocyanin aliphatic acyl transferase (AAliAT) has at least 80% identity to the anthocyanin aliphatic acyl transferase (AAliAT) encoded by the sequence set forth in SEQ ID NO: 77;
u. anthocyanidin O-methyl transferase (AOMT) has at least 80% identity to the anthocyanin O-methyl transferase (AOMT) encoded by the sequence set forth in SEQ ID NO: 97; and/or
v. anthocyanidine reductase (ANR) has at least 80% identity to the ANR encoded by the sequence set forth in SEQ ID NO: 51.

11. The host cell of claim 1, wherein the anthocyanidin is selected from pelargonidin, cyanidin, delphinidin, peonidin, petunidin, and malvidin.

12. The host cell of claim 7, wherein the anthocyanin is selected from one or more of pelargonidin-3-O-glycoside (P3G), cyanidin-3-O-glycoside (C3G) delphinidin-3-O-glycoside (D3G); peonidin-3-O-glycoside, petunidin-3-O-glycoside, malvidin-3-O-glycoside, Petunidin 3-p-coumaroylrutinoside-5-glucoside; petunidin 3-feruloylrutinoside-5-glucoside; malvidin 3-p-coumaroylrutinoside-5-glucoside; pelargonidin 3-feruloylrutinoside-5-glucoside; pelargonidin rutinoside; pelargonidin 3-rutinoside-5-glucoside; pelargonidin 3-p-coumaroylrutinoside-5-glucoside; petunidin 3-rutinoside-5-glucoside; pelargonidin 3-rutinoside-5-glucoside; peonidin 3-rutinoside-5-glucoside; malvidin 3-rutinoside-5-glucoside; petunidin 3-rutinoside; pelargonidin 3-rutinoside; malvidin 3-rutinoside; petunidin 3-caffeoylrutinoside-5-glucoside; delphinidin 3-p-coumaroylrutinoside-5-glucoside; pelargonidin feruloyl-xylosyl-glucosylgalactoside; cyanidin 3-p-coumaroylrutinoside-5-glucoside; petunidin 3-p-coumaroylrutinoside-5-glucoside; petunidin 3-feruloylrutinoside-5-glucoside; pelargonidin 3-p-coumaroylrutinoside-5-glucoside; peonidin 3-p-coumaroylrutinoside-5-glucoside; malvidin 3-p-coumaroylrutinoside-5-glucoside; pelargonidin 3-feruloylrutinoside-5-glucoside; peonidin 3-feruloylrutinoside-5-glucoside; malvidin 3-feruloylrutinoside-5-glucoside; petunidin 3-p-coumaroylrutinoside; pelargonidin 3-p-coumaroylrutinoside; Cyanidin 3-O-glucoside; cyanidin 3;7-O-diglucoside; cyanidin 3-O-(3″;6″-O-dimalonyl)-glucoside; cyanidin 3-O-6″-O-malonylglucoside; delphinidin 3-O-glucoside; delphinidin 3;5;3′-O-triglucoside; delphinidin 3-O-(3″;6″-O-dimalonyl)-glucoside; delphinidin 3-O-6″-O-malonylglucoside; delphinidin 3-O-rutinoside; delphinidin 3-O-rutinoside 7-O-glucoside; delphinidin 3-O-rutinoside 7-O-(6″-O-p-hydroxybenzoyl)-glucoside; pelargonidin 3-O-glucoside; Cyanidin 3-(6″;6′″-di-p-coumarylsophoroside)-5-(6-malonylglucoside); Cyanidin 3-(6″;6′-dicaffeylsophoroside)-5-glucoside; Cyanidin 3-(6″;6″-disinapylsophoroside)-5-glucoside; Cyanidin 3-(6″-caffeyl-6′″-ferulylsophoroside)-5-glucoside; Cyanidin 3-(6″-ferulyl-2″-sinapylsambubioside)-5-glucoside; Cyanidin 3-(6″-ferulyl-2″-sinapylsophoroside)-5-glucoside; Cyanidin 3-(6″-p-coumaryl-2″-sinapylsophoroside)-5-glucoside; Cyanidin 3-(6-malonylglucoside)-7;3′-di-(6-feruloylglucoside); Cyanidin 3-(6-malonylglucoside)-7-(6-feruloylglucoside)-3′-glucoside; Cyanidin 3-[2-(6-p-coumarylglucosyl)-6-caffeoylglucoside]-5-glucoside; Cyanidin 3-[2-(6-p-coumarylglucosyl)-6-p-coumarylglucoside)]-5-glucoside; Cyanidin 3-[6″-(4-glucosylcaffeyl isophoroside]-5-glucoside; Cyanidin 3-O-[2″-O-(2′″-O-(sinapoyl) xylosyl) 6″-O-(p-coumaroyl) glucoside] 5-O-glucoside; Cyanidin 3-O-[beta-D-glucopyranoside]-7;3′-di-O-[6-O-(sinapyl)-beta-D-glucopyranoside]; Delphinidin 3-(6″;6″-di-p-coumarylsophoroside)-5-(6-malonylglucoside); Delphinidin 3-(6-malonylglucoside)-3′;5′-di-(6-p-coumaroylglucoside); Delphinidin 3-(diferuloyl) sophoroside-5-glucoside; Delphinidin 3-[2-(6-(feruloylglucoside)-6-feruloylglucoside]-5-(6-malonylglucoside); Delphinidin 3-[2-(6-feruloylglucoside)-6-p-coumaroylglucoside]-5-(6-malonylglucoside); Delphinidin 3-glucoside-5-(6-caffeoylglucoside)-3′-(6-(E)-p-coumaroylglucoside); Delphinidin 3-glucoside-7;3′-di-(6-(E)-sinapoylglucoside); Delphinidin 3-rutinoside-7-(6-p-coumaroylglucoside)-3′-glucoside; Cyanidin 3-glucoside-5;3′-di-(caffeoylglucoside); Malvidin 3-O-[6-O-(4-O-(4-O-(6-O-(trans-caffeoyl)-beta-D-glucopyranosyl)-trans-p-coumaroyl)-alpha-L-rhamnopyranosyl)-beta-D- glucopyranoside]; Pelargonidin 3-(6″;2″-diferulylsambubioside)-5-(6-malonylglucoside); Pelargonidin 3-(6″-ferulyl-2′-sinapylsambubioside)-5-(6-malonylglucoside); Pelargonidin 3-(6″-ferulyl-2′″-sinapylsambubioside)-5-glucoside; Pelargonidin 3-(6″-p-coumaryl-2′″-sinapyl-sambubioside)-5-(6-malonylglucoside); Pelargonidin 3-(6″-p-coumaryl-2′-sinapylsambubioside)-5-glucoside; Pelargonidin 3-[6″-(3-glucosylcaffeyl) sophoroside]-5-glucoside; Pelargonidin 3-O-(6-O-malonyl-beta-D-glucopyranoside)-7-O-(6-O-(4-O-(trans-caffeyl)-beta-D-glucopyranosyl)-trans-caffeyl)-beta-D-glucopyranoside); Pelargonidin 3-O-[2-O-(2-(E)-feruloyl-beta-D-glucopyranosyl)-6-O-(E)-feruloyl-beta-D-glucopyranoside]-5-O-(beta-D-glucopyranoside); Pelargonidin 3-(2-(2-ferulylglucosyl)-6-ferulylglucoside)-5-glucoside; Pelargonidin 3-O-[2-O-(6-(E)-caffeoyl-beta-D-glucopyranosyl)-6-O-(E)-caffeoyl-beta-D-glucopyranoside]-5-O-(beta-D-glucopyranoside); Pelargonidin 3-(2-(6-caffeylglucosyl)-6-caffeylglucoside)-5-glucoside; Pelargonidin 3-O-[2-O-(6-(E)-caffeoyl-beta-D-glucopyranosyl)-6-O-(E)-feruloyl-beta-D-glucopyranoside]-5-O-(beta-D-glucopyranoside); Pelargonidin 3-(2-(6-caffeylglucosyl)-6-ferulylglucoside)-5-glucoside; Pelargonidin 3-O-[2-O-(6-(E)-caffeoyl-beta-D-glucopyranosyl)-6-O-(E)-p-coumaroyl-beta-D-glucopyranoside]-5-O-(beta-D-glucopyranoside); Pelargonidin 3-(2-(6-caffeylglucosyl)-6-p-coumarylglucoside)-5-glucoside; Pelargonidin 3-O-[2-O-(6-(E)-feruloyl-beta-D-glucopyranosyl)-6-O-(E)-caffeoyl-beta-D-glucopyranoside]-5-O-(beta-D-glucopyranoside); Pelargonidin 3-(2-(6-ferulylglucosyl)-6-caffeylglucoside)-5-glucoside; Pelargonidin 3-O-[2-O-(6-(E)-feruloyl-beta-D-glucopyranosyl)-6-O-(E)-feruloyl-beta-D-glucopyranoside]-5-O-(beta-D-glucopyranoside); Pelargonidin 3-(2-(6-ferulylglucosyl)-6-ferulylglucoside)-5-glucoside; Pelargonidin 3-O-[2-O-(6-(E)-feruloyl-beta-D-glucopyranosyl)-6-O-(E)-p-coumaroyl-beta-D-glucopyranoside]-5-O-(beta-D-glucopyranoside); Pelargonidin 3-(2-(6-ferulylglucosyl)-6-p-coumarylglucoside)-5-glucoside; Peonidin 3-[6″-(4-glucosylcaffeyl) sophoroside]-5-glucoside; and Petunidin 3-O-[6-O-(4-O-(4-O-(beta-D-glucopyranosyl)-trans-p-coumaroyl)-alpha-L-rhamnopyranosyl)-beta-D-glucopyranoside]-5-O-[beta-D-glucopyranoside].

13. The host cell of claim 1, wherein the host cell is a yeast.

14. The host cell of claim 13, wherein the yeast belongs to the genus Saccharomyces, Klyuveromyces, Candida, Pichia, Debaromyces, Hansenula, Yarrowia, Zygosaccharomyces, or Schizosaccharomyces.

15. A recombinant polynucleotide construct comprising a nucleotide sequence encoding the glutathione-S-transferase (GST) and/or the anthocyanidin synthase (ANS) of claim 1, operably linked to one or more control sequences which are heterologous to the GST and/or ANS encoding polynucleotide sequences.

16. The polynucleotide construct of claim 15, wherein the control sequence is a promoter that is recognized by the host cell for expression of the GST or ANS.

17. The polynucleotide construct of claim 16 wherein the promoter has at least 70% identity to a sequence selected from the groups consisting of SEQ ID NO: 101, SEQ ID NO: 102; SEQ ID NO: 103; SEQ ID NO: 104; SEQ ID NO: 105; SEQ ID NO: 106; SEQ ID NO: 107 and SEQ ID NO: 108.

18. An expression vector comprising the polynucleotide construct of claim 15.

19. A cell culture, comprising the host cell of claim 1 and a growth medium.

20. A method for producing an anthocyanidin or an anthocyanin comprising
a. culturing the cell culture of claim 18 at conditions allowing the host cell to convert the one or more substrates into the anthocyanidin or the anthocyanin; and
b. optionally recovering and/or isolating the anthocyanidin or the anthocyanin.

21. A composition comprising the cell culture of claim 18 and its contents of anthocyanidin or anthocyanin, wherein at least 95% of solid cellular material has been removed, said composition comprising one or more agents, additives and/or excipients.

22. Use of the composition of claim 20 in the manufacture of a food product, a dietary supplement, a pharmaceutical product, or a cosmetic product.
